SEQUENCE LISTING (370 sequences)

SEQ ID NO: 1 - RNA, 1722 nt, synthetic construct
  source 1..1722: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaac

SEQ ID NO: 2 - RNA, 1722 nt, synthetic construct; identical to SEQ ID NO: 1
  source 1..1722: mol_type = other RNA

SEQ ID NO: 3 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacccgccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 4 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgccgtgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 5 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagccgccgagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 6 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 7 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 8 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacccgccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 9 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgccgtgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 10 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagccgccgagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 11 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacccgccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 12 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgccgtgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 13 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagccgccgagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 14 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 15 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 16 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 17 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 18 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 19 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 20 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 21 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 22 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 23 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 24 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 25 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 26 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 27 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 28 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 29 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 30 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 31 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 32 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 33 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 34 - RNA, 84 nt, synthetic construct
  source 1..84: mol_type = other RNA
accgccgagaccgcgtccgccccgcgagcacagagcctcgcctttgccgatccgccgcccgtccacacccgccgccagctcacc

SEQ ID NO: 35 - RNA, 235 nt, synthetic construct
  source 1..235: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaattgccatgtgtatgtgggttcgcccacatactctgatgatccccaatcgtggcgtgtcggcctgcttcggcaggcactggcgccgggatcattcatggcaa

SEQ ID NO: 36 - RNA, 108 nt, synthetic construct
  source 1..108: mol_type = other RNA
aaaaaaaaaaaaaaaaaaaaaaaaaaaaaaaaaaaaaaaactggattctgaaaaaaaaaaaaaaaaaaaaaaaaaaaaaaaaaaaaaaaaaaaaaaaaaaaaaaaaaa

SEQ ID NO: 37 - RNA, 16 nt, synthetic construct
  source 1..16: mol_type = other RNA
ggctagcgccgccacc

SEQ ID NO: 38 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 39 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNPPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 40 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPPCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 41 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQPPSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 42 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 43 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 44 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNPPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 45 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPPCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 46 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQPPSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 47 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNPPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 48 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPPCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 49 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQPPSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 50 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 51 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 52 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 53 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 54 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 55 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 56 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 57 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 58 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 59 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPIVNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 60 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 61 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 62 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPIVNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 63 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 64 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 65 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 66 - protein, 574 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 67 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 68 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 69 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 70 - RNA, 50 nt, synthetic construct
  source 1..50: mol_type = other RNA
acatttgcttctgacacaactgtgttcactagcaacctcaaacagacacc

SEQ ID NO: 71 - RNA, 47 nt, synthetic construct
  source 1..47: mol_type = other RNA
gggaaataagagagaaaagaagagtaagaagaaatataagagccacc

SEQ ID NO: 72 - RNA, 111 nt, synthetic construct
  source 1..111: mol_type = other RNA
gctggagcctcggtggccatgcttcttgccccttgggcctccccccagcccctcctccccttcctgcacccgtacccccgtggtctttgaataaagtctgagtgggcggca

SEQ ID NO: 73 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 74 - RNA, 264 nt, synthetic construct
  source 1..264: mol_type = other RNA
gctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgc

SEQ ID NO: 75 - RNA, 60 nt, synthetic construct
  source 1..60: mol_type = other RNA
aaaaaaaaaaaaaaaaaaaaaaaaaaaaaaaaaaaaaaaaaaaaaaaaaaaaaaaaaaaa

SEQ ID NO: 76 - RNA, 80 nt, synthetic construct
  source 1..80: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 77 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 78 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 79 - RNA, 110 nt, synthetic construct
  source 1..110: mol_type = other RNA
aaaaaaaaaaaaaaaaaaaaaaaaaaaaaaaaaaaaaaaactggattctgaaaaaaaaaaaaaaaaaaaaaaaaaaaaaaaaaaaaaaaaaaaaaaaaaaaaaaaaaaaa

SEQ ID NO: 80 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaacaaagcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 81 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNKARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 82 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccaggaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 83 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARQELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 84 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacagaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 85 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKQKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 86 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgccagcgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 87 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRQRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 88 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacagcgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 89 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 90 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccagtttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 91 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRQFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 92 - protein, 1728 aa, synthetic construct
ATGGAACTGCTGATTCTGAAAGCGAACGCGATTACCACCATTCTGACCGCGGTGACCTTTTGCTTTGCGAGCGGCCAGAACATTACCGAAGAATTTTATCAGAGCACCTGCAGCGCGGTGAGCAAAGGCTATCTGAGCGCGCTGCGCACCGGCTGGTATACCAGCGTGATTACCATTGAACTGAGCAACATTAAAGAAAACAAATGCAACGGCACCGATGCGAAAGTGAAACTGATTAAACAGGAACTGGATAAATATAAAAACGCGGTGACCGAACTGCAGCTGCTGATGCAGAGCACCCCGCCGACCAACAACCGCGCGCGCCGCGAACTGCCGCGCTTTATGAACTATACCCTGAACAACGCGAAAAAAACCAACGTGACCCTGAGCAAAAAACGCAAACGCCGCTTTCTGGGCTTTCTGCAGGGCGTGGGCAGCGCGATTGCGAGCGGCGTGGCGGTGAGCAAAGTGCTGCATCTGGAAGGCGAAGTGAACAAAATTAAAAGCGCGCTGCTGAGCACCAACAAAGCGGTGGTGAGCCTGAGCAACGGCGTGAGCGTGCTGACCAGCAAAGTGCTGGATCTGAAAAACTATATTGATAAACAGCTGCTGCCGATTGTGAACAAACAGAGCTGCAGCATTAGCAACATTGAAACCGTGATTGAATTTCAGCAGAAAAACAACCGCCTGCTGGAAATTACCCGCGAATTTAGCGTGAACGCGGGCGTGACCACCCCGGTGAGCACCTATATGCTGACCAACAGCGAACTGCTGAGCCTGATTAACGATATGCCGATTACCAACGATCAGAAAAAACTGATGAGCAACAACGTGCAGATTGTGCGCCAGCAGAGCTATAGCATTATGAGCATTATTAAAGAAGAAGTGCTGGCGTATGTGGTGCAGCTGCCGCTGTATGGCGTGATTGATACCCCGTGCTGGAAACTGCATACCAGCCCGCTGTGCACCACCAACACCAAAGAAGGCAGCAACATTTGCCTGACCCGCACCGATCGCGGCTGGTATTGCGATAACGCGGGCAGCGTGAGCTTTTTTCCGCAGGCGGAAACCTGCAAAGTGCAGAGCAACCGCGTGTTTTGCGATACCATGAACAGCCTGACCCTGCCGAGCGAAATTAACCTGTGCAACGTGGATATTTTTAACCCGAAATATGATTGCAAAATTATGACCAGCAAAACCGATGTGAGCAGCAGCGTGATTACCAGCCTGGGCGCGATTGTGAGCTGCTATGGCAAAACCAAATGCACCGCGAGCAACAAAAACCGCGGCATTATTAAAACCTTTAGCAACGGCTGCGATTATGTGAGCAACAAAGGCATGGATACCGTGAGCGTGGGCAACACCCTGTATTATGTGAACAAACAGGAAGGCAAAAGCCTGTATGTGAAAGGCGAACCGATTATTAACTTTTATGATCCGCTGGTGTTTCCGAGCGATGAATTTGATGCGAGCATTAGCCAGGTGAACGAAAAAATTAACCAGAGCCTGGCGTTTATTCGCAAAAGCGATGAACTGCTGCATAACGTGAACGCGGGCAAAAGCACCACCAACATTATGATTACCACCATTATTATTGTGATTATTGTGATTCTGCTGAGCCTGATTGCGGTGGGCCTGCTGCTGTATTGCAAAGCGCGCAGCACCCCGGTGACCCTGAGCAAAGATCAGCTGAGCGGCATTAACAACATTGCGTTTAGCAACTGATAA

SEQ ID NO: 93 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLQGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 94 - protein, 1728 aa, synthetic construct
ATGGAACTGCTGATTCTGAAAGCGAACGCGATTACCACCATTCTGACCGCGGTGACCTTTTGCTTTGCGAGCGGCCAGAACATTACCGAAGAATTTTATCAGAGCACCTGCAGCGCGGTGAGCAAAGGCTATCTGAGCGCGCTGCGCACCGGCTGGTATACCAGCGTGATTACCATTGAACTGAGCAACATTAAAGAAAACAAATGCAACGGCACCGATGCGAAAGTGAAACTGATTAAACAGGAACTGGATAAATATAAAAACGCGGTGACCGAACTGCAGCTGCTGATGCAGAGCACCCCGCCGACCAACAACCGCGCGCGCCGCGAACTGCCGCGCTTTATGAACTATACCCTGAACAACGCGAAAAAAACCAACGTGACCCTGAGCAAAAAACGCAAACGCCGCTTTCTGGGCTTTCTGCTGGGCGTGGGCAGCGCGATTTGCAGCGGCGTGGCGGTGAGCAAAGTGCTGCATCTGGAAGGCGAAGTGAACAAAATTAAAAGCGCGCTGCTGAGCACCAACAAAGCGGTGGTGAGCCTGAGCAACGGCGTGAGCGTGCTGACCAGCAAAGTGCTGGATCTGAAAAACTATATTGATAAACAGCTGCTGCCGATTGTGAACAAACAGAGCTGCAGCATTAGCAACATTGAAACCGTGATTGAATTTCAGCAGAAAAACAACCGCCTGCTGGAAATTACCCGCGAATTTAGCGTGAACGCGGGCGTGACCACCCCGGTGAGCACCTATATGCTGACCAACAGCGAACTGCTGAGCCTGATTAACGATATGCCGATTACCAACGATCAGAAAAAACTGATGAGCAACAACGTGCAGATTGTGCGCCAGCAGAGCTATAGCATTATGAGCATTATTAAAGAAGAAGTGCTGGCGTATGTGGTGCAGCTGCCGCTGTATGGCGTGATTGATACCCCGTGCTGGAAACTGCATACCAGCCCGCTGTGCACCACCAACACCAAAGAAGGCAGCAACATTTGCCTGACCCGCACCGATCGCGGCTGGTATTGCGATAACGCGGGCAGCGTGAGCTTTTTTCCGCAGGCGGAAACCTGCAAAGTGCAGAGCAACCGCGTGTTTTGCGATACCATGAACAGCCTGACCCTGCCGAGCGAAATTAACCTGTGCAACGTGGATATTTTTAACCCGAAATATGATTGCAAAATTATGACCAGCAAAACCGATGTGAGCAGCAGCGTGATTACCAGCCTGGGCGCGATTGTGAGCTGCTATGGCAAAACCAAATGCACCGCGAGCAACAAAAACCGCGGCATTATTAAAACCTTTAGCAACGGCTGCGATTATGTGAGCAACAAAGGCATGGATACCGTGAGCGTGGGCAACACCCTGTATTATGTGAACAAACAGGAAGGCAAAAGCCTGTATGTGAAAGGCGAACCGATTATTAACTTTTATGATCCGCTGGTGTTTCCGAGCGATGAATTTGATGCGAGCATTAGCCAGGTGAACGAAAAAATTAACCAGAGCCTGGCGTTTATTCGCAAAAGCGATGAACTGCTGCATAACGTGAACGCGGGCAAAAGCACCACCAACATTATGATTACCACCATTATTATTGTGATTATTGTGATTCTGCTGAGCCTGATTGCGGTGGGCCTGCTGCTGTATTGCAAAGCGCGCAGCACCCCGGTGACCCTGAGCAAAGATCAGCTGAGCGGCATTAACAACATTGCGTTTAGCAACTGATAA

SEQ ID NO: 95 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAICSGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 96 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 97 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 98 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 99 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 100 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 101 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 102 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattgcgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 103 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 104 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaacggcagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 105 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINGSLAFIRKSDELLHNVNAGKSTTNIMITTIIIVIIVILLSLIAVGLLLYCKARSTPVTLSKDQLSGINNIAFSN

SEQ ID NO: 106 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaaaaactgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 107 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDEKLHNVNAGKSTTNIMITTIIIVIIVILLSLIAVGLLLYCKARSTPVTLSKDQLSGINNIAFSN

SEQ ID NO: 108 - RNA, 1728 nt, synthetic construct
  source 1..1728: mol_type = other RNA
atggatctgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 109 - protein, 574 aa, synthetic construct
MDLLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 110 - RNA, 1728 nt, synthetic construct
  source 1..1728: mol_type = other RNA
atggaactgccgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 111 - protein, 574 aa, synthetic construct
MELPILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 112 - RNA, 1728 nt, synthetic construct
  source 1..1728: mol_type = other RNA
atggaactgctgattctgaaaaccaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 113 - protein, 574 aa, synthetic construct
MELLILKTNAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 114 - RNA, 1728 nt, synthetic construct
  source 1..1728: mol_type = other RNA
atggaactgctgattctgaaagcgaacgcgattaccgcgattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 115 - protein, 574 aa, synthetic construct
MELLILKANAITAILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 116 - RNA, 1728 nt, synthetic construct
  source 1..1728: mol_type = other RNA
atggaactgctgattctgaaagcgaacgcgattaccaccatttttaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 117 - protein, 574 aa, synthetic construct
MELLILKANAITTIFTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 118 - RNA, 1728 nt, synthetic construct
  source 1..1728: mol_type = other RNA
atggaactgctgattctgaaagcgaacgcgattaccaccattctggcggcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 119 - protein, 574 aa, synthetic construct
MELLILKANAITTILAAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 120 - RNA, 1728 nt, synthetic construct
  source 1..1728: mol_type = other RNA
atggaactgctgattctgaaagcgaacgcgattaccaccattctgaccgcggtgaccctgtgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 121 - protein, 574 aa, synthetic construct
MELLILKANAITTILTAVTLCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 122 - RNA, 1728 nt, synthetic construct
  source 1..1728: mol_type = other RNA
atggaactgctgattctgaaagcgaacgcgattaccaccattctgaccgcggtgaccttttgctttgcgagcagccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 123 - protein, 574 aa, synthetic construct
MELLILKANAITTILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 124 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgtggaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 125 - protein, 574 aa, synthetic construct
MELLILKANAITTILTAVTFCFASGQNITVEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 126 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtatcatagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 127 - protein, 574 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 128 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaagcgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 129 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKSAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 130 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcggcgaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 131 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 132 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacaccaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 133 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNTKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 134 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaacaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 135 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKNTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 136 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaacaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 137 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKNNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 138 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgcaaatttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 139 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRKFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 140 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcattgcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 141 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 142 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccattaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 143 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 144 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaagcaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 145 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKSNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 146 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaaattctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 147 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 148 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgctggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 149 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 150 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagccgcaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 151 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 152 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcagcgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 153 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 154 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgctgctgattgcggtgggcctgctgctgtattgcaaagcgcgcagcaccccggtgaccctgagcaaagatcagctgagcggcattaacaacattgcgtttagcaactgataa

SEQ ID NO: 155 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLLLIAVGLLLYCKARSTPVTLSKDQLSGINNIAFSN

SEQ ID NO: 156 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgtttctgtattgcaaagcgcgcagcaccccggtgaccctgagcaaagatcagctgagcggcattaacaacattgcgtttagcaactgataa

SEQ ID NO: 157 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLFLYCKARSTPVTLSKDQLSGINNIAFSN

SEQ ID NO: 158 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 159 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 160 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgatttgcagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattgcgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 161 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAICSGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 162 - RNA, 1536 nt, synthetic construct
  source 1..1536: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccggcagcggcagcgcgatttgcagcggcgtggcggtgtgcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagccgcaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattgcgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgataa

SEQ ID NO: 163 - protein, 510 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLY

SEQ ID NO: 164 - RNA, 1584 nt, synthetic construct
  source 1..1584: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 165 - protein, 526 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRGSFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 166 - RNA, 1581 nt, synthetic construct
  source 1..1581: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgctgataa

SEQ ID NO: 167 - protein, 525 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRGSFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYC

SEQ ID NO: 168 - RNA, 1605 nt, synthetic construct
  source 1..1605: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 169 - protein, 533 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 170 - RNA, 1524 nt, synthetic construct
  source 1..1524: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaactgataa

SEQ ID NO: 171 - protein, 506 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTN

SEQ ID NO: 172 - RNA, 1674 nt, synthetic construct
  source 1..1674: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 173 - protein, 556 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 174 - RNA, 1530 nt, synthetic construct
  source 1..1530: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 175 - protein, 508 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRGSSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 176 - RNA, 1527 nt, synthetic construct
  source 1..1527: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgctgataa

SEQ ID NO: 177 - protein, 507 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRGSSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYC

SEQ ID NO: 178 - RNA, 1599 nt, synthetic construct
  source 1..1599: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 179 - protein, 531 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRGSSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 180 - RNA, 1728 nt, synthetic construct
  source 1..1728: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 181 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 182 - RNA, 1659 nt, synthetic construct
  source 1..1659: mol_type = other RNA
atggaactgctgattctgaaagcgaacgcgattaccaccattctgaccgcggtgaccttttgctttgcgagcggccagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 183 - protein, 551 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 184 - RNA, 75 nt, synthetic construct
  source 1..75: mol_type = other RNA
atggaactgctgattctgaaagcgaacgcgattaccaccattctgaccgcggtgaccttttgctttgcgagcggc

SEQ ID NO: 185 - protein, 25 aa, synthetic construct
MELLILKANAITTILTAVTFCFASG

SEQ ID NO: 186 - RNA, 75 nt, synthetic construct
  source 1..75: mol_type = other RNA
atggatctgctgattctgaaagcgaacgcgattaccaccattctgaccgcggtgaccttttgctttgcgagcggc

SEQ ID NO: 187 - protein, 25 aa, synthetic construct
MDLLILKANAITTILTAVTFCFASG

SEQ ID NO: 188 - RNA, 75 nt, synthetic construct
  source 1..75: mol_type = other RNA
atggaactgccgattctgaaagcgaacgcgattaccaccattctgaccgcggtgaccttttgctttgcgagcggc

SEQ ID NO: 189 - protein, 25 aa, synthetic construct
MELPILKANAITTILTAVTFCFASG

SEQ ID NO: 190 - RNA, 75 nt, synthetic construct
  source 1..75: mol_type = other RNA
atggaactgctgattctgaaaaccaacgcgattaccaccattctgaccgcggtgaccttttgctttgcgagcggc

SEQ ID NO: 191 - protein, 25 aa, synthetic construct
MELLILKTNAITTILTAVTFCFASG

SEQ ID NO: 192 - RNA, 75 nt, synthetic construct
  source 1..75: mol_type = other RNA
atggaactgctgattctgaaagcgaacgcgattaccgcgattctgaccgcggtgaccttttgctttgcgagcggc

SEQ ID NO: 193 - protein, 25 aa, synthetic construct
MELLILKANAITAILTAVTFCFASG

SEQ ID NO: 194 - RNA, 75 nt, synthetic construct
  source 1..75: mol_type = other RNA
atggaactgctgattctgaaagcgaacgcgattaccaccatttttaccgcggtgaccttttgctttgcgagcggc

SEQ ID NO: 195 - protein, 25 aa, synthetic construct
MELLILKANAITTIFTAVTFCFASG

SEQ ID NO: 196 - RNA, 75 nt, synthetic construct
  source 1..75: mol_type = other RNA
atggaactgctgattctgaaagcgaacgcgattaccaccattctggcggcggtgaccttttgctttgcgagcggc

SEQ ID NO: 197 - protein, 25 aa, synthetic construct
MELLILKANAITTILAAVTFCFASG

SEQ ID NO: 198 - RNA, 75 nt, synthetic construct
  source 1..75: mol_type = other RNA
atggaactgctgattctgaaagcgaacgcgattaccaccattctgaccgcggtgaccctgtgctttgcgagcggc

SEQ ID NO: 199 - protein, 25 aa, synthetic construct
MELLILKANAITTILTAVTLCFASG

SEQ ID NO: 200 - RNA, 75 nt, synthetic construct
  source 1..75: mol_type = other RNA
atggaactgctgattctgaaagcgaacgcgattaccaccattctgaccgcggtgaccttttgctttgcgagcagc

SEQ ID NO: 201 - protein, 25 aa, synthetic construct
MELLILKANAITTILTAVTFCFASS

SEQ ID NO: 202 - RNA, 1647 nt, synthetic construct
  source 1..1647: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaac

SEQ ID NO: 203 - RNA, 1647 nt, synthetic construct; identical to SEQ ID NO: 202
  source 1..1647: mol_type = other RNA

SEQ ID NO: 204 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacccgccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 205 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgccgtgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 206 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagccgccgagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 207 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 208 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 209 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacccgccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 210 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgccgtgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 211 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagccgccgagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccgccgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 212 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacccgccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 213 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgccgtgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 214 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagccgccgagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgccggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 215 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 216 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 217 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 218 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 219 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 220 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 221 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 222 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaaccgagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 223 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 224 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 225 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 226 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 227 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 228 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 229 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 230 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaatgcagctgctgcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 231 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatacctgcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgtgcaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 232 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgtgcaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgtgcgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 233 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 234 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 235 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaacaaagcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 236 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccaggaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 237 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacagaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 238 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgccagcgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 239 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacagcgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 240 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccagtttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 241 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgcagggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 242 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgatttgcagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 243 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 244 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 245 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 246 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattgcgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 247 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaacggcagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 248 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaaaaactgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 249 - RNA, 1653 nt, synthetic construct; identical to SEQ ID NO: 233
  source 1..1653: mol_type = other RNA

SEQ ID NO: 250 - RNA, 1653 nt, synthetic construct; identical to SEQ ID NO: 233
  source 1..1653: mol_type = other RNA

SEQ ID NO: 251 - RNA, 1653 nt, synthetic construct; identical to SEQ ID NO: 233
  source 1..1653: mol_type = other RNA

SEQ ID NO: 252 - RNA, 1653 nt, synthetic construct; identical to SEQ ID NO: 233
  source 1..1653: mol_type = other RNA

SEQ ID NO: 253 - RNA, 1653 nt, synthetic construct; identical to SEQ ID NO: 233
  source 1..1653: mol_type = other RNA

SEQ ID NO: 254 - RNA, 1653 nt, synthetic construct; identical to SEQ ID NO: 233
  source 1..1653: mol_type = other RNA

SEQ ID NO: 255 - RNA, 1653 nt, synthetic construct; identical to SEQ ID NO: 233
  source 1..1653: mol_type = other RNA

SEQ ID NO: 256 - RNA, 1653 nt, synthetic construct; identical to SEQ ID NO: 233
  source 1..1653: mol_type = other RNA

SEQ ID NO: 257 - RNA, 1653 nt, synthetic construct
  source 1..1653: mol_type = other RNA
cagaacattaccgtggaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 258 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtatcatagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 259 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaagcgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 260 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcggcgaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 261 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacaccaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 262 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaacaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 263 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaacaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 264 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgcaaatttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 265 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcattgcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 266 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccattaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 267 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaagcaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 268 - RNA, 1654 nt, synthetic construct
  source 1..1654: mol_type = other RNA
acagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaaattctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 269 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgctggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 270 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagccgcaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 271 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcagcgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 272 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgctgctgattgcggtgggcctgctgctgtattgcaaagcgcgcagcaccccggtgaccctgagcaaagatcagctgagcggcattaacaacattgcgtttagcaactgataa

SEQ ID NO: 273 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgtttctgtattgcaaagcgcgcagcaccccggtgaccctgagcaaagatcagctgagcggcattaacaacattgcgtttagcaactgataa

SEQ ID NO: 274 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 275 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgatttgcagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattgcgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 276 - RNA, 1461 nt, synthetic construct
  source 1..1461: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccggcgaccggcagcggcagcgcgatttgcagcggcgtggcggtgtgcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagccgcaccctgccgagcgaagtgaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcgtggataccgtgagcgtgggcaacaccctgtattgcgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgataa

SEQ ID NO: 277 - RNA, 1509 nt, synthetic construct
  source 1..1509: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 278 - RNA, 1506 nt, synthetic construct
  source 1..1506: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagctttctgggctttctgctgggcgtgggcagcgcgattgcgagcggcgtggcggtgagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgctgataa

SEQ ID NO: 279 - RNA, 1530 nt, synthetic construct
  source 1..1530: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 280 - RNA, 1449 nt, synthetic construct
  source 1..1449: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaactgataa

SEQ ID NO: 281 - RNA, 1599 nt, synthetic construct
  source 1..1599: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 282 - RNA, 1455 nt, synthetic construct
  source 1..1455: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 283 - RNA, 1452 nt, synthetic construct
  source 1..1452: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgctgataa

SEQ ID NO: 284 - RNA, 1524 nt, synthetic construct
  source 1..1524: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcggcagcagcaaagtgctgcatctggaaggcgaagtgaacaaaattaaaagcgcgctgctgagcaccaacaaagcggtggtgagcctgagcaacggcgtgagcgtgctgaccagcaaagtgctggatctgaaaaactatattgataaacagctgctgccgattgtgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgagcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 285 - RNA, 1653 nt, synthetic construct
  source 1..1653: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacattatgattaccaccattattattgtgattattgtgattctgctgagcctgattgcggtgggcctgctgctgtattgcaaagcgcgcagcaccccggtgaccctgagcaaagatcagctgagcggcattaacaacattgcgtttagcaactgataa

SEQ ID NO: 286 - RNA, 1584 nt, synthetic construct
  source 1..1584: mol_type = other RNA
cagaacattaccgaagaattttatcagagcacctgcagcgcggtgagcaaaggctatctgagcgcgctgcgcaccggctggtataccagcgtgattaccattgaactgagcaacattaaagaaaacaaatgcaacggcaccgatgcgaaagtgaaactgattaaacaggaactggataaatataaaaacgcggtgaccgaactgcagctgctgatgcagagcaccccgccgaccaacaaccgcgcgcgccgcgaactgccgcgctttatgaactataccctgaacaacgcgaaaaaaaccaacgtgaccctgagcaaaaaacgcaaacgccgctttctgggctttctgctgggcgtgggcagcgcgattgcgagcggcgtggcggtgtgcaaagtgctgcatctggaaggcgaagtgaacaaaattaaaagcgcgctgctgagcaccaacaaagcggtggtgagcctgagcaacggcgtgagcgtgctgacctttaaagtgctggatctgaaaaactatattgataaacagctgctgccgattctgaacaaacagagctgcagcattagcaacattgaaaccgtgattgaatttcagcagaaaaacaaccgcctgctggaaattacccgcgaatttagcgtgaacgcgggcgtgaccaccccggtgagcacctatatgctgaccaacagcgaactgctgagcctgattaacgatatgccgattaccaacgatcagaaaaaactgatgagcaacaacgtgcagattgtgcgccagcagagctatagcattatgtgcattattaaagaagaagtgctggcgtatgtggtgcagctgccgctgtatggcgtgattgataccccgtgctggaaactgcataccagcccgctgtgcaccaccaacaccaaagaaggcagcaacatttgcctgacccgcaccgatcgcggctggtattgcgataacgcgggcagcgtgagcttttttccgcaggcggaaacctgcaaagtgcagagcaaccgcgtgttttgcgataccatgaacagcctgaccctgccgagcgaaattaacctgtgcaacgtggatatttttaacccgaaatatgattgcaaaattatgaccagcaaaaccgatgtgagcagcagcgtgattaccagcctgggcgcgattgtgagctgctatggcaaaaccaaatgcaccgcgagcaacaaaaaccgcggcattattaaaacctttagcaacggctgcgattatgtgagcaacaaaggcatggataccgtgagcgtgggcaacaccctgtattatgtgaacaaacaggaaggcaaaagcctgtatgtgaaaggcgaaccgattattaacttttatgatccgctggtgtttccgagcgatgaatttgatgcgagcattagccaggtgaacgaaaaaattaaccagagcctggcgtttattcgcaaaagcgatgaactgctgcataacgtgaacgcgggcaaaagcaccaccaacggctatattccggaagcgccgcgcgatggccaggcgtatgtgcgcaaagatggcgaatgggtgctgctgagcacctttctgtgataa

SEQ ID NO: 287 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 288 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNPPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 289 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPPCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 290 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQPPSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 291 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 292 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 293 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNPPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 294 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPPCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 295 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQPPSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPPFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 296 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNPPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 297 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPPCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 298 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQPPSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPPVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 299 - protein, 549 aa, synthetic construct
QNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 300 - protein, 549 aa, synthetic construct
QNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 301 - protein, 549 aa, synthetic construct
QNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 302 - protein, 549 aa, synthetic construct
QNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 303 - protein, 549 aa, synthetic construct
QNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 304 - protein, 549 aa, synthetic construct
QNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 305 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 306 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 307 - protein, 549 aa, synthetic construct
QNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 308 - protein, 549 aa, synthetic construct
QNITEEFYQSTCSAVSKGYLSALRTGWYTCVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVCTSKVLDLKNYIDKQLLPIVNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 309 - protein, 549 aa, synthetic construct
QNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 310 - protein, 549 aa, synthetic construct
QNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 311 - protein, 549 aa, synthetic construct
QNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPIVNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 312 - protein, 549 aa, synthetic construct
QNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 313 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 314 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKCSCCISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 315 - protein, 549 aa, synthetic construct
QNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 316 - protein, 549 aa, synthetic construct
QNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 317 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 318 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 319 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNKARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 320 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARQELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 321 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKQKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 322 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRQRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 323 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 324 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRQFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 325 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLQGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 326 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAICSGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 327 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 328 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 329 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 330 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 331 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINGSLAFIRKSDELLHNVNAGKSTTNIMITTIIIVIIVILLSLIAVGLLLYCKARSTPVTLSKDQLSGINNIAFSN

SEQ ID NO: 332 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDEKLHNVNAGKSTTNIMITTIIIVIIVILLSLIAVGLLLYCKARSTPVTLSKDQLSGINNIAFSN

SEQ ID NO: 333 - protein, 549 aa, synthetic construct; identical to SEQ ID NO: 317

SEQ ID NO: 334 - protein, 549 aa, synthetic construct; identical to SEQ ID NO: 317

SEQ ID NO: 335 - protein, 549 aa, synthetic construct; identical to SEQ ID NO: 317

SEQ ID NO: 336 - protein, 549 aa, synthetic construct; identical to SEQ ID NO: 317

SEQ ID NO: 337 - protein, 549 aa, synthetic construct; identical to SEQ ID NO: 317

SEQ ID NO: 338 - protein, 549 aa, synthetic construct; identical to SEQ ID NO: 317

SEQ ID NO: 339 - protein, 549 aa, synthetic construct; identical to SEQ ID NO: 317

SEQ ID NO: 340 - protein, 549 aa, synthetic construct; identical to SEQ ID NO: 317

SEQ ID NO: 341 - protein, 549 aa, synthetic construct
QNITVEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 342 - protein, 549 aa, synthetic construct
QNITEEFYQSTCSAVSKGYLSALRTGWYHSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 343 - protein, 549 aa, synthetic construct
QNITEEFYQSTCSAVSKGYLSALRTGWYTSVITIELSNIKENKCNGTDAKVKLIKQELDKYKSAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 344 - protein, 549 aa, synthetic construct
QNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 345 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNTKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 346 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKNTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 347 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKNNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 348 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRKFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 349 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 350 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 351 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKSNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 352 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 353 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 354 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 355 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 356 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLLLIAVGLLLYCKARSTPVTLSKDQLSGINNIAFSN

SEQ ID NO: 357 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLFLYCKARSTPVTLSKDQLSGINNIAFSN

SEQ ID NO: 358 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 359 - protein, 549 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAICSGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 360 - protein, 485 aa, synthetic construct
QNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLY

SEQ ID NO: 361 - protein, 501 aa, synthetic construct
QNITEEFYQSTCSAVSKGYLSALRTGWYTSVITIELSNIKENKCNGTDAKVKLIKQELDKYKNAVTELQLLMQSTPPTNNRARRGSFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 362 - protein, 500 aa, synthetic construct
QNITEEFYQSTCSAVSKGYLSALRTGWYTSVITIELSNIKENKCNGTDAKVKLIKQELDKYKNAVTELQLLMQSTPPTNNRARRGSFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYC

SEQ ID NO: 363 - protein, 508 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 364 - protein, 481 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTN

SEQ ID NO: 365 - protein, 531 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 366 - protein, 483 aa, synthetic construct
QNITEEFYQSTCSAVSKGYLSALRTGWYTSVITIELSNIKENKCNGTDAKVKLIKQELDKYKNAVTELQLLMQSTPPTNNRARRGSSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

SEQ ID NO: 367 - protein, 482 aa, synthetic construct
QNITEEFYQSTCSAVSKGYLSALRTGWYTSVITIELSNIKENKCNGTDAKVKLIKQELDKYKNAVTELQLLMQSTPPTNNRARRGSSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYC

SEQ ID NO: 368 - protein, 506 aa, synthetic construct
QNITEEFYQSTCSAVSKGYLSALRTGWYTSVITIELSNIKENKCNGTDAKVKLIKQELDKYKNAVTELQLLMQSTPPTNNRARRGSSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 369 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 370 - protein, 526 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNGYIPEAPRDGQAYVRKDGEWVLLSTFL

(19) United States
(12) Patent Application Publication
(10) Pub. No.: US 2026/0263375 A1
(43) Pub. Date: Sep. 10, 2026

(54) NUCLEIC ACIDS ENCODING THERAPEUTIC POLYPEPTIDES AND LIPID NANOPARTICLE COMPOSITION COMPRISING THE NUCLEIC ACIDS

(71) Applicant: STARNA THERAPEUTICS, Suzhou, Jiangsu (CN)

(72) Inventors: Huarong BAI, Suzhou, Jiangsu (CN); Zhenzhen WANG, Suzhou, Jiangsu (CN); Rongkuan HU, Suzhou, Jiangsu (CN)

(21) Appl. No.: 19/167,732

(22) PCT Filed: Mar. 22, 2024
(86) PCT No.: PCT/CN2024/083234

§ 371 Date: Sep. 22, 2025

(30) Foreign Application Priority Data
Mar. 24, 2023 (WO) PCT/CN2023/083664

(51) Int. Cl. A61K 9/51; A61K 9/00; A61K 39/12; C12N 15/88

(52) U.S. Cl. CPC A61K 9/5146; A61K 9/0019; A61K 9/5123; A61K 39/12; C12N 15/88

(57) ABSTRACT

The disclosure provides lipid nanoparticle compositions comprising nucleic acids encoding RSV antigenic polypeptides. The present disclosure also provides novel antigenic RSV-F polypeptides as well as nucleic acids encoding the antigenic RSV-F polypeptides.

### FIELD OF THE DISCLOSURE

The present disclosure provides lipid nanoparticle compositions comprising nucleic acids encoding Respiratory Syncytial Virus (RSV) antigenic polypeptides or variants thereof. The present disclosure also provides novel RSV polypeptides and variants thereof as well as nucleic acids encoding the RSV polypeptides and the variants.

Also provided is use of the RSV polypeptides and variants thereof, the nucleic acids encoding the same, and the lipid nanoparticle compositions comprising the nucleic acids in the manufacture of medicaments, e.g., vaccines. Also provided is the use of the medicaments (e.g., vaccines) in treating or preventing RSV infection. Also provided is the use of the nucleic acids encoding the RSV polypeptides and the variants in treating or preventing RSV infection.

### TECHNICAL BACKGROUND

RSV is a common cause of acute lower respiratory tract infections (ALRis) in hospital outpatients in infants and early childhood. In the United States, for example, more than 60% of infants are infected with RSV during their first RSV season, and almost all infants have been infected with RSV by 2-3 years old. Approximately 2.1 million U.S. children under 5 years old are treated for RSV infection each year, among which 3% are inpatients, 25% are in emergency clinic, and 73% are in pediatric clinic. Globally, RSV causes an estimated 33.8 million cases of ALRis (more than 22% of all ALRis) annually in children under 5 years old, resulting in 66,000 to 199,000 deaths, 99% of which occur in developing countries. RSV is also a common cause of respiratory diseases in elderly people, resulting in as many hospitalizations as influenza in the severely influenza-immune population. RSV is spread through droplets and close contact with infected people or contaminants. In temperate climates, there is an annual winter epidemic. Infants have the highest risk of severe RSV associated disease in their first 6 months of life, and hospitalization peaks at 2-3 months old. Premature birth and cardiopulmonary disease are risk factors for severe RSV associated disease. RSV infection in infants induces partial protective immunity that appears to decrease more rapidly than immunity against other respiratory viruses. Most children infected with RSV during their first year are reinfected the following year, usually with a reduced severity of the disease. Reinfection persists throughout life, often with upper respiratory symptoms and sometimes involving the lower respiratory tract or sinuses. The recommended treatment for RSV bronchiolitis consists mainly of respiratory support and hydration. No specific antiviral therapy is commonly available. The neutralizing monoclonal antibody Palivizumab is used for prevention in infants with highest risk of severe infection, but it is too expensive to be widely used. There is currently no licensed RSV vaccine, and the development of a safe and effective RSV vaccine is a top global public health goal.

RSV virion consists of internal nucleocapsid comprising viral RNA bound to nucleoproteins (N), phosphoproteins (P), and large polymerase proteins (L). The nucleocapsid is surrounded by matrix proteins (M) and is encapsulated by a lipid bilayer into which the viral fusion (F) protein (“RSV-F protein”) and attachment (G) protein as well as the small hydrophobic protein (SH) are incorporated. There are two subtypes of RSV, A and B. The difference is mainly in the G glycoprotein, while the F glycoprotein sequence is more conserved. Neutralizing antibodies targeting RSV-F proteins have been shown to limit viral replication and cause a less severe disease course.

Therefore, there remains a need to provide mRNA sequences encoding RSV related proteins and using the same in the treatment or prevention of RSV infection, especially in infants, elderly, and immunocompromised patients, and needs to develop compositions and methods to facilitate the delivery of the mRNA sequences and/or reduce the adverse effects caused by RSV-based nucleic acid sequences.

### SUMMARY

Disclosed here are lipid nanoparticle compositions comprising nucleic acids encoding a fusion protein (F protein) of Respiratory Syncytial Virus (RSV) or a variant thereof. Disclosed here are also novel RSV polypeptides, variants thereof and nucleic acids encoding the same. The disclosure also provides use of said lipid nanoparticle compositions, the antigenic polypeptides and the nucleic acids encoding the antigenic polypeptides for inducing an antigen specific immune response or in the manufacture of a medicament (e.g., a vaccine).

In one aspect, the present disclosure provides a lipid nanoparticle composition comprising:
a target polynucleotide that comprises a nucleic acid encoding Respiratory Syncytial Virus antigenic polypeptide or a variant thereof, and
a lipid nanoparticle comprising a compound having Formula (I) below:

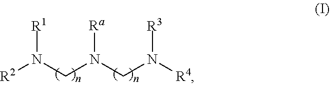

or a pharmaceutically acceptable salt thereof, wherein
   R^{a} is selected from the group consisting of hydrogen, R⁵, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted by one or more groups independently selected from the group consisting of halogen, hydroxyl, cyano, cycloalkyl, heterocyclyl, aryl, heteroaryl and R⁶;
   R¹ is

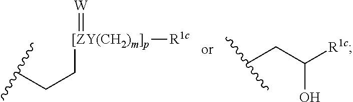

R² is

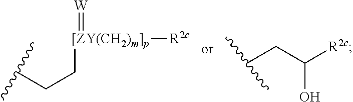

R³ is

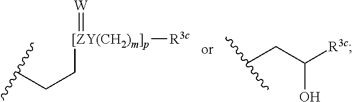

R⁴ is

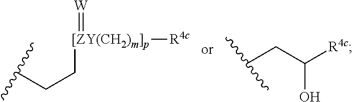

R⁵, if exists, is

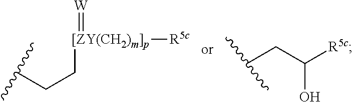

R⁶, if exists, is

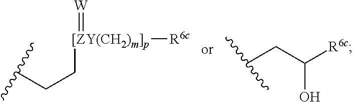

each W is independently selected from O, S or NR^{b}, and each R^{b} is independently selected from hydrogen, alkyl, alkyloxycarbonyl, acyl or sulfonyl;
each Y is independently selected from O, S, NR^{c}, N(R^{c})Z(W), N(R^{c})N(R^{c}) or
N(R^{c})N(R^{c})Z(W), and each R^{c} is independently selected from hydrogen, alkyl, alkyloxycarbonyl, acyl or sulfonyl;
each Z is independently selected from C, S or S(O);
each n is independently 0, 1, 2, 3, 4 or 5;
each m is independently 0, 1, 2 or 3;
each p is independently 1, 2, 3 or 4; and
each of R^{1c}, R^{2c}, R^{3c} and R^{4c} is independently selected from the group consisting of alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted by one or more groups independently selected from the group consisting of halogen, hydroxyl, oxo, cyano, cycloalkyl, heterocyclyl, aryl and heteroaryl, and the alkyl, alkenyl, alkynyl, heteroalkyl, heteroalkenyl and heteroalkynyl are optionally interrupted by one or more groups independently selected from the group consisting of cycloalkyl, heterocyclyl, aryl and heteroaryl.

In one aspect, the present disclosure provides a Respiratory Syncytial Virus polypeptide mutant, wherein the mutant has the Respiratory Syncytial Virus antigenic activity.

In one aspect, the present disclosure provides an isolated nucleic acid sequence encoding a Respiratory Syncytial Virus polypeptide comprising an amino acid sequence of the present disclosure, or the Respiratory Syncytial Virus polypeptide mutant of the present disclosure.

In one aspect, the present disclosure provides a Respiratory Syncytial Virus vaccine, comprising the lipid nanoparticle composition of the present disclosure, the Respiratory Syncytial Virus polypeptide mutant of the present disclosure, or the isolated nucleic acid sequence of the present disclosure.

In one aspect, the present disclosure provides a method of inducing an antigen specific immune response in a subject, comprising administering to the subject the Respiratory Syncytial Virus vaccine of the present disclosure in an amount effective to produce an antigen specific immune response.

### DESCRIPTION OF THE DRAWINGS

FIGS. 1A-1E show in vitro expression of the RSV mRNAs detected by Western Blot and ELISA.

FIGS. 2A-2B in vitro expression of the RSV mRNAs detected by FACS.

FIG. 3A shows the FLuc mRNA included in different test lipid nanoparticles effectively expressed. FIG. 3B shows RSV binding antibody titers produced in serum after the immunization with RSV mRNA included in different test lipid nanoparticles.

FIGS. 4A-4C show the RSV F protein binding antibody titers level in serum after the immunization with RSV mRNA included in different test lipid nanoparticles. FIG. 4D shows the F protein-specific T cell response (IFN-γ level) in spleen cells of mice after the immunization with RSV mRNA included in different test lipid nanoparticles.

FIG. 5A shows RSV neutralizing antibody titers produced in serum after the immunization with RSV mRNA included in different test lipid nanoparticles.

FIG. 5B shows the RSV viral load in the right lung of the test mice. FIG. 5C shows microscopic examination results.

FIG. 6 shows the RSV F protein binding antibody titers level in serum after the immunization with RSV mRNA included in different test lipid nanoparticles.

FIG. 7 shows RSV F protein binding antibody titers level in serum after the immunization with RSV mRNA vaccines.

FIG. 8 shows RSV F protein binding antibody titers level in serum after the immunization with RSV mRNA vaccines.

FIG. 9 shows RSV F protein binding antibody titers level in serum after the immunization with RSV mRNA vaccines.

FIGS. 10A-10B show RSV F protein binding antibody titers level in serum after the immunization with RSV mRNA vaccines. FIG. 10C and FIG. 10D respectively shows pre-fusion RSV F protein binding antibody titer level and post-fusion RSV F protein binding antibody titer level in serum after the immunization with RSV mRNA vaccines

FIG. 11A and FIG. 11B show the body weight and temperature of the test mice. FIG. 11c the level of IL-2, TNF-α and IFN-γ produced in the test mice. FIG. 11D shows the level of coagulation function in the test mice. FIGS. 11E-11K show the level of blood routine test in the test mice.

### DETAILED DESCRIPTION

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

### Definitions

Unless defined otherwise, all technical and scientific terms used herein have the same meaning as commonly understood by one of ordinary skill in the art to which this invention belongs. As used herein, the following terms are intended to have the following meanings:

As used in the specification and claims, the singular forms “a”, “an”, and “the” and the like includes plural references unless the context clearly dictates otherwise. Thus, for example, reference to “a compound” includes both a single compound and a plurality of different compounds.

The term “about” as used herein intends to indicate that the values quoted are not to be construed as absolute, and measurement error, inter-batches variation and/or inter-apparatus variation should also be taken into account.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

It is to be understood that the “compound” of present disclosure can exist in solvated as well as un-solvated forms, such as, for example, hydrated forms, solid forms, and the present disclosure is intended to encompass all such solvated and unsolvated forms. It is further to be understood that the “compound” of present disclosure can exist in forms of pharmaceutically acceptable salts. In some embodiments, the “compound” of present disclosure is an ionizable lipid. In some embodiments, the “compound” of present disclosure can exist as a cationic lipid at physiological pH.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e., i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₂₄” indicates 1 to 24, particularly 2 to 24, particularly 4 to 24, particularly 6 to 24, particularly 8 to 22, particularly 10 to 20, particularly 10 to 18 or particularly 12 to 18 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 24 carbon atoms. In some embodiments, alkyl groups contain 1 to 23 carbon atoms. In some embodiments, alkyl groups contain 1 to 22 carbon atoms. In some embodiments, alkyl groups contain 1 to 21 carbon atoms. In some embodiments, alkyl groups contain 1 to 20 carbon atoms, 1 to 19 carbon atoms, 1 to 18 carbon atoms, 1 to 17 carbon atoms, 1 to 16 carbon atoms, 1 to 15 carbon atoms, 1 to 14 carbon atoms, 1 to 13 carbon atoms, or 1 to 12 carbon atoms. In some embodiments, alkyl groups contain 12 to 18 carbon atoms. In some embodiments, alkyl groups contain 12 to 17 carbon atoms, 12 to 16 carbon atoms, 12 to 15 carbon atoms or 12 to 14 carbon atoms. In some embodiments, alkyl groups contain 12 to 19 carbon atoms, 12 to 20 carbon atoms, 12 to 21 carbon atoms, 12 to 22 carbon atoms, 12 to 23 carbon atoms or 12 to 24 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

The alkyl groups can be further substituted by substituents which independently replace one or more hydrogen atoms on one or more carbons of the alkyl groups. Examples of such substituents can include, but are not limited to, acyl, alkyl, alkenyl, alkynyl, oxo, halogen, hydroxyl, alkoxyl, haloalkyl, haloalkoxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfmyl, sulfonate, sulfamoyl, sulfonamido, nitro, trifluoromethyl, cyano, nitro, azido, heterocyclyl, alkylaryl, or an aromatic or heteroaromatic moiety. Alkenyl, alkynyl, cycloalkyl, heterocyclyl, aryl and heteroaryl groups as described below may also be similarly substituted.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 24 carbon atoms. In some embodiments, alkenyl groups contain 2 to 23 carbon atoms. In some embodiments, alkenyl groups contain 2 to 22 carbon atoms, 2 to 21 carbon atoms, 2 to 20 carbon atoms, 2 to 19 carbon atoms, 2 to 18 carbon atoms, 2 to 17 carbon atoms, 2 to 16 carbon atoms, 2 to 15 carbon atoms, 2 to 14 carbon atoms, 2 to 13 carbon atoms, 2 to 12 carbon atoms, 2 to 11 carbon atoms, and in some embodiments, alkenyl groups contain 2 carbon atoms. In some embodiments, alkenyl groups contain 12 to 18 carbon atoms. In some embodiments, alkenyl groups contain 12 to 17 carbon atoms, 12 to 16 carbon atoms, 12 to 15 carbon atoms or 12 to 14 carbon atoms. In some embodiments, alkenyl groups contain 12 to 19 carbon atoms, 12 to 20 carbon atoms, 12 to 21 carbon atoms, 12 to 22 carbon atoms, 12 to 23 carbon atoms or 12 to 24 carbon atoms. In some embodiments, alkenyl groups contain one or more “Z” carbon-carbon double bond. Examples of alkenyl group include, but are not limited to, ethylenyl (or vinyl), propenyl, butenyl, pentenyl, 1-methyl-2 buten-1-yl, 5-hexenyl, and the like. In some embodiments, an alkenyl group has at least one carbon-carbon double bond. In some embodiments, an alkenyl group has at least two, at least three, at least four, at least five, at least six, at least seven, at least eight, at least nine, or at least ten carbon-carbon double bonds. In some embodiments, two or more of carbon-carbon double bonds in an alkenyl group are conjugated. In some embodiments, two or more of carbon-carbon double bonds in an alkenyl group are not conjugated. In some embodiments, two or more of carbon-carbon double bonds in an alkenyl group are isolated, cumulated or conjugated. The term “alkenyl”, whether as part of another term or used independently, is also meant to include linear or branched-chain hydrocarbon radical having at least one carbon-carbon double bond and at least one carbon-carbon triple bond.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkynyl groups contain 2 to 24 carbon atoms. In some embodiments, alkynyl groups contain 2 to 23 carbon atoms. In some embodiments, alkynyl groups contain 2 to 22 carbon atoms, 2 to 21 carbon atoms, 2 to 20 carbon atoms, 2 to 19 carbon atoms, 2 to 18 carbon atoms, 2 to 17 carbon atoms, 2 to 16 carbon atoms, 2 to 15 carbon atoms, 2 to 14 carbon atoms, 2 to 13 carbon atoms, 2 to 12 carbon atoms, 2 to 10 carbon atoms, and in some embodiments, alkynyl groups contain 2 carbon atoms. In some embodiments, alkynyl groups contain 12 to 18 carbon atoms. In some embodiments, alkynyl groups contain 12 to 17 carbon atoms, 12 to 16 carbon atoms, 12 to 15 carbon atoms or 12 to 14 carbon atoms. In some embodiments, alkynyl groups contain 12 to 19 carbon atoms, 12 to 20 carbon atoms, 12 to 21 carbon atoms, 12 to 22 carbon atoms, 12 to 23 carbon atoms or 12 to 24 carbon atoms. Examples of alkynyl group include, but are not limited to, ethynyl, 1-propynyl, 2-propynyl, and the like. In some embodiments, an alkynyl group has at least one carbon-carbon triple bond. In some embodiments, an alkynyl group has at least two, at least three, at least four, at least five, at least six, at least seven, at least eight, at least nine, or at least ten carbon-carbon triple bonds. In some embodiments, two or more of carbon-carbon triple bonds in an alkynyl group are conjugated. In some embodiments, two or more of carbon-carbon triple bonds in an alkynyl group are not conjugated. The term “alkynyl”, whether as part of another term or used independently, is also meant to include linear or branched-chain hydrocarbon radical having at least one carbon-carbon triple bond and at least one carbon-carbon double bond.

As used herein, the term “alkoxyl”, whether as part of another term or used independently, refers to an alkyl group, as previously defined, attached to the parent molecule through an oxygen atom. The term “Cᵢ₋ⱼ alkoxyl” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g., n-propoxy and isopropoxy), t-butoxy, neopentoxy, n-hexoxy, and the like.

As used herein, the term “amino” refers to —NH₂. In some embodiments, amino may be substituted by any possible substituents on nitrogen.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused, bridged or spiro. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 11 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 3 to 4 ring forming carbon atoms, 4 to 12 ring forming carbon atoms, 4 to 11 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused-, spiro- or bridged-ring system. As used herein, the term “fused-ring” refers to a ring system having two rings sharing two adjacent atoms, the term “spiro-ring” refers to a ring systems having two rings connected through one single common atom, and the term “bridged-ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2,2,1]heptanyl, bicyclo[2,2,2]octanyl, bicyclo[3,3,1]nonanyl, bicyclo[3,3,3]undecanyl, and the like.

As used herein, the term “cyano” refers to —CN.

As used herein, the term “oxo” refers to ═O, which substitutes two hydrogen atoms connected to one atom. For example, an oxo substituted ethyl is CH₃C(═O)— or —C(═O)CH₂—.

As used herein, the term “halogen” refers to an atom selected from fluorine (or fluoro), chlorine (or chloro), bromine (or bromo) and iodine (or iodo).

As used herein, the term “haloalkyl”, whether as part of another term or used independently, refers to an alkyl group having one or more halogen substituents. Examples of haloalkyl group include, but are not limited to, trifluoromethyl (—CF₃), pentafluoroethyl (—C₂F₅), difluoromethyl (—CHF₂), trichloromethyl (—CCl₃), dichloromethyl (—CHCl₂), pentachloroethyl (—C₂Cl₅), and the like.

As used herein, the term “haloalkoxyl”, whether as part of another term or used independently, refers to an alkoxyl group having one or more halogen substituents. As a result, the term “halo-Cᵢ₋ⱼ alkoxyl”, whether as part of another term or used independently, refers to a Cᵢ₋ⱼ alkoxyl group having one or more halogen substituents. Examples of haloalkoxyl include, but are not limited to, —O—CF₃, —O—C₂F₅, —O—CHF₂, —O—CCl₃, —O—CHCl₂, —O—C₂Cl₅, and the like.

As used herein, the term “heteroatom” refers to nitrogen (N), oxygen (O), sulfur (S), and includes any oxidized form of nitrogen or sulfur, and any quaternized form of a basic nitrogen (including N-oxides).

As used herein, the term “heteroalkyl”, “heteroalkenyl”, or “heteroalkynyl”, whether as part of another term or used independently, refers to an alkyl, alkenyl, or alkynyl group containing one or more heteroatoms. As a result, the term “hetero-Cᵢ₋ⱼ alkyl”, “hetero-Cᵢ₋ⱼ alkenyl”, or “hetero-Cᵢ₋ⱼ alkynyl”, whether as part of another term or used independently, refers to a Cᵢ₋ⱼ alkyl, Cᵢ₋ⱼ alkenyl, or Cᵢ₋ⱼ alkynyl containing one or more heteroatoms. For example, the term “hetero-C₁₋₆ alkyl”, whether as part of another term or used independently, refers to a C₁₋₆ alkyl containing one or more heteroatoms. In some embodiments, a heteroalkyl, heteroalkenyl or heteroalkynyl group contains at least one heteroatom. In some embodiments, a heteroalkyl, heteroalkenyl or heteroalkynyl group contains at least two, at least three, at least four, at least five, at least six, at least seven, at least eight, at least nine or at least ten heteroatoms. In some embodiments, two or more heteroatoms in a heteroalkyl, heteroalkenyl or heteroalkynyl group are the same. In some embodiments, two or more heteroatoms in a heteroalkyl, heteroalkenyl or heteroalkynyl group are different. In some embodiments, two or more heteroatoms in a heteroalkyl, heteroalkenyl or heteroalkynyl group are directly bonded. In some embodiments, two or more heteroatoms in a heteroalkyl, heteroalkenyl or heteroalkynyl group are not directly bonded.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring or the other ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocyclyl group may be monocyclic. Examples of monocyclic heterocyclyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocyclyl group may be polycyclic, including the fused-, spiro- and bridged-ring systems. The fused heterocyclyl group includes radicals wherein the heterocyclyl radicals are fused with a saturated, partially unsaturated, or fully unsaturated (i.e., aromatic) carbocyclic or heterocyclic ring. Examples of fused heterocyclyl include, but are not limited to, phenyl fused-ring or pyridinyl fused-ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl and the like. Examples of bridged heterocyclyl include, but are not limited to, 3-aza-bicyclo[3,1,0]hexanyl, 8-aza-bicyclo[3,2,1]octanyl, 1-aza-bicyclo[2,2,2]octanyl, 2-aza-bicyclo[2,2,1]heptanyl, 1,4-diazabicyclo[2,2,2]octanyl, and the like.

As used herein, the term “hydroxyl” or “hydroxy” refers to —OH.

As used herein, the term “alkyloxycarbonyl” refers to alkyl-O—C(═O)—. In some embodiments, an alkyloxycarbonyl may be further substituted on alkyl by any possible substituents described above. Examples of alkyloxycarbonyl include, but not limited to, t-butoxylcarbonyl, benzyloxycarbonyl, allyloxycarbonyl and 9-fluorenylmethyloxycarbonyl.

As used herein, the term “sulfonyl” refers to R—SO₂—, wherein R is hydrogen or any possible substituents on sulfur. Examples of sulfonyl include, but not limited to, p-toluenesulfonyl, p-bromobenzenesulfonyl, 2- or 4-nitrobenzenesulfonyl, trifluoromethanesulfonyl, methanesulfonyl and 5-(dimethylamino)naphthalene-1-sulfonyl.

As used herein, the term “acyl”, whether as part of another term or used independently, refers to RC(═O)—, wherein R is hydrogen or any possible substituents on carbon. Examples of acyl include, but not limited to, formyl, acetyl, trifluoroacetyl and benzoyl. The term “alkylacyl” refers to alkyl-C(═O)—, wherein the alkyl is optionally substituted by any possible substituents described above. The term “arylacyl” refers to aryl-C(O═)-, wherein the aryl is optionally substituted by any possible substituents described above.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution”, “substituted by” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted by more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “interrupted”, whether preceded by the term “optionally” or not, means that one or more covalent bonds of the designated moiety are replaced with a suitable linking group, but not at the termini. In some embodiments, the replaced bond is a carbon-carbon bond. In some embodiments, the replaced bond is a carbon-heteroatom bond. Unless otherwise indicated, an “optionally interrupted” group may have a suitable linking group at each replaceable position of the group, and when more than one position in any given structure may be interrupted by more than one linking group selected from a specified group, the linking group may be either the same or different at every position. In some embodiments, an alkyl “interrupted” by a cycloalkyl refers to alkyl-cycloalkyl-alkyl. In some embodiments, an alkenyl “interrupted” by a cycloalkyl refers to alkenyl-cycloalkyl-alkyl, alkenyl-cycloalkyl-alkenyl, alkenyl-cycloalkyl-alkynyl, alkyl-cycloalkyl-alkenyl or alkynyl-cycloalkyl-alkenyl. In some embodiments, an alkynyl “interrupted” by a cycloalkyl refers to alkynyl-cycloalkyl-alkyl, alkynyl-cycloalkyl-alkenyl, alkynyl-cycloalkyl-alkynyl, alkyl-cycloalkyl-alkynyl or alkenyl-cycloalkyl-alkynyl. In some embodiments, an alkyl “interrupted” by a heterocyclyl refers to alkyl-heterocyclyl-alkyl. In some embodiments, an alkenyl “interrupted” by a heterocyclyl refers to alkenyl-heterocyclyl-alkyl, alkenyl-heterocyclyl-alkenyl, alkenyl-heterocyclyl-alkynyl, alkyl-heterocyclyl-alkenyl or alkynyl-heterocyclyl-alkenyl. In some embodiments, an alkynyl “interrupted” by a heterocyclyl refers to alkynyl-heterocyclyl-alkyl, alkynyl-heterocyclyl-alkenyl, alkynyl-heterocyclyl-alkynyl, alkyl-heterocyclyl-alkynyl or alkenyl-heterocyclyl-alkynyl. In some embodiments, an alkyl “interrupted” by an aryl refers to alkyl-aryl-alkyl. In some embodiments, an alkenyl “interrupted” by an aryl refers to alkenyl-aryl-alkyl, alkenyl-aryl-alkenyl, alkenyl-aryl-alkynyl, alkyl-aryl-alkenyl or alkynyl-aryl-alkenyl. In some embodiments, an alkynyl “interrupted” by an aryl refers to alkynyl-aryl-alkyl, alkynyl-aryl-alkenyl, alkynyl-aryl-alkynyl, alkyl-aryl-alkynyl or alkenyl-aryl-alkynyl. In some embodiments, an alkyl “interrupted” by a heteroaryl refers to alkyl-heteroaryl-alkyl. In some embodiments, an alkenyl “interrupted” by a heteroaryl refers to alkenyl-heteroaryl-alkyl, alkenyl-heteroaryl-alkenyl, alkenyl-heteroaryl-alkynyl, alkyl-heteroaryl-alkenyl or alkynyl-heteroaryl-alkenyl. In some embodiments, an alkynyl “interrupted” by a heteroaryl refers to alkynyl-heteroaryl-alkyl, alkynyl-heteroaryl-alkenyl, alkynyl-heteroaryl-alkynyl, alkyl-heteroaryl-alkynyl or alkenyl-heteroaryl-alkynyl.

As used herein, the term “pharmaceutically acceptable” refers to those compounds, lipid nanoparticles, lipid nanoparticle composition, materials, compositions, and/or dosage forms which are, within the scope of sound medical judgment, suitable for use in contact with the tissues of humans and other animals without excessive toxicity, irritation, allergic response, or other problem or complication, commensurate with a reasonable benefit/risk ratio. In some embodiments, compounds, lipid nanoparticles, lipid nanoparticle composition, materials, compositions, and/or dosage forms that are pharmaceutically acceptable refer to those approved by a regulatory agency (such as U.S. Food and Drug Administration, National Medical Products Administration or European Medicines Agency) or listed in generally recognized pharmacopoeia (such as U.S. Pharmacopoeia, China Pharmacopoeia or European Pharmacopoeia) for use in animals, and more particularly in humans.

As used herein, “pharmaceutically acceptable salts” or “pharmaceutical salts” refers to derivatives of a compound wherein the parent compound is modified by converting an existing acidic moiety (e.g., carboxyl and the like) or base moiety (e.g., amine, alkali and the like) to its salt form. In many cases, compounds of present disclosure are capable of forming acid addition salts and/or base salts by virtue of the presence of amino, alkali or groups similar thereto. And the “pharmaceutically acceptable salts” include acid addition salts or base salts that retain biological effectiveness and properties of the parent compound, which typically are not biologically or otherwise undesirable. Pharmaceutically acceptable salts are well known in the art. For example, Berge et al. describes pharmaceutically acceptable salts in detail in J. Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, propionic acid, glycolic acid, pyruvic acid, oxalic acid, lactic acid, trifluoracetic acid, benzoic acid, cinnamic acid, mandelic acid, ethanesulfonic acid, p-toluenesulfonic acid, salicylic acid, malonic acid, fumaric acid, citric acid, malic acid, maleic acid, tartaric acid, succinic acid, or methanesulfonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, inorganic acids from which salts can be derived include, for example, hydrochloric acid salt, sulfuric acid salt, phosphoric acid salt, and the like. In some embodiments, organic acids from which salts can be derived include, for example, maleic acid salt, fumaric acid salt, oxalic acid salt, p-toluenesulfonic acid salt, succinic acid salt, L-(+)-tartaric acid salt, mono-adipic acid salt, hemi-adipic acid salt, and the like.

The term “pharmaceutical composition” refers to a mixture of one or more compounds of the present disclosure or one or more lipid nanoparticles or lipid nanoparticle composition of present disclosure, with other chemical components, such as pharmaceutically acceptable diluent, excipient or carrier. The purpose of a pharmaceutical composition is to facilitate administration of compounds, lipid nanoparticles or lipid nanoparticle composition to a subject.

The term “pharmaceutically acceptable excipient” or “pharmaceutically acceptable carrier” as used herein refers to a pharmaceutically-acceptable material, composition or vehicle, such as a liquid or solid filler, diluent, excipient, solvent or encapsulating material, involved in carrying or transporting a compound provided herein from one location, body fluid, tissue, organ (interior or exterior), or portion of the body, to another location, body fluid, tissue, organ, or portion of the body. Pharmaceutically acceptable excipients or carriers can be vehicles, diluents, excipients, or other materials that can be used to contact the tissues of an animal without excessive toxicity or adverse effects. Non-limiting examples of pharmaceutically acceptable excipients or carriers include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-α-tocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of compounds described herein. Pharmaceutically acceptable excipients or carriers that can be employed in present disclosure includes those generally known in the art, such as those disclosed in “Remington Pharmaceutical Sciences” Mack Pub. Co., New Jersey (1991), which is incorporated herein by reference.

As used herein, “administration” of a disclosed compound, lipid nanoparticles or lipid nanoparticle composition encompasses the delivery to a subject of a compound, lipid nanoparticles or lipid nanoparticle composition as described herein, or a prodrug or other pharmaceutically acceptable derivative thereof, using any suitable formulation or route of administration, as discussed herein.

As used herein, the term “delivering”, “deliver” or “delivery” means providing an entity to a destination. For example, delivering a therapeutic and/or prophylactic agent to a subject may involve administering a lipid nanoparticle composition comprising the therapeutic and/or prophylactic agent to the subject (e.g, by an intravenous, intramuscular, intradermal, or subcutaneous route). Administration of lipid particle or a composition comprising the lipid particle to a mammal or mammalian cell may involve contacting one or more cells with the lipid particle or the composition.

As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more or at least 100-fold more) of a therapeutic and/or prophylactic agent by a lipid particle to a target tissue of interest (e.g., liver, lung, spleen or muscle) or a target cell of interest (e.g., liver cell, lung cell, spleen cell or muscle cell) compared to the level of delivery of a therapeutic and/or prophylactic agent by a control lipid particle (e.g., lipid particle comprising DLin-MC3-DMA) to a target tissue of interest (e.g., liver, lung, spleen or muscle) or a target cell of interest (e.g., liver cell, lung cell, spleen cell or muscle cell). The level of delivering a therapeutic and/or prophylactic agent to a particular tissue or cell may be measured by comparing the amount of therapeutic and/or prophylactic agent in a tissue or cell to the amount of total therapeutic and/or prophylactic agent in said tissue or cell, comparing the amount of therapeutic and/or prophylactic agent in a tissue or cell to the weight of said tissue, comparing the amount of protein produced in a tissue or cell to the amount of total protein in said tissue or cell, comparing the amount of protein produced in a tissue to the weight of said tissue or cell, or comparing the amount of therapeutic and/or prophylactic agent delivered to a tissue or cell to the amount of total administrated therapeutic and/or prophylactic agent. It will be understood that the enhanced delivery of a therapeutic and/or prophylactic agent to a target tissue or cell need not be determined in a subject being treated, it may be determined in a surrogate such as an animal model (e.g., a mice or rat model). In certain embodiments, a lipid particle composition including a compound having Formula (I) has substantively the same level of delivery enhancement regardless of administration routes. For example, certain compounds disclosed herein exhibit similar delivery enhancement when they are used for delivering a therapeutic and/or prophylactic agent either intravenously or intramuscularly.

As used herein, the term “selective delivery,” “selectively deliver,” or “selectively delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic agent by a lipid particle to a target tissue of interest (e.g., liver, lung, spleen or muscle) or a target cell of interest (e.g., liver cell, lung cell, spleen cell or muscle cell) compared to an off-target tissue (e.g., liver, lung, spleen or muscle) or an off-target cell of interest (e.g., liver cell, lung cell, spleen cell or muscle cell). The level of delivery of a therapeutic and/or prophylactic agent to a particular tissue or cell may be measured by comparing the amount of therapeutic and/or prophylactic agent in a tissue or cell to the amount of total therapeutic and/or prophylactic agent in said tissue or cell, comparing the amount of therapeutic and/or prophylactic agent in a tissue or cell to the weight of said tissue or cell, comparing the amount of protein produced in a tissue or cell to the amount of total protein in said tissue or cell, or comparing the amount of protein produced in a tissue or cell to the weight of said tissue or cell. It will be understood that the ability of a lipid particle to specifically deliver a therapeutic and/or prophylactic agent to a target tissue or cell need not be determined in a subject being treated, it may be determined in a surrogate such as an animal model (e.g., a mice or rat model).

The term “effective amount” “pharmaceutically effective amount” or “therapeutically effective amount” refers to the amount of a therapeutic and/or prophylactic agent delivered to a tissue or cell, or a compound or pharmaceutical composition described herein that is sufficient to prevent, treat, reduce and/or ameliorate the symptoms and/or underlying causes of any disorder or disease in a subject, or the amount of an agent sufficient to produce a desired effect on target cells, e.g., reduction of cell migration, an increase or inhibition of expression of a target nucleic acid in a cell in comparison to the normal expression level of the nucleic acid detected in the absence of the therapeutic and/or prophylactic agent delivered to a tissue or cell, or a compound or pharmaceutical composition described herein. In one embodiment, a “pharmaceutically effective amount” or “therapeutically effective amount” is an amount sufficient to reduce or eliminate a symptom of a disease. In another embodiment, a pharmaceutically or therapeutically effective amount is an amount sufficient to overcome the disease itself. In certain specific embodiments, a “pharmaceutically effective amount” or “therapeutically effective amount” is an amount effective for detectable killing or inhibition of the growth or spread of cancer cells, reducing in the size or number of tumors; or other measure of the level, stage, progression or severity of the cancer. The pharmaceutically or therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. The specific dose will vary depending on, for example, the particular compounds chosen, the species of the subject and their age/existing health conditions or risk for health conditions, the dosing regimen to be followed, the severity of the disease, whether it is administered in combination with other agents, timing of administration, the tissue to which it is administered, and the physical delivery system in which it is carried. Thus, a pharmaceutically or therapeutically effective amount may be administered in one or more administrations. For example, and without limitation, a pharmaceutically or therapeutically effective amount of an agent, in the context of treating cancer, refers to an amount of the agent that alleviates, ameliorates, palliates, or eliminates one or more symptoms of cancer in the patient.

The term “expression” of a nucleic acid sequence refers to one or more of the following events: (1) producing an RNA template from a DNA sequence (e.g., by transcription); (2) processing an RNA transcript (e.g., by splicing, editing, 5′cap formation, and/or 3′end processing); (3) translating an RNA into a polypeptide or protein; and (4) post-translational modification of a polypeptide or protein.

Polypeptide or polynucleotide molecules of the present disclosure may share a certain degree of sequence similarity or identity with the reference molecules (e.g., reference polypeptides or reference polynucleotides). The term “identity”, as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. In some embodiments, variants of a particular polynucleotide or polypeptide have at least 50%, 55%, 60%, 65%, 70%, 75%, 80%, 85%, 90%, 91%, 92%, 93%, 94%, 95%, 96%, 97%, 98%, 99%, 99.5%, 99.6%, 99.7%, 99.8% or 99.9% sequence identity to that particular reference polynucleotide or polypeptide as determined by sequence alignment programs and parameters described herein and known to those skilled in the art.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, rabbits, hamsters, mice, cats, and/or dogs; and/or birds, including commercially relevant birds such as chickens, ducks, geese, quail, and/or turkeys. In some embodiments, a subject has been or may be diagnosed with a disease or disorder. In some embodiments, a subject has not been diagnosed with a disease or disorder.

### Lipids

In one aspect, the present disclosure provides novel compounds useful as lipids.

A lipid has at least one features of the following: a hydrophilic head group with varying pKa, a cationic, mono-, di-, tri-amine, oligoamine/polyamine, an imidazole, a pyridine, a guanidinium, and hydrophobic tails. In some embodiments, a lipid is an ionizable lipid. In some embodiments, a lipid is a cationic lipid.

As used herein, the term “cationic lipid” includes lipids having an amino head and one or more aliphatic chains, which may be protonated to form a cationic lipid as physiological pH. In some embodiments, a cationic lipid is an amino lipid. Lipids having one or more protonatable or deprotonatable group, or which are zwitterionic are also included. In some embodiments, a lipid of the present disclosure has at least one protonatable group, such that the lipid is positively charged at a first pH at or below physiological pH (e.g., at or below pH 7.4), and neutral at a second pH (e.g., at or above physiological pH). In some embodiments, a lipid of the present disclosure has at least two or at least three protonatable groups. It shall be understood that the addition or removal of protons as a function of pH is an equilibrium process, and that the reference to a charged (e.g., protonated) or a neutral lipid refers to the nature of the predominant species (e.g., more than 50%, 60%, 70%, 80%, 90%, 95% or 99%) and does not require that all of the lipid be present in the charged or neutral form.

In some embodiments, cationic lipids of the present disclosure have a pKa of the protonatable group in the range of about 4 to about 11. In some embodiments, lipids have a pKa of about 4 to about 7, about 5 to about 7, or about 5.5 to about 6.8, when incorporated into lipid particles. In some embodiments, lipids having such pKa will be cationic at a lower pH, while particles will be largely (though not completely) surface neutralized at physiological pH (e.g., at pH 7.4). In some embodiments, at least some nucleic acid associated with the outside surface of the particle comprising lipids having such pKa will lose its electrostatic interaction at physiological pH and be removed by simple dialysis; thus greatly reducing the particle's susceptibility to clearance. pKa measurements of lipids within lipid particles can be performed, for example, by using the fluorescent probe 2-(p-toluidino)-6-napthalene sulfonic acid (TNS), using method described in Cullis et al., (1986) Chem Phys Lipids 40, 127-144.

In some embodiments, lipids of the present disclosure are advantageously used in lipid nanoparticles. In some embodiments, a lipid nanoparticle is used in in vivo delivery of therapeutic agents to cells. In some embodiments, a lipid nanoparticle is used in in vivo delivery of therapeutic agents to a tissue.

In another aspect, a lipid of the present disclosure is a compound having Formula (I) below:

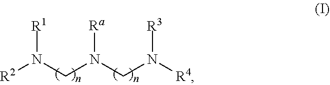

or a pharmaceutically acceptable salt thereof, wherein
   R^{a} is selected from the group consisting of hydrogen, R⁵, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted by one or more groups independently selected from the group consisting of halogen, hydroxyl, cyano, cycloalkyl, heterocyclyl, aryl, heteroaryl and R⁶;
   R¹ is

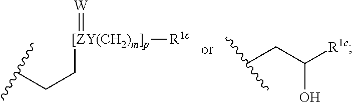

R² is

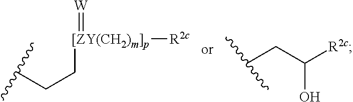

R³ is

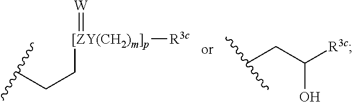

R⁴ is

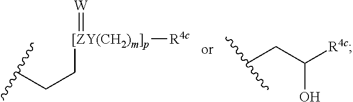

R⁵, if exists, is

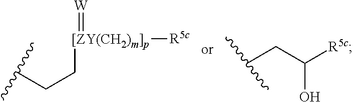

R⁶, if exists, is

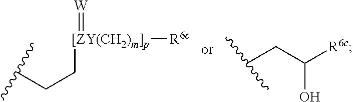

each W is independently selected from O, S or NR^{b}, and each R^{b} is independently selected from hydrogen, alkyl, alkyloxycarbonyl, acyl or sulfonyl;
each Y is independently selected from O, S, NR^{c}, N(R^{c})Z(W), N(R^{c})N(R^{c}) or N(R^{c})N(R^{c})Z(W), and each R^{c} is independently selected from hydrogen, alkyl, alkyloxycarbonyl, acyl or sulfonyl;
each Z is independently selected from C, S or S(O);
each n is independently 0, 1, 2, 3, 4 or 5;
each m is independently 0, 1, 2 or 3;
each p is independently 1, 2, 3 or 4; and
each of R^{1c}, R^{2c}, R^{3c} and R^{4c} is independently selected from the group consisting of alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted by one or more groups independently selected from the group consisting of halogen, hydroxyl, oxo, cyano, cycloalkyl, heterocyclyl, aryl and heteroaryl, and the alkyl, alkenyl, alkynyl, heteroalkyl, heteroalkenyl and heteroalkynyl are optionally interrupted by one or more groups independently selected from the group consisting of cycloalkyl, heterocyclyl, aryl and heteroaryl.

In some embodiments, a lipid of the present disclosure is a compound having Formula (A) or (B) below:

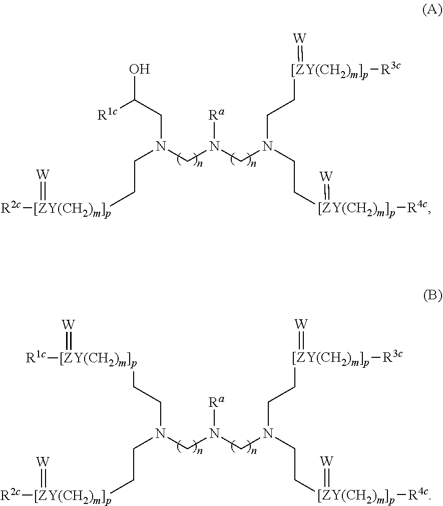

In some embodiments, at least two of R², R³ and R⁴ are the same. In some embodiments, at least three of R², R³ and R⁴ are the same.

In some embodiments, one or more of W is O.

In some embodiments, one or more of Y is O, NR^{c} or N(R^{c})N(R^{c})Z(W).

In some embodiments, one or more of Z is C or S(O).

In some embodiments, one or more of R^{2c}, R^{3c} and R^{4c} is alkyl or alkenyl.

In some embodiments, one or more of R^{2c}, R^{3c} and R^{4c} is C₈₋₂₄ alkyl or alkenyl.

In some embodiments, one or more of R^{2c}, R^{3c} and R^{4c} is C₁₀₋₂₄ alkyl or alkenyl.

In some embodiments, one or more of R^{2c}, R^{3c} and R^{4c} is alkenyl comprising one, two or three C═C double bond.

In some embodiments, wherein one or more of R^{2c}, R^{3c} and R^{4c} is alkenyl comprising one or more Z-olefin.

In some embodiments, wherein R^{1c} is alkyl.

In some embodiments, R^{1c} is C₁₋₁₂ alkyl.

In some embodiments, R^{1c} is C₄₋₁₀ alkyl.

In some embodiments, R^{a} is C₁₋₆ alkyl optionally substituted by one or more groups independently selected from the group consisting of hydroxyl, cycloalkyl and heteroaryl.

In some embodiments, R^{a} is methyl, ethyl, propyl, butyl or pentyl.

In some embodiments, one or more of

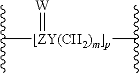

independently selected from the group consisting of

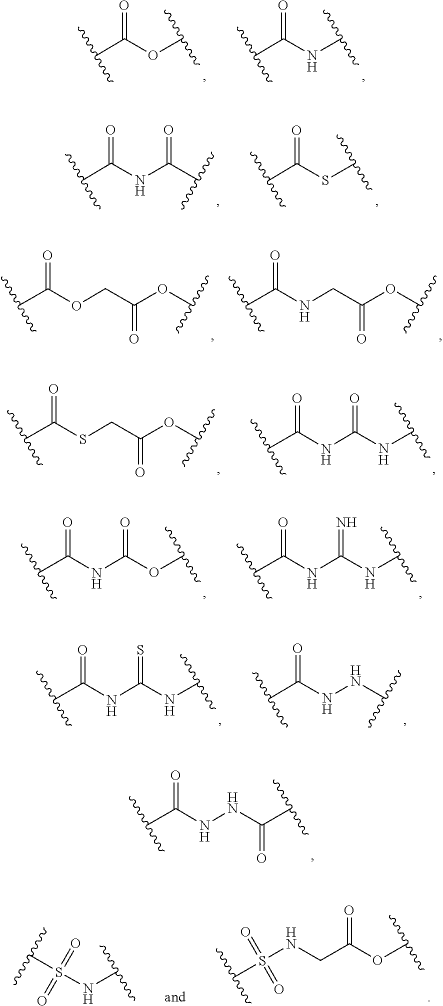

In some embodiments, one or more of R^{1c}, R^{2c}, R^{3c} and R^{4c} is independently selected from the group consisting of

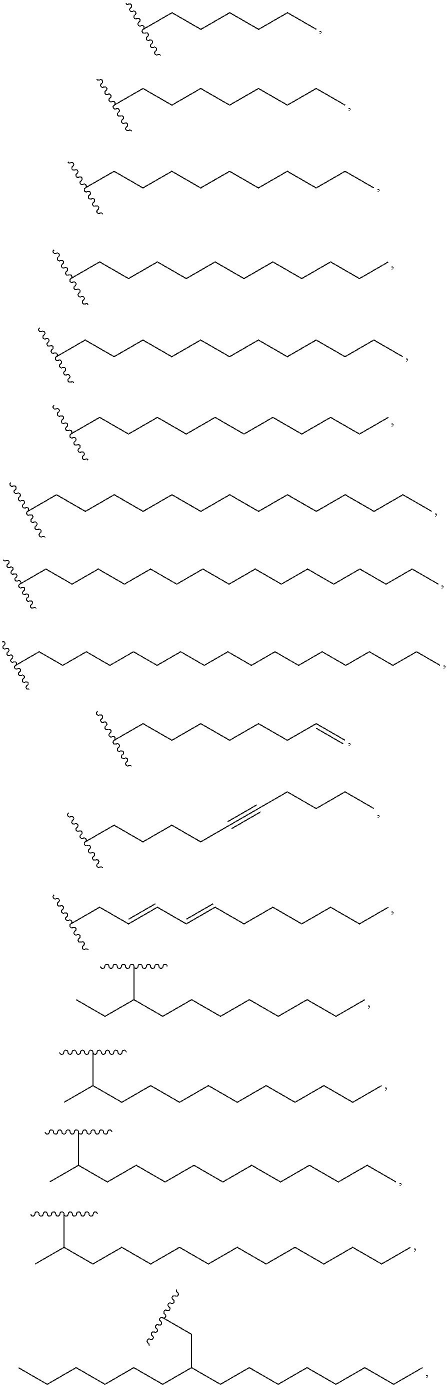

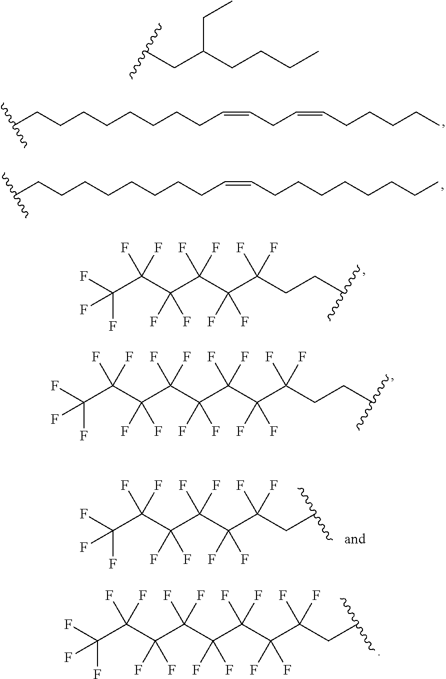

In some embodiments, each of R^{1c}, R^{2c}, R^{3c}, R^{4c}, R^{5c} and R^{6c}, if exists, does not comprise two heteroatoms directly bonded to each other.

In some embodiments, each of R^{1c}, R^{2c}, R^{3c}, R^{4c}, R^{5c} and R^{6c}, if exists, comprises —N(R^{c})—N(R^{c})— or —S(O)₂—N(R^{c})—.

In some embodiments, a compound having Formula (I) is a compound listed in Table 1.

For the purpose of illustration, exemplary compounds of the present disclosure and their Structure Code are set forth in Table 1 below.

TABLE 1
Exemplary Compounds

| Compd. | |
|---|---|
| No. | Structure |
| 1 | 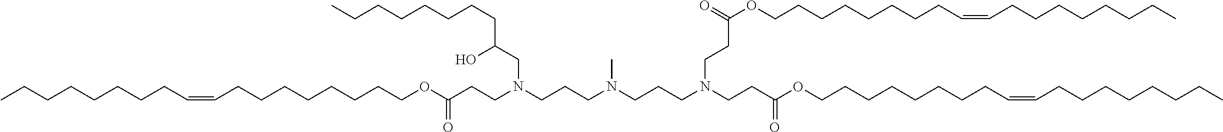 [SMILES: CCCCCCCC/C=C\CCCCCCCCOC(=O)CCN(CCCN(C)CCCN(CCC(=O)OCCCCCCCC/C=C\CCCCCCCC)CC(O)CCCCCCCC)CCC(=O)OCCCCCCCC/C=C\CCCCCCCC] |
| 2 | 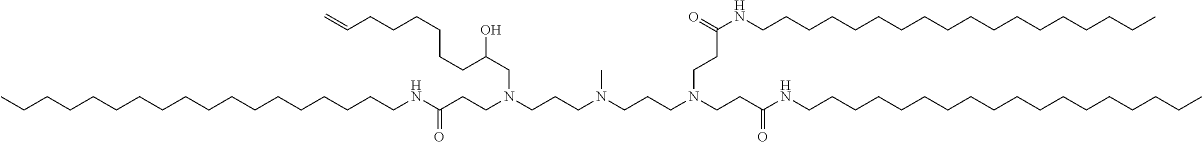 [SMILES: C=CCCCCCCC(O)CN(CCCN(C)CCCN(CCC(=O)NCCCCCCCCCCCCCCCCCC)CCC(=O)NCCCCCCCCCCCCCCCCCC)CCC(=O)NCCCCCCCCCCCCCCCCCC] |
| 3 | 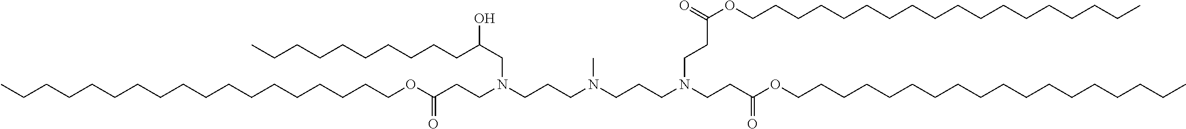 [SMILES: CCCCCCCCCCCCCCCCCCOC(=O)CCN(CCCN(C)CCCN(CCC(=O)OCCCCCCCCCCCCCCCCCC)CC(O)CCCCCCCCCC)CCC(=O)OCCCCCCCCCCCCCCCCCC] |
| 4 | 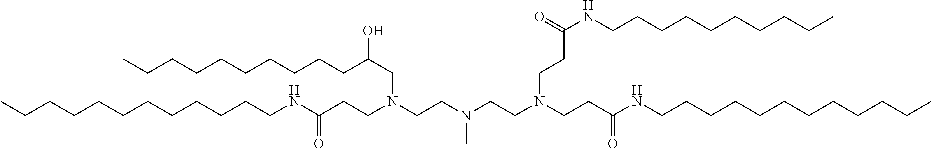 [SMILES: CCCCCCCCCCCCNC(=O)CCN(CCC(=O)NCCCCCCCCCC)CCN(C)CCN(CCC(=O)NCCCCCCCCCCCC)CC(O)CCCCCCCCCC] |
| 5 | 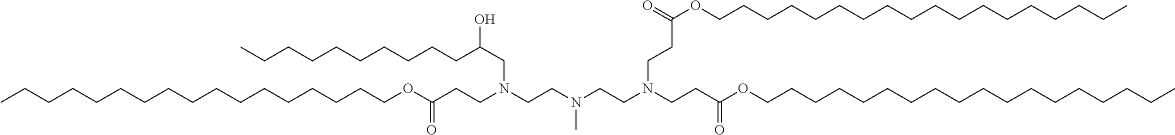 [SMILES: CCCCCCCCCCCCCCCCCCOC(=O)CCN(CCC(=O)OCCCCCCCCCCCCCCCCCC)CCN(C)CCN(CCC(=O)OCCCCCCCCCCCCCCCCC)CC(O)CCCCCCCCCC] |
| 6 | 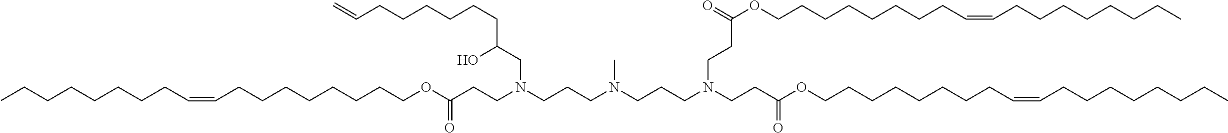 [SMILES: C=CCCCCCCC(O)CN(CCCN(C)CCCN(CCC(=O)OCCCCCCCC/C=C\CCCCCCCC)CCC(=O)OCCCCCCCC/C=C\CCCCCCCC)CCC(=O)OCCCCCCCC/C=C\CCCCCCCC] |
| 7 | 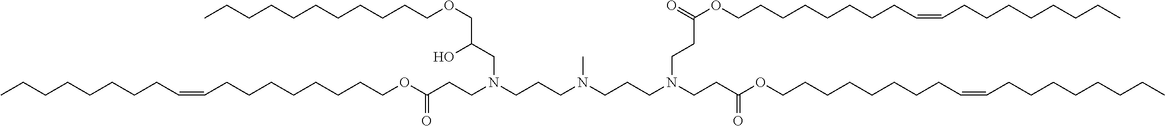 [SMILES: CCCCCCCC/C=C\CCCCCCCCOC(=O)CCN(CCCN(C)CCCN(CCC(=O)OCCCCCCCC/C=C\CCCCCCCC)CC(O)COCCCCCCCCCCC)CCC(=O)OCCCCCCCC/C=C\CCCCCCCC] |
| 8 | 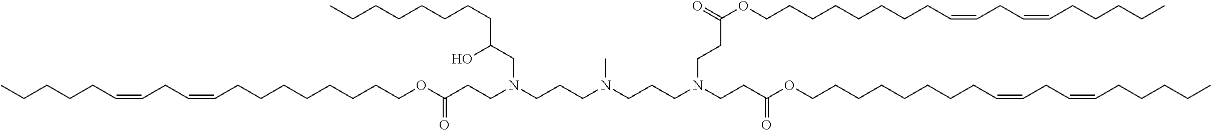 [SMILES: CCCCC/C=C\C/C=C\CCCCCCCCOC(=O)CCN(CCCN(C)CCCN(CCC(=O)OCCCCCCCC/C=C\C/C=C\CCCCC)CC(O)CCCCCCCC)CCC(=O)OCCCCCCCC/C=C\C/C=C\CCCCC] |
| 9 | 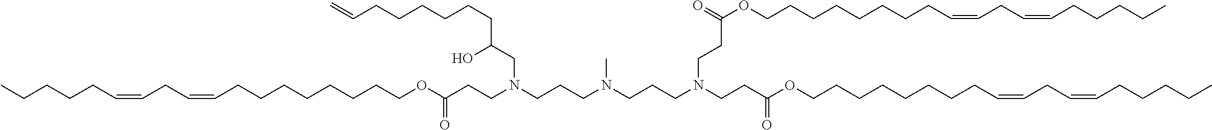 [SMILES: C=CCCCCCCC(O)CN(CCCN(C)CCCN(CCC(=O)OCCCCCCCC/C=C\C/C=C\CCCCC)CCC(=O)OCCCCCCCC/C=C\C/C=C\CCCCC)CCC(=O)OCCCCCCCC/C=C\C/C=C\CCCCC] |
| 10 | 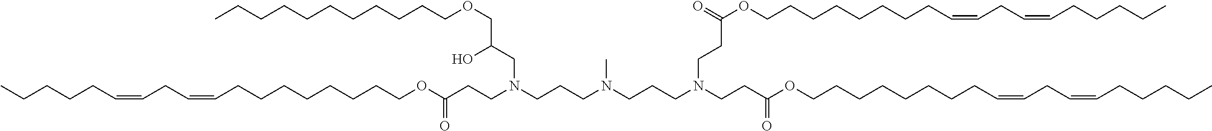 [SMILES: CCCCC/C=C\C/C=C\CCCCCCCCOC(=O)CCN(CCCN(C)CCCN(CCC(=O)OCCCCCCCC/C=C\C/C=C\CCCCC)CC(O)COCCCCCCCCCCC)CCC(=O)OCCCCCCCC/C=C\C/C=C\CCCCC] |
| 11 | 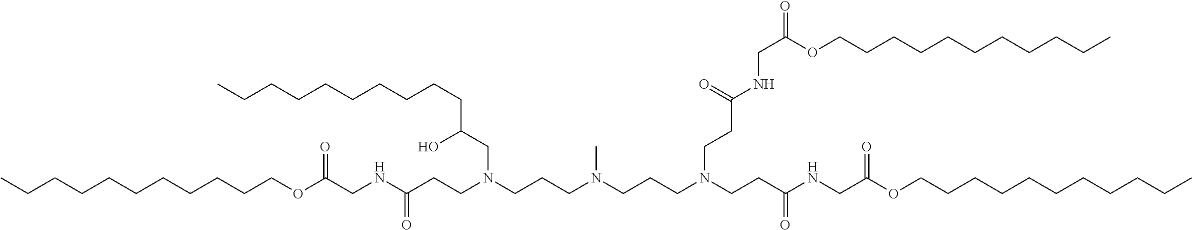 [SMILES: CCCCCCCCCCCOC(=O)CNC(=O)CCN(CCCN(C)CCCN(CCC(=O)NCC(=O)OCCCCCCCCCCC)CC(O)CCCCCCCCCC)CCC(=O)NCC(=O)OCCCCCCCCCCC] |
| 12 | 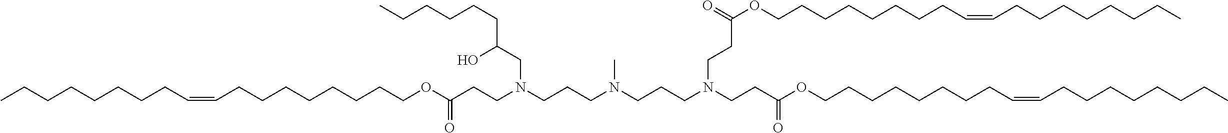 [SMILES: CCCCCCCC/C=C\CCCCCCCCOC(=O)CCN(CCCN(C)CCCN(CCC(=O)OCCCCCCCC/C=C\CCCCCCCC)CC(O)CCCCCC)CCC(=O)OCCCCCCCC/C=C\CCCCCCCC] |
| 13 | 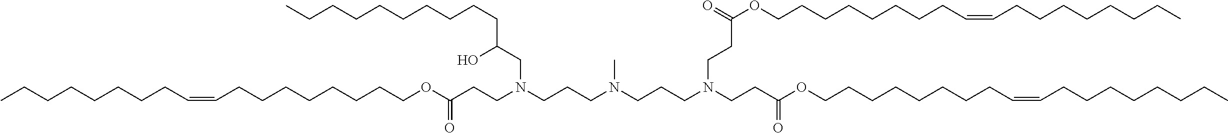 [SMILES: CCCCCCCC/C=C\CCCCCCCCOC(=O)CCN(CCCN(C)CCCN(CCC(=O)OCCCCCCCC/C=C\CCCCCCCC)CC(O)CCCCCCCCCC)CCC(=O)OCCCCCCCC/C=C\CCCCCCCC] |
| 14 | 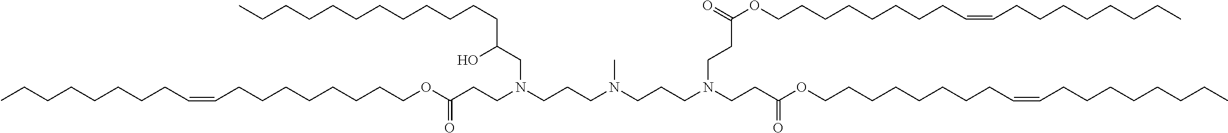 [SMILES: CCCCCCCC/C=C\CCCCCCCCOC(=O)CCN(CCCN(C)CCCN(CCC(=O)OCCCCCCCC/C=C\CCCCCCCC)CC(O)CCCCCCCCCCCC)CCC(=O)OCCCCCCCC/C=C\CCCCCCCC] |
| 15 | 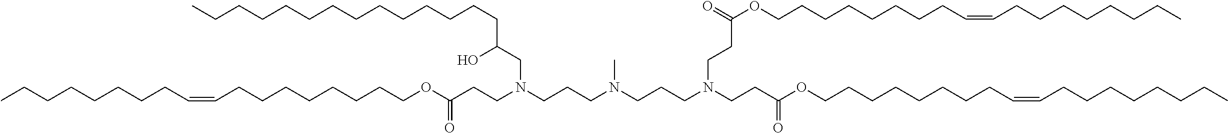 [SMILES: CCCCCCCC/C=C\CCCCCCCCOC(=O)CCN(CCCN(C)CCCN(CCC(=O)OCCCCCCCC/C=C\CCCCCCCC)CC(O)CCCCCCCCCCCCCC)CCC(=O)OCCCCCCCC/C=C\CCCCCCCC] |
| 16 | 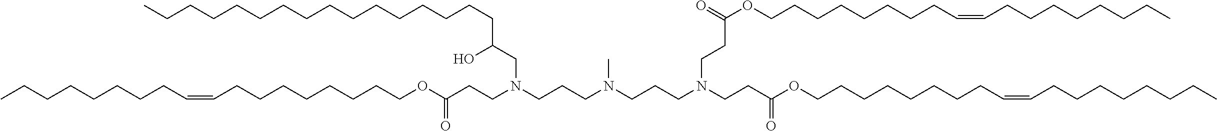 [SMILES: CCCCCCCC/C=C\CCCCCCCCOC(=O)CCN(CCCN(C)CCCN(CCC(=O)OCCCCCCCC/C=C\CCCCCCCC)CC(O)CCCCCCCCCCCCCCCC)CCC(=O)OCCCCCCCC/C=C\CCCCCCCC] |
| 17 | 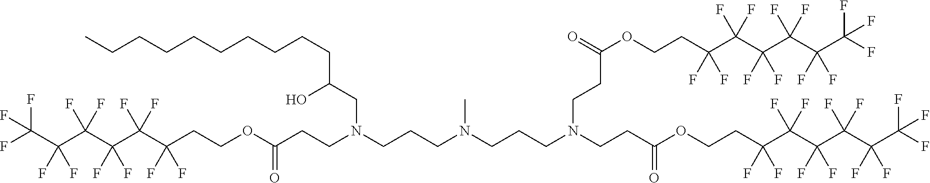 [SMILES: CCCCCCCCCCC(O)CN(CCCN(C)CCCN(CCC(=O)OCCC(F)(F)C(F)(F)C(F)(F)C(F)(F)C(F)(F)C(F)(F)F)CCC(=O)OCCC(F)(F)C(F)(F)C(F)(F)C(F)(F)C(F)(F)C(F)(F)F)CCC(=O)OCCC(F)(F)C(F)(F)C(F)(F)C(F)(F)C(F)(F)C(F)(F)F] |
| 18 | 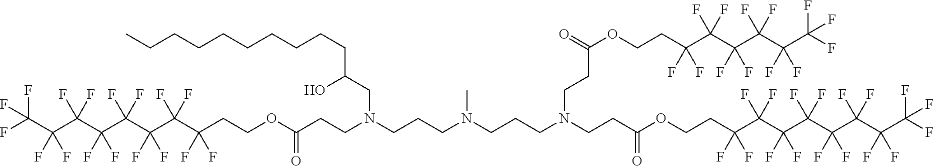 [SMILES: CCCCCCCCCCC(O)CN(CCCN(C)CCCN(CCC(=O)OCCC(F)(F)C(F)(F)C(F)(F)C(F)(F)C(F)(F)C(F)(F)F)CCC(=O)OCCC(F)(F)C(F)(F)C(F)(F)C(F)(F)C(F)(F)C(F)(F)C(F)(F)C(F)(F)F)CCC(=O)OCCC(F)(F)C(F)(F)C(F)(F)C(F)(F)C(F)(F)C(F)(F)C(F)(F)C(F)(F)F] |
| 19 | 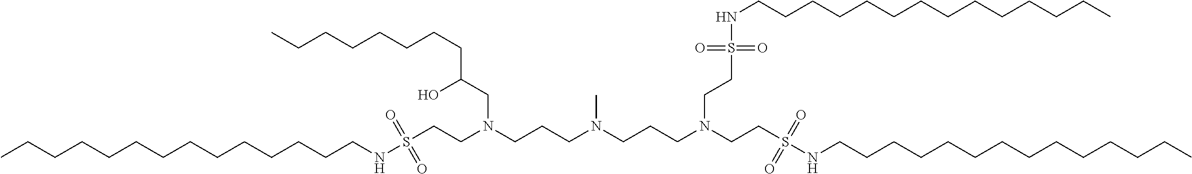 [SMILES: CCCCCCCCCCCCCCNS(=O)(=O)CCN(CCCN(C)CCCN(CCS(=O)(=O)NCCCCCCCCCCCCCC)CC(O)CCCCCCCC)CCS(=O)(=O)NCCCCCCCCCCCCCC] |
| 20 | 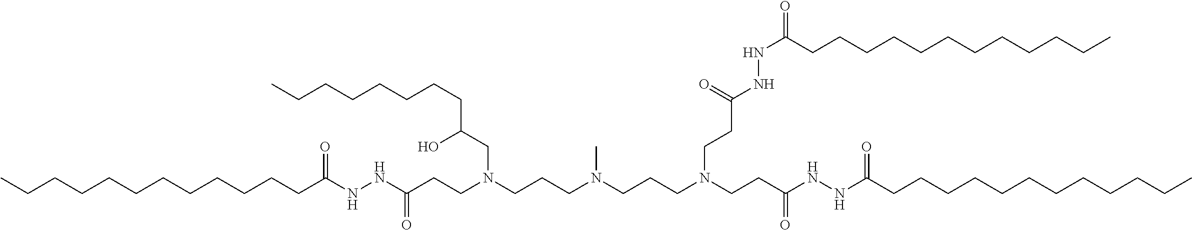 [SMILES: CCCCCCCCCCCCC(=O)NNC(=O)CCN(CCCN(C)CCCN(CCC(=O)NNC(=O)CCCCCCCCCCCC)CC(O)CCCCCCCC)CCC(=O)NNC(=O)CCCCCCCCCCCC] |
| 21 | 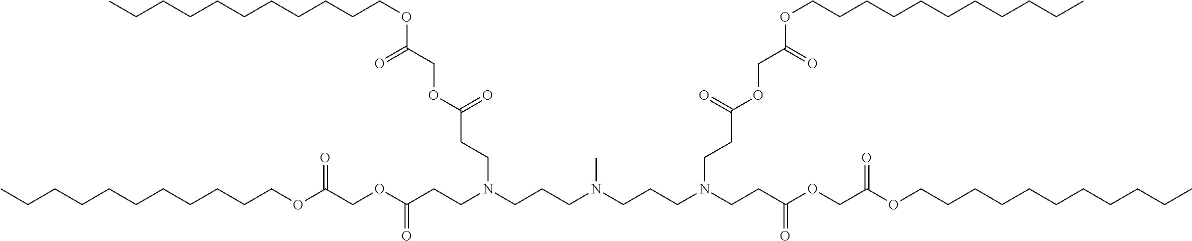 [SMILES: CCCCCCCCCCCOC(=O)COC(=O)CCN(CCCN(C)CCCN(CCC(=O)OCC(=O)OCCCCCCCCCCC)CCC(=O)OCC(=O)OCCCCCCCCCCC)CCC(=O)OCC(=O)OCCCCCCCCCCC] |
| 22 | 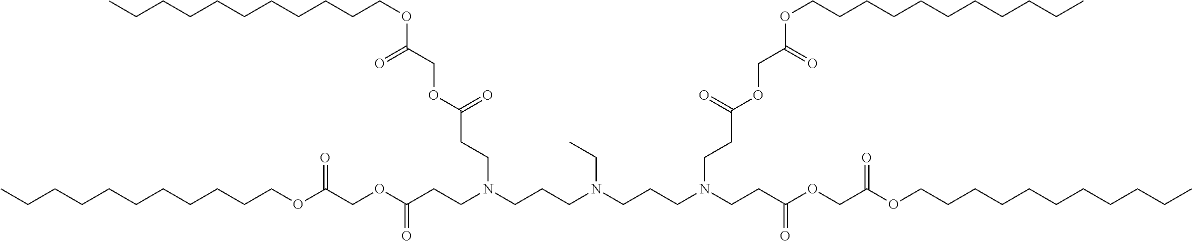 [SMILES: CCCCCCCCCCCOC(=O)COC(=O)CCN(CCCN(CC)CCCN(CCC(=O)OCC(=O)OCCCCCCCCCCC)CCC(=O)OCC(=O)OCCCCCCCCCCC)CCC(=O)OCC(=O)OCCCCCCCCCCC] |
| 23 | 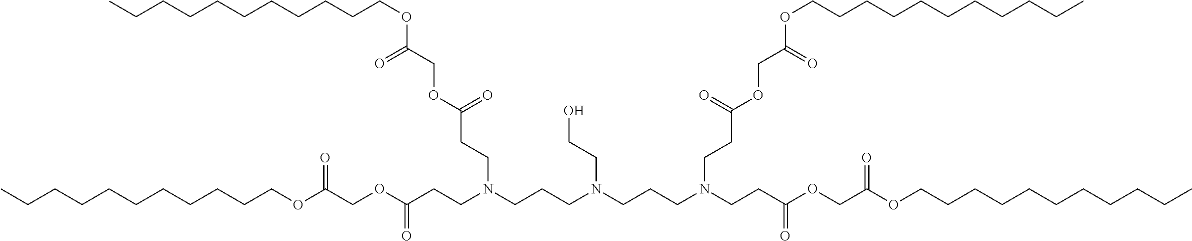 [SMILES: CCCCCCCCCCCOC(=O)COC(=O)CCN(CCCN(CCO)CCCN(CCC(=O)OCC(=O)OCCCCCCCCCCC)CCC(=O)OCC(=O)OCCCCCCCCCCC)CCC(=O)OCC(=O)OCCCCCCCCCCC] |
| 24 | 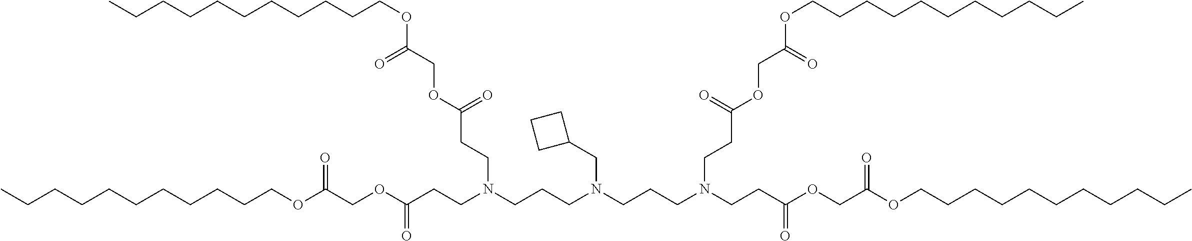 [SMILES: CCCCCCCCCCCOC(=O)COC(=O)CCN(CCCN(CCCN(CCC(=O)OCC(=O)OCCCCCCCCCCC)CCC(=O)OCC(=O)OCCCCCCCCCCC)CC1CCC1)CCC(=O)OCC(=O)OCCCCCCCCCCC] |
| 25 | 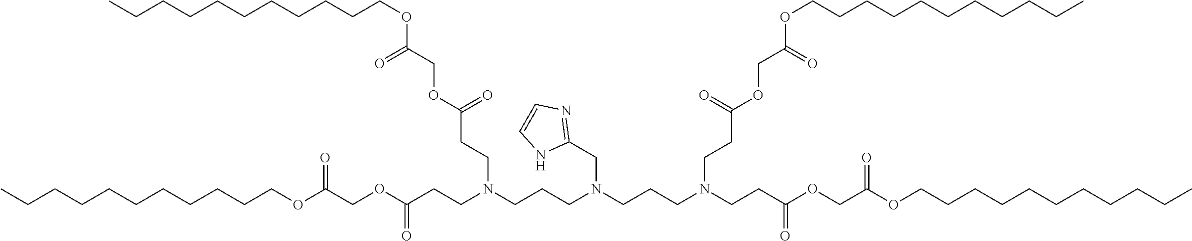 [SMILES: CCCCCCCCCCCOC(=O)COC(=O)CCN(CCCN(CCCN(CCC(=O)OCC(=O)OCCCCCCCCCCC)CCC(=O)OCC(=O)OCCCCCCCCCCC)Cc1ncc[nH]1)CCC(=O)OCC(=O)OCCCCCCCCCCC] |
| 26 | 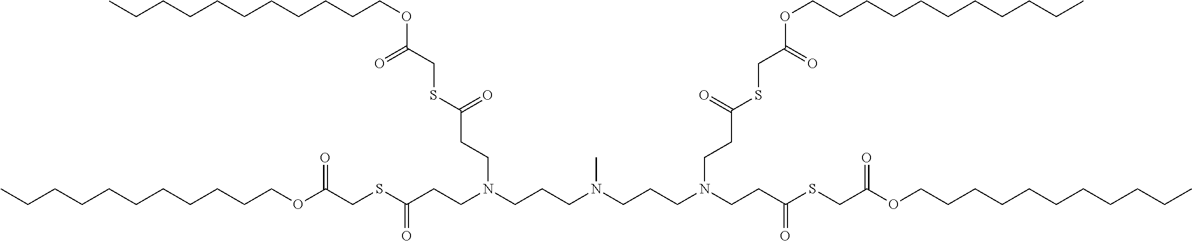 [SMILES: CCCCCCCCCCCOC(=O)CSC(=O)CCN(CCCN(C)CCCN(CCC(=O)SCC(=O)OCCCCCCCCCCC)CCC(=O)SCC(=O)OCCCCCCCCCCC)CCC(=O)SCC(=O)OCCCCCCCCCCC] |
| 27 | 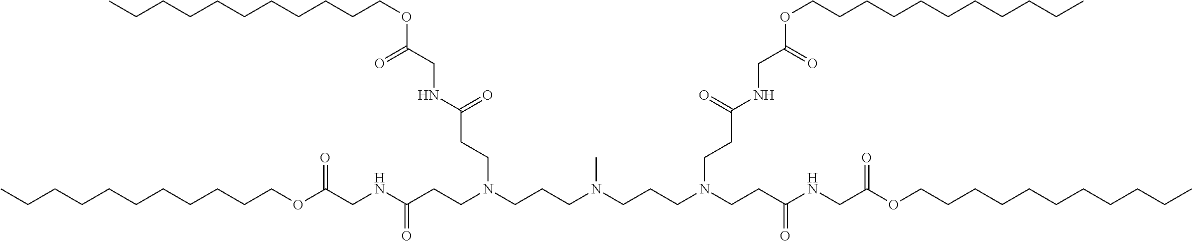 [SMILES: CCCCCCCCCCCOC(=O)CNC(=O)CCN(CCCN(C)CCCN(CCC(=O)NCC(=O)OCCCCCCCCCCC)CCC(=O)NCC(=O)OCCCCCCCCCCC)CCC(=O)NCC(=O)OCCCCCCCCCCC] |
| 28 | 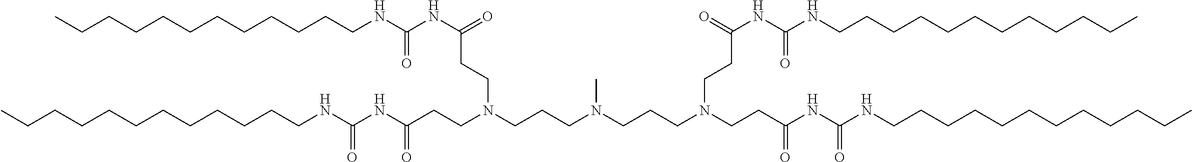 [SMILES: CCCCCCCCCCCCNC(=O)NC(=O)CCN(CCCN(C)CCCN(CCC(=O)NC(=O)NCCCCCCCCCCCC)CCC(=O)NC(=O)NCCCCCCCCCCCC)CCC(=O)NC(=O)NCCCCCCCCCCCC] |
| 29 | 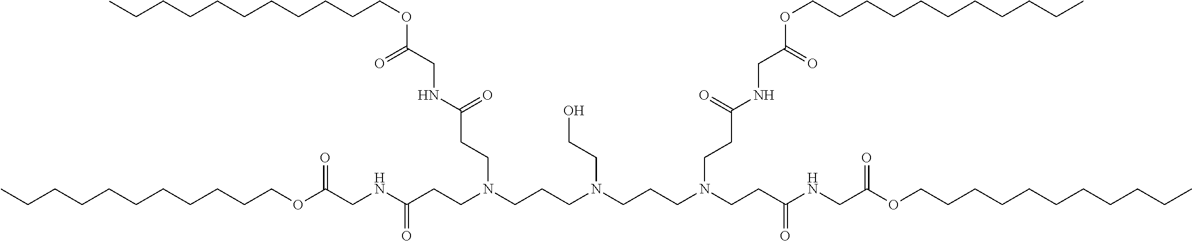 [SMILES: CCCCCCCCCCCOC(=O)CNC(=O)CCN(CCCN(CCO)CCCN(CCC(=O)NCC(=O)OCCCCCCCCCCC)CCC(=O)NCC(=O)OCCCCCCCCCCC)CCC(=O)NCC(=O)OCCCCCCCCCCC] |
| 30 | 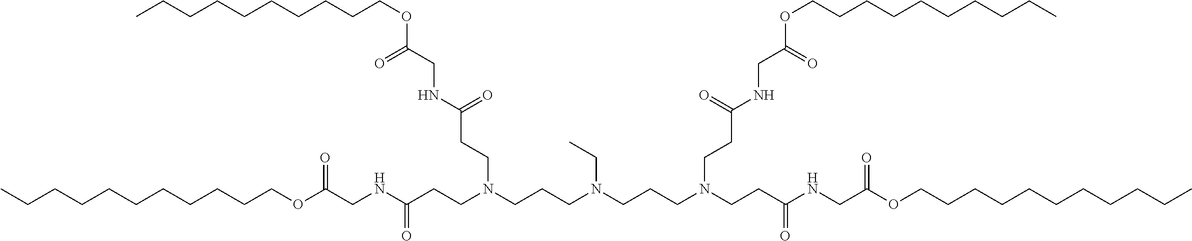 [SMILES: CCCCCCCCCCCOC(=O)CNC(=O)CCN(CCCN(CC)CCCN(CCC(=O)NCC(=O)OCCCCCCCCCC)CCC(=O)NCC(=O)OCCCCCCCCCCC)CCC(=O)NCC(=O)OCCCCCCCCCC] |
| 31 | 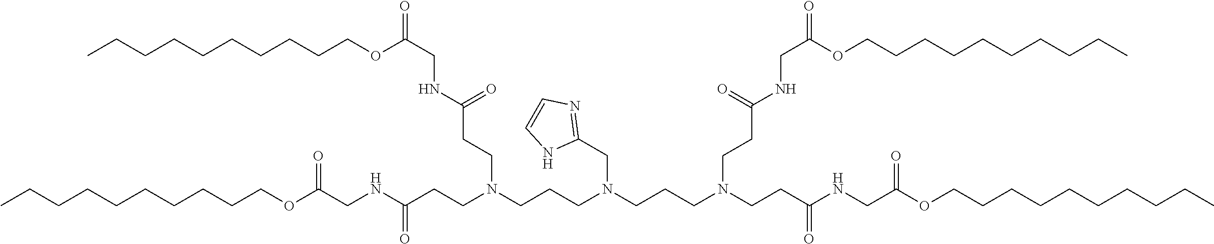 [SMILES: CCCCCCCCCCOC(=O)CNC(=O)CCN(CCCN(CCCN(CCC(=O)NCC(=O)OCCCCCCCCCC)CCC(=O)NCC(=O)OCCCCCCCCCC)Cc1ncc[nH]1)CCC(=O)NCC(=O)OCCCCCCCCCC] |
| 32 | 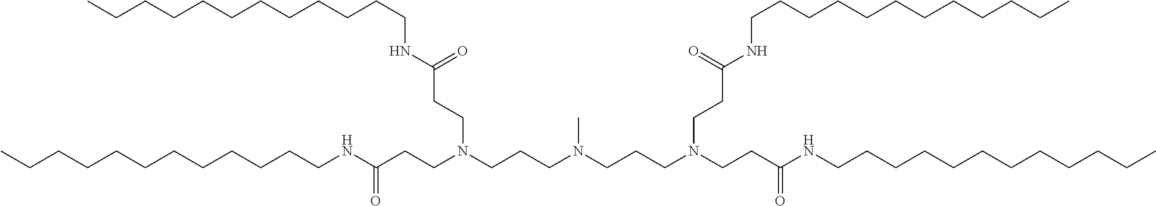 [SMILES: CCCCCCCCCCCCNC(=O)CCN(CCCN(C)CCCN(CCC(=O)NCCCCCCCCCCCC)CCC(=O)NCCCCCCCCCCCC)CCC(=O)NCCCCCCCCCCCC] |
| 33 | 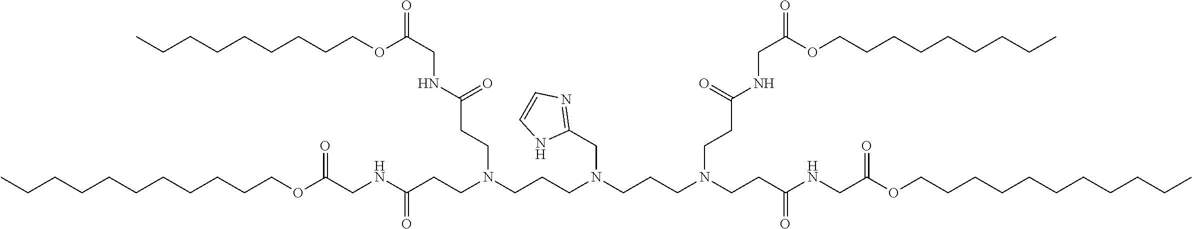 [SMILES: CCCCCCCCCCCOC(=O)CNC(=O)CCN(CCCN(CCCN(CCC(=O)NCC(=O)OCCCCCCCCC)CCC(=O)NCC(=O)OCCCCCCCCCCC)Cc1ncc[nH]1)CCC(=O)NCC(=O)OCCCCCCCCC] |
| 34 | 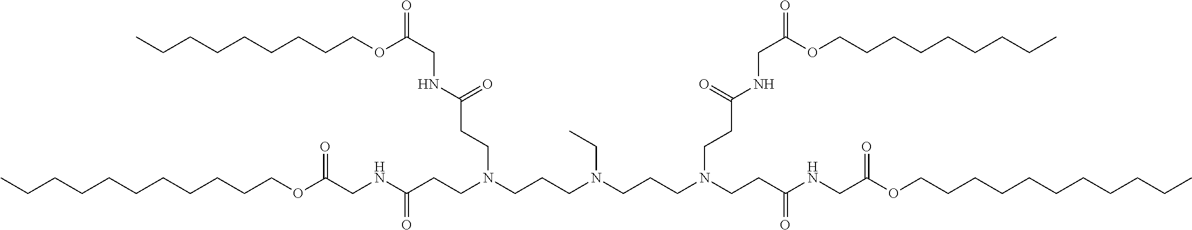 [SMILES: CCCCCCCCCCCOC(=O)CNC(=O)CCN(CCCN(CC)CCCN(CCC(=O)NCC(=O)OCCCCCCCCC)CCC(=O)NCC(=O)OCCCCCCCCCCC)CCC(=O)NCC(=O)OCCCCCCCCC] |
| 35 | 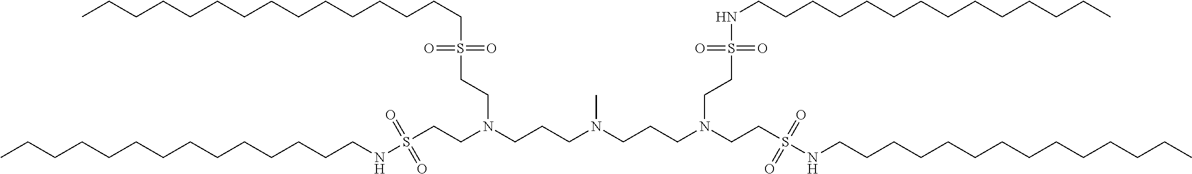 [SMILES: CCCCCCCCCCCCCCCS(=O)(=O)CCN(CCCN(C)CCCN(CCS(=O)(=O)NCCCCCCCCCCCCCC)CCS(=O)(=O)NCCCCCCCCCCCCCC)CCS(=O)(=O)NCCCCCCCCCCCCCC] |
| 36 | 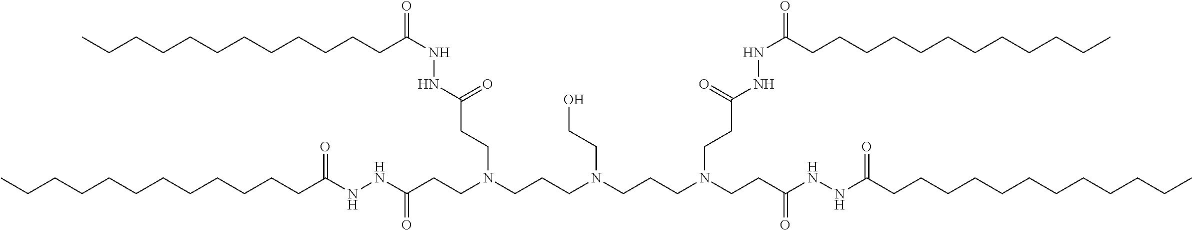 [SMILES: CCCCCCCCCCCCC(=O)NNC(=O)CCN(CCCN(CCO)CCCN(CCC(=O)NNC(=O)CCCCCCCCCCCC)CCC(=O)NNC(=O)CCCCCCCCCCCC)CCC(=O)NNC(=O)CCCCCCCCCCCC] |
| 37 | 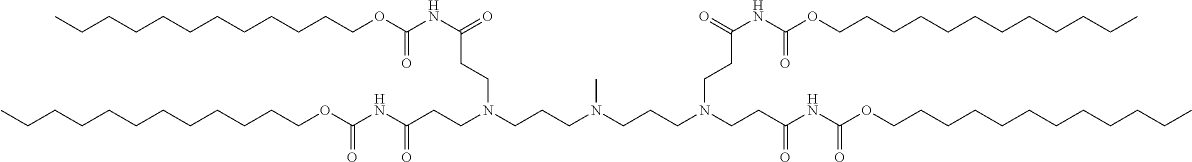 [SMILES: CCCCCCCCCCCCOC(=O)NC(=O)CCN(CCCN(C)CCCN(CCC(=O)NC(=O)OCCCCCCCCCCCC)CCC(=O)NC(=O)OCCCCCCCCCCCC)CCC(=O)NC(=O)OCCCCCCCCCCCC] |
| 38 | 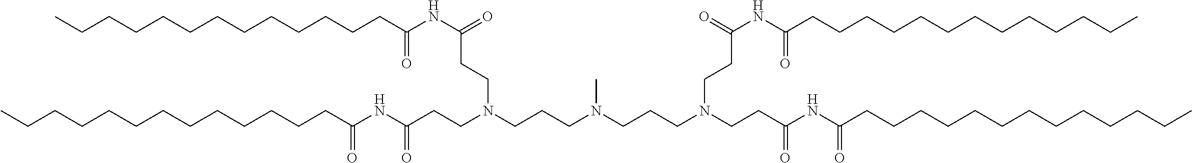 [SMILES: CCCCCCCCCCCCCC(=O)NC(=O)CCN(CCCN(C)CCCN(CCC(=O)NC(=O)CCCCCCCCCCCCC)CCC(=O)NC(=O)CCCCCCCCCCCCC)CCC(=O)NC(=O)CCCCCCCCCCCCC] |
| 39 | 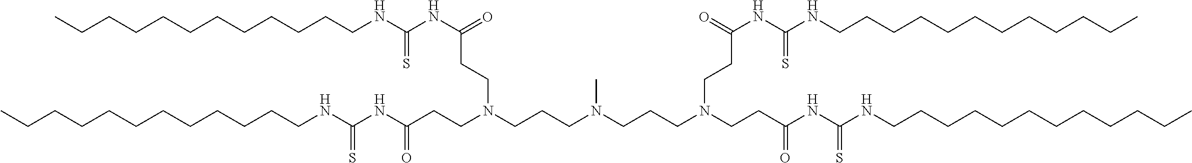 [SMILES: CCCCCCCCCCCCNC(=S)NC(=O)CCN(CCCN(C)CCCN(CCC(=O)NC(=S)NCCCCCCCCCCCC)CCC(=O)NC(=S)NCCCCCCCCCCCC)CCC(=O)NC(=S)NCCCCCCCCCCCC] |

Compounds provided herein are described with reference to both generic formula and specific compounds. In addition, the compounds of the present disclosure may exist in a number of different forms or derivatives, including but not limited to, stereoisomers, racemic mixtures, regioisomers, tautomers, salts, prodrugs, soft drugs, active metabolic derivatives (active metabolites), solvated forms, different crystal forms or polymorphs, all within the scope of the present disclosure.

The compounds of present disclosure can comprise one or more asymmetric centers, and thus can exist in various stereoisomeric forms, e.g., enantiomers and/or diastereomers. Thus, the compounds of present disclosure and compositions thereof may be in the form of an individual enantiomer, diastereomer or geometric isomer, or may be in the form of a mixture of stereoisomers. In certain embodiments, the compounds of the present disclosure are enantiopure compounds. In certain embodiments, mixtures of enantiomers or diastereomers are provided.

The term “enantiomer” refers to two stereoisomers of a compound which are non-superimposable mirror images of one another. The term “diastereomer” refers to a pair of optical isomers which are not mirror images of one another. Diastereomers have different physical properties, e.g., melting points, boiling points, spectral properties, and reactivities.

Furthermore, certain compounds, as described herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of enantiomers. In addition to the above-mentioned compounds per se, this disclosure also encompasses compositions comprising one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a stereoisomer may, in some embodiments, be provided substantially free of one or more corresponding stereoisomers, and may also be referred to as “stereochemically enriched”.

Where a particular enantiomer is preferred, it may, in some embodiments be provided substantially free of the opposite enantiomer, and may also be referred to as “optically enriched”. “Optically enriched”, as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high performance liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The compounds of the present disclosure may also exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵C, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The structures of the compounds in the examples are characterized by nuclear magnetic resonance (NMR) or/and liquid chromatography-mass spectrometry (LC-MS). NMR chemical shift (δ) is given in the unit of 10⁻⁶ (ppm). ¹H-NMR spectra is recorded in CDCl₃, CD₃OD or DMSO-d₆ solutions (reported in ppm) on a Bruker instrument (400 MHz or 500 MHz), using tetramethylsilane (TMS) as the reference standard (0.0 ppm).

Unless otherwise specified, the reactions of the present disclosure were typically done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

### Lipid Nanoparticles

The present disclosure also provides lipid particles comprising one or more of the lipids described above. Lipid particles include, but are not limited to, lipid nanoparticles (LNPs), liposomes, lipoplexes, and lipopolyplex (LPP). In some embodiments, the lipid particles are lipid nanoparticles. The present disclosure also provides a method for preparation of the lipid particles.

The lipid nanoparticles of the present disclosure may further comprise one or more additional lipids and/or other components such as a sterol. Other lipids may be included in the lipid nanoparticles of the present disclosure for a variety of purposes, such as to prevent lipid oxidation or to attach ligands onto the particle surface. Any lipid may be present in lipid nanoparticles of the present disclosure, including amphipathic, neutral, cationic, and anionic lipids, which can be used alone or in combination. Examples of additional lipid components that may be present are described below.

In some embodiments, the lipid nanoparticles comprise a lipid of the present disclosure (e.g., compound having Formula (I) or any compound of Table 1). In some embodiments, the lipid nanoparticles comprise two or more lipids of the present disclosure. In some embodiments, the lipid of the present disclosure (e.g., compound having Formula (I) or any compound of Table 1) is in a molar fraction of about 5 to 75%, about 10 to 75%, about 10 to 70%, about 10 to 65%, about 15 to 65%, about 20 to 65%, about 25 to 65%, about 30 to 65%, about 35 to 65%, about 40 to 65%, about 45 to 65%, about 40%-60%, about 40%-55% or about 40%-50% of the total lipids present in the lipid nanoparticle. In some embodiments, the lipid of the present disclosure (e.g., compound having Formula (I) or any compound of Table 1) is in a molar fraction of about 40-50% of the total lipids present in the lipid nanoparticle. In some embodiments, the lipid of the present disclosure (e.g., compound having Formula (I) or any compound of Table 1) is in a molar fraction of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64% or 65% of the total lipids present in the lipid nanoparticle.

In some embodiments, the lipid nanoparticles comprise a neutral lipid. The term “neutral lipid” refers to any of a number of lipid species that exist either in an uncharged or neutral zwitterionic form at a selected pH. In some embodiments, the neutral lipid is a phospholipid. Examples of phospholipid include, but not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-Distearoyl-sn-glycero-3-phosphorylethanolamine (DSPE), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (Cl6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any mixtures thereof. In some embodiments, the lipid nanoparticles comprise one neutral lipid. In some embodiments, the lipid nanoparticles comprise two or more neutral lipids. In some embodiments, the neutral lipid is selected from the group consisting of 1,2-dioleoyl-sn-glycero-3-phosphoethanolamine (DOPE), 1,2-distearoyl-sn-glycero-3-phosphocholine (DSPC), 1,2-dimyristoyl-sn-glycero-3-phosphocholine (DMPC), 1-palmitoyl-2-oleoyl-glycero-3-phosphocholine (POPC), 1,2-dioleoyl-sn-glycero-3-phosphocholine (DOPC), 1,2-dipalmitoyl-sn-glycero-3-phosphocholine (DPPC), and any mixtures thereof. In some embodiments, the neutral lipid is in a molar fraction of about 1 to 40%. In some embodiments, the neutral lipid is in a molar fraction of about 1 to 35%, about 5 to 30%, about 5 to 25%, about 5 to 20%, about 5 to 15% or about 5 to 10% of the total lipids present in the lipid nanoparticle. In some embodiments, the neutral lipid is in a molar fraction of about 1%, 2%, 3%, 4%, 5%, 6%, 7%, 8%, 9%, 10%, 11%, 12%, 15%, 20%, 25% or 30% of the total lipids present in the lipid nanoparticle.

In some embodiments, the lipid nanoparticles comprise a structural lipid. In some embodiments, the structural lipid is a sterol, a sterol derivative or any mixtures thereof. Examples of a sterol or a sterol derivative include, but are not limited to, cholesterol, fecosterol, sitosterol, beta-sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof. In some embodiments, the lipid nanoparticle comprises cholesterol. In some embodiments, the structural lipid is in a molar fraction of about 5 to 50% of the total lipids present in the lipid nanoparticle. In some embodiments, the structural lipid is in a molar fraction of about 10 to 50%, about 15 to 50%, about 25 to 50% or about 40 to 50% of the total lipids present in the lipid nanoparticle. In some embodiments, the structural lipid is in a molar fraction of about 10 to 45%, about 15 to 45%, about 25 to 45%, about 30 to 45% or about 35 to 45% of the total lipids present in the lipid nanoparticle. In some embodiments, the structural lipid is in a molar fraction of about 25%, 26%, 27%, 28%, 29%, 30%, 31%, 32%, 33%, 34%, 35%, 36%, 37%, 38%, 39%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4%, 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9% or 55% of the total lipids present in the lipid nanoparticle.

In some embodiments, the lipid nanoparticles comprise a lipid selected to reduce aggregation of lipid nanoparticles during formation, which may result from steric stabilization of particles which prevents charge-induced aggregation during formation. Examples of lipids that reduce aggregation of particles during formation include, but not limited to, polyethylene glycol (PEG)-modified lipids, monosialoganglioside Gm1, and polyamide oligomers (PAO). Other compounds with uncharged, hydrophilic, steric-barrier moieties, which prevent aggregation during formulation, like PEG or Gm1 can also be coupled to lipids for use as in the methods and compositions of the present disclosure. In some embodiments, lipids that reduce aggregation of particles during formation is a surfactant. In some embodiments, the surfactant is a PEG-modified lipid. Examples of PEG-modified lipid include, but not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, the PEG-modified lipid is 1,2-dimyristoyl-rac-glycero-3-methoxypolyethylene glycol (DMG-PEG), 1,2-distearoyl-rac-glycero-3-methoxypolyethylene glycol (DSG-PEG), N-(methylpolyoxyethylene-carbonyl)-1,2-distearoyl-sn-glycero-3-phosphoethanolamine (PEG-DSPE), 1,2-dioleoyl-sn-glycero-3-phosphoethanolamine-N-[(polyethylene glycol)](DOPE-PEG), or any mixtures thereof. Typically, the lipid component selected to reduce aggregation is in a molar fraction of about 1 to 15% of the total lipids present in the lipid nanoparticle. In some embodiments, the lipid component selected to reduce aggregation is in a molar fraction of about 1 to 10%, about 1 to 7%, about 1 to 5% or about 0.5 to 5% of the total lipids present in the lipid nanoparticle. In some embodiments, the lipid component selected to reduce aggregation is in a molar fraction of about 1 to 5%, about 1 to 2.5% or about 1.5 to 2% of the total lipids present in the lipid nanoparticle. In some embodiments, the lipid component selected to reduce aggregation is in a molar fraction of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or 4% of the total lipids present in the lipid nanoparticle.

As used herein, the term “PEG”, whether as part of another term or used independently, refers to polyethylene glycol. The term “PEGm” or “PEG-m”, wherein m is an integer, refers to a polyethylene glycol molecule or moiety with molecular weight of m. For example, “PEG2000” or “PEG-2000” refers to a polyethylene glycol molecule or moiety with molecular weight of 2000. In some embodiments, a PEG is a PEG700, PEG800, PEG900, PEG1000, PEG1100, PEG1200, PEG1300, PEG1400, PEG1500, PEG1600, PEG1700, PEG1800, PEG1900, PEG2000, PEG2100, PEG2200, PEG2300, PEG2400, PEG2500, PEG2600, PEG2700, PEG2800, PEG2900 or PEG3000. In some embodiments, a PEG is a PEG2000.

In some embodiments, the lipid nanoparticle of the present disclosure comprises: a compound having Formula (I) in a molar fraction of about 10-65%; a neutral lipid of the present disclosure in a molar fraction of about 5-30%; a structural lipid of the present disclosure in a molar fraction of about 15-50%; a lipid component selected to reduce aggregation of the present disclosure in a molar fraction of about 0.5-5%, based on the total lipids present in the lipid nanoparticle.

In some embodiments, the lipid nanoparticle of the present disclosure comprises: a compound having Formula (I) in a molar fraction of about 20-65%; a neutral lipid of the present disclosure in a molar fraction of about 5-25%; a structural lipid of the present disclosure in a molar fraction of about 25-50%; a lipid component selected to reduce aggregation of the present disclosure in a molar fraction of about 1-5%, based on the total lipids present in the lipid nanoparticle.

In some embodiments, the lipid nanoparticle of the present disclosure comprises: a compound having Formula (I) in a molar fraction of about 40-65%; a neutral lipid of the present disclosure in a molar fraction of about 5-15%; a structural lipid of the present disclosure in a molar fraction of about 25-50%; a lipid component selected to reduce aggregation of the present disclosure in a molar fraction of about 1-2.5%, based on the total lipids present in the lipid nanoparticle.

In some embodiments, the lipid nanoparticle of the present disclosure comprises: a compound having Formula (I) in a molar fraction of about 40-50%; a neutral lipid of the present disclosure in a molar fraction of about 5-15%; a structural lipid of the present disclosure in a molar fraction of about 40-50%; a lipid component selected to reduce aggregation of the present disclosure in a molar fraction of about 1.5-2%, based on the total lipids present in the lipid nanoparticle.

In some embodiments, the lipid nanoparticle of the present disclosure comprises:
a compound having Formula (I) in a molar fraction of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63% 64% or 65%;
a neutral lipid of the present disclosure in a molar fraction of about 1%, 2%, 3%, 4%, 5%, 6%, 7%, 8%, 9%, 10%, 11%, 12%, 15%, 20%, 25% or 30%;
a structural lipid of the present disclosure in a molar fraction of about 25%, 26%, 27%, 28%, 29%, 30%, 31%, 32%, 33%, 34%, 35%, 36%, 37%, 38%, 39%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4%, 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9% or 55%; and
a lipid component selected to reduce aggregation of the present disclosure in a molar fraction of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or 4%, based on the total lipids present in the lipid nanoparticle.

In some embodiments, the lipid nanoparticle of the present disclosure comprises components in molar fractions as listed in the table below.

| | | | A lipid component |
|---|---|---|---|
| A compound having | | A structural | selected to reduce |
| Formula (I) | A neutral lipid | lipid | aggregation |
| 49% | 8% | 40.7% | 2.3% |
| 46% | 8% | 43.9% | 2.1% |
| 46% | 12% | 39.7% | 2.3% |
| 50% | 10% | 38.5% | 1.5% |
| 40% | 6% | 52.4% | 1.6% |
| 40% | 8% | 50.1% | 1.9% |
| 43% | 6% | 49.1% | 1.9% |
| 43% | 8% | 47.4% | 1.6% |
| 46% | 6% | 45.9% | 2.1% |
| 46% | 8% | 43.6% | 2.4% |
| 46% | 10% | 42.4% | 1.6% |
| 46% | 12% | 40.1% | 1.9% |
| 49% | 6% | 42.6% | 2.4% |
| 49% | 8% | 40.9% | 2.1% |
| 49% | 12% | 37.4% | 1.6% |
| 44% | 7% | 47.5% | 1.5% |
| 45% | 6% | 47.5% | 1.5% |
| 45% | 8% | 45.3% | 1.7% |
| 47% | 7% | 44.4% | 1.6% |
| 47% | 8% | 43.5% | 1.5% |
| 46% | 8% | 44.5% | 1.5% |
| 46% | 8% | 43.9% | 2.1% |
| 43% | 8% | 47.4% | 1.6% |
| 46% | 10% | 42.4% | 1.6% |
| 45% | 8% | 45.3% | 1.7% |
| 47% | 7% | 44.4% | 1.6% |
| 45% | 6% | 47.4% | 1.6% |

Lipid nanoparticles may be characterized by a variety of methods. For example, microscopy (e.g., transmission electron microscopy (TEM) or scanning electron microscopy (SEM)) may be used to examine the morphology and size distribution of a lipid nanoparticle composition. Dynamic light scattering (DLS) or potentiometry (e.g., potentiometric titrations) may be used to measure zeta potentials. DLS may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential. The error of measurement by DLS may depend on various factor such as scattering angle and multiple scattering. The typical error of measurement by DLS is 5%.

The mean size of lipid nanoparticles may be between 10s of nm and 100s of nm, e.g., measured by DLS. For example, the mean size of lipid nanoparticles may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of lipid nanoparticles may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the mean size of lipid nanoparticles may be from about 70 nm to about 100 nm. In some embodiments, the mean size of lipid nanoparticles may be about 80 nm. In some embodiments, the mean size of lipid nanoparticles may be about 100 nm.

Lipid nanoparticles may be relatively homogenous. A polydispersity index (PDI) may be used to indicate the homogeneity of lipid nanoparticles, e.g., the particle size distribution of the lipid nanoparticles. A small (e.g., less than 0.3) PDI generally indicates a narrow particle size distribution. In some embodiments, the lipid nanoparticles of the present disclosure may have a PDI from about 0 to about 0.30, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29 or 0.30. In some embodiments, the PDI of the lipid nanoparticles of the present disclosure may be from about 0.05 to about 0.20.

The zeta potential of lipid nanoparticles may be used to indicate the electrokinetic potential. For example, the zeta potential may describe the surface charge of the lipid nanoparticles. Lipid nanoparticles with relatively low charges, positive or negative, are generally desirable, as more highly charged species may interact undesirably with cells, tissues, and other elements in the body. In some embodiments, the zeta potential of the lipid nanoparticles of the present disclosure may be from about −10 mV to about +25 mV, from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +25 mV, from about −5 mV to about +20 mV, from about −5 mV to about +15 mV, from about −5 mV to about +10 mV, from about −5 mV to about +5 mV, from about −5 mV to about 0 mV, from about 0 mV to about +25 mV, from about 0 mV to about +20 mV, from about 0 mV to about +15 mV, from about 0 mV to about +10 mV, from about 0 mV to about +5 mV, from about +5 mV to about +25 mV, from about +5 mV to about +20 mV, from about +5 mV to about +15 mV, or from about +5 mV to about +10 mV.

### Lipid Nanoparticle Compositions

In another aspect, the present disclosure also provides a lipid nanoparticle composition, which refers to a composition comprising one or more lipid particles as discussed above and a target polynucleotide that comprises a nucleic acid encoding a Respiratory Syncytial Virus antigenic polypeptide or a variant thereof. In some embodiments, the Respiratory Syncytial Virus antigenic polypeptide is Respiratory Syncytial Virus fusion protein (“RSV-F protein”). In some embodiments, the Respiratory Syncytial Virus antigenic polypeptide is Respiratory Syncytial Virus attachment protein (“RSV-G protein”). In some embodiments, the RSV-F protein is pre-fusion RSV-F protein. In some embodiments, the RSV-F protein is post-fusion RSV-F protein.

As used herein, the term “polynucleotide” refers to a polymer of nucleotide or nucleoside monomers consisting of naturally occurring bases, sugars and intersugar (backbone) linkages. The term “polynucleotide” also includes polymers comprising non-naturally occurring monomers, or portions thereof, which function similarly. Such modified or substituted polynucleotide are often preferred over native forms because of properties such as, for example, enhanced cellular uptake and increased stability in the presence of nucleases. As used herein, the term “nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Generally, the polynucleotide comprises more than 50 nucleotide or nucleoside monomers.

The polynucleotide that is present in a composition (e.g., a lipid nanoparticle composition comprising one or more target polynucleotides) of the present disclosure includes any form of nucleic acid that is known. The polynucleotide used herein include, but not limited to, single-stranded DNA or RNA, or double-stranded DNA or RNA, and DNA-RNA hybrids. Examples of double-stranded DNA include, but not limited to, structural genes, genes including control and termination regions, and self-replicating systems such as viral or plasmid DNA. Examples of double-stranded RNA include, but not limited to, siRNA and other RNA interference reagents. Single-stranded nucleic acids include, but not limited to, messenger RNA (mRNA), antisense oligonucleotides, ribozymes, microRNA, and triplex-forming oligonucleotides. The polynucleotide used herein also include nucleic acids containing known nucleotide analogs or modified backbone residues or linkages, which are synthetic, naturally occurring, or non-naturally occurring, and which have similar binding properties as the reference nucleic acid. Examples of such analogs include, but not limited to, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. The nucleic acid that is present in a composition of the present disclosure may include one or more modifications.

Nucleic acids of the present disclosure may be of various lengths, generally dependent upon the particular form of nucleic acid. In some embodiments, the nucleic acid has 10-5000 nucleotides in length. In some embodiments, the nucleic acid has about 4000 nucleotides in length. In some embodiments, the nucleic acid has about 3000 nucleotides in length. In some embodiments, the nucleic acid has about 2500 nucleotides in length. In some embodiments, the nucleic acid has about 2400, 2300, 2200, 2100, 2000, 1900, 1850, 1800, 1750, 1700, 1650, 1600, 1550, 1500, 1450, 1400, 1350, 1300, 1250, 1200, 1100, 1000,900, 800, 700, 600, 500, 400, 300, 250, 240, 230, 220, 210, 200, 190, 180, 170, 160, 150, 140, 130, 120, 110, 100, 90, 80, 70, 60 or 50 in length.

As used herein, the term “variant” of a polypeptide refers to molecules which differ in their amino acid sequence from a native or reference sequence. The amino acid sequence variants may possess substitutions, mutations, deletions, and/or insertions at certain positions within the amino acid sequence, as compared to a native or reference sequence. In some embodiments, variants possess at least 50%, or at least 60%, or at least 70% sequence identity to a native or reference sequence. In some embodiments, variants share at least 80% identity, or at least 85%, or at least 90% or at least 95%, or at least 96%, or at least 97%, or at least 98% sequence identity with a native or reference sequence.

In some embodiments, the target polynucleotide is encapsulated within an interior of the lipid nanoparticle. In some embodiments, the target polynucleotide is present within one or more lipid layers of the lipid nanoparticle. In some embodiments, the target polynucleotide is bound to the exterior or interior lipid surface of the lipid nanoparticle.

As used herein, “encapsulation”, “encapsulated”, “encapsulating” and “loaded”, and “associated” may refer to complete, substantial, or partial enclosure, confinement, surrounding, or encasement. As used herein, “encapsulation” or “association” may refer to the process of confining an individual nucleic acid within a lipid nanoparticle and/or establishing a physiochemical relationship between an individual nucleic acid and a lipid nanoparticle.

The “efficiency of encapsulation” of a target polynucleotide refers to the amount of target polynucleotide that is encapsulated or otherwise associated with a lipid nanoparticle after preparation, relative to the initial amount provided. The encapsulation efficiency is desirably high (e.g., more than 80%, more than 85%, more than 90% or more than 95%). The encapsulation efficiency may be measured, for example, by comparing the amount of target polynucleotide in a solution containing the lipid particle before and after breaking up the lipid nanoparticle with one or more organic solvents or detergents. Fluorescence may be used to measure the amount of free target polynucleotide (e.g., RNA) in a solution. In some embodiments, the encapsulation efficiency of a target polynucleotide may be at least 50%, for example, at least 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

The amount of a target polynucleotide in lipid nanoparticles may depend on the size, composition, desired target and/or application, or other properties of the lipid nanoparticles as well as on the properties of the target polynucleotide. For example, the amount of an RNA useful in a lipid particle may depend on the size, sequence, and other characteristics of the RNA. The relative amounts of a target polynucleotide and other elements (e.g., lipids) in a lipid nanoparticle may also vary. In some embodiments, the mass ratio of the lipids (e.g., cationic lipid, neutral lipid, structural lipid such as sterol and a lipid selected to reduce aggregation such as a surfactant) to a target polynucleotide (e.g., RNA) in a lipid nanoparticle composition may be from about 5:1 to about 60:1, such as about 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the mass ratio of lipids to a target polynucleotide may be from about 10:1 to about 50:1. In some embodiments, the mass ratio of lipids to a target polynucleotide is about 40:1. In some embodiments, the mass ratio of lipids to a target polynucleotide is about 20:1. In some embodiments, the mass ratio of lipids to a target polynucleotide is calculated by total mass of cationic lipid, neutral lipid, structural lipid such as sterol and a surfactant without solvents divided by mass of a dry target polynucleotide (e.g., RNA). The amount of a target polynucleotide in a lipid nanoparticle composition may, for example, be measured using absorption spectroscopy (e.g., ultraviolet-visible spectroscopy).

In some embodiments, a target polynucleotide is an RNA. In some embodiments, a target polynucleotide is a messenger RNA (mRNA). An mRNA may be naturally or may include modified and/or non-naturally occurring components such as one or more nucleobases, nucleosides, nucleotides, or linkers. In some embodiments, a target polynucleotide is an mRNA that comprises a nucleic acid encoding a RSV antigenic polypeptide or a variant thereof. In some embodiments, the RSV antigenic polypeptide is RSV-F protein. In some embodiments, the RSV-F protein is pre-fusion RSV-F protein. In some embodiments, the RSV-F protein is post-fusion RSV-F protein.

A “messenger RNA” (mRNA) refers to any polynucleotide that encodes at least one polypeptide (e.g., a naturally occurring, non-naturally occurring, or modified polymer of amino acids) and can be translated to produce the encoded polypeptide in vitro, in vivo, in situ or ex vivo. Translation of the mRNA, for example, in vivo translation of an mRNA inside a mammalian cell, may produce a polypeptide. A skilled person in the art will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

An mRNA of present disclosure can be transcribed in vitro from a template DNA. In vitro transcription of RNA is known in the art, and a person skilled in the art would easily and certainly obtain the mRNA sequence based on a provided template DNA sequence. In some embodiments, the RNA transcript is synthesized from a non-amplified, linear DNA template coding for the gene of interest via an enzymatic in vitro transcription reaction utilizing a T7 phage, RNA polymerase and nucleotide triphosphates of the desired chemistry.

An mRNA molecule may include a cap structure, a chain terminating nucleoside, a stem loop, a polyA sequence, and/or a polyadenylation signal. In some embodiments, the basic components of an mRNA molecule include at least a coding region, a 5′-untranslated region (5′-UTR), a 3′UTR and a polyA sequence. In some embodiments, a 5′ untranslated region (UTR) comprising a nucleic acid selected from the group consisting of SEQ ID NOs: 34, 37 and 70-71. In some embodiments, a 3′ untranslated region (UTR) comprising a nucleic acid selected from the group consisting of SEQ ID NOs: 35 and 72-74. In some embodiments, a poly-A region comprises 50-120 nucleotides in length. For example, a polyA region may contain 10, 20, 30, 40, 50, 60, 70, 80, 90, 100, 110 or 120 adenosine monophosphates. In some embodiments, the mRNA of present disclosure also includes a 5′ terminal cap. In some embodiments, a 5′ terminal cap can be selected from the group consisting of cap0GG, cap1GG and cap1AG. In some embodiments, the mRNA of present disclosure comprises one or more modified nucleotides selected from the group consisting of pseudouridine (Ψ), N1-methylpseudouridine (m1Ψ) 5-methyluridine (m5U), 2-thiouridine (s2U), 5-methylcytidine (m5C) and 5-methoxyuridine (5moU).

In some embodiments, the target polynucleotide encodes the Respiratory Syncytial Virus antigenic polypeptide comprising a) an amino acid sequence of amino acid residues 26-574 of SEQ ID NO: 38, or b) a mutant of (a) having at least one amino acid substitution selected from the group consisting of S55C, P102A, T103C, R106K, R109Q, R133Q, K134Q, R135Q, R136Q, L142Q, I148C, A149C, S155C, L188C, S190F, V207L, K209P, Q210P, Q210C, S211P, C212P, S290C, I379V, M447V, Y458C, L481P, V482P, Q501G and L512K according to SEQ ID NO 38, wherein the mutant has the Respiratory Syncytial Virus antigenic activity.

In some embodiments, the target polynucleotide also included a nucleic acid encoding a signal peptide linked to the Respiratory Syncytial Virus antigenic polypeptide. In some embodiments, the target polynucleotide also included termination sequences and linker encoding sequences. The term “signal peptide” used herein refers to a peptide comprising N-terminal 15-60 amino acids of a protein, which is typically needed for the translocation across the membrane on the secretory pathway and thus universally control the entry of most proteins both in eukaryotes and prokaryotes to the secretory pathway. Signal peptides generally include of three regions: an N-terminal region of differing length, which usually comprises positively charged amino acids; a hydrophobic region; and a short carboxy-terminal peptide region.

In some embodiments, the signal peptide used herein may comprises an amino acid sequence selected from the group consisting of SEQ ID NOs: 185, 187, 189, 191, 193, 195, 197, 199 and 201.

TABLE 2
Signal Peptides

| SEQ ID NO | Amino acid sequences |
|---|---|
| SEQ ID NO: 185 | MELLILKANAITTILTAVTFCFASG |
| SEQ ID NO: 187 | MDLLILKANAITTILTAVTFCFASG |
| SEQ ID NO: 189 | MELPILKANAITTILTAVTFCFASG |
| SEQ ID NO: 191 | MELLILKTNAITTILTAVTFCFASG |
| SEQ ID NO: 193 | MELLILKANAITAILTAVTFCFASG |
| SEQ ID NO: 195 | MELLILKANAITTIFTAVTFCFASG |
| SEQ ID NO: 197 | MELLILKANAITTILAAVTFCFASG |
| SEQ ID NO: 199 | MELLILKANAITTILTAVTLCFASG |
| SEQ ID NO: 201 | MELLILKANAITTILTAVTFCFASS |

In some embodiments, the signal peptide is encoded by a nucleic acid sequence selected from the group consisting of SEQ ID NOs: 184, 186, 188, 190, 192, 194, 196, 198 and 200.

TABLE 3
Signal Peptide Encoding Sequences

| SEQ ID NO | Nucleic acid sequences |
|---|---|
| SEQ ID NO: 184 | ATGGAACTGCTGATTCTGAAAGCGAACGCGATTACCACCATTCT |
| | GACCGCGGTGACCTTTTGCTTTGCGAGCGGC |
| SEQ ID NO: 186 | ATGGATCTGCTGATTCTGAAAGCGAACGCGATTACCACCATTCT |
| | GACCGCGGTGACCTTTTGCTTTGCGAGCGGC |
| SEQ ID NO: 188 | ATGGAACTGCCGATTCTGAAAGCGAACGCGATTACCACCATTCT |
| | GACCGCGGTGACCTTTTGCTTTGCGAGCGGC |
| SEQ ID NO: 190 | ATGGAACTGCTGATTCTGAAAACCAACGCGATTACCACCATTCT |
| | GACCGCGGTGACCTTTTGCTTTGCGAGCGGC |
| SEQ ID NO: 192 | ATGGAACTGCTGATTCTGAAAGCGAACGCGATTACCGCGATTCT |
| | GACCGCGGTGACCTTTTGCTTTGCGAGCGGC |
| SEQ ID NO: 194 | ATGGAACTGCTGATTCTGAAAGCGAACGCGATTACCACCATTTT |
| | TACCGCGGTGACCTTTTGCTTTGCGAGCGGC |
| SEQ ID NO: 196 | ATGGAACTGCTGATTCTGAAAGCGAACGCGATTACCACCATTCT |
| | GGCGGCGGTGACCTTTTGCTTTGCGAGCGGC |
| SEQ ID NO: 198 | ATGGAACTGCTGATTCTGAAAGCGAACGCGATTACCACCATTCT |
| | GACCGCGGTGACCCTGTGCTTTGCGAGCGGC |
| SEQ ID NO: 200 | ATGGAACTGCTGATTCTGAAAGCGAACGCGATTACCACCATTCT |
| | GACCGCGGTGACCTTTTGCTTTGCGAGCAGC |

In some embodiments, the target polynucleotide (e.g., mRNA) encodes the Respiratory Syncytial Virus antigenic polypeptide comprising an amino acid sequence of SEQ ID NO: 38 or SEQ ID NOs: 39-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183 and 287-370, or an amino acid sequence at least 95% (e.g., 96%, 97%, 98%, 99%, 99.5%, 99.6%, 99.7%, 99.8% or 99.9%) identical to SEQ ID NO: 38 or SEQ ID NOs: 39-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183 and 287-370.

In some embodiments, the target polynucleotide (e.g., mRNA) comprises a nucleic acid sequence of SEQ ID NOs: 1-33, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 182, 174, 176, 178, 180, 182 and 202-286, or a nucleic acid sequence at least 85% (e.g., 86%, 87%, 88%, 89%, 90%, 91%, 92%, 93%, 94%, 95%, 96%, 97%, 98%, 99%, 99.5%, 99.6%, 99.7%, 99.8% or 99.9%) identical to SEQ ID NOs: 1-33, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 182, 174, 176, 178, 180, 182 and 202-286 which encodes a polypeptide having Respiratory Syncytial Virus antigenic activity.

In some embodiments, the target polynucleotide (e.g., mRNA) comprises a nucleic acid of SEQ ID NOs: 2, 4, 14, 16, 18, 24, 26, 30, 31, 32 or 33, or a nucleic acid sequence at least 85% identical to SEQ ID NOs: 2, 4, 14, 16, 18, 24, 26, 30, 31, 32 or 33 which encodes a polypeptide having Respiratory Syncytial Virus antigenic activity.

In some embodiments, the target polynucleotide (e.g., mRNA) comprises a nucleic acid of SEQ ID NOs: 2, 4, 14, 16, 18, 24, 26, 30, 31, 32, 33, 203, 205, 215, 217, 219, 225, 227, 231, 232, 233 or 234, or a nucleic acid sequence at least 85% (e.g., 86%, 87%, 88%, 89%, 90%, 91%, 92%, 93%, 94%, 95%, 96%, 97%, 98%, 99%, 99.5%, 99.6%, 99.7%, 99.8% or 99.9%) identical to SEQ ID NOs: 2, 4, 14, 16, 18, 24, 26, 30, 31, 32, 33, 203, 205, 215, 217, 219, 225, 227, 231, 232, 233 or 234 which encodes a polypeptide having Respiratory Syncytial Virus antigenic activity.

In some embodiments, the target polynucleotide (e.g., mRNA) comprises:
(i) a 5′ untranslated region (UTR) comprising a nucleic acid selected from the group consisting of SEQ ID NOs: 34, 37 and 70-71; and/or
(ii) a 3′ untranslated region (UTR) comprising a nucleic acid of selected from the group consisting of SEQ ID NOs: 35 and 72-74; and/or
(iii) a poly-A region of 50-120 nucleotides in length or a poly-A region comprising a nucleic acid of selected from the group consisting of SEQ ID NOs: 36 and 75-79.

TABLE 4
UTRs and PolyA
5'UTR

| | |
|---|---|
| SEQ ID NO: 34 | ACCGCCGAGACCGCGTCCGCCCCGCGAGCACAGAGCCTCGCCTTTG |
| | CCGATCCGCCGCCCGTCCACACCCGCCGCCAGCTCACC |
| SEQ ID NO: 37 | GGCTAGCGCCGCCACC |
| SEQ ID NO: 70 | ACATTTGCTTCTGACACAACTGTGTTCACTAGCAACCTCAAACAGA |
| | CACC |
| SEQ ID NO: 71 | GGGAAATAAGAGAGAAAAGAAGAGTAAGAAGAAATATAAGAGCCAC |
| | C |

3'UTR

| | |
|---|---|
| SEQ ID NO: 35 | GCTCGCTTTCTTGCTGTCCAATTTCTATTAAAGGTTCCTTTGTTCC |
| | CTAAGTCCAACTACTAAACTGGGGGATATTATGAAGGGCCTTGAGC |
| | ATCTGGATTCTGCCTAATAAAAAACATTTATTTTCATTGCAATTGC |
| | CATGTGTATGTGGGTTCGCCCACATACTCTGATGATCCCCAATCGT |
| | GGCGTGTCGGCCTGCTTCGGCAGGCACTGGCGCCGGGATCATTCAT |
| | GGCAA |
| SEQ ID NO: 72 | GCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAAT |
| | AAAGTCTGAGTGGGCGGCA |
| SEQ ID NO: 73 | GCTCGCTTTCTTGCTGTCCAATTTCTATTAAAGGTTCCTTTGTTCC |
| | CTAAGTCCAACTACTAAACTGGGGGATATTATGAAGGGCCTTGAGC |
| | ATCTGGATTCTGCCTAATAAAAAACATTTATTTTCATTGC |
| SEQ ID NO: 74 | GCTCGCTTTCTTGCTGTCCAATTTCTATTAAAGGTTCCTTTGTTCC |
| | CTAAGTCCAACTACTAAACTGGGGGATATTATGAAGGGCCTTGAGC |
| | ATCTGGATTCTGCCTAATAAAAAACATTTATTTTCATTGCGCTCGC |
| | TTTCTTGCTGTCCAATTTCTATTAAAGGTTCCTTTGTTCCCTAAGT |
| | CCAACTACTAAACTGGGGGATATTATGAAGGGCCTTGAGCATCTGG |
| | ATTCTGCCTAATAAAAAACATTTATTTTCATTGC |

PolyA

| | |
|---|---|
| SEQ ID NO: 36 | AAAAAAAAAAAAAAAAAAAAAAAAAAAAAAAAAAAAAAAACTGGAT |
| | TCTGAAAAAAAAAAAAAAAAAAAAAAAAAAAAAAAAAAAAAAAAAA |
| | AAAAAAAAAAAAAAAA |
| SEQ ID NO: 75 | AAAAAAAAAAAAAAAAAAAAAAAAAAAAAAAAAAAAAAAAAAAAAA |
| | AAAAAAAAAAAAAA |
| SEQ ID NO: 76 | AAAAAAAAAAAAAAAAAAAAAAAAAAAAAAAAAAAAAAAAAAAAAA |
| | AAAAAAAAAAAAAAAAAAAAAAAAAAAAAAAAAA |
| SEQ ID NO: 77 | AAAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAAA |
| | AAAAAAAA |
| SEQ ID NO: 78 | AAAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAAA |
| | AAAAAAAAAAAAAAAAAAAAAAAAAAAA |
| SEQ ID NO: 79 | AAAAAAAAAAAAAAAAAAAAAAAAAAAAAAAAAAAAAAAACTGGAT |
| | TCTGAAAAAAAAAAAAAAAAAAAAAAAAAAAAAAAAAAAAAAAAAA |
| | AAAAAAAAAAAAAAAAAA |

### Respiratory Syncytial Virus Polypeptide Mutants

In another aspect, the present disclosure provides Respiratory Syncytial Virus polypeptide mutant useful in inducing an antigen specific immune response.

In some embodiments, the Respiratory Syncytial Virus polypeptide mutant comprises an amino acid sequence of amino acid residues 26-574 of SEQ ID NO: 38 with at least one amino acid residue substitution to the amino acid sequence of amino acid residues 26-574 of SEQ ID NO: 38, wherein the at least one amino acid substitution is selected from the group consisting of S55C, P102A, T103C, R106K, R109Q, R133Q, K134Q, R135Q, R136Q, L142Q, I148C, A149C, S155C, L188C, S190F, V207L, K209P, Q210P, Q210C, S211P, C212P, S290C, I379V, M447V, Y458C, L481P, V482P, Q501G and L512K according to SEQ ID NO 38, wherein the mutant has the Respiratory Syncytial Virus antigenic activity.

In some embodiments, the Respiratory Syncytial Virus polypeptide mutant comprises at least two amino acid residue substitution to amino acid sequence of amino acid residues 26-574 of SEQ ID NO: 38, wherein the at least one amino acid substitution is selected from the group consisting of S55C, P102A, T103C, R106K, R109Q, R133Q, K134Q, R135Q, R136Q, L142Q, I148C, A149C, S155C, L188C, S190F, V207L, K209P, Q210P, Q210C, S211P, C212P, S290C, I379V, M447V, Y458C, L481P, V482P, Q501G and L512K according to SEQ ID NO 38, wherein the mutant has the Respiratory Syncytial Virus antigenic activity.

In some embodiments, the Respiratory Syncytial Virus polypeptide mutant comprises an amino acid sequence of amino acid residues 26-574 of SEQ ID NO: 38, and wherein the amino acid sequence comprises one or more amino acid residues substitutions and/or one or more deletions of amino acid residues as below:
a) a 27-amino acid residues fragment from E110 to R136 was substituted by residues -GS-
b) a 45-amino acid residues fragment from E110 to V154 was substituted by residues -GS-;
c) a 50-amino acid residues fragment from I525 to N574 was substituted by residues GYIPEAPRDGQAYVRKDGEWVLLSTFL;
d) a deletion of a 18-amino acid residues fragment from F137 to V154;
e) a deletion of a 24-amino acid residues fragment from K551 to N574;
f) a deletion of a 50-amino acid residues fragment from I525 to N574.

In some embodiments, the Respiratory Syncytial Virus polypeptide mutant comprises at least one amino acid residue substitution to amino acid sequence of amino acid residues 26-574 of SEQ ID NO: 38, comprising amino acid substitutions combination selected from the group consisting of:
i) K209P and Q210P;
ii) Q210P and S211P;
iii) S211P and C212P;
iv) L481P;
v) L481P and V482P;
vi) K209P, Q210P, L481P and V482P;
vii) Q210P, S211P, L481P and V482P;
viii) S211P, C212P, L481P and V482P;
ix) K209P, Q210P, and L481P;
x) Q210P, S211P, and L481P;
xi) S211P, C212P, and L481P;
xii) S55C, P102A, T103C, I148C, L188C, V207L Q210P, I379V and M447V;
xiii) S55C, P102A, T103C, I148C, L188C, Q210P, I379V and M447V; xiv) P102A, T103C, I148C, V207L, Q210P, I379V and M447V; xv) S55C, P102A, L188C, V207L Q210P, I379V and M447V;
xvi) P102A, T103C, I148C, Q210P, I379V and M447V;
xvii) S55C, P102A, L188C, Q210P, I379V and M447V;
xviii) P102A, V207L, Q210P, I379V and M447V;
xix) V207L and Q210P;
xx) S55C, P102A, T103C, I148C, L188C, V207L, Q210C, S213C, I379V and M447V;
xxi) S55C, P102A, T103C, I148C, L188C, Q210C, S213C, I379V and M447V;
xxii) P102A, T103C, I148C, V207L, Q210C, S213C, I379V and M447V;
xxiii) S55C, P102A, L188C, V207L, Q210C, S213C, I379V and M447V;
xxiv) P102A, T103C, I148C, Q210C, S213C, I379V and M447V;
xxv) S55C, P102A, L188C, Q210C, S213C, I379V and M447V;
xxvi) P102A, V207L, Q210C, S213C, I379V and M447V;
xxvii) V207L, Q210C and S213C;
xxviii) S55C, P102A, L188C, I379V and M447V;
xxix) P102A, T103C, I148C, I379V and M447V;
xxx) P102A, I379V and M447V
xxxi) P102A and M447V;
xxxii) E30V, P102A, I379V and M447V;
xxxiii) T54H, P102A, I379V and M447V;
xxxiv) N88S, P102A, I379V and M447V;
xxxv) P102A, T103A, I379V and M447V;
xxxvi) P102A, A122T, I379V and M447V;
xxxvii) P102A, K124N, I379V and M447V;
xxxviii) P102A, T125N, I379V and M447V;
xxxix) P102A, R136K, I379V and M447V;
xxxx) P102A, V152I, I379V and M447V;
xxxxi) P102A, S190I, I379V and M447V;
xxxxii) P102A, N227S, I379V and M447V;
xxxxiii) P102A, V296I, I379V and M447V;
xxxxiv) P102A, Q354L, I379V and M447V;
xxxxv) P102A, L373R, I379V and M447V;
xxxxvi) P102A, I379V, M447V and D486S;
xxxxvii) P102A, I379V, M447V and S540L;
xxxxviii) P102A, I379V, M447V and L547F;
xxxxix) S155C, P102A, S290C, I379V and M447V;
xxxxx) P102A, A149C, I379V, M447V and Y458C;
xxxxxi) a 27-amino acid residues fragment from E110 to R136 was substituted by residues -GS-, and a 50-amino acid residues fragment from I525 to N574 was substituted by residues GYIPEAPRDGQAYVRKDGEWVLLSTFL;
xxxxxii) a 27-amino acid residues fragment from E110 to R136 was substituted by residues -GS-, and a deletion of a 24-amino acid residues fragment from K551 to N574;
xxxxxiii) a deletion of a 18-amino acid residues fragment from F137 to V154, and a 50-amino acid residues fragment from I525 to N574 was substituted by residues GYIPEAPRDGQAYVRKDGEWVLLSTFL;
xxxxxiv) a deletion of a 18-amino acid residues fragment from F137 to V154, and a deletion of a 50-amino acid residues fragment from I525 to N574;
xxxxxv) a deletion of a 18-amino acid residues fragment from F137 to V154;
xxxxxvi) a 45-amino acid residues fragment from E110 to V154 was substituted by residues -GS-, and a 50-amino acid residues fragment from I525 to N574 was substituted by residues GYIPEAPRDGQAYVRKDGEWVLLSTFL;
xxxxxvii) a 45-amino acid residues fragment from E110 to V154 was substituted by residues -GS-, and a deletion of a 24-amino acid residues fragment from K551 to N574; and
xxxxxviii) a 45-amino acid residues fragment from E110 to V154 was substituted by residues -GS-.

In some embodiments, the Respiratory Syncytial Virus polypeptide mutant comprises an amino acid sequence selected from the group consisting of SEQ ID NOs: 287-370.

Further provided herein are Respiratory Syncytial Virus polypeptides comprising a suitable signal peptide and Respiratory Syncytial Virus polypeptide mutant. The signal peptide may be fused to the N- or C-terminal of the Respiratory Syncytial Virus polypeptide mutant. In some embodiments, the signal peptide comprises an amino acid sequence selected from the group consisting of SEQ ID NOs: 185, 187, 189, 191, 193, 195, 197, 199 and 201. In some embodiments, the Respiratory Syncytial Virus polypeptide mutant comprises an amino acid sequence selected from the group consisting of SEQ ID NOs: 39-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183.

### Nucleic Acids

In another aspect, the present disclosure provides nucleic acid useful in inducing an antigen specific immune response. In some embodiments, the nucleic acid of the present disclosure is an isolated nucleic acid sequence.

In another aspect, the present disclosure provides an isolated nucleic acid sequence encoding a Respiratory Syncytial Virus polypeptide comprising an amino acid sequence of selected from the group consisting of SEQ ID NOs: 39-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183 and 287-370.

In some embodiments, the isolated nucleic acid comprises a nucleic acid sequence selected from the group consisting of SEQ ID NOs: 202-286. In some embodiments, the isolated nucleic acid comprises a nucleic acid sequence having at least 85% (e.g., 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%) sequence identity to a nucleic acid sequence selected from the group consisting of SEQ ID NOs: 202-286.

In some embodiments, the isolated nucleic acid also includes a nucleic acid encoding a signal peptide linked to the Respiratory Syncytial Virus polypeptide. In some embodiments, the isolated nucleic acid also includes termination sequences and linker encoding sequences. In some embodiments, the signal peptide encoding nucleic acid sequence comprises a nucleic acid sequence selected from the group consisting of SEQ ID NOs: 184, 186, 188, 190, 192, 194, 196, 198 and 200. In some embodiments, the isolated nucleic acid comprises a nucleic acid sequence selected from the group consisting of SEQ ID NOs: 1-33, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 182, 174, 176, 178, 180 and 182.

### Sequences

TABLE 5
Nucleic Acid Sequences and Amino acid Sequences of RSV
RSV-WT (v1) (RSV-F Protein)

| | |
|---|---|
| SEQ ID | CAGAACATTACCGAAGAATTTTATCAGAGCACCTGCAGCGCGGTGAGCAAAGGCT |
| NO: 202 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAAC |
| SEQ ID | QNITEEFYQSTCSAVSKGYLSALRTGWYTSVITIELSNIKENKCNGTDAKVKLIKQELD |
| NO: 287 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN |

RSV-WT (v2) (RSV-F Protein)

| | |
|---|---|
| SEQ ID | CAGAACATTACCGAAGAATTTTATCAGAGCACCTGCAGCGCGGTGAGCAAAGGCT |
| NO: 203 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAAC |
| SEQ ID | QNITEEFYQSTCSAVSKGYLSALRTGWYTSVITIELSNIKENKCNGTDAKVKLIKQELD |
| NO: 287 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence (wild | LSLINDMPITNDQKKLMSNNVQIVRQQSYSIMSIIKEEVLAYVVQLPLYGVIDTPCWK |
| type RSV-F | LHTSPLCTTNTKEGSNICLTRTDRGWYCDNAGSVSFFPQAETCKVQSNRVFCDTMNS |
| protein) | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-P1

| | |
|---|---|
| SEQ ID | CAGAACATTACCGAAGAATTTTATCAGAGCACCTGCAGCGCGGTGAGCAAAGGCT |
| NO: 204 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACCCGCCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 288 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNPPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPLVFPSDEFDA |
| | SISQVNEKINQSLAFIRKSDELLHNVNAGKSTTNIMITTIIIVIIVILLSLIAVGLLLYCKA |
| | RSTPVTLSKDQLSGINNIAFSN |

RSV-P2

| | |
|---|---|
| SEQ ID | CAGAACATTACCGAAGAATTTTATCAGAGCACCTGCAGCGCGGTGAGCAAAGGCT |
| NO: 205 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACCGCCGTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 289 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKPPCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPLVFPSDEFDA |
| | SISQVNEKINQSLAFIRKSDELLHNVNAGKSTTNIMITTIIIVIIVILLSLIAVGLLLYCKA |
| | RSTPVTLSKDQLSGINNIAFSN |

RSV-P3

| | |
|---|---|
| SEQ ID | CAGAACATTACCGAAGAATTTTATCAGAGCACCTGCAGCGCGGTGAGCAAAGGCT |
| NO: 206 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGCCGCCGAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 290 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQPPSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-P4

| | |
|---|---|
| SEQ ID | CAGAACATTACCGAAGAATTTTATCAGAGCACCTGCAGCGCGGTGAGCAAAGGCT |
| NO: 207 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 291 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPPVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-P5

| | |
|---|---|
| SEQ ID | CAGAACATTACCGAAGAATTTTATCAGAGCACCTGCAGCGCGGTGAGCAAAGGCT |
| NO: 208 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGCCGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 292 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPPPFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN |

RSV-P6

| | |
|---|---|
| SEQ ID | CAGAACATTACCGAAGAATTTTATCAGAGCACCTGCAGCGCGGTGAGCAAAGGCT |
| NO: 209 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACCCGCCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGCCGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 293 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNPPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPPPFPSDEFDAS |
| | ISQVNEKINQSLAFIRKSDELLHNVNAGKSTTNIMITTIIIVIIVILLSLIAVGLLLYC |
| | KARSTPVTLSKDQLSGINNIAFSN |

RSV-P7

| | |
|---|---|
| SEQ ID | CAGAACATTACCGAAGAATTTTATCAGAGCACCTGCAGCGCGGTGAGCAAAGGCT |
| NO: 210 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACCGCCGTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGCCGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 294 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKPPCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPPPFPSDEFDAS |
| | ISQVNEKINQSLAFIRKSDELLHNVNAGKSTTNIMITTIIIVIIVILLSLIAVGLLLY |
| | CKARSTPVTLSKDQLSGINNIAFSN |

RSV-P8

| | |
|---|---|
| | CAGAACATTACCGAAGAATTTTATCAGAGCACCTGCAGCGCGGTGAGCAAAGGCT |
| SEQ ID | ATCTGAGCGCGCTGCGCACCGGCTGGTATACCAGCGTGATTACCATTGAACTGAG |
| NO: 211 | CAACATTAAAGAAAACAAATGCAACGGCACCGATGCGAAAGTGAAACTGATTAA |
| mRNA | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGCCGCCGAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGCCGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 295 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQPPSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPPPFPSDEFD |
| | ASISQVNEKINQSLAFIRKSDELLHNVNAGKSTTNIMITTIIIVIIVILLSLIAVGL |
| | LLYCKARSTPVTLSKDQLSGINNIAFSN |

RSV-P9

| | |
|---|---|
| SEQ ID | CAGAACATTACCGAAGAATTTTATCAGAGCACCTGCAGCGCGGTGAGCAAAGGCT |
| NO: 212 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACCCGCCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 296 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNPPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPPVFPSDEFDA |
| | SISQVNEKINQSLAFIRKSDELLHNVNAGKSTTNIMITTIIIVIIVILLSLIAVGLL |
| | LYCKARSTPVTLSKDQLSGINNIAFSN |

RSV-P10

| | |
|---|---|
| SEQ ID | CAGAACATTACCGAAGAATTTTATCAGAGCACCTGCAGCGCGGTGAGCAAAGGCT |
| NO: 213 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACCGCCGTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 297 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKPPCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPPVFPSDEFDA |
| | SISQVNEKINQSLAFIRKSDELLHNVNAGKSTTNIMITTIIIVIIVILLSLIAVGLLL |
| | YCKARSTPVTLSKDQLSGINNIAFSN |

RSV-P11

| | |
|---|---|
| SEQ ID | CAGAACATTACCGAAGAATTTTATCAGAGCACCTGCAGCGCGGTGAGCAAAGGCT |
| NO: 214 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGCCGCCGAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CCGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 298 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQPPSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPPVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-T-S-V

| | |
|---|---|
| SEQ ID | CAGAACATTACCGAAGAATTTTATCAGAGCACCTGCAGCGCGGTGAGCAAAGGCT |
| NO: 215 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTCT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 299 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVCTSKVLDLKN |
| amino acid | YIDKQLLPILNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-T-S

| | |
|---|---|
| SEQ ID | CAGAACATTACCGAAGAATTTTATCAGAGCACCTGCAGCGCGGTGAGCAAAGGCT |
| NO: 216 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTGT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 300 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVCTSKVLDLKN |
| amino acid | YIDKQLLPIVNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-T-V

| | |
|---|---|
| SEQ ID | CAGAACATTACCGAAGAATTTTATCAGAGCACCTGCAGCGCGGTGAGCAAAGGCT |
| NO: 217 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 301 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVLTSKVLDLKN |
| amino acid | YIDKQLLPILNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-S-V

| | |
|---|---|
| SEQ ID | CAGAACATTACCGAAGAATTTTATCAGAGCACCTGCAGCGCGGTGAGCAAAGGCT |
| NO: 218 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTCT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 302 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVCTSKVLDLKN |
| amino acid | YIDKQLLPILNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-T

| | |
|---|---|
| SEQ ID | CAGAACATTACCGAAGAATTTTATCAGAGCACCTGCAGCGCGGTGAGCAAAGGCT |
| NO: 219 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 303 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVLTSKVLDLKN |
| amino acid | YIDKQLLPIVNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGL |
| | LLYCKARSTPVTLSKDQLSGINNIAFSN |

RSV-210-S

| | |
|---|---|
| SEQ ID | CAGAACATTACCGAAGAATTTTATCAGAGCACCTGCAGCGCGGTGAGCAAAGGCT |
| NO: 220 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTGT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 304 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVCTSKVLDLKN |
| amino acid | YIDKQLLPIVNKPSCSISNIETVIEFQQKNNRLLEITREFSVNAGVTTPVSTYMLTNSE |
| sequence | LLSLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN |

RSV-210-V-3

| | |
|---|---|
| SEQ ID | CAGAACATTACCGAAGAATTTTATCAGAGCACCTGCAGCGCGGTGAGCAAAGGCT |
| NO: 221 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 305 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPILNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGL |
| | LLYCKARSTPVTLSKDQLSGINNIAFSN |

RSV-210-V

| | |
|---|---|
| SEQ ID | CAGAACATTACCGAAGAATTTTATCAGAGCACCTGCAGCGCGGTGAGCAAAGGCT |
| NO: 222 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAACCGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 306 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKNYI |
| amino acid | DKQLLPILNKPSCSISNIETVIEFQQKNNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEINLCNVDIFNPKYDCKIMTSKTDVSSSVITSLGAIVSCYGKTKCTASNKNRGIIK |
| | TFSNGCDYVSNKGMDTVSVGNTLYYVNKQEGKSLYVKGEPIINFYDPLVFPSDEFDA |
| | SISQVNEKINQSLAFIRKSDELLHNVNAGKSTTNIMITTIIIVIIVILLSLIAVGLLL |
| | YCKARSTPVTLSKDQLSGINNIAFSN |

RSV-210-213-T-S-V

| | |
|---|---|
| SEQ ID | CAGAACATTACCGAAGAATTTTATCAGAGCACCTGCAGCGCGGTGAGCAAAGGCT |
| NO: 223 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTCT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 307 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVCTSKVLDLKN |
| amino acid | YIDKQLLPILNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN |

RSV-210-213-T-S

| | |
|---|---|
| SEQ ID | CAGAACATTACCGAAGAATTTTATCAGAGCACCTGCAGCGCGGTGAGCAAAGGCT |
| NO: 224 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTGT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 308 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVCTSKVLDLKN |
| amino acid | YIDKQLLPIVNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN |

RSV-210-213-T-V

| | |
|---|---|
| SEQ ID | CAGAACATTACCGAAGAATTTTATCAGAGCACCTGCAGCGCGGTGAGCAAAGGCT |
| NO: 225 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 309 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVLTSKVLDLKN |
| amino acid | YIDKQLLPILNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-213-S-V

| | |
|---|---|
| SEQ ID | CAGAACATTACCGAAGAATTTTATCAGAGCACCTGCAGCGCGGTGAGCAAAGGCT |
| NO: 226 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTCT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 310 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVCTSKVLDLKN |
| amino acid | YIDKQLLPILNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-213-T

| | |
|---|---|
| SEQ ID | CAGAACATTACCGAAGAATTTTATCAGAGCACCTGCAGCGCGGTGAGCAAAGGCT |
| NO: 227 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 311 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVLTSKVLDLKN |
| amino acid | YIDKQLLPIVNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-213-S

| | |
|---|---|
| | CAGAACATTACCGAAGAATTTTATCAGAGCACCTGCAGCGCGGTGAGCAAAGGCT |
| SEQ ID | ATCTGAGCGCGCTGCGCACCGGCTGGTATACCTGCGTGATTACCATTGAACTGAG |
| NO: 228 | CAACATTAAAGAAAACAAATGCAACGGCACCGATGCGAAAGTGAAACTGATTAA |
| mRNA | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTGT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 312 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVCTSKVLDLKN |
| amino acid | YIDKQLLPIVNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-213-V-3

| | |
|---|---|
| SEQ ID | CAGAACATTACCGAAGAATTTTATCAGAGCACCTGCAGCGCGGTGAGCAAAGGCT |
| NO: 229 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 313 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPILNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-210-213-V

| | |
|---|---|
| SEQ ID | CAGAACATTACCGAAGAATTTTATCAGAGCACCTGCAGCGCGGTGAGCAAAGGCT |
| NO: 230 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAATGCAGCTGCTGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 314 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPILNKCSCCISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLL |
| | YCKARSTPVTLSKDQLSGINNIAFSN |

RSV-S55C + L188C

| | |
|---|---|
| SEQ ID | CAGAACATTACCGAAGAATTTTATCAGAGCACCTGCAGCGCGGTGAGCAAAGGCT |
| NO: 231 | ATCTGAGCGCGCTGCGCACCGGCTGGTATACCTGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGTGCA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTCVITIELSNIKENKCNGTDAKVKLIKQELD |
| NO: 315 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVCTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGL |
| | LLYCKARSTPVTLSKDQLSGINNIAFSN |

RSV-T103C + I148C

| | |
|---|---|
| SEQ ID | CAGAACATTACCGAAGAATTTTATCAGAGCACCTGCAGCGCGGTGAGCAAAGGCT |
| NO: 232 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGTGCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGTGCGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 316 | KYKNAVTELQLLMQSTPACNNRARRELPRFMNYTLNNAKKTNVTLSKKRKRRFLGF |
| Corresponding | LLGVGSACASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGL |
| | LLYCKARSTPVTLSKDQLSGINNIAFSN |

RSV-P102A + I379V + M447V

| | |
|---|---|
| SEQ ID | CAGAACATTACCGAAGAATTTTATCAGAGCACCTGCAGCGCGGTGAGCAAAGGCT |
| NO: 233 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 317 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-V207L

| | |
|---|---|
| SEQ ID | CAGAACATTACCGAAGAATTTTATCAGAGCACCTGCAGCGCGGTGAGCAAAGGCT |
| NO: 234 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTCT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 318 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPILNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-R106K

| | |
|---|---|
| SEQ ID | CAGAACATTACCGAAGAATTTTATCAGAGCACCTGCAGCGCGGTGAGCAAAGGCT |
| NO: 235 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACAAAGCGCGCCGCGAACTGCCGCGCTTTATGAA |
| | CTATACCCTGAACAACGCGAAAAAAACCAACGTGACCCTGAGCAAAAAACGCAA |
| | ACGCCGCTTTCTGGGCTTTCTGCTGGGCGTGGGCAGCGCGATTGCGAGCGGCGTG |
| | GCGGTGAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACATTATGATTACCACCATTATTATTGTGATTAT |
| | TGTGATTCTGCTGAGCCTGATTGCGGTGGGCCTGCTGCTGTATTGCAAAGCGCGC |
| | AGCACCCCGGTGACCCTGAGCAAAGATCAGCTGAGCGGCATTAACAACATTGCGT |
| | TTAGCAACTGATAA |
| SEQ ID | QNITEEFYQSTCSAVSKGYLSALRTGWYTSVITIELSNIKENKCNGTDAKVKLIKQELD |
| NO: 319 | KYKNAVTELQLLMQSTPPTNNKARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-R109Q

| | |
|---|---|
| SEQ ID | CAGAACATTACCGAAGAATTTTATCAGAGCACCTGCAGCGCGGTGAGCAAAGGCT |
| NO: 236 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCAGGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 320 | KYKNAVTELQLLMQSTPPTNNRARQELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-R133Q

| | |
|---|---|
| SEQ ID | CAGAACATTACCGAAGAATTTTATCAGAGCACCTGCAGCGCGGTGAGCAAAGGCT |
| NO: 237 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACAGAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 321 | KYKNAVTELQLLMQSTPPTNNRARRELPRFMNYTLNNAKKTNVTLSKKQKRRFLGF |
| Corresponding | LLGVGSAIASGVAVSKVLHLEGEVNKIKSALLSTNKAVVSLSNGVSVLTSKVLDLKNY |
| amino acid | IDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGI |
| | IKTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN |

RSV-K134Q

| | |
|---|---|
| SEQ ID | CAGAACATTACCGAAGAATTTTATCAGAGCACCTGCAGCGCGGTGAGCAAAGGCT |
| NO: 238 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCCAG |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 322 | KYKNAVTELQLLMQSTPPTNNRARRELPRFMNYTLNNAKKTNVTLSKKRQRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGL |
| | LLYCKARSTPVTLSKDQLSGINNIAFSN |

RSV-R135Q

| | |
|---|---|
| SEQ ID | CAGAACATTACCGAAGAATTTTATCAGAGCACCTGCAGCGCGGTGAGCAAAGGCT |
| NO: 239 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CAGCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 323 | KYKNAVTELQLLMQSTPPTNNRARRELPRFMNYTLNNAKKTNVTLSKKRKQRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLL |
| | LYCKARSTPVTLSKDQLSGINNIAFSN** |

RSV-R136Q

| | |
|---|---|
| SEQ ID | CAGAACATTACCGAAGAATTTTATCAGAGCACCTGCAGCGCGGTGAGCAAAGGCT |
| NO: 240 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCAGTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 324 | KYKNAVTELQLLMQSTPPTNNRARRELPRFMNYTLNNAKKTNVTLSKKRKRQFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-L142Q

| | |
|---|---|
| SEQ ID | CAGAACATTACCGAAGAATTTTATCAGAGCACCTGCAGCGCGGTGAGCAAAGGCT |
| NO: 241 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCAGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 325 | KYKNAVTELQLLMQSTPPTNNRARRELPRFMNYTLNNAKKTNVTLSKKRKRRFLGF |
| Corresponding | LQGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-A149C

| | |
|---|---|
| SEQ ID | CAGAACATTACCGAAGAATTTTATCAGAGCACCTGCAGCGCGGTGAGCAAAGGCT |
| NO: 242 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTTGCAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 326 | KYKNAVTELQLLMQSTPPTNNRARRELPRFMNYTLNNAKKTNVTLSKKRKRRFLGF |
| Corresponding | LLGVGSAICSGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-S155C

| | |
|---|---|
| SEQ ID | CAGAACATTACCGAAGAATTTTATCAGAGCACCTGCAGCGCGGTGAGCAAAGGCT |
| NO: 243 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGTGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 327 | KYKNAVTELQLLMQSTPPTNNRARRELPRFMNYTLNNAKKTNVTLSKKRKRRFLGF |
| Corresponding | LLGVGSAIASGVAVCKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-S190F

| | |
|---|---|
| SEQ ID | CAGAACATTACCGAAGAATTTTATCAGAGCACCTGCAGCGCGGTGAGCAAAGGCT |
| NO: 244 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCTTTAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 328 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTFKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-S290C

| | |
|---|---|
| SEQ ID | CAGAACATTACCGAAGAATTTTATCAGAGCACCTGCAGCGCGGTGAGCAAAGGCT |
| NO: 245 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGTGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 329 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMCIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-Y458C

| | |
|---|---|
| SEQ ID | CAGAACATTACCGAAGAATTTTATCAGAGCACCTGCAGCGCGGTGAGCAAAGGCT |
| NO: 246 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTGCGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 330 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYCVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-Q501G

| | |
|---|---|
| SEQ ID | CAGAACATTACCGAAGAATTTTATCAGAGCACCTGCAGCGCGGTGAGCAAAGGCT |
| NO: 247 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACGGCAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 331 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINGSLAFIRKSDELLHNVNAGKSTTNIMITTIIIVIIVILLSLIAVGLLLYCK |
| | ARSTPVTLSKDQLSGINNIAFSN |

RSV-L512K

| | |
|---|---|
| SEQ ID | CAGAACATTACCGAAGAATTTTATCAGAGCACCTGCAGCGCGGTGAGCAAAGGCT |
| NO: 248 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAAAAACTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 332 | KYKNAVTELQLLMQSTPPTNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDEKLHNVNAGKSTTNIMITTIIIVIIVILLSLIAVGLLLYCK |
| | ARSTPVTLSKDQLSGINNIAFSN |

RSV-E2D

| | |
|---|---|
| SEQ ID | CAGAACATTACCGAAGAATTTTATCAGAGCACCTGCAGCGCGGTGAGCAAAGGCT |
| NO: 249 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 333 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-L4P

| | |
|---|---|
| SEQ ID | CAGAACATTACCGAAGAATTTTATCAGAGCACCTGCAGCGCGGTGAGCAAAGGCT |
| NO: 250 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 334 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-A8T

| | |
|---|---|
| SEQ ID | CAGAACATTACCGAAGAATTTTATCAGAGCACCTGCAGCGCGGTGAGCAAAGGCT |
| NO: 251 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 335 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-T13A

| | |
|---|---|
| SEQ ID | CAGAACATTACCGAAGAATTTTATCAGAGCACCTGCAGCGCGGTGAGCAAAGGCT |
| NO: 252 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 336 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-L15F

| | |
|---|---|
| SEQ ID | CAGAACATTACCGAAGAATTTTATCAGAGCACCTGCAGCGCGGTGAGCAAAGGCT |
| NO: 253 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 337 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-T16A

| | |
|---|---|
| SEQ ID | CAGAACATTACCGAAGAATTTTATCAGAGCACCTGCAGCGCGGTGAGCAAAGGCT |
| NO: 254 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 338 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-F20L

| | |
|---|---|
| SEQ ID | CAGAACATTACCGAAGAATTTTATCAGAGCACCTGCAGCGCGGTGAGCAAAGGCT |
| NO: 255 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 339 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-G25S

| | |
|---|---|
| SEQ ID | CAGAACATTACCGAAGAATTTTATCAGAGCACCTGCAGCGCGGTGAGCAAAGGCT |
| NO: 256 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 340 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-E30V

| | |
|---|---|
| SEQ ID | CAGAACATTACCGTGGAATTTTATCAGAGCACCTGCAGCGCGGTGAGCAAAGGCT |
| NO: 257 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITVEFYQSTCSAVSKGYLSALRTGWYTSVITIELSNIKENKCNGTDAKVKLIKQELD |
| NO: 341 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-T54H

| | |
|---|---|
| SEQ ID | CAGAACATTACCGAAGAATTTTATCAGAGCACCTGCAGCGCGGTGAGCAAAGGCT |
| NO: 258 | ATCTGAGCGCGCTGCGCACCGGCTGGTATCATAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYHSVITIELSNIKENKCNGTDAKVKLIKQELD |
| NO: 342 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-N88S

| | |
|---|---|
| SEQ ID | CAGAACATTACCGAAGAATTTTATCAGAGCACCTGCAGCGCGGTGAGCAAAGGCT |
| NO: 259 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAGCGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 343 | KYKSAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-T103A

| | |
|---|---|
| SEQ ID | CAGAACATTACCGAAGAATTTTATCAGAGCACCTGCAGCGCGGTGAGCAAAGGCT |
| NO: 260 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGGCGAACAACCGCGCGCGCCGCGAACTGCCGCGCTTTATGAA |
| | CTATACCCTGAACAACGCGAAAAAAACCAACGTGACCCTGAGCAAAAAACGCAA |
| | ACGCCGCTTTCTGGGCTTTCTGCTGGGCGTGGGCAGCGCGATTGCGAGCGGCGTG |
| | GCGGTGAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAGTGAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCGTGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACATTATGATTACCACCATTATTATTGTGATTAT |
| | TGTGATTCTGCTGAGCCTGATTGCGGTGGGCCTGCTGCTGTATTGCAAAGCGCGC |
| | AGCACCCCGGTGACCCTGAGCAAAGATCAGCTGAGCGGCATTAACAACATTGCGT |
| | TTAGCAACTGATAA |
| SEQ ID | QNITEEFYQSTCSAVSKGYLSALRTGWYTSVITIELSNIKENKCNGTDAKVKLIKQELD |
| NO: 344 | KYKNAVTELQLLMQSTPAANNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-A122T

| | |
|---|---|
| SEQ ID | CAGAACATTACCGAAGAATTTTATCAGAGCACCTGCAGCGCGGTGAGCAAAGGCT |
| NO: 261 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACACCAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 345 | KYKNAVTELQLLMQSTPATNNRARRELPRFMNYTLNNTKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-K124N

| | |
|---|---|
| SEQ ID | CAGAACATTACCGAAGAATTTTATCAGAGCACCTGCAGCGCGGTGAGCAAAGGCT |
| NO: 262 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAACACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 346 | KYKNAVTELQLLMQSTPATNNRARRELPRFMNYTLNNAKNTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-T125N

| | |
|---|---|
| SEQ ID | CAGAACATTACCGAAGAATTTTATCAGAGCACCTGCAGCGCGGTGAGCAAAGGCT |
| NO: 263 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAAACAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 347 | KYKNAVTELQLLMQSTPATNNRARRELPRFMNYTLNNAKKNNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-R136K

| | |
|---|---|
| SEQ ID | CAGAACATTACCGAAGAATTTTATCAGAGCACCTGCAGCGCGGTGAGCAAAGGCT |
| NO: 264 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCAAATTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 348 | KYKNAVTELQLLMQSTPATNNRARRELPRFMNYTLNNAKKTNVTLSKKRKRKFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-V152I

| | |
|---|---|
| SEQ ID | CAGAACATTACCGAAGAATTTTATCAGAGCACCTGCAGCGCGGTGAGCAAAGGCT |
| NO: 265 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCATTG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 349 | KYKNAVTELQLLMQSTPATNNRARRELPRFMNYTLNNAKKTNVTLSKKRKRRFLGF |
| Corresponding | LLGVGSAIASGIAVSKVLHLEGEVNKIKSALLSTNKAVVSLSNGVSVLTSKVLDLKNY |
| amino acid | IDKQLLPIVNKQSCSISNIETVIEFQQKNNRLLEITREFSVNAGVTTPVSTYMLTNSELLS |
| sequence | LINDMPITNDQKKLMSNNVQIVRQQSYSIMSIIKEEVLAYVVQLPLYGVIDTPCWKLH |
| | TSPLCTTNTKEGSNICLTRTDRGWYCDNAGSVSFFPQAETCKVQSNRVFCDTMNSLT |
| | LPSEVNLCNVDIFNPKYDCKIMTSKTDVSSSVITSLGAIVSCYGKTKCTASNKNRGIIK |
| | TFSNGCDYVSNKGVDTVSVGNTLYYVNKQEGKSLYVKGEPIINFYDPLVFPSDEFDAS |
| | ISQVNEKINQSLAFIRKSDELLHNVNAGKSTTNIMITTIIIVIIVILLSLIAVGLLLYCKAR |
| | STPVTLSKDQLSGINNIAFSN |

RSV-S190I

| | |
|---|---|
| SEQ ID | CAGAACATTACCGAAGAATTTTATCAGAGCACCTGCAGCGCGGTGAGCAAAGGCT |
| NO: 266 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCATTAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 350 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTIKVLDLKNY |
| amino acid | IDKQLLPIVNKQSCSISNIETVIEFQQKNNRLLEITREFSVNAGVTTPVSTYMLTNSELLS |
| sequence | LINDMPITNDQKKLMSNNVQIVRQQSYSIMSIIKEEVLAYVVQLPLYGVIDTPCWKLH |
| | TSPLCTTNTKEGSNICLTRTDRGWYCDNAGSVSFFPQAETCKVQSNRVFCDTMNSLT |
| | LPSEVNLCNVDIFNPKYDCKIMTSKTDVSSSVITSLGAIVSCYGKTKCTASNKNRGIIK |
| | TFSNGCDYVSNKGVDTVSVGNTLYYVNKQEGKSLYVKGEPIINFYDPLVFPSDEFDAS |
| | ISQVNEKINQSLAFIRKSDELLHNVNAGKSTTNIMITTIIIVIIVILLSLIAVGLLLYCKAR |
| | STPVTLSKDQLSGINNIAFSN |

RSV-N227S

| | |
|---|---|
| SEQ ID | CAGAACATTACCGAAGAATTTTATCAGAGCACCTGCAGCGCGGTGAGCAAAGGCT |
| NO: 267 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAGCAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 351 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKSNRLLEITREFSVNAGVTTPVSTYMLTNSELL |
| sequence | SLINDMPITNDQKKLMSNNVQIVRQQSYSIMSIIKEEVLAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-V296I

| | |
|---|---|
| SEQ ID | ACAGAACATTACCGAAGAATTTTATCAGAGCACCTGCAGCGCGGTGAGCAAAGG |
| NO: 268 | CTATCTGAGCGCGCTGCGCACCGGCTGGTATACCAGCGTGATTACCATTGAACTG |
| mRNA | AGCAACATTAAAGAAAACAAATGCAACGGCACCGATGCGAAAGTGAAACTGATT |
| | AAACAGGAACTGGATAAATATAAAAACGCGGTGACCGAACTGCAGCTGCTGATG |
| | CAGAGCACCCCGGCGACCAACAACCGCGCGCGCCGCGAACTGCCGCGCTTTATGA |
| | ACTATACCCTGAACAACGCGAAAAAAACCAACGTGACCCTGAGCAAAAAACGCA |
| | AACGCCGCTTTCTGGGCTTTCTGCTGGGCGTGGGCAGCGCGATTGCGAGCGGCGT |
| | GGCGGTGAGCAAAGTGCTGCATCTGGAAGGCGAAGTGAACAAAATTAAAAGCGC |
| | GCTGCTGAGCACCAACAAAGCGGTGGTGAGCCTGAGCAACGGCGTGAGCGTGCT |
| | GACCAGCAAAGTGCTGGATCTGAAAAACTATATTGATAAACAGCTGCTGCCGATT |
| | GTGAACAAACAGAGCTGCAGCATTAGCAACATTGAAACCGTGATTGAATTTCAGC |
| | AGAAAAACAACCGCCTGCTGGAAATTACCCGCGAATTTAGCGTGAACGCGGGCG |
| | TGACCACCCCGGTGAGCACCTATATGCTGACCAACAGCGAACTGCTGAGCCTGAT |
| | TAACGATATGCCGATTACCAACGATCAGAAAAAACTGATGAGCAACAACGTGCA |
| | GATTGTGCGCCAGCAGAGCTATAGCATTATGAGCATTATTAAAGAAGAAATTCTG |
| | GCGTATGTGGTGCAGCTGCCGCTGTATGGCGTGATTGATACCCCGTGCTGGAAAC |
| | TGCATACCAGCCCGCTGTGCACCACCAACACCAAAGAAGGCAGCAACATTTGCCT |
| | GACCCGCACCGATCGCGGCTGGTATTGCGATAACGCGGGCAGCGTGAGCTTTTTT |
| | CCGCAGGCGGAAACCTGCAAAGTGCAGAGCAACCGCGTGTTTTGCGATACCATGA |
| | ACAGCCTGACCCTGCCGAGCGAAGTGAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCGTGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACATTATGATTACCACCATTATTATTGTGATTAT |
| | TGTGATTCTGCTGAGCCTGATTGCGGTGGGCCTGCTGCTGTATTGCAAAGCGCGC |
| | AGCACCCCGGTGACCCTGAGCAAAGATCAGCTGAGCGGCATTAACAACATTGCGT |
| | TTAGCAACTGATAA |
| SEQ ID | QNITEEFYQSTCSAVSKGYLSALRTGWYTSVITIELSNIKENKCNGTDAKVKLIKQELD |
| NO: 352 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEILAYVVQLPLYGVIDTPCWKL |
| | HTSPLCTTNTKEGSNICLTRTDRGWYCDNAGSVSFFPQAETCKVQSNRVFCDTMNSL |
| | TLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLL |
| | YCKARSTPVTLSKDQLSGINNIAFSN |

RSV-Q354L

| | |
|---|---|
| SEQ ID | CAGAACATTACCGAAGAATTTTATCAGAGCACCTGCAGCGCGGTGAGCAAAGGCT |
| NO: 269 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCTGGCGGAAACCTGCAAAGTGCAGAGCAACCGCGTGTTTTGCGATACCATGAAC |
| | AGCCTGACCCTGCCGAGCGAAGTGAACCTGTGCAACGTGGATATTTTTAACCCGA |
| | AATATGATTGCAAAATTATGACCAGCAAAACCGATGTGAGCAGCAGCGTGATTAC |
| | CAGCCTGGGCGCGATTGTGAGCTGCTATGGCAAAACCAAATGCACCGCGAGCAA |
| | CAAAAACCGCGGCATTATTAAAACCTTTAGCAACGGCTGCGATTATGTGAGCAAC |
| | AAAGGCGTGGATACCGTGAGCGTGGGCAACACCCTGTATTATGTGAACAAACAG |
| | GAAGGCAAAAGCCTGTATGTGAAAGGCGAACCGATTATTAACTTTTATGATCCGC |
| | TGGTGTTTCCGAGCGATGAATTTGATGCGAGCATTAGCCAGGTGAACGAAAAAAT |
| | TAACCAGAGCCTGGCGTTTATTCGCAAAAGCGATGAACTGCTGCATAACGTGAAC |
| | GCGGGCAAAAGCACCACCAACATTATGATTACCACCATTATTATTGTGATTATTGT |
| | GATTCTGCTGAGCCTGATTGCGGTGGGCCTGCTGCTGTATTGCAAAGCGCGCAGC |
| | ACCCCGGTGACCCTGAGCAAAGATCAGCTGAGCGGCATTAACAACATTGCGTTTA |
| | GCAACTGATAA |
| SEQ ID | QNITEEFYQSTCSAVSKGYLSALRTGWYTSVITIELSNIKENKCNGTDAKVKLIKQELD |
| NO: 353 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPLAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-L373R

| | |
|---|---|
| SEQ ID | CAGAACATTACCGAAGAATTTTATCAGAGCACCTGCAGCGCGGTGAGCAAAGGCT |
| NO: 270 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCGCACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 354 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | RTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-D486S

| | |
|---|---|
| SEQ ID | CAGAACATTACCGAAGAATTTTATCAGAGCACCTGCAGCGCGGTGAGCAAAGGCT |
| NO: 271 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCAGCGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 355 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSSEFDA |
| | SISQVNEKINQSLAFIRKSDELLHNVNAGKSTTNIMITTIIIVIIVILLSLIAVGLLLYCKA |
| | RSTPVTLSKDQLSGINNIAFSN |

RSV-S540L

| | |
|---|---|
| SEQ ID | CAGAACATTACCGAAGAATTTTATCAGAGCACCTGCAGCGCGGTGAGCAAAGGCT |
| NO: 272 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGCTGCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 356 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLLLIAVGLLLYCK |
| | ARSTPVTLSKDQLSGINNIAFSN |

RSV-L547F

| | |
|---|---|
| SEQ ID | CAGAACATTACCGAAGAATTTTATCAGAGCACCTGCAGCGCGGTGAGCAAAGGCT |
| NO: 273 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGTTTCTGTATTGCAAAGCGCGCAG |
| | CACCCCGGTGACCCTGAGCAAAGATCAGCTGAGCGGCATTAACAACATTGCGTTT |
| | AGCAACTGATAA |
| SEQ ID | QNITEEFYQSTCSAVSKGYLSALRTGWYTSVITIELSNIKENKCNGTDAKVKLIKQELD |
| NO: 357 | KYKNAVTELQLLMQSTPATNNRARRELPRFMNYTLNNAKKTNVTLSKKRKRRFLGF |
| Corresponding | LLGVGSAIASGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLFLYCK |
| | ARSTPVTLSKDQLSGINNIAFSN |

RSV-S155C + S290C

| | |
|---|---|
| SEQ ID | CAGAACATTACCGAAGAATTTTATCAGAGCACCTGCAGCGCGGTGAGCAAAGGCT |
| NO: 274 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGTGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGTGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 358 | KYKNAVTELQLLMQSTPATNNRARRELPRFMNYTLNNAKKTNVTLSKKRKRRFLGF |
| Corresponding | LLGVGSAIASGVAVCKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMCIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-A149C + Y458C

| | |
|---|---|
| SEQ ID | CAGAACATTACCGAAGAATTTTATCAGAGCACCTGCAGCGCGGTGAGCAAAGGCT |
| NO: 275 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTTGCAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAGTGAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCGTGGATACCGTGAGCGTGGGCAACACCCTGTATTGCGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 359 | KYKNAVTELQLLMQSTPATNNRARRELPRFMNYTLNNAKKTNVTLSKKRKRRFLGF |
| Corresponding | LLGVGSAICSGVAVSKVLHLEGEVNKIKSALLSTNKAVVSLSNGVSVLTSKVLDLKN |
| amino acid | YIDKQLLPIVNKQSCSISNIETVIEFQQKNNRLLEITREFSVNAGVTTPVSTYMLTNSEL |
| sequence | LSLINDMPITNDQKKLMSNNVQIVRQQSYSIMSIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEVNLCNVDIFNPKYDCKIMTSKTDVSSSVITSLGAIVSCYGKTKCTASNKNRGII |
| | KTFSNGCDYVSNKGVDTVSVGNTLYCVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-mRNA-1345

| | |
|---|---|
| SEQ ID | CAGAACATTACCGAAGAATTTTATCAGAGCACCTGCAGCGCGGTGAGCAAAGGCT |
| NO: 276 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGGCGACCGGCAGCGGCAGCGCGATTTGCAGCGGCGTGGCGGTGTG |
| | CAAAGTGCTGCATCTGGAAGGCGAAGTGAACAAAATTAAAAGCGCGCTGCTGAG |
| | CACCAACAAAGCGGTGGTGAGCCTGAGCAACGGCGTGAGCGTGCTGACCTTTAA |
| | AGTGCTGGATCTGAAAAACTATATTGATAAACAGCTGCTGCCGATTCTGAACAAA |
| | CAGAGCTGCAGCATTAGCAACATTGAAACCGTGATTGAATTTCAGCAGAAAAACA |
| | ACCGCCTGCTGGAAATTACCCGCGAATTTAGCGTGAACGCGGGCGTGACCACCCC |
| | GGTGAGCACCTATATGCTGACCAACAGCGAACTGCTGAGCCTGATTAACGATATG |
| | CCGATTACCAACGATCAGAAAAAACTGATGAGCAACAACGTGCAGATTGTGCGC |
| | CAGCAGAGCTATAGCATTATGTGCATTATTAAAGAAGAAGTGCTGGCGTATGTGG |
| | TGCAGCTGCCGCTGTATGGCGTGATTGATACCCCGTGCTGGAAACTGCATACCAG |
| | CCCGCTGTGCACCACCAACACCAAAGAAGGCAGCAACATTTGCCTGACCCGCACC |
| | GATCGCGGCTGGTATTGCGATAACGCGGGCAGCGTGAGCTTTTTTCCGCAGGCGG |
| | AAACCTGCAAAGTGCAGAGCAACCGCGTGTTTTGCGATACCATGAACAGCCGCAC |
| | CCTGCCGAGCGAAGTGAACCTGTGCAACGTGGATATTTTTAACCCGAAATATGAT |
| | TGCAAAATTATGACCAGCAAAACCGATGTGAGCAGCAGCGTGATTACCAGCCTGG |
| | GCGCGATTGTGAGCTGCTATGGCAAAACCAAATGCACCGCGAGCAACAAAAACC |
| | GCGGCATTATTAAAACCTTTAGCAACGGCTGCGATTATGTGAGCAACAAAGGCGT |
| | GGATACCGTGAGCGTGGGCAACACCCTGTATTGCGTGAACAAACAGGAAGGCAA |
| | AAGCCTGTATGTGAAAGGCGAACCGATTATTAACTTTTATGATCCGCTGGTGTTTC |
| | CGAGCGATGAATTTGATGCGAGCATTAGCCAGGTGAACGAAAAAATTAACCAGA |
| | GCCTGGCGTTTATTCGCAAAAGCGATGAACTGCTGCATAACGTGAACGCGGGCAA |
| | AAGCACCACCAACATTATGATTACCACCATTATTATTGTGATTATTGTGATTCTGC |
| | TGAGCCTGATTGCGGTGGGCCTGCTGCTGTATTGATAA |
| SEQ ID | QNITEEFYQSTCSAVSKGYLSALRTGWYTSVITIELSNIKENKCNGTDAKVKLIKQELD |
| NO: 360 | KYKNAVTELQLLMQSTPATGSGSAICSGVAVCKVLHLEGEVNKIKSALLSTNKAVVS |
| Corresponding | LSNGVSVLTFKVLDLKNYIDKQLLPILNKQSCSISNIETVIEFQQKNNRLLEITREFSVN |
| amino acid | AGVTTPVSTYMLTNSELLSLINDMPITNDQKKLMSNNVQIVRQQSYSIMCIIKEEVLA |
| sequence | YVVQLPLYGVIDTPCWKLHTSPLCTTNTKEGSNICLTRTDRGWYCDNAGSVSFFPQA |
| | ETCKVQSNRVFCDTMNSRTLPSEVNLCNVDIFNPKYDCKIMTSKTDVSSSVITSLGAIV |
| | SCYGKTKCTASNKNRGIIKTFSNGCDYVSNKGVDTVSVGNTLYCVNKQEGKSLYVK |
| | GEPIINFYDPLVFPSDEFDASISQVNEKINQSLAFIRKSDELLHNVNAGKSTTNIMITTIII |
| | VIIVILLSLIAVGLLLY |

RSV-FP-T4

| | |
|---|---|
| SEQ ID | CAGAACATTACCGAAGAATTTTATCAGAGCACCTGCAGCGCGGTGAGCAAAGGCT |
| NO: 277 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGGCAGCTTTCTGGGCTTTCTG |
| | CTGGGCGTGGGCAGCGCGATTGCGAGCGGCGTGGCGGTGAGCAAAGTGCTGCAT |
| | CTGGAAGGCGAAGTGAACAAAATTAAAAGCGCGCTGCTGAGCACCAACAAAGCG |
| | GTGGTGAGCCTGAGCAACGGCGTGAGCGTGCTGACCAGCAAAGTGCTGGATCTG |
| | AAAAACTATATTGATAAACAGCTGCTGCCGATTGTGAACAAACAGAGCTGCAGCA |
| | TTAGCAACATTGAAACCGTGATTGAATTTCAGCAGAAAAACAACCGCCTGCTGGA |
| | AATTACCCGCGAATTTAGCGTGAACGCGGGCGTGACCACCCCGGTGAGCACCTAT |
| | ATGCTGACCAACAGCGAACTGCTGAGCCTGATTAACGATATGCCGATTACCAACG |
| | ATCAGAAAAAACTGATGAGCAACAACGTGCAGATTGTGCGCCAGCAGAGCTATA |
| | GCATTATGAGCATTATTAAAGAAGAAGTGCTGGCGTATGTGGTGCAGCTGCCGCT |
| | GTATGGCGTGATTGATACCCCGTGCTGGAAACTGCATACCAGCCCGCTGTGCACC |
| | ACCAACACCAAAGAAGGCAGCAACATTTGCCTGACCCGCACCGATCGCGGCTGGT |
| | ATTGCGATAACGCGGGCAGCGTGAGCTTTTTTCCGCAGGCGGAAACCTGCAAAGT |
| | GCAGAGCAACCGCGTGTTTTGCGATACCATGAACAGCCTGACCCTGCCGAGCGAA |
| | ATTAACCTGTGCAACGTGGATATTTTTAACCCGAAATATGATTGCAAAATTATGA |
| | CCAGCAAAACCGATGTGAGCAGCAGCGTGATTACCAGCCTGGGCGCGATTGTGA |
| | GCTGCTATGGCAAAACCAAATGCACCGCGAGCAACAAAAACCGCGGCATTATTA |
| | AAACCTTTAGCAACGGCTGCGATTATGTGAGCAACAAAGGCATGGATACCGTGAG |
| | CGTGGGCAACACCCTGTATTATGTGAACAAACAGGAAGGCAAAAGCCTGTATGTG |
| | AAAGGCGAACCGATTATTAACTTTTATGATCCGCTGGTGTTTCCGAGCGATGAATT |
| | TGATGCGAGCATTAGCCAGGTGAACGAAAAAATTAACCAGAGCCTGGCGTTTATT |
| | CGCAAAAGCGATGAACTGCTGCATAACGTGAACGCGGGCAAAAGCACCACCAAC |
| | GGCTATATTCCGGAAGCGCCGCGCGATGGCCAGGCGTATGTGCGCAAAGATGGC |
| | GAATGGGTGCTGCTGAGCACCTTTCTGTGATAA |
| SEQ ID | QNITEEFYQSTCSAVSKGYLSALRTGWYTSVITIELSNIKENKCNGTDAKVKLIKQELD |
| NO: 361 | KYKNAVTELQLLMQSTPPTNNRARRGSFLGFLLGVGSAIASGVAVSKVLHLEGEVNK |
| Corresponding | IKSALLSTNKAVVSLSNGVSVLTSKVLDLKNYIDKQLLPIVNKQSCSISNIETVIEFQQK |
| amino acid | NNRLLEITREFSVNAGVTTPVSTYMLTNSELLSLINDMPITNDQKKLMSNNVQIVRQQ |
| sequence | SYSIMSIIKEEVLAYVVQLPLYGVIDTPCWKLHTSPLCTTNTKEGSNICLTRTDRGWYC |
| | DNAGSVSFFPQAETCKVQSNRVFCDTMNSLTLPSEINLCNVDIFNPKYDCKIMTSKTD |
| | VSSSVITSLGAIVSCYGKTKCTASNKNRGIIKTFSNGCDYVSNKGMDTVSVGNTLYYV |
| | NKQEGKSLYVKGEPIINFYDPLVFPSDEFDASISQVNEKINQSLAFIRKSDELLHNVNA |
| | GKSTTNGYIPEAPRDGQAYVRKDGEWVLLSTFL |

RSV-GS-FP-TM

| | |
|---|---|
| SEQ ID | CAGAACATTACCGAAGAATTTTATCAGAGCACCTGCAGCGCGGTGAGCAAAGGCT |
| NO: 278 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGGCAGCTTTCTGGGCTTTCTG |
| | CTGGGCGTGGGCAGCGCGATTGCGAGCGGCGTGGCGGTGAGCAAAGTGCTGCAT |
| | CTGGAAGGCGAAGTGAACAAAATTAAAAGCGCGCTGCTGAGCACCAACAAAGCG |
| | GTGGTGAGCCTGAGCAACGGCGTGAGCGTGCTGACCAGCAAAGTGCTGGATCTG |
| | AAAAACTATATTGATAAACAGCTGCTGCCGATTGTGAACAAACAGAGCTGCAGCA |
| | TTAGCAACATTGAAACCGTGATTGAATTTCAGCAGAAAAACAACCGCCTGCTGGA |
| | AATTACCCGCGAATTTAGCGTGAACGCGGGCGTGACCACCCCGGTGAGCACCTAT |
| | ATGCTGACCAACAGCGAACTGCTGAGCCTGATTAACGATATGCCGATTACCAACG |
| | ATCAGAAAAAACTGATGAGCAACAACGTGCAGATTGTGCGCCAGCAGAGCTATA |
| | GCATTATGAGCATTATTAAAGAAGAAGTGCTGGCGTATGTGGTGCAGCTGCCGCT |
| | GTATGGCGTGATTGATACCCCGTGCTGGAAACTGCATACCAGCCCGCTGTGCACC |
| | ACCAACACCAAAGAAGGCAGCAACATTTGCCTGACCCGCACCGATCGCGGCTGGT |
| | ATTGCGATAACGCGGGCAGCGTGAGCTTTTTTCCGCAGGCGGAAACCTGCAAAGT |
| | GCAGAGCAACCGCGTGTTTTGCGATACCATGAACAGCCTGACCCTGCCGAGCGAA |
| | ATTAACCTGTGCAACGTGGATATTTTTAACCCGAAATATGATTGCAAAATTATGA |
| | CCAGCAAAACCGATGTGAGCAGCAGCGTGATTACCAGCCTGGGCGCGATTGTGA |
| | GCTGCTATGGCAAAACCAAATGCACCGCGAGCAACAAAAACCGCGGCATTATTA |
| | AAACCTTTAGCAACGGCTGCGATTATGTGAGCAACAAAGGCATGGATACCGTGAG |
| | CGTGGGCAACACCCTGTATTATGTGAACAAACAGGAAGGCAAAAGCCTGTATGTG |
| | AAAGGCGAACCGATTATTAACTTTTATGATCCGCTGGTGTTTCCGAGCGATGAATT |
| | TGATGCGAGCATTAGCCAGGTGAACGAAAAAATTAACCAGAGCCTGGCGTTTATT |
| | CGCAAAAGCGATGAACTGCTGCATAACGTGAACGCGGGCAAAAGCACCACCAAC |
| | ATTATGATTACCACCATTATTATTGTGATTATTGTGATTCTGCTGAGCCTGATTGC |
| | GGTGGGCCTGCTGCTGTATTGCTGATAA |
| SEQ ID | QNITEEFYQSTCSAVSKGYLSALRTGWYTSVITIELSNIKENKCNGTDAKVKLIKQELD |
| NO: 362 | KYKNAVTELQLLMQSTPPTNNRARRGSFLGFLLGVGSAIASGVAVSKVLHLEGEVNK |
| Corresponding | IKSALLSTNKAVVSLSNGVSVLTSKVLDLKNYIDKQLLPIVNKQSCSISNIETVIEFQQK |
| amino acid | NNRLLEITREFSVNAGVTTPVSTYMLTNSELLSLINDMPITNDQKKLMSNNVQIVRQQ |
| sequence | SYSIMSIIKEEVLAYVVQLPLYGVIDTPCWKLHTSPLCTTNTKEGSNICLTRTDRGWYC |
| | DNAGSVSFFPQAETCKVQSNRVFCDTMNSLTLPSEINLCNVDIFNPKYDCKIMTSKTD |
| | VSSSVITSLGAIVSCYGKTKCTASNKNRGIIKTFSNGCDYVSNKGMDTVSVGNTLYYV |
| | NKQEGKSLYVKGEPIINFYDPLVFPSDEFDASISQVNEKINQSLAFIRKSDELLHNVNA |
| | GKSTTNIMITTIIIVIIVILLSLIAVGLLLYC |

RSV-p27-T4

| | |
|---|---|
| SEQ ID | CAGAACATTACCGAAGAATTTTATCAGAGCACCTGCAGCGCGGTGAGCAAAGGCT |
| NO: 279 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACGGCTATATTCCGGAAGCGCCGCGCGATGGCC |
| | AGGCGTATGTGCGCAAAGATGGCGAATGGGTGCTGCTGAGCACCTTTCTGTGATA |
| | A |
| SEQ ID | QNITEEFYQSTCSAVSKGYLSALRTGWYTSVITIELSNIKENKCNGTDAKVKLIKQELD |
| NO: 363 | KYKNAVTELQLLMQSTPPTNNRARRELPRFMNYTLNNAKKTNVTLSKKRKRRSKVL |
| Corresponding | HLEGEVNKIKSALLSTNKAVVSLSNGVSVLTSKVLDLKNYIDKQLLPIVNKQSCSISNI |
| amino acid | ETVIEFQQKNNRLLEITREFSVNAGVTTPVSTYMLTNSELLSLINDMPITNDQKKLMSN |
| sequence | NVQIVRQQSYSIMSIIKEEVLAYVVQLPLYGVIDTPCWKLHTSPLCTTNTKEGSNICLT |
| | RTDRGWYCDNAGSVSFFPQAETCKVQSNRVFCDTMNSLTLPSEINLCNVDIFNPKYD |
| | CKIMTSKTDVSSSVITSLGAIVSCYGKTKCTASNKNRGIIKTFSNGCDYVSNKGMDTV |
| | SVGNTLYYVNKQEGKSLYVKGEPIINFYDPLVFPSDEFDASISQVNEKINQSLAFIRKS |
| | DELLHNVNAGKSTTNGYIPEAPRDGQAYVRKDGEWVLLSTFL |

RSV-p27-TM

| | |
|---|---|
| SEQ ID | CAGAACATTACCGAAGAATTTTATCAGAGCACCTGCAGCGCGGTGAGCAAAGGCT |
| NO: 280 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACTGATAA |
| SEQ ID | QNITEEFYQSTCSAVSKGYLSALRTGWYTSVITIELSNIKENKCNGTDAKVKLIKQELD |
| NO: 364 | KYKNAVTELQLLMQSTPPTNNRARRELPRFMNYTLNNAKKTNVTLSKKRKRRSKVL |
| Corresponding | HLEGEVNKIKSALLSTNKAVVSLSNGVSVLTSKVLDLKNYIDKQLLPIVNKQSCSISNI |
| amino acid | ETVIEFQQKNNRLLEITREFSVNAGVTTPVSTYMLTNSELLSLINDMPITNDQKKLMSN |
| sequence | NVQIVRQQSYSIMSIIKEEVLAYVVQLPLYGVIDTPCWKLHTSPLCTTNTKEGSNICLT |
| | RTDRGWYCDNAGSVSFFPQAETCKVQSNRVFCDTMNSLTLPSEINLCNVDIFNPKYD |
| | CKIMTSKTDVSSSVITSLGAIVSCYGKTKCTASNKNRGIIKTFSNGCDYVSNKGMDTV |
| | SVGNTLYYVNKQEGKSLYVKGEPIINFYDPLVFPSDEFDASISQVNEKINQSLAFIRKS |
| | DELLHNVNAGKSTTN |

RSV-p27-TM-CT

| | |
|---|---|
| SEQ ID | CAGAACATTACCGAAGAATTTTATCAGAGCACCTGCAGCGCGGTGAGCAAAGGCT |
| NO: 281 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACATTATGATTACCACCATTATTATTGTGATTAT |
| | TGTGATTCTGCTGAGCCTGATTGCGGTGGGCCTGCTGCTGTATTGCAAAGCGCGC |
| | AGCACCCCGGTGACCCTGAGCAAAGATCAGCTGAGCGGCATTAACAACATTGCGT |
| | TTAGCAACTGATAA |
| SEQ ID | QNITEEFYQSTCSAVSKGYLSALRTGWYTSVITIELSNIKENKCNGTDAKVKLIKQELD |
| NO: 365 | KYKNAVTELQLLMQSTPPTNNRARRELPRFMNYTLNNAKKTNVTLSKKRKRRSKVL |
| Corresponding | HLEGEVNKIKSALLSTNKAVVSLSNGVSVLTSKVLDLKNYIDKQLLPIVNKQSCSISNI |
| amino acid | ETVIEFQQKNNRLLEITREFSVNAGVTTPVSTYMLTNSELLSLINDMPITNDQKKLMSN |
| sequence | NVQIVRQQSYSIMSIIKEEVLAYVVQLPLYGVIDTPCWKLHTSPLCTTNTKEGSNICLT |
| | RTDRGWYCDNAGSVSFFPQAETCKVQSNRVFCDTMNSLTLPSEINLCNVDIFNPKYD |
| | CKIMTSKTDVSSSVITSLGAIVSCYGKTKCTASNKNRGIIKTFSNGCDYVSNKGMDTV |
| | SVGNTLYYVNKQEGKSLYVKGEPIINFYDPLVFPSDEFDASISQVNEKINQSLAFIRKS |
| | DELLHNVNAGKSTTNIMITTIIIVIIVILLSLIAVGLLLYCKARSTPVTLSKDQLSGINNIA |
| | FSN |

RSV-GS-T4

| | |
|---|---|
| SEQ ID | CAGAACATTACCGAAGAATTTTATCAGAGCACCTGCAGCGCGGTGAGCAAAGGCT |
| NO: 282 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGGCAGCAGCAAAGTGCTGCA |
| | TCTGGAAGGCGAAGTGAACAAAATTAAAAGCGCGCTGCTGAGCACCAACAAAGC |
| | GGTGGTGAGCCTGAGCAACGGCGTGAGCGTGCTGACCAGCAAAGTGCTGGATCT |
| | GAAAAACTATATTGATAAACAGCTGCTGCCGATTGTGAACAAACAGAGCTGCAGC |
| | ATTAGCAACATTGAAACCGTGATTGAATTTCAGCAGAAAAACAACCGCCTGCTGG |
| | AAATTACCCGCGAATTTAGCGTGAACGCGGGCGTGACCACCCCGGTGAGCACCTA |
| | TATGCTGACCAACAGCGAACTGCTGAGCCTGATTAACGATATGCCGATTACCAAC |
| | GATCAGAAAAAACTGATGAGCAACAACGTGCAGATTGTGCGCCAGCAGAGCTAT |
| | AGCATTATGAGCATTATTAAAGAAGAAGTGCTGGCGTATGTGGTGCAGCTGCCGC |
| | TGTATGGCGTGATTGATACCCCGTGCTGGAAACTGCATACCAGCCCGCTGTGCAC |
| | CACCAACACCAAAGAAGGCAGCAACATTTGCCTGACCCGCACCGATCGCGGCTG |
| | GTATTGCGATAACGCGGGCAGCGTGAGCTTTTTTCCGCAGGCGGAAACCTGCAAA |
| | GTGCAGAGCAACCGCGTGTTTTGCGATACCATGAACAGCCTGACCCTGCCGAGCG |
| | AAATTAACCTGTGCAACGTGGATATTTTTAACCCGAAATATGATTGCAAAATTAT |
| | GACCAGCAAAACCGATGTGAGCAGCAGCGTGATTACCAGCCTGGGCGCGATTGT |
| | GAGCTGCTATGGCAAAACCAAATGCACCGCGAGCAACAAAAACCGCGGCATTAT |
| | TAAAACCTTTAGCAACGGCTGCGATTATGTGAGCAACAAAGGCATGGATACCGTG |
| | AGCGTGGGCAACACCCTGTATTATGTGAACAAACAGGAAGGCAAAAGCCTGTAT |
| | GTGAAAGGCGAACCGATTATTAACTTTTATGATCCGCTGGTGTTTCCGAGCGATG |
| | AATTTGATGCGAGCATTAGCCAGGTGAACGAAAAAATTAACCAGAGCCTGGCGTT |
| | TATTCGCAAAAGCGATGAACTGCTGCATAACGTGAACGCGGGCAAAAGCACCAC |
| | CAACGGCTATATTCCGGAAGCGCCGCGCGATGGCCAGGCGTATGTGCGCAAAGAT |
| | GGCGAATGGGTGCTGCTGAGCACCTTTCTGTGATAA |
| SEQ ID | QNITEEFYQSTCSAVSKGYLSALRTGWYTSVITIELSNIKENKCNGTDAKVKLIKQELD |
| NO: 366 | KYKNAVTELQLLMQSTPPTNNRARRGSSKVLHLEGEVNKIKSALLSTNKAVVSLSNG |
| Corresponding | VSVLTSKVLDLKNYIDKQLLPIVNKQSCSISNIETVIEFQQKNNRLLEITREFSVNAGVT |
| amino acid | TPVSTYMLTNSELLSLINDMPITNDQKKLMSNNVQIVRQQSYSIMSIIKEEVLAYVVQL |
| sequence | PLYGVIDTPCWKLHTSPLCTTNTKEGSNICLTRTDRGWYCDNAGSVSFFPQAETCKV |
| | QSNRVFCDTMNSLTLPSEINLCNVDIFNPKYDCKIMTSKTDVSSSVITSLGAIVSCYGK |
| | TKCTASNKNRGIIKTFSNGCDYVSNKGMDTVSVGNTLYYVNKQEGKSLYVKGEPIIN |
| | FYDPLVFPSDEFDASISQVNEKINQSLAFIRKSDELLHNVNAGKSTINGYIPEAPRDGQ |
| | AYVRKDGEWVLLSTFL |

RSV-GS-TM

| | |
|---|---|
| SEQ ID | CAGAACATTACCGAAGAATTTTATCAGAGCACCTGCAGCGCGGTGAGCAAAGGCT |
| NO: 283 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGGCAGCAGCAAAGTGCTGCA |
| | TCTGGAAGGCGAAGTGAACAAAATTAAAAGCGCGCTGCTGAGCACCAACAAAGC |
| | GGTGGTGAGCCTGAGCAACGGCGTGAGCGTGCTGACCAGCAAAGTGCTGGATCT |
| | GAAAAACTATATTGATAAACAGCTGCTGCCGATTGTGAACAAACAGAGCTGCAGC |
| | ATTAGCAACATTGAAACCGTGATTGAATTTCAGCAGAAAAACAACCGCCTGCTGG |
| | AAATTACCCGCGAATTTAGCGTGAACGCGGGCGTGACCACCCCGGTGAGCACCTA |
| | TATGCTGACCAACAGCGAACTGCTGAGCCTGATTAACGATATGCCGATTACCAAC |
| | GATCAGAAAAAACTGATGAGCAACAACGTGCAGATTGTGCGCCAGCAGAGCTAT |
| | AGCATTATGAGCATTATTAAAGAAGAAGTGCTGGCGTATGTGGTGCAGCTGCCGC |
| | TGTATGGCGTGATTGATACCCCGTGCTGGAAACTGCATACCAGCCCGCTGTGCAC |
| | CACCAACACCAAAGAAGGCAGCAACATTTGCCTGACCCGCACCGATCGCGGCTG |
| | GTATTGCGATAACGCGGGCAGCGTGAGCTTTTTTCCGCAGGCGGAAACCTGCAAA |
| | GTGCAGAGCAACCGCGTGTTTTGCGATACCATGAACAGCCTGACCCTGCCGAGCG |
| | AAATTAACCTGTGCAACGTGGATATTTTTAACCCGAAATATGATTGCAAAATTAT |
| | GACCAGCAAAACCGATGTGAGCAGCAGCGTGATTACCAGCCTGGGCGCGATTGT |
| | GAGCTGCTATGGCAAAACCAAATGCACCGCGAGCAACAAAAACCGCGGCATTAT |
| | TAAAACCTTTAGCAACGGCTGCGATTATGTGAGCAACAAAGGCATGGATACCGTG |
| | AGCGTGGGCAACACCCTGTATTATGTGAACAAACAGGAAGGCAAAAGCCTGTAT |
| | GTGAAAGGCGAACCGATTATTAACTTTTATGATCCGCTGGTGTTTCCGAGCGATG |
| | AATTTGATGCGAGCATTAGCCAGGTGAACGAAAAAATTAACCAGAGCCTGGCGTT |
| | TATTCGCAAAAGCGATGAACTGCTGCATAACGTGAACGCGGGCAAAAGCACCAC |
| | CAACATTATGATTACCACCATTATTATTGTGATTATTGTGATTCTGCTGAGCCTGA |
| | TTGCGGTGGGCCTGCTGCTGTATTGCTGATAA |
| SEQ ID | QNITEEFYQSTCSAVSKGYLSALRTGWYTSVITIELSNIKENKCNGTDAKVKLIKQELD |
| NO: 367 | KYKNAVTELQLLMQSTPPTNNRARRGSSKVLHLEGEVNKIKSALLSTNKAVVSLSNG |
| Corresponding | VSVLTSKVLDLKNYIDKQLLPIVNKQSCSISNIETVIEFQQKNNRLLEITREFSVNAGVT |
| amino acid | TPVSTYMLTNSELLSLINDMPITNDQKKLMSNNVQIVRQQSYSIMSIIKEEVLAYVVQL |
| sequence | PLYGVIDTPCWKLHTSPLCTTNTKEGSNICLTRTDRGWYCDNAGSVSFFPQAETCKV |
| | QSNRVFCDTMNSLTLPSEINLCNVDIFNPKYDCKIMTSKTDVSSSVITSLGAIVSCYGK |
| | TKCTASNKNRGIIKTFSNGCDYVSNKGMDTVSVGNTLYYVNKQEGKSLYVKGEPIIN |
| | FYDPLVFPSDEFDASISQVNEKINQSLAFIRKSDELLHNVNAGKSTTNIMITTIIIV |
| | IIVILLSLIAVGLLLYC |

RSV-GS-TM-CT

| | |
|---|---|
| SEQ ID | CAGAACATTACCGAAGAATTTTATCAGAGCACCTGCAGCGCGGTGAGCAAAGGCT |
| NO: 284 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGGCAGCAGCAAAGTGCTGCA |
| | TCTGGAAGGCGAAGTGAACAAAATTAAAAGCGCGCTGCTGAGCACCAACAAAGC |
| | GGTGGTGAGCCTGAGCAACGGCGTGAGCGTGCTGACCAGCAAAGTGCTGGATCT |
| | GAAAAACTATATTGATAAACAGCTGCTGCCGATTGTGAACAAACAGAGCTGCAGC |
| | ATTAGCAACATTGAAACCGTGATTGAATTTCAGCAGAAAAACAACCGCCTGCTGG |
| | AAATTACCCGCGAATTTAGCGTGAACGCGGGCGTGACCACCCCGGTGAGCACCTA |
| | TATGCTGACCAACAGCGAACTGCTGAGCCTGATTAACGATATGCCGATTACCAAC |
| | GATCAGAAAAAACTGATGAGCAACAACGTGCAGATTGTGCGCCAGCAGAGCTAT |
| | AGCATTATGAGCATTATTAAAGAAGAAGTGCTGGCGTATGTGGTGCAGCTGCCGC |
| | TGTATGGCGTGATTGATACCCCGTGCTGGAAACTGCATACCAGCCCGCTGTGCAC |
| | CACCAACACCAAAGAAGGCAGCAACATTTGCCTGACCCGCACCGATCGCGGCTG |
| | GTATTGCGATAACGCGGGCAGCGTGAGCTTTTTTCCGCAGGCGGAAACCTGCAAA |
| | GTGCAGAGCAACCGCGTGTTTTGCGATACCATGAACAGCCTGACCCTGCCGAGCG |
| | AAATTAACCTGTGCAACGTGGATATTTTTAACCCGAAATATGATTGCAAAATTAT |
| | GACCAGCAAAACCGATGTGAGCAGCAGCGTGATTACCAGCCTGGGCGCGATTGT |
| | GAGCTGCTATGGCAAAACCAAATGCACCGCGAGCAACAAAAACCGCGGCATTAT |
| | TAAAACCTTTAGCAACGGCTGCGATTATGTGAGCAACAAAGGCATGGATACCGTG |
| | AGCGTGGGCAACACCCTGTATTATGTGAACAAACAGGAAGGCAAAAGCCTGTAT |
| | GTGAAAGGCGAACCGATTATTAACTTTTATGATCCGCTGGTGTTTCCGAGCGATG |
| | AATTTGATGCGAGCATTAGCCAGGTGAACGAAAAAATTAACCAGAGCCTGGCGTT |
| | TATTCGCAAAAGCGATGAACTGCTGCATAACGTGAACGCGGGCAAAAGCACCAC |
| | CAACATTATGATTACCACCATTATTATTGTGATTATTGTGATTCTGCTGAGCCTGA |
| | TTGCGGTGGGCCTGCTGCTGTATTGCAAAGCGCGCAGCACCCCGGTGACCCTGAG |
| | CAAAGATCAGCTGAGCGGCATTAACAACATTGCGTTTAGCAACTGATAA |
| SEQ ID | QNITEEFYQSTCSAVSKGYLSALRTGWYTSVITIELSNIKENKCNGTDAKVKLIKQELD |
| NO: 368 | KYKNAVTELQLLMQSTPPTNNRARRGSSKVLHLEGEVNKIKSALLSTNKAVVSLSNG |
| Corresponding | VSVLTSKVLDLKNYIDKQLLPIVNKQSCSISNIETVIEFQQKNNRLLEITREFSVNAGVT |
| amino acid | TPVSTYMLTNSELLSLINDMPITNDQKKLMSNNVQIVRQQSYSIMSIIKEEVLAYVVQL |
| sequence | PLYGVIDTPCWKLHTSPLCTTNTKEGSNICLTRTDRGWYCDNAGSVSFFPQAETCKV |
| | QSNRVFCDTMNSLTLPSEINLCNVDIFNPKYDCKIMTSKTDVSSSVITSLGAIVSCYGK |
| | TKCTASNKNRGIIKTFSNGCDYVSNKGMDTVSVGNTLYYVNKQEGKSLYVKGEPIIN |
| | FYDPLVFPSDEFDASISQVNEKINQSLAFIRKSDELLHNVNAGKSTTNIMITTIII |
| | VIIVILLSLIAVGLLLYCKARSTPVTLSKDQLSGINNIAFSN** |

RSV-mDS-Cav1

| | |
|---|---|
| SEQ ID | CAGAACATTACCGAAGAATTTTATCAGAGCACCTGCAGCGCGGTGAGCAAAGGCT |
| NO: 285 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGTGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCTTTAAAGTGCTGGATCTGAAAAACTATATTGATAAACAGCTGCTGCCGATTCT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGTGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCAAAGCGCGCA |
| | GCACCCCGGTGACCCTGAGCAAAGATCAGCTGAGCGGCATTAACAACATTGCGTT |
| | TAGCAACTGATAA |
| SEQ ID | QNITEEFYQSTCSAVSKGYLSALRTGWYTSVITIELSNIKENKCNGTDAKVKLIKQELD |
| NO: 369 | KYKNAVTELQLLMQSTPPTNNRARRELPRFMNYTLNNAKKTNVTLSKKRKRRFLGF |
| Corresponding | LLGVGSAIASGVAVCKVLHLEGEVNKIKSALLSTNKAVVSLSNGVSVLTFKVLDLKN |
| amino acid | YIDKQLLPILNKQSCSISNIETVIEFQQKNNRLLEITREFSVNAGVTTPVSTYMLTNSEL |
| sequence | LSLINDMPITNDQKKLMSNNVQIVRQQSYSIMCIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTTNIMITTIIIVIIVILLSLIAVGLLLYCK |
| | ARSTPVTLSKDQLSGINNIAFSN |

RSV-sDS-Cav1

| | |
|---|---|
| SEQ ID | CAGAACATTACCGAAGAATTTTATCAGAGCACCTGCAGCGCGGTGAGCAAAGGCT |
| NO: 286 | ATCTGAGCGCGCTGCGCACCGGCTGGTATACCAGCGTGATTACCATTGAACTGAG |
| mRNA | CAACATTAAAGAAAACAAATGCAACGGCACCGATGCGAAAGTGAAACTGATTAA |
| | ACAGGAACTGGATAAATATAAAAACGCGGTGACCGAACTGCAGCTGCTGATGCA |
| | GAGCACCCCGCCGACCAACAACCGCGCGCGCCGCGAACTGCCGCGCTTTATGAAC |
| | TATACCCTGAACAACGCGAAAAAAACCAACGTGACCCTGAGCAAAAAACGCAAA |
| | CGCCGCTTTCTGGGCTTTCTGCTGGGCGTGGGCAGCGCGATTGCGAGCGGCGTGG |
| | CGGTGTGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCTTTAAAGTGCTGGATCTGAAAAACTATATTGATAAACAGCTGCTGCCGATTCT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGTGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACGGCTATATTCCGGAAGCGCCGCGCGATGGCCA |
| | GGCGTATGTGCGCAAAGATGGCGAATGGGTGCTGCTGAGCACCTTTCTGTGATAA |
| SEQ ID | QNITEEFYQSTCSAVSKGYLSALRTGWYTSVITIELSNIKENKCNGTDAKVKLIKQELD |
| NO: 370 | KYKNAVTELQLLMQSTPPTNNRARRELPRFMNYTLNNAKKTNVTLSKKRKRRFLGF |
| Corresponding | LLGVGSAIASGVAVCKVLHLEGEVNKIKSALLSTNKAVVSLSNGVSVLTFKVLDLKN |
| amino acid | YIDKQLLPILNKQSCSISNIETVIEFQQKNNRLLEITREFSVNAGVTTPVSTYMLTNSEL |
| sequence | LSLINDMPITNDQKKLMSNNVQIVRQQSYSIMCIIKEEVLAYVVQLPLYGVIDTPCWK |
| | LHTSPLCTTNTKEGSNICLTRTDRGWYCDNAGSVSFFPQAETCKVQSNRVFCDTMNS |
| | LTLPSEINLCNVDIFNPKYDCKIMTSKTDVSSSVITSLGAIVSCYGKTKCTASNKNRGII |
| | KTFSNGCDYVSNKGMDTVSVGNTLYYVNKQEGKSLYVKGEPIINFYDPLVFPSDEFD |
| | ASISQVNEKINQSLAFIRKSDELLHNVNAGKSTINGYIPEAPRDGQAYVRKDGEWVLL |
| | STFL** |

TABLE 6
Nucleic Acid Sequences and Amino acid Sequences of RSV comprising
signal peptide as indicated underline
WT (v1) -naturally occurring nucleic acid sequence

| | |
|---|---|
| SEQ ID NO: 1 | ATGGAACTGCTGATTCTGAAAGCGAACGCGATTACCACCATTCTGACCGCGGTGA |
| (1722 bp) | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAAC |
| SEQ ID NO: | MELLILKANAITTILTAVTFCFASGQNITEEFYQSTCSAVSKGYLSALRTGWYTSVITIE |
| 38 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| (wild type | EEVLAYVVQLPLYGVIDTPCWKLHTSPLCTTNTKEGSNICLTRTDRGWYCDNAGSVS |
| RSV-F | FFPQAETCKVQSNRVFCDTMNSLTLPSEINLCNVDIFNPKYDCKIMTSKTDVSSSVITS |
| protein) | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

WT (v2) - codon optimized sequence

| | |
|---|---|
| SEQ ID NO: 2 | ATGGAACTGCTGATTCTGAAAGCGAACGCGATTACCACCATTCTGACCGCGGTGA |
| (1722 bp) | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAAC |
| SEQ ID NO: | MELLILKANAITTILTAVTFCFASGQNITEEFYQSTCSAVSKGYLSALRTGWYTSVITIE |
| 38 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence (wild | REFSVNAGVTTPVSTYMLTNSELLSLINDMPITNDQKKLMSNNVQIVRQQSYSIMSIIK |
| type RSV-F | EEVLAYVVQLPLYGVIDTPCWKLHTSPLCTTNTKEGSNICLTRTDRGWYCDNAGSVS |
| protein) | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P1

| | |
|---|---|
| SEQ ID NO: 3 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACCCGCCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 39 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNPPSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P2

| | |
|---|---|
| SEQ ID NO: 4 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACCGCCGTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 40 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKPPCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P3

| | |
|---|---|
| SEQ ID NO: 5 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGCCGCCGAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 41 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQPPSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P4

| | |
|---|---|
| SEQ ID NO: 6 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCCGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 42 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P5

| | |
|---|---|
| SEQ ID NO: 7 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCCGCCGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 43 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPPFPSDEFDASISQVNEKINQSLAFIRKSDELLHNVNAGKSTTNIMI |
| | TTIIIVIIVILLSLIAVGLLLYCKARSTPVTLSKDQLSGINNIAFSN |

P6

| | |
|---|---|
| SEQ ID NO: 8 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACCCGCCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCCGCCGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 44 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNPPSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPPFPSDEFDASISQVNEKINQSLAFIRKSDELLHNVNAGKSTTNIMI |
| | TTIIIVIIVILLSLIAVGLLLYCKARSTPVTLSKDQLSGINNIAFSN |

P7

| | |
|---|---|
| SEQ ID NO: 9 | ATGGAACTGCTGATTCTGAAAGCGAACGCGATTACCACCATTCTGACCGCGGTGA |
| (1728 bp) ORF | CCTTTTGCTTTGCGAGCGGCCAGAACATTACCGAAGAATTTTATCAGAGCACCTG |
| of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACCGCCGTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCCGCCGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 45 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKPPCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPPFPSDEFDASISQVNEKINQSLAFIRKSDELLHNVNAGKSTTNIMI |
| | TTIIIVIIVILLSLIAVGLLLYCKARSTPVTLSKDQLSGINNIAFSN |

P8

| | |
|---|---|
| SEQ ID NO: | ATGGAACTGCTGATTCTGAAAGCGAACGCGATTACCACCATTCTGACCGCGGTGA |
| 10 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGCCGCCGAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCCGCCGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 46 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQPPSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPPFPSDEFDASISQVNEKINQSLAFIRKSDELLHNVNAGKSTTNIMI |
| | TTIIIVIIVILLSLIAVGLLLYCKARSTPVTLSKDQLSGINNIAFSN |

P9

| | |
|---|---|
| SEQ ID NO: | ATGGAACTGCTGATTCTGAAAGCGAACGCGATTACCACCATTCTGACCGCGGTGA |
| 11 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACCCGCCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCCGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 47 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNPPSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P10

| | |
|---|---|
| SEQ ID NO: | ATGGAACTGCTGATTCTGAAAGCGAACGCGATTACCACCATTCTGACCGCGGTGA |
| 12 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACCGCCGTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCCGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 48 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKPPCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

P11

| | |
|---|---|
| SEQ ID NO: | ATGGAACTGCTGATTCTGAAAGCGAACGCGATTACCACCATTCTGACCGCGGTGA |
| 13 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGCCGCCGAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCCGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 49 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQPPSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPPVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

210-T-S-V

| | |
|---|---|
| SEQ ID NO: | ATGGAACTGCTGATTCTGAAAGCGAACGCGATTACCACCATTCTGACCGCGGTGA |
| 14 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTCTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 50 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVCTSKVLDLKNYIDKQLLPILNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-T-S

| | |
|---|---|
| SEQ ID NO: | ATGGAACTGCTGATTCTGAAAGCGAACGCGATTACCACCATTCTGACCGCGGTGA |
| 15 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTGTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 51 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVCTSKVLDLKNYIDKQLLPIVNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-T-V

| | |
|---|---|
| SEQ ID NO: | ATGGAACTGCTGATTCTGAAAGCGAACGCGATTACCACCATTCTGACCGCGGTGA |
| 16 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 52 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVLTSKVLDLKNYIDKQLLPILNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-S-V

| | |
|---|---|
| SEQ ID NO: | ATGGAACTGCTGATTCTGAAAGCGAACGCGATTACCACCATTCTGACCGCGGTGA |
| 17 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTCTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 53 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVCTSKVLDLKNYIDKQLLPILNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-T

| | |
|---|---|
| SEQ ID NO: | ATGGAACTGCTGATTCTGAAAGCGAACGCGATTACCACCATTCTGACCGCGGTGA |
| 18 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 54 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVLTSKVLDLKNYIDKQLLPIVNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-S

| | |
|---|---|
| SEQ ID NO: | ATGGAACTGCTGATTCTGAAAGCGAACGCGATTACCACCATTCTGACCGCGGTGA |
| 19 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTGTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 55 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVCTSKVLDLKNYIDKQLLPIVNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-V-3

| | |
|---|---|
| SEQ ID NO: | ATGGAACTGCTGATTCTGAAAGCGAACGCGATTACCACCATTCTGACCGCGGTGA |
| 20 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 56 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPILNKPSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-V

| | |
|---|---|
| SEQ ID NO: | ATGGAACTGCTGATTCTGAAAGCGAACGCGATTACCACCATTCTGACCGCGGTGA |
| 21 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAACCGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 57 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPILNKPSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

210-213-T-S-V

| | |
|---|---|
| SEQ ID NO: | ATGGAACTGCTGATTCTGAAAGCGAACGCGATTACCACCATTCTGACCGCGGTGA |
| 22 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTCTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 58 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVCTSKVLDLKNYIDKQLLPILNKCSCCISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-T-S

| | |
|---|---|
| SEQ ID NO: | ATGGAACTGCTGATTCTGAAAGCGAACGCGATTACCACCATTCTGACCGCGGTGA |
| 23 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTGTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 59 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVCTSKVLDLKNYIDKQLLPIVNKCSCCISNIETVIEFQQKNNRLLE |
| sequence | ITREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-T-V

| | |
|---|---|
| SEQ ID NO: | ATGGAACTGCTGATTCTGAAAGCGAACGCGATTACCACCATTCTGACCGCGGTGA |
| 24 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 60 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVLTSKVLDLKNYIDKQLLPILNKCSCCISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-S-V

| | |
|---|---|
| SEQ ID NO: | ATGGAACTGCTGATTCTGAAAGCGAACGCGATTACCACCATTCTGACCGCGGTGA |
| 25 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTCTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 61 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVCTSKVLDLKNYIDKQLLPILNKCSCCISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-T

| | |
|---|---|
| SEQ ID NO: | ATGGAACTGCTGATTCTGAAAGCGAACGCGATTACCACCATTCTGACCGCGGTGA |
| 26 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 62 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVLTSKVLDLKNYIDKQLLPIVNKCSCCISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-S

| | |
|---|---|
| SEQ ID NO: | ATGGAACTGCTGATTCTGAAAGCGAACGCGATTACCACCATTCTGACCGCGGTGA |
| 27 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTGTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 63 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVCTSKVLDLKNYIDKQLLPIVNKCSCCISNIETVIEFQQKNNRLLE |
| sequence | ITREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-V-3

| | |
|---|---|
| SEQ ID NO: | ATGGAACTGCTGATTCTGAAAGCGAACGCGATTACCACCATTCTGACCGCGGTGA |
| 28 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAGTGAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCGTGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 64 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPILNKCSCCISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

210-213-V

| | |
|---|---|
| SEQ ID NO: | ATGGAACTGCTGATTCTGAAAGCGAACGCGATTACCACCATTCTGACCGCGGTGA |
| 29 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAATGCAGCTGCTGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 65 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPILNKCSCCISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

S55C + L188C

| | |
|---|---|
| SEQ ID NO: | ATGGAACTGCTGATTCTGAAAGCGAACGCGATTACCACCATTCTGACCGCGGTGA |
| 30 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCTGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGTGCACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTCVITIE |
| 66 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVCTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

T103C + I148C

| | |
|---|---|
| SEQ ID NO: | ATGGAACTGCTGATTCTGAAAGCGAACGCGATTACCACCATTCTGACCGCGGTGA |
| 31 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGTGCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGTGCGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 67 | LSNIKENKCNGTDAKVKLIKQELDKYKNAVTELQLLMQSTPACNNRARRELPRFMN |
| Corresponding | YTLNNAKKTNVTLSKKRKRRFLGFLLGVGSACASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

P102A + I379V + M447V

| | |
|---|---|
| SEQ ID NO: | ATGGAACTGCTGATTCTGAAAGCGAACGCGATTACCACCATTCTGACCGCGGTGA |
| 32 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 68 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

V207L

| | |
|---|---|
| SEQ ID NO: | ATGGAACTGCTGATTCTGAAAGCGAACGCGATTACCACCATTCTGACCGCGGTGA |
| 33 | CCTTTTGCTTTGCGAGCGGCCAGAACATTACCGAAGAATTTTATCAGAGCACCTG |
| (1728 bp) | CAGCGCGGTGAGCAAAGGCTATCTGAGCGCGCTGCGCACCGGCTGGTATACCAGC |
| ORF of mRNA | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTCTGAACAAACAGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGAGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACATTATGATTACCACCA |
| | TTATTATTGTGATTATTGTGATTCTGCTGAGCCTGATTGCGGTGGGCCTGCTGCTG |
| | TATTGCAAAGCGCGCAGCACCCCGGTGACCCTGAGCAAAGATCAGCTGAGCGGC |
| | ATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 69 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPILNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

R106K

| | |
|---|---|
| SEQ ID NO: | ATGGAACTGCTGATTCTGAAAGCGAACGCGATTACCACCATTCTGACCGCGGTGA |
| 80 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACAAAGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 81 | LSNIKENKCNGTDAKVKLIKQELDKYKNAVTELQLLMQSTPPTNNKARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | EEVLAYVVQLPLYGVIDTPCWKLHTSPLCTTNTKEGSNICLTRTDRGWYCDNAGSVS |
| | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

R109Q

| | |
|---|---|
| SEQ ID NO: 82 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCAG |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: 83 | MELLILKANAITTILTAVTFCFASGQNITEEFYQSTCSAVSKGYLSALRTGWYTSVITIE |
| Corresponding | LSNIKENKCNGTDAKVKLIKQELDKYKNAVTELQLLMQSTPPTNNRARQELPRFMNY |
| amino acid | TLNNAKKTNVTLSKKRKRRFLGFLLGVGSAIASGVAVSKVLHLEGEVNKIKSALLST |
| sequence | NKAVVSLSNGVSVLTSKVLDLKNYIDKQLLPIVNKQSCSISNIETVIEFQQKNNRLLEIT |
| | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

R133Q

| | |
|---|---|
| SEQ ID NO: 84 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACAGAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: 85 | MELLILKANAITTILTAVTFCFASGQNITEEFYQSTCSAVSKGYLSALRTGWYTSVITIE |
| Corresponding | LSNIKENKCNGTDAKVKLIKQELDKYKNAVTELQLLMQSTPPTNNRARRELPRFMNY |
| amino acid | TLNNAKKTNVTLSKKQKRRFLGFLLGVGSAIASGVAVSKVLHLEGEVNKIKSALLST |
| sequence | NKAVVSLSNGVSVLTSKVLDLKNYIDKQLLPIVNKQSCSISNIETVIEFQQKNNRLLEIT |
| | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

K134Q

| | |
|---|---|
| SEQ ID NO: 86 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCCAGCGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 87 | LSNIKENKCNGTDAKVKLIKQELDKYKNAVTELQLLMQSTPPTNNRARRELPRFMNY |
| Corresponding | TLNNAKKTNVTLSKKRQRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

R135Q

| | |
|---|---|
| SEQ ID NO: 88 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACAGCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 89 | LSNIKENKCNGTDAKVKLIKQELDKYKNAVTELQLLMQSTPPTNNRARRELPRFMNY |
| Corresponding | TLNNAKKTNVTLSKKRKQRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN ** |

R136Q

| | |
|---|---|
| SEQ ID NO: 90 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCAGTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 91 | LSNIKENKCNGTDAKVKLIKQELDKYKNAVTELQLLMQSTPPTNNRARRELPRFMNY |
| Corresponding | TLNNAKKTNVTLSKKRKRQFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

L142Q

| | |
|---|---|
| SEQ ID NO: | ATGGAACTGCTGATTCTGAAAGCGAACGCGATTACCACCATTCTGACCGCGGTGA |
| 92 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCAGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: 93 | MELLILKANAITTILTAVTFCFASGQNITEEFYQSTCSAVSKGYLSALRTGWYTSVITIE |
| Corresponding | LSNIKENKCNGTDAKVKLIKQELDKYKNAVTELQLLMQSTPPTNNRARRELPRFMNY |
| amino acid | TLNNAKKTNVTLSKKRKRRFLGFLQGVGSAIASGVAVSKVLHLEGEVNKIKSALLST |
| sequence | NKAVVSLSNGVSVLTSKVLDLKNYIDKQLLPIVNKQSCSISNIETVIEFQQKNNRLLEIT |
| | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

A149C

| | |
|---|---|
| SEQ ID NO: 94 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTTGCAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 95 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAICSGVAVSKVLHLEGEVNKIKSALLSTN |
| amino acic | KAVVSLSNGVSVLTSKVLDLKNYIDKQLLPIVNKQSCSISNIETVIEFQQKNNRLLEITR |
| sequence | EFSVNAGVTTPVSTYMLTNSELLSLINDMPITNDQKKLMSNNVQIVRQQSYSIMSIIKE |
| | EVLAYVVQLPLYGVIDTPCWKLHTSPLCTTNTKEGSNICLTRTDRGWYCDNAGSVSF |
| | FPQAETCKVQSNRVFCDTMNSLTLPSEINLCNVDIFNPKYDCKIMTSKTDVSSSVITSL |
| | GAIVSCYGKTKCTASNKNRGIIKTFSNGCDYVSNKGMDTVSVGNTLYYVNKQEGKSL |
| | YVKGEPIINFYDPLVFPSDEFDASISQVNEKINQSLAFIRKSDELLHNVNAGKSTTNIMI |
| | TTIIIVIIVILLSLIAVGLLLYCKARSTPVTLSKDQLSGINNIAFSN |

S155C

| | |
|---|---|
| SEQ ID NO: 96 | ATGGAACTGCTGATTCTGAAAGCGAACGCGATTACCACCATTCTGACCGCGGTGA |
| ORF of mRNA | CCTTTTGCTTTGCGAGCGGCCAGAACATTACCGAAGAATTTTATCAGAGCACCTG |
| | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGTGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 97 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVCKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

S190F

| | |
|---|---|
| SEQ ID NO: | ATGGAACTGCTGATTCTGAAAGCGAACGCGATTACCACCATTCTGACCGCGGTGA |
| 98 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCTTTAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 99 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTFKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

S290C

| | |
|---|---|
| SEQ ID NO: | ATGGAACTGCTGATTCTGAAAGCGAACGCGATTACCACCATTCTGACCGCGGTGA |
| 100 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGTGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 101 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMCIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

Y458C

| | |
|---|---|
| SEQ ID NO: | ATGGAACTGCTGATTCTGAAAGCGAACGCGATTACCACCATTCTGACCGCGGTGA |
| 102 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTGCGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 103 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYCVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

Q501G

| | |
|---|---|
| SEQ ID | ATGGAACTGCTGATTCTGAAAGCGAACGCGATTACCACCATTCTGACCGCGGTGA |
| NO: 104 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACGGCAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 105 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINGSLAFIRKSDELLHNVNAGKSTTNIM |
| | ITTIIIVIIVILLSLIAVGLLLYCKARSTPVTLSKDQLSGINNIAFSN |

L512K

| | |
|---|---|
| SEQ ID | ATGGAACTGCTGATTCTGAAAGCGAACGCGATTACCACCATTCTGACCGCGGTGA |
| NO: 106 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | AAAACTGCATAACGTGAACGCGGGCAAAAGCACCACCAACATTATGATTACCAC |
| | CATTATTATTGTGATTATTGTGATTCTGCTGAGCCTGATTGCGGTGGGCCTGCTGC |
| | TGTATTGCAAAGCGCGCAGCACCCCGGTGACCCTGAGCAAAGATCAGCTGAGCG |
| | GCATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 107 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | EEVLAYVVQLPLYGVIDTPCWKLHTSPLCTTNTKEGSNICLTRTDRGWYCDNAGSVS |
| | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDEKLHNVNAGKSTTNIM |
| | ITTIIIVIIVILLSLIAVGLLLYCKARSTPVTLSKDQLSGINNIAFSN |

E2D

| | |
|---|---|
| SEQ ID | ATGGATCTGCTGATTCTGAAAGCGAACGCGATTACCACCATTCTGACCGCGGTGA |
| NO: 108 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MDLLILKANAITTILTAVTFCFASGQNITEEFYQSTCSAVSKGYLSALRTGWYTSVITIE |
| 109 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

L4P

| | |
|---|---|
| SEQ ID | ATGGAACTGCCGATTCTGAAAGCGAACGCGATTACCACCATTCTGACCGCGGTGA |
| NO: 110 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELPILKANAITTILTAVTFCFASGQNITEEFYQSTCSAVSKGYLSALRTGWYTSVITIE |
| 111 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

A8T

| | |
|---|---|
| SEQ ID | ATGGAACTGCTGATTCTGAAAACCAACGCGATTACCACCATTCTGACCGCGGTGA |
| NO: 112 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID | MELLILKTNAITTILTAVTFCFASGQNITEEFYQSTCSAVSKGYLSALRTGWYTSVITIE |
| NO: 113 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

T13A

| | |
|---|---|
| SEQ ID NO: | ATGGAACTGCTGATTCTGAAAGCGAACGCGATTACCGCGATTCTGACCGCGGTGA |
| 114 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITAILTAVTFCFASGQNITEEFYQSTCSAVSKGYLSALRTGWYTSVITIE |
| 115 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

L15F

| | |
|---|---|
| SEQ ID | ATGGAACTGCTGATTCTGAAAGCGAACGCGATTACCACCATTTTTACCGCGGTGA |
| NO: 116 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTIFTAVTFCFASGQNITEEFYQSTCSAVSKGYLSALRTGWYTSVITIE |
| 117 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

T16A

| | |
|---|---|
| SEQ ID | ATGGAACTGCTGATTCTGAAAGCGAACGCGATTACCACCATTCTGGCGGCGGTGA |
| NO: 118 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILAAVTFCFASGQNITEEFYQSTCSAVSKGYLSALRTGWYTSVITIE |
| 119 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

F20L

| | |
|---|---|
| SEQ ID | ATGGAACTGCTGATTCTGAAAGCGAACGCGATTACCACCATTCTGACCGCGGTGA |
| NO: 120 | CCCTGTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTLCFASGQNITEEFYQSTCSAVSKGYLSALRTGWYTSVITIE |
| 121 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

G25S

| | |
|---|---|
| SEQ ID | ATGGAACTGCTGATTCTGAAAGCGAACGCGATTACCACCATTCTGACCGCGGTGA |
| NO: 122 | CCTTTTGCTTTGCGAGCAGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| SEQ ID NO: | AAACAGCTGCTGCCGATTGTGAACAAACAGAGCTGCAGCATTAGCAACATTGAA |
| 123 | ACCGTGATTGAATTTCAGCAGAAAAACAACCGCCTGCTGGAAATTACCCGCGAAT |
| Corresponding | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| amino acid | MELLILKANAITTILTAVTFCFASSQNITEEFYQSTCSAVSKGYLSALRTGWYTSVITIE |
| sequence | LSNIKENKCNGTDAKVKLIKQELDKYKNAVTELQLLMQSTPATNNRARRELPRFMN |
| | YTLNNAKKTNVTLSKKRKRRFLGFLLGVGSAIASGVAVSKVLHLEGEVNKIKSALLS |
| | TNKAVVSLSNGVSVLTSKVLDLKNYIDKQLLPIVNKQSCSISNIETVIEFQQKNNRLLEI |
| | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

E30V

| | |
|---|---|
| SEQ ID | ATGGAACTGCTGATTCTGAAAGCGAACGCGATTACCACCATTCTGACCGCGGTGA |
| NO: 124 | CCTTTTGCTTTGCGAGCGGCCAGAACATTACCGTGGAATTTTATCAGAGCACCTGC |
| ORF of mRNA | AGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITVEFYQSTCSAVSKGYLSALRTGWYTSVITIE |
| 125 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

T54H

| | |
|---|---|
| SEQ ID | ATGGAACTGCTGATTCTGAAAGCGAACGCGATTACCACCATTCTGACCGCGGTGA |
| NO: 126 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATCATAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYHSVITIE |
| 127 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

N88S

| | |
|---|---|
| SEQ ID | ATGGAACTGCTGATTCTGAAAGCGAACGCGATTACCACCATTCTGACCGCGGTGA |
| NO: 128 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAGCGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 129 | LSNIKENKCNGTDAKVKLIKQELDKYKSAVTELQLLMQSTPATNNRARRELPRFMNY |
| Corresponding | TLNNAKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEVNLCNVDIFNPKYDCKIMTSKTDVSSSVITS |
| | LGAIVSCYGKTKCTASNKNRGIIKTFSNGCDYVSNKGVDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

T103A

| | |
|---|---|
| SEQ ID | ATGGAACTGCTGATTCTGAAAGCGAACGCGATTACCACCATTCTGACCGCGGTGA |
| NO: 130 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGGCGAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 131 | LSNIKENKCNGTDAKVKLIKQELDKYKNAVTELQLLMQSTPAANNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

A122T

| | |
|---|---|
| SEQ ID | ATGGAACTGCTGATTCTGAAAGCGAACGCGATTACCACCATTCTGACCGCGGTGA |
| NO: 132 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACACCAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 133 | LSNIKENKCNGTDAKVKLIKQELDKYKNAVTELQLLMQSTPATNNRARRELPRFMN |
| Corresponding | YTLNNTKKTNVTLSKKRKRRFLGFLLGVGSAIASGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEVNLCNVDIFNPKYDCKIMTSKTDVSSSVITS |
| | LGAIVSCYGKTKCTASNKNRGIIKTFSNGCDYVSNKGVDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

K124N

| | |
|---|---|
| SEQ ID | ATGGAACTGCTGATTCTGAAAGCGAACGCGATTACCACCATTCTGACCGCGGTGA |
| NO: 134 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAACACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 135 | LSNIKENKCNGTDAKVKLIKQELDKYKNAVTELQLLMQSTPATNNRARRELPRFMN |
| Corresponding | YTLNNAKNTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

T125N

| | |
|---|---|
| SEQ ID | ATGGAACTGCTGATTCTGAAAGCGAACGCGATTACCACCATTCTGACCGCGGTGA |
| NO: 136 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAAACAACGTG |
| | ACCCTGAGCAAAAAACGCAAACGCCGCTTTCTGGGCTTTCTGCTGGGCGTGGGCA |
| | GCGCGATTGCGAGCGGCGTGGCGGTGAGCAAAGTGCTGCATCTGGAAGGCGAAG |
| | TGAACAAAATTAAAAGCGCGCTGCTGAGCACCAACAAAGCGGTGGTGAGCCTGA |
| | GCAACGGCGTGAGCGTGCTGACCAGCAAAGTGCTGGATCTGAAAAACTATATTGA |
| | TAAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID | MELLILKANAITTILTAVTFCFASGQNITEEFYQSTCSAVSKGYLSALRTGWYTSVITIE |
| NO: 137 | LSNIKENKCNGTDAKVKLIKQELDKYKNAVTELQLLMQSTPATNNRARRELPRFMN |
| Corresponding | YTLNNAKKNNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

R136K

| | |
|---|---|
| SEQ ID | ATGGAACTGCTGATTCTGAAAGCGAACGCGATTACCACCATTCTGACCGCGGTGA |
| NO: 138 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCAAATTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 139 | LSNIKENKCNGTDAKVKLIKQELDKYKNAVTELQLLMQSTPATNNRARRELPRFMN |
| Corresponding | YTLNNAKKTNVTLSKKRKRKFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

V152I

| | |
|---|---|
| SEQ ID | ATGGAACTGCTGATTCTGAAAGCGAACGCGATTACCACCATTCTGACCGCGGTGA |
| NO: 140 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCATTGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 141 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGIAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEVNLCNVDIFNPKYDCKIMTSKTDVSSSVITS |
| | LGAIVSCYGKTKCTASNKNRGIIKTFSNGCDYVSNKGVDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

S190I

| | |
|---|---|
| SEQ ID | ATGGAACTGCTGATTCTGAAAGCGAACGCGATTACCACCATTCTGACCGCGGTGA |
| NO: 142 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCATTAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 143 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTIKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

N227S

| | |
|---|---|
| SEQ ID | ATGGAACTGCTGATTCTGAAAGCGAACGCGATTACCACCATTCTGACCGCGGTGA |
| NO: 144 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAGCAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 145 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKSNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

V296I

| | |
|---|---|
| SEQ ID | ATGGAACTGCTGATTCTGAAAGCGAACGCGATTACCACCATTCTGACCGCGGTGA |
| NO: 146 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAATTCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 147 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEILAYVVQLPLYGVIDTPCWKLHTSPLCTTNTKEGSNICLTRTDRGWYCDNAGSVS |
| | FFPQAETCKVQSNRVFCDTMNSLTLPSEVNLCNVDIFNPKYDCKIMTSKTDVSSSVITS |
| | LGAIVSCYGKTKCTASNKNRGIIKTFSNGCDYVSNKGVDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

Q354L

| | |
|---|---|
| SEQ ID | ATGGAACTGCTGATTCTGAAAGCGAACGCGATTACCACCATTCTGACCGCGGTGA |
| NO: 148 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCTGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 149 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPLAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

L373R

| | |
|---|---|
| SEQ ID | ATGGAACTGCTGATTCTGAAAGCGAACGCGATTACCACCATTCTGACCGCGGTGA |
| NO: 150 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCGCACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 151 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSRTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN |

D486S

| | |
|---|---|
| SEQ ID | ATGGAACTGCTGATTCTGAAAGCGAACGCGATTACCACCATTCTGACCGCGGTGA |
| NO: 152 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCAGCGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID | MELLILKANAITTILTAVTFCFASGQNITEEFYQSTCSAVSKGYLSALRTGWYTSVITIE |
| NO: 153 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSSEFDASISQVNEKINQSLAFIRKSDELLHNVNAGKSTTNI |
| | MITTIIIVIIVILLSLIAVGLLLYCKARSTPVTLSKDQLSGINNIAFSN |

S540L

| | |
|---|---|
| SEQ ID | ATGGAACTGCTGATTCTGAAAGCGAACGCGATTACCACCATTCTGACCGCGGTGA |
| NO: 154 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGCTGCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 155 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLLLIAVGLLLYCKARSTPVTLSKDQLSGINNIAFSN |

L547F

| | |
|---|---|
| SEQ ID | ATGGAACTGCTGATTCTGAAAGCGAACGCGATTACCACCATTCTGACCGCGGTGA |
| NO: 156 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGTTTCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 157 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVSKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMSII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLFLYCKARSTPVTLSKDQLSGINNIAFSN |

S155C + S290C

| | |
|---|---|
| SEQ ID | ATGGAACTGCTGATTCTGAAAGCGAACGCGATTACCACCATTCTGACCGCGGTGA |
| NO: 158 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGTGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGTGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID | MELLILKANAITTILTAVTFCFASGQNITEEFYQSTCSAVSKGYLSALRTGWYTSVITIE |
| NO: 159 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAIASGVAVCKVLHLEGEVNKIKSALLS |
| amino acid | TNKAVVSLSNGVSVLTSKVLDLKNYIDKQLLPIVNKQSCSISNIETVIEFQQKNNRLLEI |
| sequence | TREFSVNAGVTTPVSTYMLTNSELLSLINDMPITNDQKKLMSNNVQIVRQQSYSIMCII |
| | KEEVLAYVVQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSKTDVSSSVIT |
| | SLGAIVSCYGKTKCTASNKNRGIIKTFSNGCDYVSNKGVDTVSVGNTLYYVNKQEGK |
| | SLYVKGEPIINFYDPLVFPSDEFDASISQVNEKINQSLAFIRKSDELLHNVNAGKSTTNI |
| | MITTIIIVIIVILLSLIAVGLLLYCKARSTPVTLSKDQLSGINNIAFSN ** |

A149C + Y458C

| | |
|---|---|
| SEQ ID | ATGGAACTGCTGATTCTGAAAGCGAACGCGATTACCACCATTCTGACCGCGGTGA |
| NO: 160 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTTGCAGCGGCGTGGCGGTGAGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCAGCAAAGTGCTGGATCTGAAAAACTATATTGAT |
| | AAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAGTGAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCGTGGATACCGTGAGCGTGGGCAACACCCTG |
| | TATTGCGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID NO: | MELLILKANAITTILTAVTFCFASGQNITEEFYQSTCSAVSKGYLSALRTGWYTSVITIE |
| 161 | LSNIKENKCNGTDAKVKLIKQELDKYKNAVTELQLLMQSTPATNNRARRELPRFMN |
| Corresponding | YTLNNAKKTNVTLSKKRKRRFLGFLLGVGSAICSGVAVSKVLHLEGEVNKIKSALLST |
| amino acid | NKAVVSLSNGVSVLTSKVLDLKNYIDKQLLPIVNKQSCSISNIETVIEFQQKNNRLLEIT |
| sequence | REFSVNAGVTTPVSTYMLTNSELLSLINDMPITNDQKKLMSNNVQIVRQQSYSIMSIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEVNLCNVDIFNPKYDCKIMTSKTDVSSSVITS |
| | LGAIVSCYGKTKCTASNKNRGIIKTFSNGCDYVSNKGVDTVSVGNTLYCVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

mRNA-1345

| | |
|---|---|
| SEQ ID | ATGGAACTGCTGATTCTGAAAGCGAACGCGATTACCACCATTCTGACCGCGGTGA |
| NO: 162 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGGCGACCGGCAGCGGCAGCGCGATT |
| | TGCAGCGGCGTGGCGGTGTGCAAAGTGCTGCATCTGGAAGGCGAAGTGAACAAA |
| | ATTAAAAGCGCGCTGCTGAGCACCAACAAAGCGGTGGTGAGCCTGAGCAACGGC |
| | GTGAGCGTGCTGACCTTTAAAGTGCTGGATCTGAAAAACTATATTGATAAACAGC |
| | TGCTGCCGATTCTGAACAAACAGAGCTGCAGCATTAGCAACATTGAAACCGTGAT |
| | TGAATTTCAGCAGAAAAACAACCGCCTGCTGGAAATTACCCGCGAATTTAGCGTG |
| | AACGCGGGCGTGACCACCCCGGTGAGCACCTATATGCTGACCAACAGCGAACTGC |
| | TGAGCCTGATTAACGATATGCCGATTACCAACGATCAGAAAAAACTGATGAGCAA |
| | CAACGTGCAGATTGTGCGCCAGCAGAGCTATAGCATTATGTGCATTATTAAAGAA |
| | GAAGTGCTGGCGTATGTGGTGCAGCTGCCGCTGTATGGCGTGATTGATACCCCGT |
| | GCTGGAAACTGCATACCAGCCCGCTGTGCACCACCAACACCAAAGAAGGCAGCA |
| | ACATTTGCCTGACCCGCACCGATCGCGGCTGGTATTGCGATAACGCGGGCAGCGT |
| | GAGCTTTTTTCCGCAGGCGGAAACCTGCAAAGTGCAGAGCAACCGCGTGTTTTGC |
| | GATACCATGAACAGCCGCACCCTGCCGAGCGAAGTGAACCTGTGCAACGTGGAT |
| | ATTTTTAACCCGAAATATGATTGCAAAATTATGACCAGCAAAACCGATGTGAGCA |
| | GCAGCGTGATTACCAGCCTGGGCGCGATTGTGAGCTGCTATGGCAAAACCAAATG |
| | CACCGCGAGCAACAAAAACCGCGGCATTATTAAAACCTTTAGCAACGGCTGCGAT |
| | TATGTGAGCAACAAAGGCGTGGATACCGTGAGCGTGGGCAACACCCTGTATTGCG |
| | TGAACAAACAGGAAGGCAAAAGCCTGTATGTGAAAGGCGAACCGATTATTAACT |
| | TTTATGATCCGCTGGTGTTTCCGAGCGATGAATTTGATGCGAGCATTAGCCAGGTG |
| | AACGAAAAAATTAACCAGAGCCTGGCGTTTATTCGCAAAAGCGATGAACTGCTGC |
| | ATAACGTGAACGCGGGCAAAAGCACCACCAACATTATGATTACCACCATTATTAT |
| | TGTGATTATTGTGATTCTGCTGAGCCTGATTGCGGTGGGCCTGCTGCTGTATTGAT |
| | AA |
| SEQ ID | MELLILKANAITTILTAVTFCFASGQNITEEFYQSTCSAVSKGYLSALRTGWYTSVITIE |
| NO: 163 | LSNIKENKCNGTDAKVKLIKQELDKYKNAVTELQLLMQSTPATGSGSAICSGVAVCK |
| Corresponding | VLHLEGEVNKIKSALLSTNKAVVSLSNGVSVLTFKVLDLKNYIDKQLLPILNKQSCSIS |
| amino acid | NIETVIEFQQKNNRLLEITREFSVNAGVTTPVSTYMLTNSELLSLINDMPITNDQKKLM |
| sequence | SNNVQIVRQQSYSIMCIIKEEVLAYVVQLPLYGVIDTPCWKLHTSPLCTTNTKEGSNIC |
| | LTRTDRGWYCDNAGSVSFFPQAETCKVQSNRVFCDTMNSRTLPSEVNLCNVDIFNPK |
| | YDCKIMTSKTDVSSSVITSLGAIVSCYGKTKCTASNKNRGIIKTFSNGCDYVSNKGVD |
| | TVSVGNTLYCVNKQEGKSLYVKGEPIINFYDPLVFPSDEFDASISQVNEKINQSLAFIR |
| | KSDELLHNVNAGKSTTNIMITTIIIVIIVILLSLIAVGLLLY |

FP-T4

| | |
|---|---|
| SEQ ID | ATGGAACTGCTGATTCTGAAAGCGAACGCGATTACCACCATTCTGACCGCGGTGA |
| NO: 164 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GGCAGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACGGCTATATTCCGGAAGCGCCGCGCGATGGCCA |
| | GGCGTATGTGCGCAAAGATGGCGAATGGGTGCTGCTGAGCACCTTTCTGTGATAA |
| SEQ ID NO: | MELLILKANAITTILTAVTFCFASGQNITEEFYQSTCSAVSKGYLSALRTGWYTSVITIE |
| 165 | LSNIKENKCNGTDAKVKLIKQELDKYKNAVTELQLLMQSTPPTNNRARRGSFLGFLL |
| Corresponding | GVGSAIASGVAVSKVLHLEGEVNKIKSALLSTNKAVVSLSNGVSVLTSKVLDLKNYID |
| amino acid | KQLLPIVNKQSCSISNIETVIEFQQKNNRLLEITREFSVNAGVTTPVSTYMLTNSELLSLI |
| sequence | NDMPITNDQKKLMSNNVQIVRQQSYSIMSIIKEEVLAYVVQLPLYGVIDTPCWKLHTS |
| | PLCTTNTKEGSNICLTRTDRGWYCDNAGSVSFFPQAETCKVQSNRVFCDTMNSLTLPS |
| | EINLCNVDIFNPKYDCKIMTSKTDVSSSVITSLGAIVSCYGKTKCTASNKNRGIIKTFSN |
| | GCDYVSNKGMDTVSVGNTLYYVNKQEGKSLYVKGEPIINFYDPLVFPSDEFDASISQ |
| | VNEKINQSLAFIRKSDELLHNVNAGKSTTNGYIPEAPRDGQAYVRKDGEWVLLSTFL |

GS-FP-TM

| | |
|---|---|
| SEQ ID | ATGGAACTGCTGATTCTGAAAGCGAACGCGATTACCACCATTCTGACCGCGGTGA |
| NO: 166 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GGCAGCTTTCTGGGCTTTCTGCTGGGCGTGGGCAGCGCGATTGCGAGCGGCGTGG |
| | CGGTGAGCAAAGTGCTGCATCTGGAAGGCGAAGTGAACAAAATTAAAAGCGCGC |
| | TGCTGAGCACCAACAAAGCGGTGGTGAGCCTGAGCAACGGCGTGAGCGTGCTGA |
| | CCAGCAAAGTGCTGGATCTGAAAAACTATATTGATAAACAGCTGCTGCCGATTGT |
| | GAACAAACAGAGCTGCAGCATTAGCAACATTGAAACCGTGATTGAATTTCAGCAG |
| | AAAAACAACCGCCTGCTGGAAATTACCCGCGAATTTAGCGTGAACGCGGGCGTG |
| | ACCACCCCGGTGAGCACCTATATGCTGACCAACAGCGAACTGCTGAGCCTGATTA |
| | ACGATATGCCGATTACCAACGATCAGAAAAAACTGATGAGCAACAACGTGCAGA |
| | TTGTGCGCCAGCAGAGCTATAGCATTATGAGCATTATTAAAGAAGAAGTGCTGGC |
| | GTATGTGGTGCAGCTGCCGCTGTATGGCGTGATTGATACCCCGTGCTGGAAACTG |
| | CATACCAGCCCGCTGTGCACCACCAACACCAAAGAAGGCAGCAACATTTGCCTGA |
| | CCCGCACCGATCGCGGCTGGTATTGCGATAACGCGGGCAGCGTGAGCTTTTTTCC |
| | GCAGGCGGAAACCTGCAAAGTGCAGAGCAACCGCGTGTTTTGCGATACCATGAA |
| | CAGCCTGACCCTGCCGAGCGAAATTAACCTGTGCAACGTGGATATTTTTAACCCG |
| | AAATATGATTGCAAAATTATGACCAGCAAAACCGATGTGAGCAGCAGCGTGATTA |
| | CCAGCCTGGGCGCGATTGTGAGCTGCTATGGCAAAACCAAATGCACCGCGAGCA |
| | ACAAAAACCGCGGCATTATTAAAACCTTTAGCAACGGCTGCGATTATGTGAGCAA |
| | CAAAGGCATGGATACCGTGAGCGTGGGCAACACCCTGTATTATGTGAACAAACA |
| | GGAAGGCAAAAGCCTGTATGTGAAAGGCGAACCGATTATTAACTTTTATGATCCG |
| | CTGGTGTTTCCGAGCGATGAATTTGATGCGAGCATTAGCCAGGTGAACGAAAAAA |
| | TTAACCAGAGCCTGGCGTTTATTCGCAAAAGCGATGAACTGCTGCATAACGTGAA |
| | CGCGGGCAAAAGCACCACCAACATTATGATTACCACCATTATTATTGTGATTATT |
| | GTGATTCTGCTGAGCCTGATTGCGGTGGGCCTGCTGCTGTATTGCTGATAA |
| SEQ ID NO: | MELLILKANAITTILTAVTFCFASGQNITEEFYQSTCSAVSKGYLSALRTGWYTSVITIE |
| 167 | LSNIKENKCNGTDAKVKLIKQELDKYKNAVTELQLLMQSTPPTNNRARRGSFLGFLL |
| Corresponding | GVGSAIASGVAVSKVLHLEGEVNKIKSALLSTNKAVVSLSNGVSVLTSKVLDLKNYID |
| amino acid | KQLLPIVNKQSCSISNIETVIEFQQKNNRLLEITREFSVNAGVTTPVSTYMLTNSELLSLI |
| sequence | NDMPITNDQKKLMSNNVQIVRQQSYSIMSIIKEEVLAYVVQLPLYGVIDTPCWKLHTS |
| | PLCTTNTKEGSNICLTRTDRGWYCDNAGSVSFFPQAETCKVQSNRVFCDTMNSLTLPS |
| | EINLCNVDIFNPKYDCKIMTSKTDVSSSVITSLGAIVSCYGKTKCTASNKNRGIIKTFSN |
| | GCDYVSNKGMDTVSVGNTLYYVNKQEGKSLYVKGEPIINFYDPLVFPSDEFDASISQ |
| | VNEKINQSLAFIRKSDELLHNVNAGKSTTNIMITTIIIVIIVILLSLIAVGLLLYC |

p27-T4

| | |
|---|---|
| SEQ ID | ATGGAACTGCTGATTCTGAAAGCGAACGCGATTACCACCATTCTGACCGCGGTGA |
| NO: 168 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCAGCAAAGTGCTGCATCTGGAAGGCGAAG |
| | TGAACAAAATTAAAAGCGCGCTGCTGAGCACCAACAAAGCGGTGGTGAGCCTGA |
| | GCAACGGCGTGAGCGTGCTGACCAGCAAAGTGCTGGATCTGAAAAACTATATTGA |
| | TAAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACGGCTATATTCCGGA |
| | AGCGCCGCGCGATGGCCAGGCGTATGTGCGCAAAGATGGCGAATGGGTGCTGCT |
| | GAGCACCTTTCTGTGATAA |
| SEQ ID | MELLILKANAITTILTAVTFCFASGQNITEEFYQSTCSAVSKGYLSALRTGWYTSVITIE |
| NO: 169 | LSNIKENKCNGTDAKVKLIKQELDKYKNAVTELQLLMQSTPPTNNRARRELPRFMNY |
| Corresponding | TLNNAKKTNVTLSKKRKRRSKVLHLEGEVNKIKSALLSTNKAVVSLSNGVSVLTSKV |
| amino acid | LDLKNYIDKQLLPIVNKQSCSISNIETVIEFQQKNNRLLEITREFSVNAGVTTPVSTYML |
| sequence | TNSELLSLINDMPITNDQKKLMSNNVQIVRQQSYSIMSIIKEEVLAYVVQLPLYGVIDT |
| | PCWKLHTSPLCTTNTKEGSNICLTRTDRGWYCDNAGSVSFFPQAETCKVQSNR VFCD |
| | TMNSLTLPSEINLCNVDIFNPKYDCKIMTSKTDVSSSVITSLGAIVSCYGKTKCTASNK |
| | NRGIIKTFSNGCDYVSNKGMDTVSVGNTLYYVNKQEGKSLYVKGEPIINFYDPLVFPS |
| | DEFDASISQVNEKINQSLAFIRKSDELLHNVNAGKSTINGYIPEAPRDGQAYVRKDGE |
| | WVLLSTFL |

p27-TM

| | |
|---|---|
| SEQ ID | ATGGAACTGCTGATTCTGAAAGCGAACGCGATTACCACCATTCTGACCGCGGTGA |
| NO: 170 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCAGCAAAGTGCTGCATCTGGAAGGCGAAG |
| | TGAACAAAATTAAAAGCGCGCTGCTGAGCACCAACAAAGCGGTGGTGAGCCTGA |
| | GCAACGGCGTGAGCGTGCTGACCAGCAAAGTGCTGGATCTGAAAAACTATATTGA |
| | TAAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACTGATAA |
| SEQ ID | MELLILKANAITTILTAVTFCFASGQITEEFYQSTCSAVSKGYLSALRTGWYTSVITIE |
| NO: 171 | LSNIKENKCNGTDAKVKLIKQELDKYKNAVTELQLLMQSTPPTNNRARRELPRFMNY |
| Corresponding | TLNNAKKTNVTLSKKRKRRSKVLHLEGEVNKIKSALLSTNKAVVSLSNGVSVLTSKV |
| amino acid | LDLKNYIDKQLLPIVNKQSCSISNIETVIEFQQKNNRLLEITREFSVNAGVTTPVSTYML |
| sequence | TNSELLSLINDMPITNDQKKLMSNNVQIVRQQSYSIMSIIKEEVLAYVVQLPLYGVIDT |
| | PCWKLHTSPLCTTNTKEGSNICLTRTDRGWYCDNAGSVSFFPQAETCKVQSNRVFCD |
| | TMNSLTLPSEINLCNVDIFNPKYDCKIMTSKTDVSSSVITSLGAIVSCYGKTKCTASNK |
| | NRGIIKTFSNGCDYVSNKGMDTVSVGNTLYYVNKQEGKSLYVKGEPIINFYDPLVFPS |
| | DEFDASISQVNEKINQSLAFIRKSDELLHNVNAGKSTTN |

p27-TM-CT

| | |
|---|---|
| SEQ ID | ATGGAACTGCTGATTCTGAAAGCGAACGCGATTACCACCATTCTGACCGCGGTGA |
| NO: 172 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCAGCAAAGTGCTGCATCTGGAAGGCGAAG |
| | TGAACAAAATTAAAAGCGCGCTGCTGAGCACCAACAAAGCGGTGGTGAGCCTGA |
| | GCAACGGCGTGAGCGTGCTGACCAGCAAAGTGCTGGATCTGAAAAACTATATTGA |
| | TAAACAGCTGCTGCCGATTGTGAACAAACAGAGCTGCAGCATTAGCAACATTGAA |
| | ACCGTGATTGAATTTCAGCAGAAAAACAACCGCCTGCTGGAAATTACCCGCGAAT |
| | TTAGCGTGAACGCGGGCGTGACCACCCCGGTGAGCACCTATATGCTGACCAACAG |
| | CGAACTGCTGAGCCTGATTAACGATATGCCGATTACCAACGATCAGAAAAAACTG |
| | ATGAGCAACAACGTGCAGATTGTGCGCCAGCAGAGCTATAGCATTATGAGCATTA |
| | TTAAAGAAGAAGTGCTGGCGTATGTGGTGCAGCTGCCGCTGTATGGCGTGATTGA |
| | TACCCCGTGCTGGAAACTGCATACCAGCCCGCTGTGCACCACCAACACCAAAGAA |
| | GGCAGCAACATTTGCCTGACCCGCACCGATCGCGGCTGGTATTGCGATAACGCGG |
| | GCAGCGTGAGCTTTTTTCCGCAGGCGGAAACCTGCAAAGTGCAGAGCAACCGCGT |
| | GTTTTGCGATACCATGAACAGCCTGACCCTGCCGAGCGAAATTAACCTGTGCAAC |
| | GTGGATATTTTTAACCCGAAATATGATTGCAAAATTATGACCAGCAAAACCGATG |
| | TGAGCAGCAGCGTGATTACCAGCCTGGGCGCGATTGTGAGCTGCTATGGCAAAAC |
| | CAAATGCACCGCGAGCAACAAAAACCGCGGCATTATTAAAACCTTTAGCAACGG |
| | CTGCGATTATGTGAGCAACAAAGGCATGGATACCGTGAGCGTGGGCAACACCCTG |
| | TATTATGTGAACAAACAGGAAGGCAAAAGCCTGTATGTGAAAGGCGAACCGATT |
| | ATTAACTTTTATGATCCGCTGGTGTTTCCGAGCGATGAATTTGATGCGAGCATTAG |
| | CCAGGTGAACGAAAAAATTAACCAGAGCCTGGCGTTTATTCGCAAAAGCGATGA |
| | ACTGCTGCATAACGTGAACGCGGGCAAAAGCACCACCAACATTATGATTACCACC |
| | ATTATTATTGTGATTATTGTGATTCTGCTGAGCCTGATTGCGGTGGGCCTGCTGCT |
| | GTATTGCAAAGCGCGCAGCACCCCGGTGACCCTGAGCAAAGATCAGCTGAGCGG |
| | CATTAACAACATTGCGTTTAGCAACTGATAA |
| SEQ ID | MELLILKANAITTILTAVTFCFASGQNITEEFYQSTCSAVSKGYLSALRTGWYTSVITIE |
| NO: 173 | LSNIKENKCNGTDAKVKLIKQELDKYKNAVTELQLLMQSTPPTNNRARRELPRFMNY |
| Corresponding | TLNNAKKTNVTLSKKRKRRSKVLHLEGEVNKIKSALLSTNKAVVSLSNGVSVLTSKV |
| amino acid | LDLKNYIDKQLLPIVNKQSCSISNIETVIEFQQKNNRLLEITREFSVNAGVTTPVSTYML |
| sequence | TNSELLSLINDMPITNDQKKLMSNNVQIVRQQSYSIMSIIKEEVLAYVVQLPLYGVIDT |
| | PCWKLHTSPLCTTNTKEGSNICLTRTDRGWYCDNAGSVSFFPQAETCKVQSNRVFCD |
| | TMNSLTLPSEINLCNVDIFNPKYDCKIMTSKTDVSSSVITSLGAIVSCYGKTKCTASNK |
| | NRGIIKTFSNGCDYVSNKGMDTVSVGNTLYYVNKQEGKSLYVKGEPIINFYDPLVFPS |
| | DEFDASISQVNEKINQSLAFIRKSDELLHNVNAGKSTTNIMITTIIIVIIVILLSLIAVGLLL |
| | YCKARSTPVTLSKDQLSGINNIAFSN |

GS-T4

| | |
|---|---|
| SEQ ID | ATGGAACTGCTGATTCTGAAAGCGAACGCGATTACCACCATTCTGACCGCGGTGA |
| NO: 174 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GGCAGCAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACGGCTATATTCCGGAAGCGCCGCGCGATGGCC |
| | AGGCGTATGTGCGCAAAGATGGCGAATGGGTGCTGCTGAGCACCTTTCTGTGATA |
| | A |
| SEQ ID NO: | MELLILKANAITTILTAVTFCFASGQNITEEFYQSTCSAVSKGYLSALRTGWYTSVITIE |
| 175 | LSNIKENKCNGTDAKVKLIKQELDKYKNAVTELQLLMQSTPPTNNRARRGSSKVLHL |
| Corresponding | EGEVNKIKSALLSTNKAVVSLSNGVSVLTSKVLDLKNYIDKQLLPIVNKQSCSISNIET |
| amino acid | VIEFQQKNNRLLEITREFSVNAGVTTPVSTYMLTNSELLSLINDMPITNDQKKLMSNN |
| sequence | VQIVRQQSYSIMSIIKEEVLAYVVQLPLYGVIDTPCWKLHTSPLCTTNTKEGSNICLTR |
| | TDRGWYCDNAGSVSFFPQAETCKVQSNRVFCDTMNSLTLPSEINLCNVDIFNPKYDC |
| | KIMTSKTDVSSSVITSLGAIVSCYGKTKCTASNKNRGIIKTFSNGCDYVSNKGMDTVS |
| | VGNTLYYVNKQEGKSLYVKGEPIINFYDPLVFPSDEFDASISQVNEKINQSLAFIRKSD |
| | ELLHNVNAGKSTTNGYIPEAPRDGQAYVRKDGEWVLLSTFL |

GS-TM

| | |
|---|---|
| SEQ ID | ATGGAACTGCTGATTCTGAAAGCGAACGCGATTACCACCATTCTGACCGCGGTGA |
| NO: 176 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GGCAGCAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACATTATGATTACCACCATTATTATTGTGATTAT |
| | TGTGATTCTGCTGAGCCTGATTGCGGTGGGCCTGCTGCTGTATTGCTGATAA |
| SEQ ID NO: | MELLILKANAITTILTAVTFCFASGQNITEEFYQSTCSAVSKGYLSALRTGWYTSVITIE |
| 177 | LSNIKENKCNGTDAKVKLIKQELDKYKNAVTELQLLMQSTPPTNNRARRGSSKVLHL |
| Corresponding | EGEVNKIKSALLSTNKAVVSLSNGVSVLTSKVLDLKNYIDKQLLPIVNKQSCSISNIET |
| amino acid | VIEFQQKNNRLLEITREFSVNAGVTTPVSTYMLTNSELLSLINDMPITNDQKKLMSNN |
| sequence | VQIVRQQSYSIMSIIKEEVLAYVVQLPLYGVIDTPCWKLHTSPLCTTNTKEGSNICLTR |
| | TDRGWYCDNAGSVSFFPQAETCKVQSNRVFCDTMNSLTLPSEINLCNVDIFNPKYDC |
| | KIMTSKTDVSSSVITSLGAIVSCYGKTKCTASNKNRGIIKTFSNGCDYVSNKGMDTVS |
| | VGNTLYYVNKQEGKSLYVKGEPIINFYDPLVFPSDEFDASISQVNEKINQSLAFIRKSD |
| | ELLHNVNAGKSTTNIMITTIIIVIIVILLSLIAVGLLLYC |

GS-TM-CT

| | |
|---|---|
| SEQ ID | ATGGAACTGCTGATTCTGAAAGCGAACGCGATTACCACCATTCTGACCGCGGTGA |
| NO: 178 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GGCAGCAGCAAAGTGCTGCATCTGGAAGGCGAAGTGAACAAAATTAAAAGCGCG |
| | CTGCTGAGCACCAACAAAGCGGTGGTGAGCCTGAGCAACGGCGTGAGCGTGCTG |
| | ACCAGCAAAGTGCTGGATCTGAAAAACTATATTGATAAACAGCTGCTGCCGATTG |
| | TGAACAAACAGAGCTGCAGCATTAGCAACATTGAAACCGTGATTGAATTTCAGCA |
| | GAAAAACAACCGCCTGCTGGAAATTACCCGCGAATTTAGCGTGAACGCGGGCGT |
| | GACCACCCCGGTGAGCACCTATATGCTGACCAACAGCGAACTGCTGAGCCTGATT |
| | AACGATATGCCGATTACCAACGATCAGAAAAAACTGATGAGCAACAACGTGCAG |
| | ATTGTGCGCCAGCAGAGCTATAGCATTATGAGCATTATTAAAGAAGAAGTGCTGG |
| | CGTATGTGGTGCAGCTGCCGCTGTATGGCGTGATTGATACCCCGTGCTGGAAACT |
| | GCATACCAGCCCGCTGTGCACCACCAACACCAAAGAAGGCAGCAACATTTGCCTG |
| | ACCCGCACCGATCGCGGCTGGTATTGCGATAACGCGGGCAGCGTGAGCTTTTTTC |
| | CGCAGGCGGAAACCTGCAAAGTGCAGAGCAACCGCGTGTTTTGCGATACCATGA |
| | ACAGCCTGACCCTGCCGAGCGAAATTAACCTGTGCAACGTGGATATTTTTAACCC |
| | GAAATATGATTGCAAAATTATGACCAGCAAAACCGATGTGAGCAGCAGCGTGATT |
| | ACCAGCCTGGGCGCGATTGTGAGCTGCTATGGCAAAACCAAATGCACCGCGAGC |
| | AACAAAAACCGCGGCATTATTAAAACCTTTAGCAACGGCTGCGATTATGTGAGCA |
| | ACAAAGGCATGGATACCGTGAGCGTGGGCAACACCCTGTATTATGTGAACAAAC |
| | AGGAAGGCAAAAGCCTGTATGTGAAAGGCGAACCGATTATTAACTTTTATGATCC |
| | GCTGGTGTTTCCGAGCGATGAATTTGATGCGAGCATTAGCCAGGTGAACGAAAAA |
| | ATTAACCAGAGCCTGGCGTTTATTCGCAAAAGCGATGAACTGCTGCATAACGTGA |
| | ACGCGGGCAAAAGCACCACCAACATTATGATTACCACCATTATTATTGTGATTAT |
| | TGTGATTCTGCTGAGCCTGATTGCGGTGGGCCTGCTGCTGTATTGCAAAGCGCGC |
| | AGCACCCCGGTGACCCTGAGCAAAGATCAGCTGAGCGGCATTAACAACATTGCGT |
| | TTAGCAACTGATAA |
| SEQ ID NO: | MELLILKANAITTILTAVTFCFASGQNITEEFYQSTCSAVSKGYLSALRTGWYTSVITIE |
| 179 | LSNIKENKCNGTDAKVKLIKQELDKYKNAVTELQLLMQSTPPTNNRARRGSSKVLHL |
| Corresponding | EGEVNKIKSALLSTNKAVVSLSNGVSVLTSKVLDLKNYIDKQLLPIVNKQSCSISNIET |
| amino acid | VIEFQQKNNRLLEITREFSVNAGVTTPVSTYMLTNSELLSLINDMPITNDQKKLMSNN |
| sequence | VQIVRQQSYSIMSIIKEEVLAYVVQLPLYGVIDTPCWKLHTSPLCTTNTKEGSNICLTR |
| | TDRGWYCDNAGSVSFFPQAETCKVQSNRVFCDTMNSLTLPSEINLCNVDIFNPKYDC |
| | KIMTSKTDVSSSVITSLGAIVSCYGKTKCTASNKNRGIIKTFSNGCDYVSNKGMDTVS |
| | VGNTLYYVNKQEGKSLYVKGEPIINFYDPLVFPSDEFDASISQVNEKINQSLAFIRKSD |
| | ELLHNVNAGKSTTNIMITTIIIVIIVILLSLIAVGLLLYCKARSTPVTLSKDQLSGINNIAF |
| | SN** |

mDS-Cav1

| | |
|---|---|
| SEQ ID | atggaactgctgattctgaaagcgaacgcgattaccaccattctgaccgcggtgaccttttgctttgcgagcggccag |
| NO: 180 | aacattaccgaagaattttatcagagcacctgcagcgcggtgagcaaaggctatctgagcgcgctgcgcaccggctgg |
| ORF of mRNA | tataccagcgtgattaccattgaactgagcaacattaaagaaaacaaatgcaacggcaccgatgcgaaagtgaaactg |
| | attaaacaggaactggataaatataaaaacgcggtgaccgaactgcagctgctgatgcagagcaccccgccgaccaac |
| | aaccgcgcgcgccgcgaactgccgcgctttatgaactataccctgaacaacgcgaaaaaaaccaacgtgaccctgagc |
| | aaaaaacgcaaacgccgctttctgggctttctgctgggcgtgggcagcgcgattgcgagcggcgtggcggtgtgcaaa |
| | gtgctgcatctggaaggcgaagtgaacaaaattaaaagcgcgctgctgagcaccaacaaagcggtggtgagcctgagc |
| | aacggcgtgagcgtgctgacctttaaagtgctggatctgaaaaactatattgataaacagctgctgccgattctgaac |
| | aaacagagctgcagcattagcaacattgaaaccgtgattgaatttcagcagaaaaacaaccgcctgctggaaattacc |
| | cgcgaatttagcgtgaacgcgggcgtgaccaccccggtgagcacctatatgctgaccaacagcgaactgctgagcctg |
| | attaacgatatgccgattaccaacgatcagaaaaaactgatgagcaacaacgtgcagattgtgcgccagcagagctat |
| | agcattatgtgcattattaaagaagaagtgctggcgtatgtggtgcagctgccgctgtatggcgtgattgataccccg |
| | tgctggaaactgcataccagcccgctgtgcaccaccaacaccaaagaaggcagcaacatttgcctgacccgcaccgat |
| | cgcggctggtattgcgataacgcgggcagcgtgagcttttttccgcaggcggaaacctgcaaagtgcagagcaaccgc |
| | gtgttttgcgataccatgaacagcctgaccctgccgagcgaaattaacctgtgcaacgtggatatttttaacccgaaa |
| | tatgattgcaaaattatgaccagcaaaaccgatgtgagcagcagcgtgattaccagcctgggcgcgattgtgagctgc |
| | tatggcaaaaccaaatgcaccgcgagcaacaaaaaccgcggcattattaaaacctttagcaacggctgcgattatgtg |
| | agcaacaaaggcatggataccgtgagcgtgggcaacaccctgtattatgtgaacaaacaggaaggcaaaagcctgtat |
| | gtgaaaggcgaaccgattattaacttttatgatccgctggtgtttccgagcgatgaatttgatgcgagcattagccag |
| | gtgaacgaaaaaattaaccagagcctggcgtttattcgcaaaagcgatgaactgctgcataacgtgaacgcgggcaaa |
| | agcaccaccaacattatgattaccaccattattattgtgattattgtgattctgctgagcctgattgcggtgggcctg |
| | ctgctgtattgcaaagcgcgcagcaccccggtgaccctgagcaaagatcagctgagcggcattaacaacattgcgttt |
| | agcaactgataa |
| SEQ ID | MELLILKANAITTILTAVTFCFASGQNITEEFYQSTCSAVSKGYLSALRTGWYTSVITIE |
| NO: 181 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVCKVLHLEGEVNKIKSALLST |
| amino acid | NKAVVSLSNGVSVLTFKVLDLKNYIDKQLLPILNKQSCSISNIETVIEFQQKNNRLLEIT |
| sequence | REFSVNAGVTTPVSTYMLTNSELLSLINDMPITNDQKKLMSNNVQIVRQQSYSIMCIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTTNIM |
| | ITTIIIVIIVILLSLIAVGLLLYCKARSTPVTLSKDQLSGINNIAFSN |

sDS-Cav1

| | |
|---|---|
| SEQ ID | ATGGAACTGCTGATTCTGAAAGCGAACGCGATTACCACCATTCTGACCGCGGTGA |
| NO: 182 | CCTTTTGCTTTGCGAGCGGCCAGAACATTACCGAAGAATTTTATCAGAGCACCTG |
| ORF of mRNA | CAGCGCGGTGAGCAAAGGCTATCTGAGCGCGCTGCGCACCGGCTGGTATACCAGC |
| | GTGATTACCATTGAACTGAGCAACATTAAAGAAAACAAATGCAACGGCACCGAT |
| | GCGAAAGTGAAACTGATTAAACAGGAACTGGATAAATATAAAAACGCGGTGACC |
| | GAACTGCAGCTGCTGATGCAGAGCACCCCGCCGACCAACAACCGCGCGCGCCGC |
| | GAACTGCCGCGCTTTATGAACTATACCCTGAACAACGCGAAAAAAACCAACGTGA |
| | CCCTGAGCAAAAAACGCAAACGCCGCTTTCTGGGCTTTCTGCTGGGCGTGGGCAG |
| | CGCGATTGCGAGCGGCGTGGCGGTGTGCAAAGTGCTGCATCTGGAAGGCGAAGT |
| | GAACAAAATTAAAAGCGCGCTGCTGAGCACCAACAAAGCGGTGGTGAGCCTGAG |
| | CAACGGCGTGAGCGTGCTGACCTTTAAAGTGCTGGATCTGAAAAACTATATTGAT |
| | AAACAGCTGCTGCCGATTCTGAACAAACAGAGCTGCAGCATTAGCAACATTGAAA |
| | CCGTGATTGAATTTCAGCAGAAAAACAACCGCCTGCTGGAAATTACCCGCGAATT |
| | TAGCGTGAACGCGGGCGTGACCACCCCGGTGAGCACCTATATGCTGACCAACAGC |
| | GAACTGCTGAGCCTGATTAACGATATGCCGATTACCAACGATCAGAAAAAACTGA |
| | TGAGCAACAACGTGCAGATTGTGCGCCAGCAGAGCTATAGCATTATGTGCATTAT |
| | TAAAGAAGAAGTGCTGGCGTATGTGGTGCAGCTGCCGCTGTATGGCGTGATTGAT |
| | ACCCCGTGCTGGAAACTGCATACCAGCCCGCTGTGCACCACCAACACCAAAGAAG |
| | GCAGCAACATTTGCCTGACCCGCACCGATCGCGGCTGGTATTGCGATAACGCGGG |
| | CAGCGTGAGCTTTTTTCCGCAGGCGGAAACCTGCAAAGTGCAGAGCAACCGCGTG |
| | TTTTGCGATACCATGAACAGCCTGACCCTGCCGAGCGAAATTAACCTGTGCAACG |
| | TGGATATTTTTAACCCGAAATATGATTGCAAAATTATGACCAGCAAAACCGATGT |
| | GAGCAGCAGCGTGATTACCAGCCTGGGCGCGATTGTGAGCTGCTATGGCAAAACC |
| | AAATGCACCGCGAGCAACAAAAACCGCGGCATTATTAAAACCTTTAGCAACGGCT |
| | GCGATTATGTGAGCAACAAAGGCATGGATACCGTGAGCGTGGGCAACACCCTGT |
| | ATTATGTGAACAAACAGGAAGGCAAAAGCCTGTATGTGAAAGGCGAACCGATTA |
| | TTAACTTTTATGATCCGCTGGTGTTTCCGAGCGATGAATTTGATGCGAGCATTAGC |
| | CAGGTGAACGAAAAAATTAACCAGAGCCTGGCGTTTATTCGCAAAAGCGATGAA |
| | CTGCTGCATAACGTGAACGCGGGCAAAAGCACCACCAACGGCTATATTCCGGAA |
| | GCGCCGCGCGATGGCCAGGCGTATGTGCGCAAAGATGGCGAATGGGTGCTGCTG |
| | AGCACCTTTCTGTGATAA |
| SEQ ID NO: | MELLILKANAITTILTAVTFCFASGQNITEEFYQSTCSAVSKGYLSALRTGWYTSVITIE |
| 183 | LSNIKENKCNGTDAKVKLIKQELDKYKNAVTELQLLMQSTPPTNNRARRELPRFMNY |
| Corresponding | TLNNAKKTNVTLSKKRKRRFLGFLLGVGSAIASGVAVCKVLHLEGEVNKIKSALLST |
| amino acid | NKAVVSLSNGVSVLTFKVLDLKNYIDKQLLPILNKQSCSISNIETVIEFQQKNNRLLEIT |
| sequence | REFSVNAGVTTPVSTYMLTNSELLSLINDMPITNDQKKLMSNNVQIVRQQSYSIMCIIK |
| | EEVLAYVVQLPLYGVIDTPCWKLHTSPLCTTNTKEGSNICLTRTDRGWYCDNAGSVS |
| | FFPQAETCKVQSNRVFCDTMNSLTLPSEINLCNVDIFNPKYDCKIMTSKTDVSSSVITS |
| | LGAIVSCYGKTKCTASNKNRGIIKTFSNGCDYVSNKGMDTVSVGNTLYYVNKQEGKS |
| | LYVKGEPIINFYDPLVFPSDEFDASISQVNEKINQSLAFIRKSDELLHNVNAGKSTING |
| | YIPEAPRDGQAYVRKDGEWVLLSTFL** |

### Pharmaceutical Compositions

In another aspect, the present disclosure also provides a pharmaceutical composition comprising the lipid nanoparticle composition provided herein and a pharmaceutically acceptable excipient.

The pharmaceutically acceptable excipient are conventional medicinal excipients in the art which can be prepared in a manner well known in the pharmaceutical art. Some examples of materials which can serve as pharmaceutically acceptable excipient or carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and propane alcohol; (20) phosphate buffer solutions; and (21) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The form of pharmaceutical compositions depends on a number of criteria, including, but not limited to, route of administration, extent of disease, or dose to be administered.

The pharmaceutical compositions can be formulated for oral, nasal, rectal, percutaneous, intravenous, intradermal, intramuscular, intranasal, and/or subcutaneous administration. In accordance to the desired route of administration, the pharmaceutical compositions can be formulated in the form of tablets, capsule, pill, dragee, powder, granule, sachets, cachets, lozenges, suspensions, emulsions, solutions, syrups, aerosols (as a solid or in a liquid medium), spray, ointment, paste, cream, lotion, gel, patches, inhalant, or suppository.

The pharmaceutical compositions can be formulated to provide quick, sustained or delayed release of the active ingredient after administration to the patient by employing procedures known in the art. In some embodiments, the pharmaceutical composition is formulated in a sustained released form. In some embodiments, the prolonged period of time can be about 1 hour to 24 hours, 2 hours to 12 hours, 3 hours to 8 hours, 4 hours to 6 hours, 1 to 2 days or more. In certain embodiments, the prolonged period of time is at least about 4 hours, at least about 8 hours, at least about 12 hours, or at least about 24 hours. The pharmaceutical composition can be formulated in the form of tablet. For example, release rate of the active agent can not only be controlled by dissolution of the active agent in gastrointestinal fluid and subsequent diffusion out of the tablet or pills independent of pH, but can also be influenced by physical processes of disintegration and erosion of the tablet. In some embodiments, polymeric materials as disclosed in “Medical Applications of Controlled Release,” Langer and Wise (eds.), CRC Pres., Boca Raton, Florida (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228:190; During et al., 1989, Ann. Neurol. 25:351; Howard et al., 1989, J. Neurosurg. 71:105 can be used for sustained release. The above references are incorporated herein by reference in their entirety.

In certain embodiments, the pharmaceutical compositions of present disclosure may be administrated at dosage level sufficient to deliver from about 0.0001 mg/kg to about 10 mg/kg (e.g. from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg) of the therapeutical agent in a given dose, wherein a dose of 1 mg/kg provides 1 mg of a therapeutic and/or prophylactic per 1 kg of subject body weight.

In certain embodiments, the pharmaceutical compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical carrier. In some embodiments, a dose of about 0.001 mg/kg to about 10 mg/kg of a therapeutical agent may be administered. In other embodiments, a dose of about 0.005 mg/kg to about 2.5 mg/kg of a therapeutical agent may be administered. In certain embodiments, a dose of about 0.1 mg/kg to about 1 mg/kg a therapeutical agent may be administered. In other embodiments, a dose of about 0.05 mg/kg to about 0.25 mg/kg a therapeutical agent may be administered. A dose may be administered one or more times per day, in the same or a different amount, to obtain a desired level of therapeutic, diagnostic, prophylactic, or imaging effect. The desired dosage may be delivered, for example, three times a day, two times a day, once a day, every other day, every third day, every week, every two weeks, every three weeks, or every four weeks.

### Respiratory Syncytial Virus Vaccines

In another aspect, the present disclosure provides a Respiratory Syncytial Virus vaccine (“RSV vaccine”), comprising the lipid nanoparticle composition of the present disclosure, the Respiratory Syncytial Virus polypeptide mutant of the present disclosure, or the isolated nucleic acid sequence of the present disclosure.

RSV vaccines of present disclosure can be used as therapeutic or prophylactic agents. It may be used to prevent and/or treat infectious disease. In some embodiments, the RSV vaccines of the present disclosure are used to provide prophylactic protection from RSV. Prophylactic protection from RSV can be achieved following administration of an RSV vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

### Methods of Inducing An Antigen Specific Immune Response

In another aspect, the present disclosure provides a method of inducing an antigen specific immune response in a subject, comprising administering to the subject an effective amount of the lipid nanoparticle composition of present disclosure, an effective amount of the RSV polypeptide or variant thereof of present disclosure, an effective amount of the isolated nucleic acid sequence of present disclosure or the Respiratory Syncytial Virus vaccine of present disclosure. The method of present disclosure elicits an immune response in a subject against a RSV infection, wherein anti-antigenic polypeptide antibody titer in the subject is increased.

In another aspect, the present disclosure provides use of the lipid nanoparticle composition of the present disclosure, the RSV polypeptide or variant thereof of present disclosure, or the isolated nucleic acid sequence of present disclosure, or for the manufacture of a vaccine.

In some embodiments, an antigen specific immune response comprises a T cell response or a B cell response or both. In some embodiments, an antigen specific immune response comprises a situation that an anti-RSV antibody (e.g., anti-RSV-F antibody) titer produced in a subject immunized with the RSV vaccine of present disclosure is increased as compared with that in a subject immunized with a control (e.g., a control vaccine) or without immunization. In some embodiments, the anti-RSV antibody titer produced in the subject is increased 1.1, 1.2, 1.3, 1.4, 1.5, 1.6, 1.7, 1.8, 1.9, 2, 3, 4, 5, 6, 7, 8, 9, 10 times as compared with a control.

In some embodiments, the method of inducing an antigen specific immune response involves a single administration of the Respiratory Syncytial Virus vaccine. In some embodiments, the method of inducing an antigen specific immune response further comprising administering a booster dose of the Respiratory Syncytial Virus vaccine. A booster vaccine used herein may include any RSV vaccine of present disclosure and may be the same as the RSV vaccine initially administered. In some embodiments, the same RSV vaccine is administered annually for every RSV season.

### EXAMPLES

For the purpose of illustration, the following examples are included. However, it is to be understood that these examples do not limit the invention and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the present disclosure.

For illustrative purposes, the following shows general synthetic schemes for preparing the compounds of the present disclosure as well as key intermediates. Those skilled in the art will appreciate that other synthetic schemes may be used to synthesize the inventive compounds. Although specific starting materials and reagents are depicted in the General Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Example 1: General Scheme 1 for Chemical Synthesis

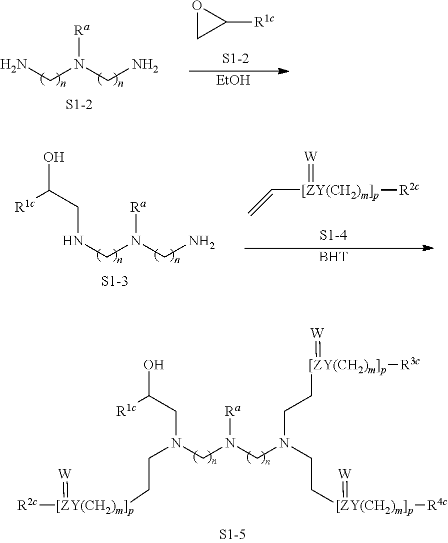

wherein
each W is independently selected from O, S and NH;
each Y is independently selected from O, S and NH;
each n is independently 1, 2 or 3;
each m is 0 or 1;
each p is 1 or 2.

### General Procedure

General Step 1: a solution of compound S1-1 in ethanol was heated at 60-80° C. To the mixture compound S1-2 was added. The mixture was cooled to room temperature after completion of reaction, and then washed and dried to afford selective mono-substituted intermediate S1-3.

General Step 2: a mixture of intermediate S1-3, compound S1-4 (with ACN and/or acetic acid) and BHT was added into a sealed container. The mixture was heated at 60-80° C. for 12-24 hours and then cooled to room temperature. The mixture was purified by column chromatography to afford compound S1-5.

### Example 1.1: Synthesis of Compound 1

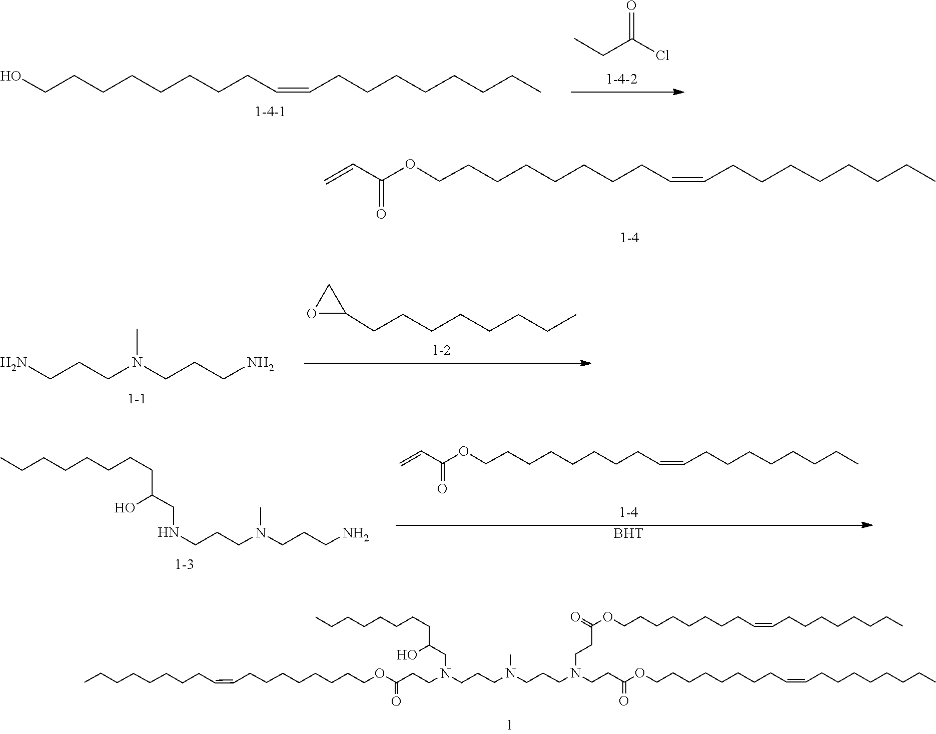

Step 1: to a container were added compound 1-4-1 (280 mg, 1.0 mmol) dry DCM (6 mL) and compound 1-4-2 (104 mg, 1.1 mmol) in sequence at room temperature. The mixture was stirred for 5 minutes before cooled in an ice bath. To the mixture was added triethylamine (211 mg, 2.1 mmol). The mixture was allowed to warm to room temperature and stirred at the same temperature for 5 hours. TLC (eluent: petroleum ether/DCM=3/1, KMnO₄ stain) indicated substantial completion of reaction. The mixture was purified by column chromatography on silica (eluent: petroleum ether with 0-33% DCM, v/v) and concentrated under reduced pressure to afford compound 11-4 as a colorless oil (220 mg, 65% yield).

¹H NMR of compound 1-4: (400 MHz, Chloroform-d) δ 6.33 (dd, J=17.3, 1.6 Hz, 1H), 6.05 (dd, J=17.4, 10.4 Hz, 1H), 5.74 (dd, J=10.4, 1.5 Hz, 1H), 5.34-5.20 (m, 2H), 4.08 (t, J=6.8 Hz, 2H), 1.94 (q, J=6.4 Hz, 4H), 1.59 (p, J=6.8 Hz, 2H), 1.22 (q, J=8.0, 5.5 Hz, 24H), 0.81 (t, J=6.7 Hz, 3H).

Step 2: to a solution of compound 1-1 (9.46 g, 65.13 mmol) in ethanol (600 mL) heated at 65° C. (internal), compound 1-2 (2.0 g, 12.80 mmol) was added. The mixture was heated at 70-75° C. (external) overnight. The mixture was cooled to room temperature then concentrated under reduced pressure. The residue was diluted with DCM (500 mL), and brine (400 mL) was added thereto under stirring. The mixture was heated at 35° C. (internal) and stirred for 10 minutes then let stand and separated. The aqueous layer was extracted with DCM (500 mL) again, repeated for 4-5 times. TLC (eluent: DCM/MeOH=5:1, with 3 drops of NH₄OH) indicated completion of extraction. Combined organic layer was dried over Na₂SO₄ and concentrated under reduced pressure to afford compound 1-3 (3.47 g, 97% yield).

Step 3: to compound 1-3 (80 mg, 0.27 mmol) in a 4 mL container were added compound 1-4 (308 mg, 0.95 mmol) and BHT (5 mg) under N₂ atmosphere. The mixture was heated at 70° C. for 48 hours. TLC indicated completion of reaction. The mixture was purified by column chromatography on silica (eluent: DCM with 0-10% methanol, v/v) and concentrated under reduced pressure to afford compound 1 (250 mg, 74% yield).

¹H NMR of compound 1: (400 MHz, Chloroform-d) δ 5.44-5.26 (m, 5H), 4.05 (q, J=6.4 Hz, 6H), 3.58 (dd, J=6.9, 3.4 Hz, 1H), 2.97-2.83 (m, 1H), 2.72 (dt, J=24.8, 7.0 Hz, 5H), 2.57 (dt, J=14.3, 7.3 Hz, 1H), 2.01 (q, J=6.4 Hz, 12H), 1.61 (t, J=7.0 Hz, 10H), 1.28 (q, J=8.2, 4.7 Hz, 91H), 0.87 (t, J=6.6 Hz, 14H).

### Example 1.2: Synthesis of Compound 2

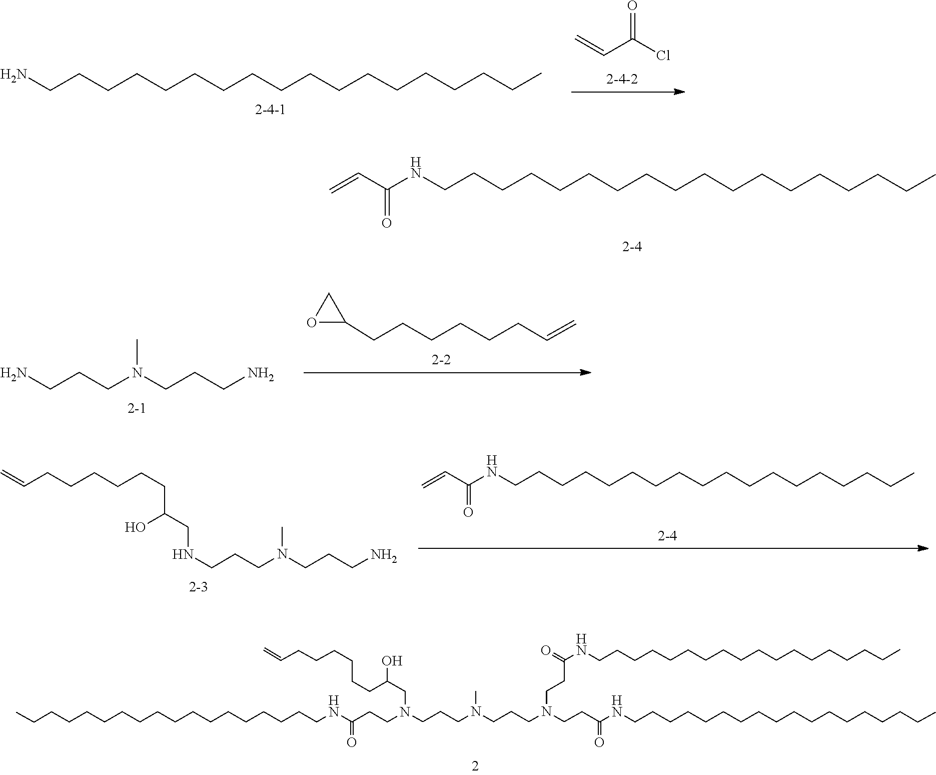

Step 1: to a container were added dry DCM (200 mL), NaHCO₃ (7.43 g, 88.5 mmol) compound 2-4-1 (9.6 g, 35.7 mmol) and compound 2-4-2 (4 g, 44.2 mmol) in sequence in an ice bath. The mixture was allowed to warm to room temperature and stirred at the same temperature for 2 hours. TLC (I₂ stain) indicated completion of reaction. The reaction was quenched with water (200 mL) and stirred for 10 minutes. The organic layer was washed with saturated aqueous NaHCO₃ solution (200 mL) twice, followed by water (200 mL). The organic layer was dried and concentrated to afford crude compound 2-4 (11.4 g, 87%).

¹H NMR of compound 2-4: (400 MHz, Chloroform-d) δ 6.29 (dd, J=17.0, 1.5 Hz, 1H), 6.10 (dd, J=17.0, 10.3 Hz, 1H), 5.68-5.48 (m, 2H), 3.39-3.31 (m, 2H), 1.56 (p, J=7.1 Hz, 2H), 1.28 (s, 29H), 0.90 (t, J=6.7 Hz, 3H).

Step 2: to a solution of compound 2-1 (9.46 g, 65.13 mmol) in ethanol (600 mL) heated at 65° C. (internal), compound 2-2 (2.0 g, 12.97 mmol) was added. The mixture was heated at 70-75° C. (external) overnight. The mixture was cooled to room temperature then concentrated under reduced pressure. The residue was diluted with DCM (500 mL), and brine (400 mL) was added thereto under stirring. The mixture was heated at 35° C. (internal) and stirred for 10 minutes then let stand and separated. The aqueous layer was extracted with DCM (500 mL) again, repeated for 4-5 times. TLC (eluent: DCM/MeOH=5:1, with 3 drops of NH₄OH) indicated completion of extraction. Combined organic layer was dried over Na₂SO₄ and concentrated under reduced pressure to afford compound 2-3 (3.4 g, 95% yield).

Step 3: to compound 2-3 (100 mg, 0.33 mmol) were added compound 2-4 (384 mg, 1.19 mmol) and BHT (5 mg). The mixture was heated for 48 hours. TLC indicated completion of reaction. The mixture was diluted with DCM (10 mL) and concentrated under reduced pressure. The residue was purified by column chromatography on silica (eluent: DCM with 0-10% methanol, v/v) and concentrated under reduced pressure to afford compound 2 as a colorless oil (140 mg, 45% yield).

¹H NMR of compound 2: (400 MHz, Chloroform-d) δ 5.88-5.71 (m, 1H), 5.49-5.25 (m, 6H), 5.05-4.81 (m, 2H), 4.05 (q, J=6.4 Hz, 7H), 3.58 (td, J=7.0, 3.6 Hz, 1H), 2.90 (dt, J=14.5, 7.4 Hz, 1H), 2.73 (dt, J=25.2, 6.9 Hz, 6H), 2.57 (dt, J=14.4, 7.3 Hz, 2H), 2.43 (q, J=7.0, 6.4 Hz, 13H), 2.36-1.91 (m, 27H), 1.61 (t, J=7.1 Hz, 12H), 1.30 (ddd, J=17.8, 10.8, 5.4 Hz, 96H), 0.88 (t, J=6.7 Hz, 12H).

### Example 1.3: Synthesis of Compound 3

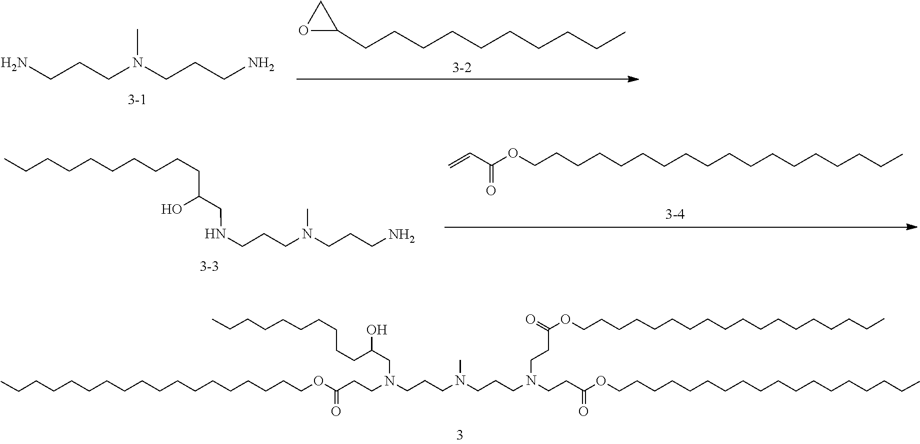

Step 1: to a solution of compound 3-1 (9.46 g, 65.13 mmol) in ethanol (600 mL) heated at 65° C. (internal), compound 3-2 (2.2 g, 11.94 mmol) was added. The mixture was heated at 70-75° C. (external) overnight. The mixture was cooled to room temperature then concentrated under reduced pressure. The residue was diluted with DCM (500 mL), and brine (400 mL) was added thereto under stirring. The mixture was heated at 35° C. (internal) and stirred for 10 minutes then let stand and separated. The aqueous layer was extracted with DCM (500 mL) again, repeated for 4-5 times. TLC (eluent: DCM/MeOH=5:1, with 3 drops of NH₄OH) indicated completion of extraction. Combined organic layer was dried over Na₂SO₄ and concentrated under reduced pressure to afford compound 3-3 (3.5 g, 97% yield).

Step 2: to compound 3-3 (100 mg, 0.30 mmol) were added compound 3-4 (350 mg, 1.08 mmol) and BHT (5 mg). The mixture was heated for 48 hours. TLC indicated completion of reaction. The mixture was diluted with DCM (10 mL) and concentrated under reduced pressure. The residue was purified by column chromatography on silica (eluent: DCM with 0-5% methanol, v/v) and concentrated under reduced pressure to afford compound 3 as a colorless oil (198 mg, 70% yield).

¹H NMR of compound 3: (400 MHz, Chloroform-d) δ 4.05 (q, J=6.6 Hz, 6H), 3.58 (s, 1H), 2.91 (dt, J=14.3, 7.5 Hz, 1H), 2.72 (dt, J=23.3, 6.7 Hz, 7H), 2.42 (h, J=6.4 Hz, 10H), 2.24 (d, J=48.6 Hz, 6H), 1.68-1.52 (m, 9H), 1.25 (s, 112H), 0.88 (t, J=6.8 Hz, 12H).

### Example 1.4: Synthesis of Compound 4

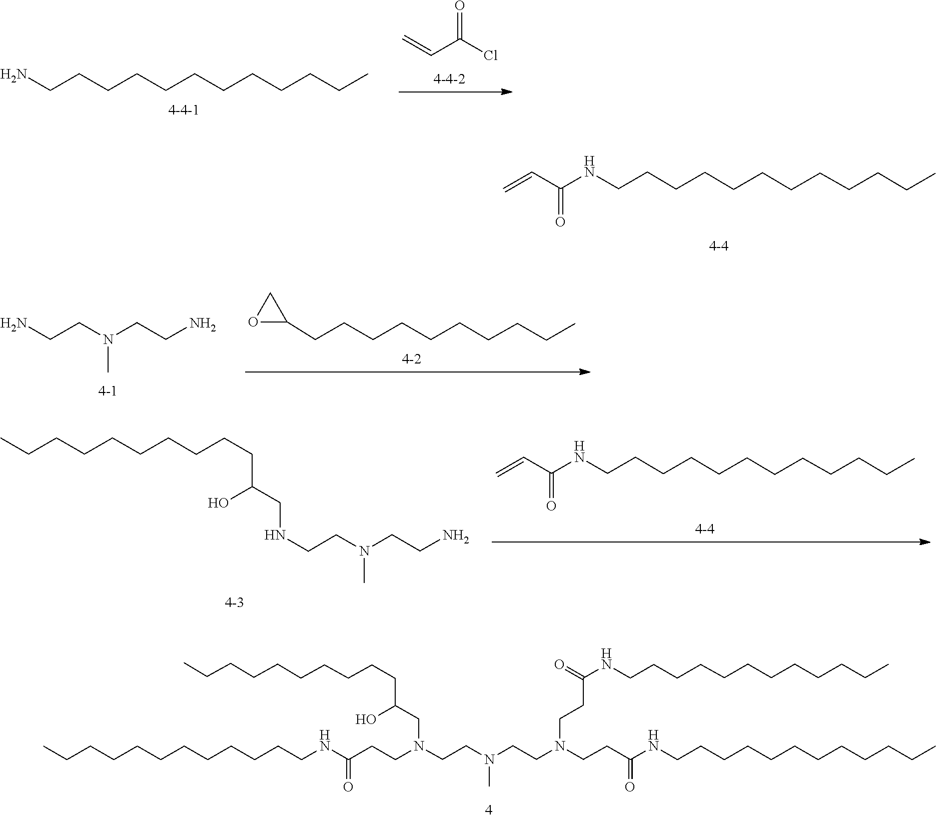

Step 1: to a container were added dry DCM (300 mL), NaHCO₃ (11.14 g, 132.6 mmol), compound 4-4-1 (14.15 g, 76.3 mmol) and compound 4-4-2 (6.0 g, 66.3 mmol) in sequence in an ice bath. The mixture was allowed to warm to room temperature and stirred at the same temperature for 2 hours. TLC (I₂ stain) indicated completion of reaction. The reaction was quenched with water (300 mL) and stirred for 10 minutes. The organic layer was washed with saturated aqueous NaHCO₃ solution (300 mL) twice, followed by water (300 mL). The organic layer was dried and concentrated to afford crude compound 4-4 (8.4 g, 47%).

¹H NMR of compound 4-4: (400 MHz, Chloroform-d) δ 6.27 (d, J=16.9 Hz, 1H), 6.09 (dd, J=17.0, 10.2 Hz, 1H), 5.64 (t, J=11.1 Hz, 2H), 3.32 (q, J=6.8 Hz, 2H), 1.53 (p, J=7.1 Hz, 2H), 1.26 (s, 24H), 0.88 (t, J=6.6 Hz, 3H).

Step 2: to a solution of compound 4-1 (1.145 g, 9.77 mmol) in ethanol (100 mL) heated at 65° C. (internal), compound 4-2 (0.30 g, 1.63 mmol) was added. The mixture was heated at 70-75° C. (external) overnight. The mixture was cooled to room temperature then concentrated under reduced pressure. The residue was diluted with DCM (300 mL), and brine (300 mL) was added thereto under stirring. The mixture was heated at 35° C. (internal) and stirred for 10 minutes then let stand and separated. Extraction was repeated for 3-4 times. TLC (eluent: DCM/MeOH=5:1, with 2 drop of NH₄OH) indicated completion of extraction. Combined organic layer was dried over Na₂SO₄ and concentrated under reduced pressure to afford compound 4-3 (0.47 g, 95% yield).

Step 3: to compound 4-3 (20 mg, 0.07 mmol) in a 2 mL container were added compound 4-4 (61 mg, 0.25 mmol, BHT (15 mg), and acetic acid (3 μL). The mixture was heated at 80° C. for 105 hours. TLC (eluent: DCM/MeOH=10/1 with NH₄OH) indicated substantial completion of reaction. The mixture was purified by column chromatography on silica (eluent: DCM with 1-1.3% methanol and 0.5% NH₄OH, v/v) and concentrated under reduced pressure to afford compound 4 (45 mg, 66% yield).

¹H NMR of compound 4: (400 MHz, Chloroform-d) δ 7.12-6.96 (m, 1H), 6.93 (t, J=5.5 Hz, 1H), 3.67-3.48 (m, 1H), 3.10 (dq, J=26.9, 6.6 Hz, 6H), 2.91-2.81 (m, 1H), 2.54 (d, J=37.5 Hz, 9H), 2.44-2.22 (m, 11H), 2.16 (dd, J=14.0, 6.4 Hz, 2H), 2.08-1.85 (m, 2H), 1.49-1.30 (m, 9H), 1.20 (d, J=11.0 Hz, 80H), 0.81 (t, J=6.8 Hz, 14H).

### Example 1.5: Synthesis of Compound 5

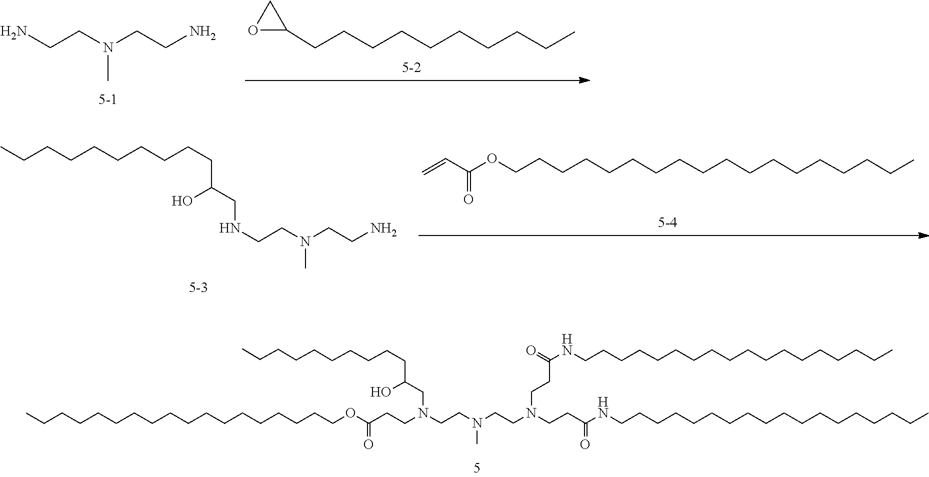

Step 1: compound 5-3 was prepared by the method of Example 1.4 Step 1.

Step 2: to compound 5-3 (19.6 mg, 0.07 mmol) in a 2 mL container were added compound 5-4 (78.6 mg, 0.24 mmol) and BHT (17.2 mg). The mixture was heated at 70° C. for 67 hours then at 90° C. for 23 hours. TLC (eluent: DCM/MeOH=20/1) indicated substantial completion of reaction. The mixture was purified by flash column chromatography on silica (eluent: DCM with 0-5% methanol, v/v) and concentrated under reduced pressure to afford compound 5 (40.8 mg, 49% yield).

¹H NMR of compound 5: (400 MHz, Chloroform-d) δ 4.05 (q, J=7.2 Hz, 5H), 3.62 (dt, J=11.9, 6.5 Hz, 1H), 2.94 (dt, J=14.2, 7.3 Hz, 2H), 2.84 (q, J=6.6 Hz, 2H), 2.77 (t, J=6.9 Hz, 5H), 2.65-2.58 (m, 1H), 2.47 (dq, J=13.8, 7.5, 7.0 Hz, 7H), 2.40-2.17 (m, 2H), 1.71-1.53 (m, 6H), 1.25 (s, 106H), 0.88 (t, J=6.8 Hz, 12H).

### Example 1.6: Synthesis of Compound 6

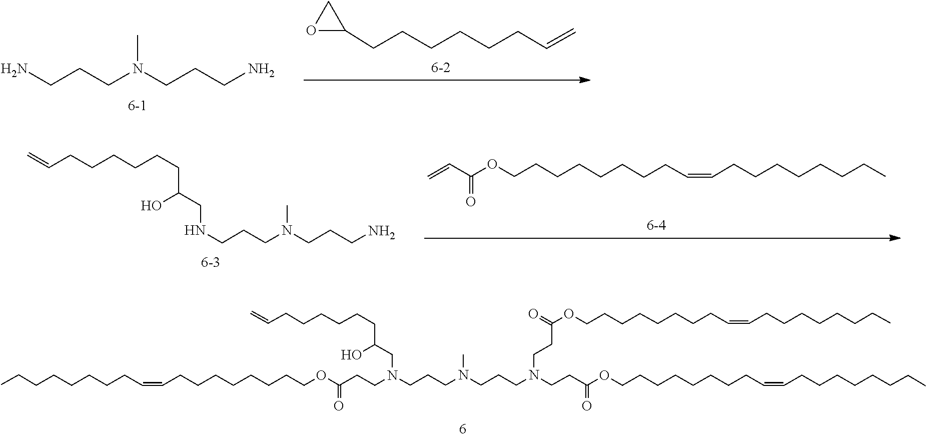

Step 1: compound 6-3 was prepared by the methods of Example 1.2 Step 2.

Step 2: to compound 6-3 (80 mg, 0.27 mmol) in a 4 mL container were added compound 6-4 (310 mg, 0.96 mmol, prepared by the method of Example 1.1 Step 1) and BHT (5 mg) under N₂ atmosphere. The mixture was heated at 70° C. for 48 hours. TLC indicated completion of reaction. The mixture was purified by column chromatography on silica (eluent: DCM with 0-10% methanol, v/v) and concentrated under reduced pressure to afford compound 6 (230 mg, 68% yield).

¹H NMR of compound 6: (400 MHz, Chloroform-d) δ 5.88-5.71 (m, 1H), 5.49-5.25 (m, 6H), 5.05-4.81 (m, 2H), 4.05 (q, J=6.4 Hz, 7H), 3.58 (td, J=7.0, 3.6 Hz, 1H), 2.90 (dt, J=14.5, 7.4 Hz, 1H), 2.73 (dt, J=25.2, 6.9 Hz, 6H), 2.57 (dt, J=14.4, 7.3 Hz, 2H), 2.43 (q, J=7.0, 6.4 Hz, 13H), 2.36-1.91 (m, 27H), 1.61 (t, J=7.1 Hz, 12H), 1.30 (ddd, J=17.8, 10.8, 5.4 Hz, 96H), 0.88 (t, J=6.7 Hz, 12H).

### Example 1.7: Synthesis of Compound 7

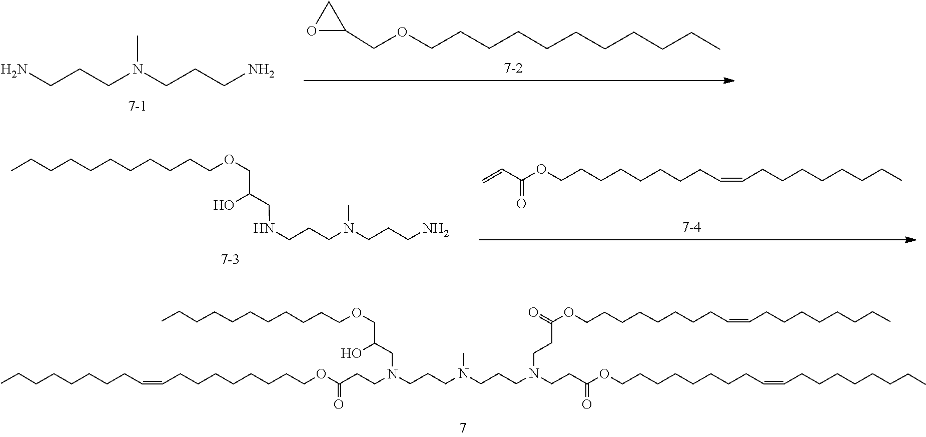

Step 1: to a solution of compound 7-1 (1.44 g, 9.91 mmol) in ethanol (100 mL) heated at 65° C. (internal), compound 7-2 (0.40 g, 1.75 mmol) was added. The mixture was heated at 70-75° C. (external) overnight. The mixture was cooled to room temperature then concentrated under reduced pressure. The residue was diluted with DCM (200 mL), and water (200 mL) was added thereto under stirring. The mixture was heated at 35° C. (internal) and stirred for 10 minutes then let stand and separated. Extraction was repeated for 3-4 times. TLC (eluent: DCM/MeOH=5:1, with 3 drop of NH₄OH) indicated completion of extraction. Combined organic layer was dried over Na₂SO₄ and concentrated under reduced pressure to afford compound 7-3 (0.43 g, 66% yield).

Step 2: to compound 7-3 (100 mg, 0.27 mmol) in a 4 mL container were added compound 7-4 (270 mg, 0.94 mmol, prepared by the method of Example 1.1 Step 1) and BHT (5 mg) under N₂ atmosphere. The mixture was heated at 70° C. for 48 hours. TLC indicated completion of reaction. The mixture was purified by column chromatography on silica (eluent: DCM with 0-10% methanol, v/v) and concentrated under reduced pressure to afford compound 7 (220 mg, 67% yield).

¹H NMR of compound 7: (400 MHz, Chloroform-d) δ 5.45-5.21 (m, 5H), 4.05 (td, J=6.8, 4.3 Hz, 6H), 3.89-3.74 (m, 1H), 3.53-3.34 (m, 4H), 2.98-2.82 (m, 1H), 2.74 (td, J=7.0, 4.5 Hz, 6H), 2.62-2.35 (m, 16H), 2.00 (hept, J=5.9, 5.2 Hz, 12H), 1.58 (dt, J=18.9, 7.0 Hz, ^{12H}), 1.47-1.07 (m, 90H), 0.87 (t, J=6.7 Hz, 13H).

### Example 1.8: Synthesis of Compound 8

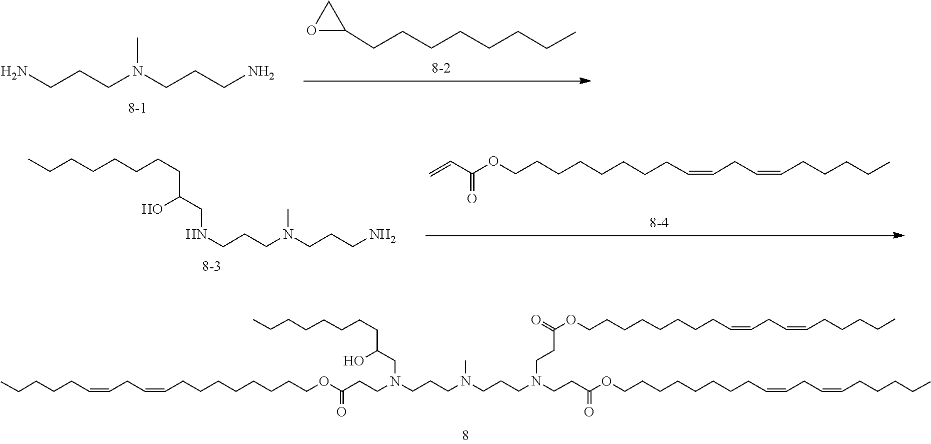

Step 1: compound 8-3 was prepared by the method of Example 1.1 Step 2.

Step 2: to compound 8-3 (100 mg, 0.33 mmol) in a 4 mL container were added compound 8-4 (350 mg, 1.09 mmol) and BHT (10 mg) under N₂ atmosphere. The mixture was heated at 70° C. for 48 hours. TLC indicated completion of reaction. The mixture was purified by column chromatography on silica (eluent: DCM with 0-10% methanol, v/v) and concentrated under reduced pressure to afford compound 8 (230 mg, 55% yield).

¹H NMR of compound 8: (400 MHz, Chloroform-d) δ 5.80 (ddt, J=16.9, 10.2, 6.7 Hz, 1H), 5.46-5.23 (m, 13H), 5.05-4.88 (m, 2H), 4.17-3.94 (m, 6H), 3.58 (ddt, J=10.4, 6.9, 3.4 Hz, 1H), 3.02-2.83 (m, 1H), 2.82-2.65 (m, 12H), 2.56 (q, J=7.4, 6.8 Hz, 2H), 2.51-2.11 (m, 21H), 2.05 (q, J=6.7 Hz, 16H), 1.61 (dd, J=10.6, 4.3 Hz, 12H), 1.49-1.10 (m, 71H), 0.89 (t, J=6.8 Hz, 12H).

### Example 1.9: Synthesis of Compound 9

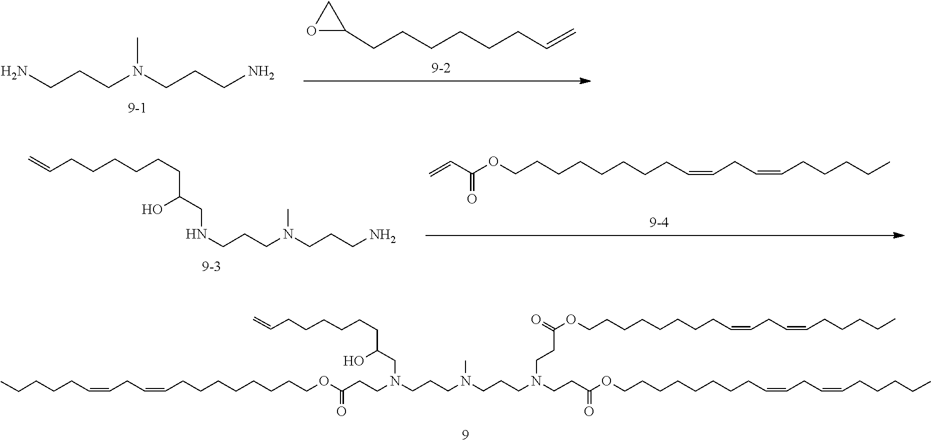

Step 1: compound 9-3 was prepared by the methods of Example 1.2 Step 2.

Step 2: to compound 9-3 (100 mg, 0.33 mmol) in a 4 mL container were added compound 9-4 (360 mg, 1.12 mmol) and BHT (10 mg) under N₂ atmosphere. The mixture was heated at 70° C. for 48 hours. TLC indicated completion of reaction. The mixture was purified by column chromatography on silica (eluent: DCM with 0-10% methanol, v/v) and concentrated under reduced pressure to afford compound 9 (221 mg, 56% yield).

¹H NMR of compound 9: (400 MHz, Chloroform-d) δ 5.45-5.26 (m, 12H), 4.05 (q, J=7.1 Hz, 6H), 3.60 (s, 1H), 2.93 (dt, J=13.9, 7.5 Hz, 3H), 2.75 (dt, J=17.7, 6.7 Hz, 15H), 2.54-2.20 (m, 14H), 2.05 (q, J=6.8 Hz, 14H), 1.88 (s, 5H), 1.62 (p, J=6.7 Hz, 9H), 1.46-1.19 (m, 72H), 0.88 (td, J=6.8, 4.5 Hz, 14H).

### Example 1.10: Synthesis of Compound 10

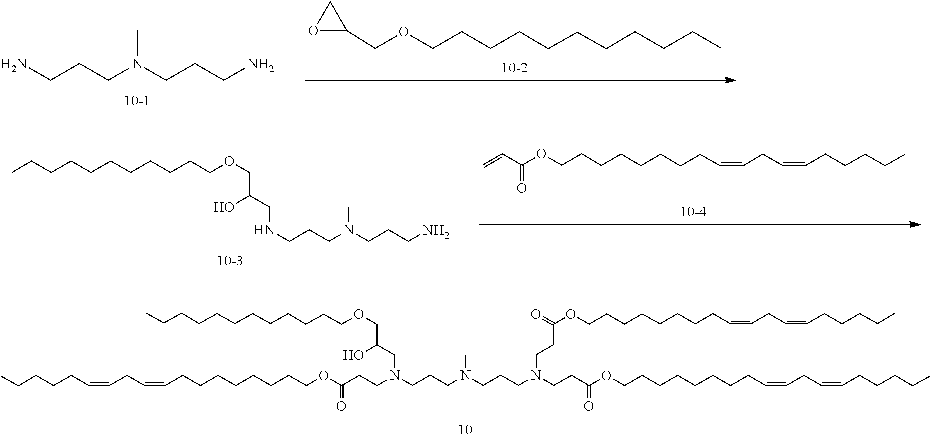

Step 1: compound 10-3 was prepared by the method of Example 1.7 Step 1.

Step 2: to compound 10-3 (120 mg, 0.32 mmol) in a 4 mL container were added compound 10-4 (350 mg, 1.09 mmol) and BHT (10 mg) under N₂ atmosphere. The mixture was heated at 70° C. for 48 hours. TLC indicated completion of reaction. The mixture was purified by column chromatography on silica (eluent: DCM with 0-10% methanol, v/v) and concentrated under reduced pressure to afford compound 10 (205 mg, 50% yield).

¹H NMR of compound 10: (400 MHz, Chloroform-d) δ 5.48-5.24 (m, 14H), 4.06 (q, J=7.2 Hz, 6H), 3.79 (dq, J=9.9, 5.1 Hz, 1H), 2.87 (dq, J=17.0, 9.7, 8.5 Hz, 3H), 2.77 (t, J=6.4 Hz, 7H), 2.68 (dt, J=13.0, 6.3 Hz, 5H), 2.44 (dt, J=13.2, 7.7 Hz, 7H), 2.29 (d, J=6.9 Hz, 4H), 2.13-2.03 (m, 12H), 1.59 (dt, J=17.9, 6.9 Hz, 18H), 1.43-1.20 (m, 88H), 0.88 (td, J=6.8, 4.1 Hz, 16H).

### Example 1.11: Synthesis of Compound 11

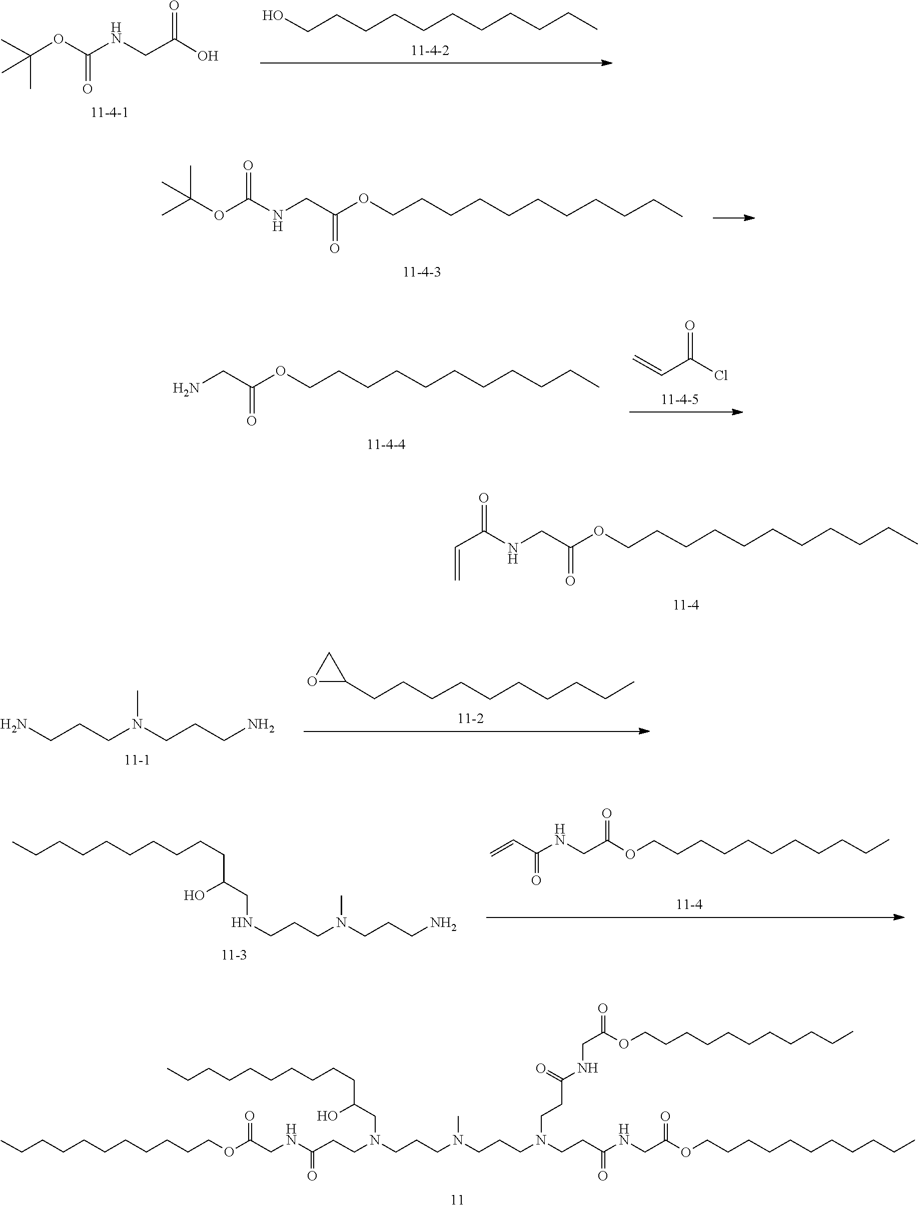

Step 1: to a solution of compound 11-4-1 (10 g, 57.1 mmol) and compound 11-4-2 (10.3 g, 59.9 mmol) in DCM (100 mL) were added DMAP (698 mg, 5.7 mmol). To the mixture was added 1-ethyl-3-(3-dimethylaminopropyl)carbodiimide (EDCI, 21.9 g, 114.2 mmol) in portions under N₂ atmosphere at <10° C. The mixture was allowed to warm to room temperature and stirred at the same temperature for 16 hours. The reaction was quenched with saturated aqueous NaHCO₃ solution (100 mL). The aqueous layer was extracted with ethyl acetate (200 mL). The combined organic layer was washed with 5% aqueous citric acid solution (100 mL) and brine (100 mL), followed by brine (100 mL). The organic layer was dried over Na₂SO₄ and concentrated to afford 18.5 g crude compound 11-4-3. The compound 11-4-3 was used directly without further purification.

¹H NMR of compound 11-4-3: (400 MHz, DMSO-d6) δ 7.15 (t, J=6.2 Hz, 1H), 4.02 (t, J=6.5 Hz, 2H), 3.64 (d, J=6.2 Hz, 2H), 1.55 (t, J=6.9 Hz, 2H), 1.38 (s, 8H), 1.34 (s, 2H), 1.24 (s, 19H), 0.85 (t, J=6.5 Hz, 4H).

Step 2: to a solution of compound 11-4-3 (18.5 g, 56.1 mmol, crude) in ethyl acetate (93 mL) was added HCl (4 M in ethyl acetate, 93 mL) under N₂ atmosphere at <10° C. The mixture was allowed to warm to room temperature and stirred at the same temperature for 16 hours. TLC indicated the consumption of starting materials. The mixture was cooled to 5° C. and stirred at the same temperature for 2 hours. The mixture was filtrated, and the residue was washed with ethyl acetate and dried to afford 12.5 g crude compound 11-4-4. The compound 11-4-4 was used directly without further purification.

¹H NMR of compound 11-4-4: (400 MHz, DMSO-d6) δ 8.63 (s, 3H), 4.11 (t, J=6.6 Hz, 2H), 3.73 (s, 2H), 1.57 (p, J=6.7 Hz, 2H), 1.23 (s, 17H), 0.84 (t, J=6.6 Hz, 3H).

Step 3: to a solution of compound 11-4-4 (5 g, 18.9 mmol) and compound 11-4-5 (1.9 g, 20.7 mmol) in DCM (50 mL) was added DMAP (698 mg 5.7 mmol) under N₂ atmosphere at <10° C. To the mixture was added a solution of N,N-diisopropylethylamine (DIEA, 4.9 g, 37.8 mmol) in DCM (5 mL) dropwise at the same temperature. The mixture was allowed to warm to room temperature and stirred at the same temperature for 3 hours. TCL indicated the consumption of starting materials. The reaction was quenched with saturated aqueous NaHCO₃ solution (50 mL). The aqueous layer was extracted with ethyl acetate (100 mL). The combined organic layer was washed with 5% aqueous citric acid solution (50 mL) and brine (50 mL), followed by brine (100 mL). The organic layer was dried over Na₂SO₄ and concentrated to afford crude product. The crude product was purified by column chromatography on silica (eluent: petroleum ether with 20% ethyl acetate, v/v) and concentrated under reduced pressure to afford compound 11-4 (4.4 g, 27% yield for 3 steps).

¹H NMR of compound 11-4:(400 MHz, DMSO-d6) δ 8.51 (t, J=6.0 Hz, 1H), 6.29 (dd, J=17.1, 10.2 Hz, 1H), 6.11 (dd, J=17.1, 2.1 Hz, 1H), 5.63 (dd, J=10.2, 2.1 Hz, 1H), 4.04 (t, J=6.6 Hz, 2H), 3.90 (d, J=5.9 Hz, 2H), 1.57 (s, 2H), 1.24 (s, 16H), 0.85 (t, J=6.6 Hz, 3H).

Step 4: compound 11-3 was prepared by the method of Example 1.3 Step 1.

Step 5: to compound 11-3 (400 mg, 1.2 mmol), BHT (53.4 mg) and compound 11-4 (1.24 g, 4.4 mmol) in a 2 mL container was added acetic acid (14.6 mg). The mixture was heated at 80° C. for 42 hours. TLC (eluent: DCM/MeOH/NH₄OH=10/1/1) indicated substantial completion of reaction. The mixture was purified by flash column chromatography on silica (eluent: DCM with 1.4-24% methanol and 0.5% NH₄OH, v/v) and concentrated under reduced pressure to afford compound 11 (390 mg, 27% yield).

¹H NMR of compound 11: (400 MHz, Chloroform-d) δ 7.87 (t, J=5.5 Hz, 1H), 7.68 (t, J=5.5 Hz, 2H), 4.15-4.01 (m, 6H), 4.01-3.88 (m, 4H), 3.68 (dt, J=17.8, 8.6 Hz, 1H), 3.03-2.91 (m, 1H), 2.74 (t, J=6.0 Hz, 4H), 2.66 (dt, J=14.1, 7.3 Hz, 2H), 2.59-2.39 (m, 9H), 2.40-2.19 (m, 5H), 1.81-1.66 (m, 4H), 1.62 (p, J=6.9 Hz, 6H), 1.52-1.04 (m, 68H), 0.87 (q, J=5.9, 5.5 Hz, 12H). HR-MS: [M+H]⁺=1180.00680.

### Example 1.12: Synthesis of Compound 12

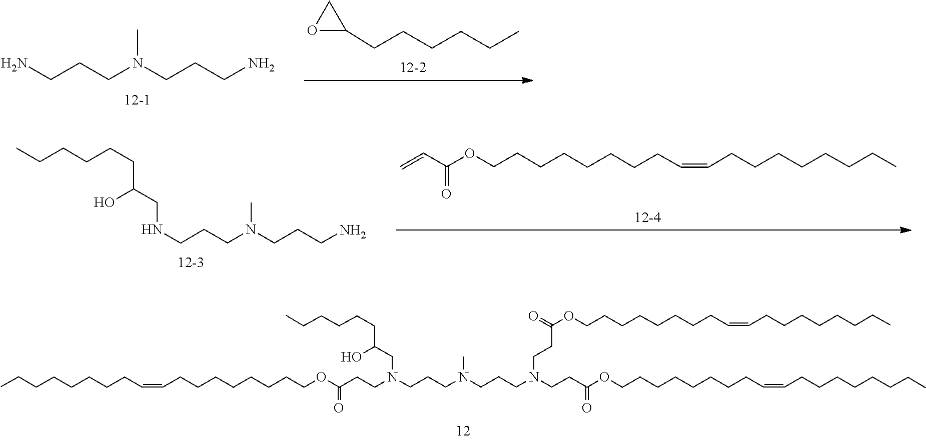

Step 1: compound 12-3 was prepared by similar method as Example 1.1 Step 2.

Step 2: to compound 12-3 (80 mg, 0.29 mmol) in a 4 mL container were added BHT (5 mg) and compound 12-4 (340 mg, 1.05 mmol, prepared by the method of Example 1.1 Step 1) under N₂ atmosphere. The mixture was heated at 70° C. for 48 hours. TLC indicated completion of reaction. The mixture was purified by flash column chromatography on silica (eluent: DCM with 0-10% methanol, v/v) and concentrated under reduced pressure to afford compound 12 (295 mg, 81% yield).

¹H NMR of compound 12: (400 MHz, Chloroform-d) δ 5.44-5.26 (m, 5H), 4.05 (q, J=6.4 Hz, 6H), 3.58 (dd, J=6.9, 3.4 Hz, 1H), 2.97-2.83 (m, 1H), 2.72 (dt, J=24.8, 7.0 Hz, 5H), 2.57 (dt, J=14.3, 7.3 Hz, 1H), 2.01 (q, J=6.4 Hz, 12H), 1.61 (t, J=7.0 Hz, 10H), 1.28 (q, J=8.2, 4.7 Hz, 91H), 0.87 (t, J=6.6 Hz, 14H). HR-MS: [M+H]⁺=1240.15427.

### Example 1.13: Synthesis of Compound 13

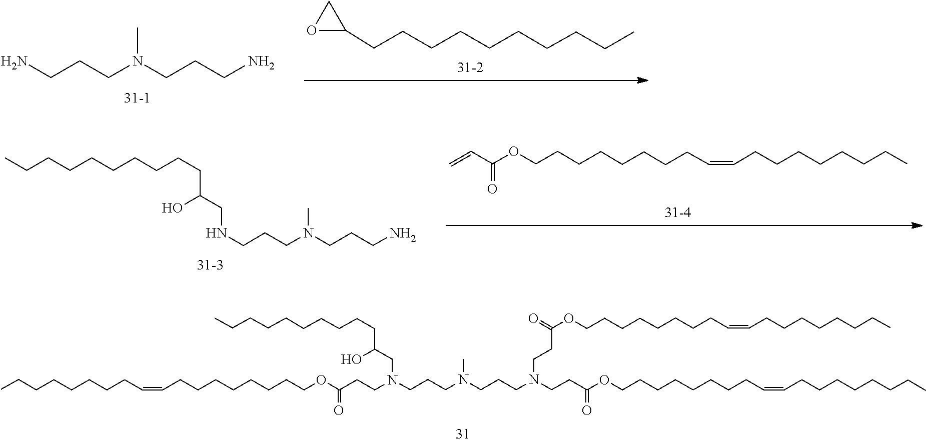

Step 1: compound 13-3 was prepared by the method of Example 1.3 Step 1.

Step 2: to compound 13-3 (80 mg, 0.24 mmol) in a 4 mL container were added BHT (5 mg) and compound 13-4 (308 mg, 0.95 mmol, prepared by the method of Example 1.1 Step 1) under N₂ atmosphere. The mixture was heated at 70° C. for 48 hours. TLC indicated completion of reaction. The mixture was purified by flash column chromatography on silica (eluent: DCM with 0-10% methanol, v/v) and concentrated under reduced pressure to afford compound 13 (250 mg, 74% yield).

¹H NMR of compound 13: (400 MHz, Chloroform-d) δ 5.49-5.22 (m, 6H), 4.05 (q, J=6.5 Hz, 6H), 3.58 (td, J=6.8, 3.4 Hz, 1H), 2.91 (dt, J=14.3, 7.5 Hz, 1H), 2.72 (dt, J=23.7, 6.9 Hz, 6H), 2.43 (q, J=7.6 Hz, 13H), 2.34-2.12 (m, 6H), 2.00 (dh, J=11.6, 6.5 Hz, 11H), 1.63 (s, 10H), 1.30 (dt, J=16.3, 9.4 Hz, 91H), 0.88 (t, J=6.7 Hz, 13H). [M+H]⁺=1297.21294.

### Example 1.14: Synthesis of Compound 14

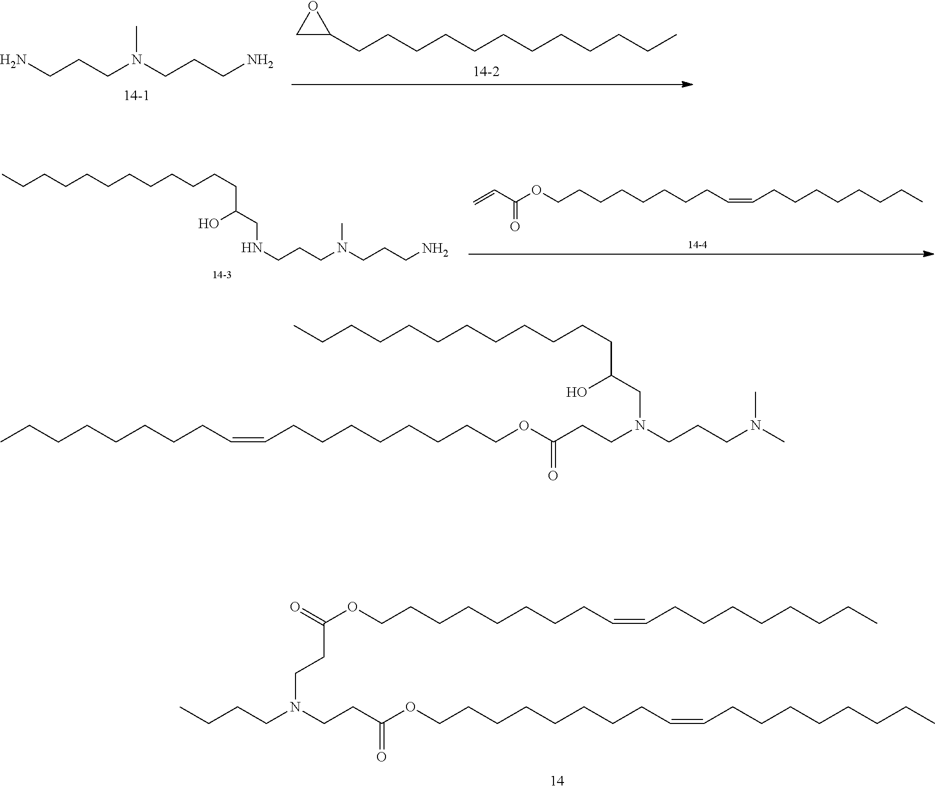

Step 1: compound 14-3 was prepared by similar method as Example 1.3 Step 1.

Step 2: to compound 14-3 (80 mg, 0.22 mmol) in a 4 mL container were added BHT (5 mg) and compound 14-4 (259 mg, 0.80 mmol, prepared by the method of Example 1.1 Step 1) under N₂ atmosphere. The mixture was heated at 70° C. for 48 hours. TLC indicated completion of reaction. The mixture was purified by flash column chromatography on silica (eluent: DCM with 0-10% methanol, v/v) and concentrated under reduced pressure to afford compound 14 (220 mg, 74% yield).

¹H NMR of compound 14: (400 MHz, Chloroform-d) δ 5.45-5.23 (m, 6H), 4.05 (q, J=6.5 Hz, 6H), 3.58 (td, J=6.7, 3.4 Hz, 1H), 2.90 (dt, J=14.3, 7.5 Hz, 1H), 2.80-2.36 (m, 19H), 2.33-1.87 (m, 20H), 1.60 (q, J=7.0 Hz, 11H), 1.51-1.06 (m, 102H), 0.88 (t, J=6.7 Hz, 14H). [M+H]+=1325.24099.

### Example 1.15: Synthesis of Compound 15

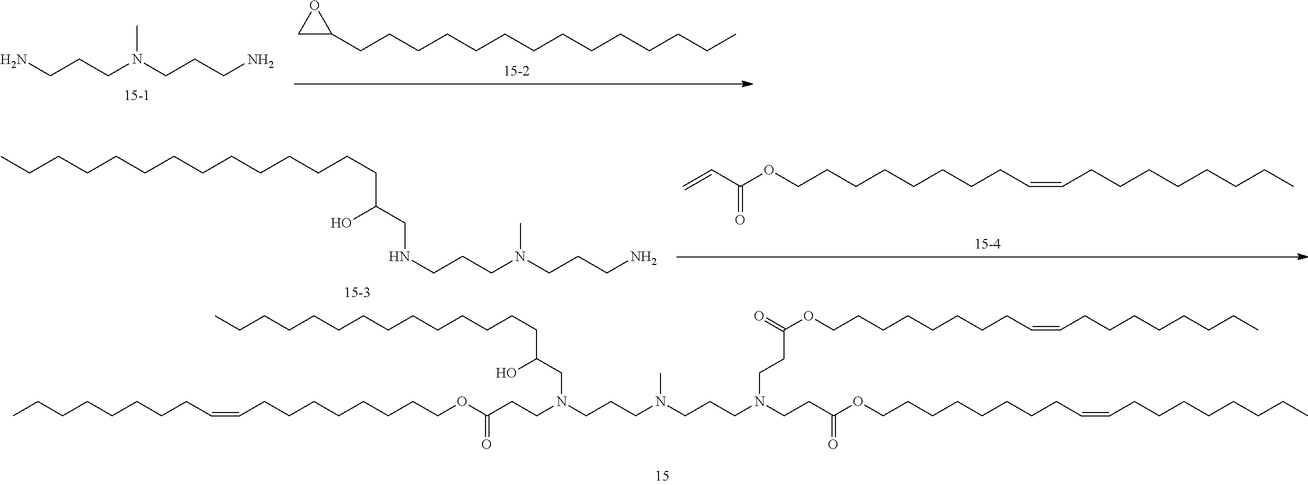

Step 1: compound 15-3 was prepared by similar method as Example 1.3 Step 1.

Step 2: to compound 15-3 (100 mg, 0.26 mmol) in a 4 mL container were added BHT (5 mg) and compound 15-4 (301 mg, 0.93 mmol, prepared by the method of Example 1.1 Step 1) under N₂ atmosphere. The mixture was heated at 70° C. for 48 hours. TLC indicated completion of reaction. The mixture was purified by flash column chromatography on silica (eluent: DCM with 0-10% methanol, v/v) and concentrated under reduced pressure to afford compound 15 (250 mg, 69% yield).

¹H NMR of compound 15: (400 MHz, Chloroform-d) δ 5.49-5.12 (m, 5H), 4.05 (q, J=6.6 Hz, 6H), 3.58 (td, J=6.9, 3.5 Hz, 1H), 2.91 (dt, J=14.3, 7.5 Hz, 1H), 2.72 (dt, J=23.2, 6.8 Hz, 6H), 2.64-2.52 (m, 2H), 2.52-2.19 (m, 17H), 2.01 (q, J=6.4 Hz, 14H), 1.61 (t, J=7.0 Hz, 11H), 1.28 (dd, J=18.7, 7.3 Hz, 99H), 0.88 (t, J=6.7 Hz, 13H). [M+H]+=1297.21294.

### Example 1.16: Synthesis of Compound 16

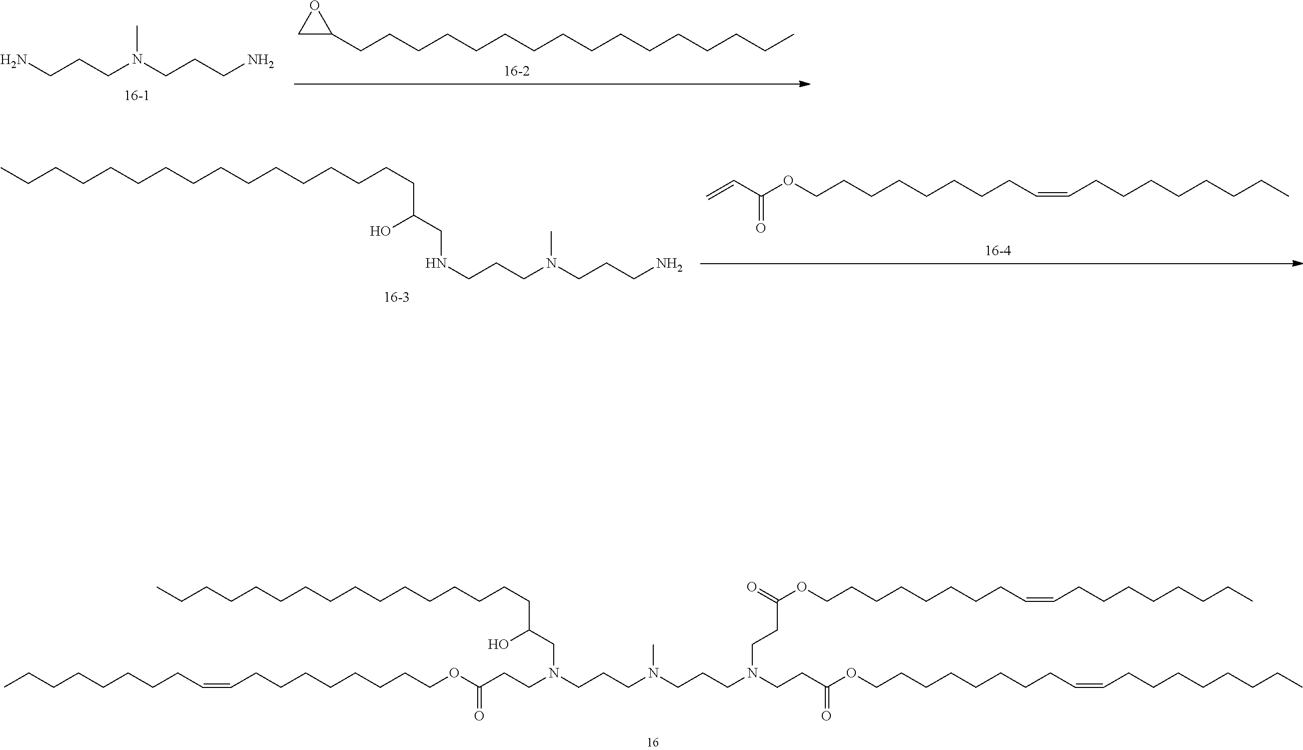

Step 1: compound 16-3 was prepared by similar method as Example 1.3 Step 1.

Step 2: to compound 16-3 (100 mg, 0.24 mmol) in a 4 mL container were added BHT (5 mg) and compound 16-4 (280 mg, 0.87 mmol, prepared by the method of Example 1.1 Step 1) under N₂ atmosphere. The mixture was heated at 70° C. for 48 hours. TLC indicated completion of reaction. The mixture was purified by flash column chromatography on silica (eluent: DCM with 0-10% methanol, v/v) and concentrated under reduced pressure to afford compound 16 (197 mg, 59% yield).

¹H NMR of compound 16: (400 MHz, Chloroform-d) δ 5.48-5.23 (m, 5H), 4.05 (q, J=6.6 Hz, 6H), 3.58 (td, J=7.5, 7.1, 3.7 Hz, 1H), 2.91 (dt, J=14.5, 7.5 Hz, 1H), 2.83-2.14 (m, 26H), 2.01 (q, J=6.4 Hz, 12H), 1.80-1.53 (m, 10H), 1.28 (dd, J=20.0, 7.6 Hz, 109H), 0.87 (t, J=6.7 Hz, 14H). [M+H]⁺=1381.31640.

### Example 1.17: Synthesis of Compound 17

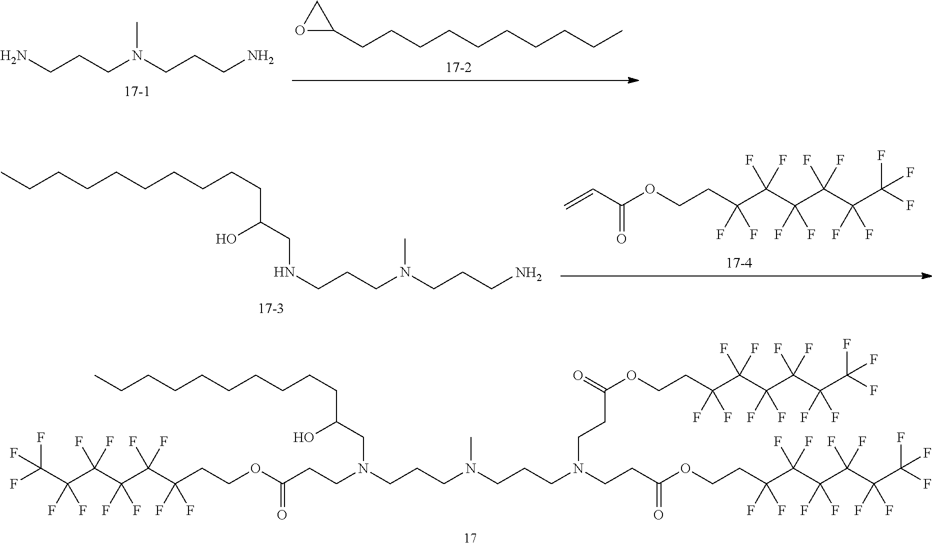

Step 1: compound 17-3 was prepared by similar method as Example 1.3 Step 1.

Step 2: to compound 17-3 (30 mg, 0.09 mmol) in a vial were added BHT (1 mg), compound 17-4 (137 mg, 0.32 mmol) and ACN (0.3 mL). The mixture was vented and decanted for N₂ atmosphere thrice. The mixture was heated at 70° C. for 2-3 days. TLC indicated completion of reaction. The mixture was purified by column chromatography on silica (eluent: DCM with 0-3% methanol, v/v) and concentrated under reduced pressure to afford compound 17 (110 mg, 76% yield).

¹H NMR of compound 17: (400 MHz, Chloroform-d) δ 4.37 (q, J=6.3 Hz, 6H), 3.57 (s, 1H), 2.91 (dt, J=14.3, 7.4 Hz, 1H), 2.83-2.63 (m, 5H), 2.63-2.08 (m, 25H), 1.25 (s, 23H), 0.87 (t, J=6.8 Hz, 3H).

### Example 1.18: Synthesis of Compound 18

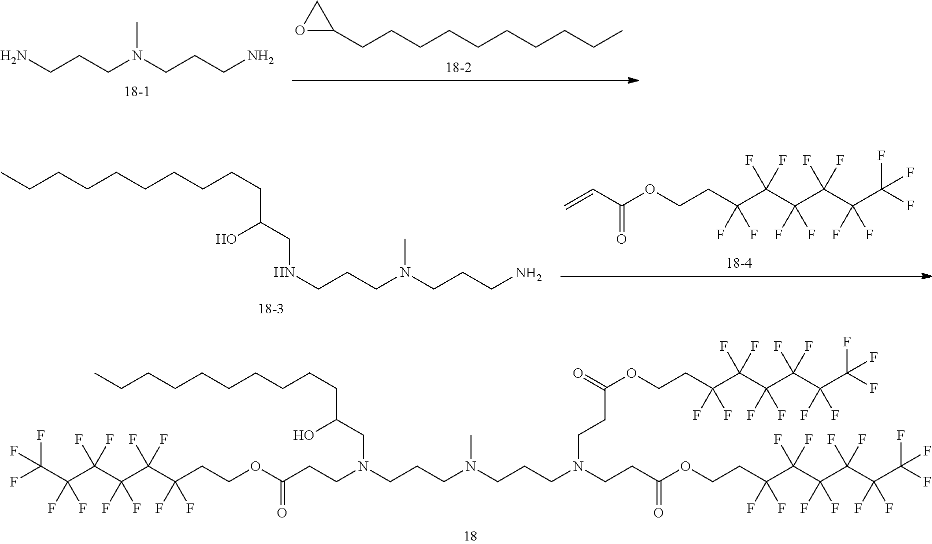

Step 1: compound 18-3 was prepared by similar method as Example 1.3 Step 1.

Step 2: to compound 18-3 (30 mg, 0.09 mmol) in a vial were added BHT (1 mg), compound 18-4 (170 mg, 0.33 mmol) and ACN (0.3 mL). The mixture was vented and decanted for N₂ atmosphere thrice. The mixture was heated at 70° C. for 2-3 days. TLC indicated completion of reaction. The mixture was purified by column chromatography on silica (eluent: DCM with 0-3% methanol, v/v) and concentrated under reduced pressure to afford compound 18 (100 mg, 58% yield).

¹H NMR of compound 18: (400 MHz, Chloroform-d) δ 4.37 (q, J=6.4 Hz, 6H), 3.57 (s, 1H), 2.91 (dt, J=14.0, 7.4 Hz, 1H), 2.78-2.66 (m, 5H), 2.56-2.15 (m, 25H), 1.25 (s, 24H), 0.88-0.84 (m, 3H).

### Example 1.19: Synthesis of Compound 19

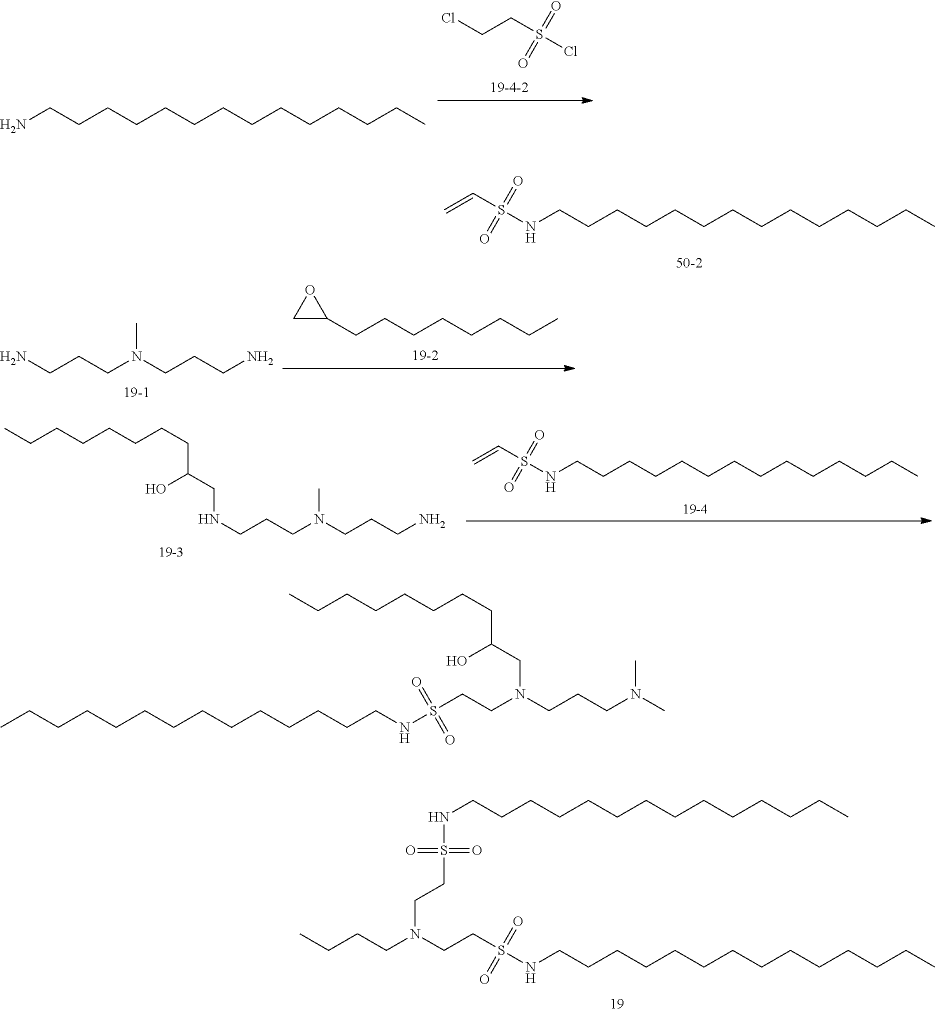

Step 1: to DCM (20 mL) were added compound 19-4-1 (1.6 g, 7.3 mmol) and triethylamine (1.86 g, 18.4 mmol) under N₂ atmosphere. The mixture was cooled to 5° C. Compound 19-4-2 (1.0 g, 6.1 mmol) was added dropwise. The mixture was allowed to warm to room temperature and stirred at the same temperature for 16 hours. TLC (eluent: DCM) indicated formation of product. The mixture was purified by column chromatography on silica to afford compound 19-4 (0.7 g, 37% yield).

¹H NMR of compound 19-4: (400 MHz, Chloroform-d) δ 6.51 (dd, J=16.5, 9.9 Hz, 1H), 6.23 (d, J=16.6 Hz, 1H), 5.93 (d, J=9.9 Hz, 1H), 4.44 (s, 1H), 3.00 (q, J=6.8 Hz, 2H), 1.53 (t, J=7.2 Hz, 2H), 1.25 (s, 24H), 0.87 (t, J=6.7 Hz, 3H).

Step 2: compound 19-3 was prepared by the method of Example 1.1 Step 2.

Step 3: to a solution of compound 19-3 (30 mg, 0.096 mmol) compound 19-4 (117.3 mg, 0.386 mmol) in ACN (0.2 mL) were added BHT (15 mg) and acetic acid (7 mg) at room temperature under N₂ atmosphere. The mixture was heated at 75° C. for 40 hours. TLC (eluent: DCM/MeOH=10/1) indicated substantial consumption of starting materials. The mixture was purified by column chromatography on silica and concentrated under reduced pressure to afford compound 19 (67 mg, 55% yield).

¹H NMR of compound 19: (400 MHz, Chloroform-d) δ 5.78 (t, J=6.1 Hz, 1H), 3.65 (td, J=8.7, 7.5, 4.0 Hz, 1H), 3.44-3.26 (m, 2H), 3.19 (dt, J=19.3, 5.2 Hz, 7H), 3.07 (q, J=7.1 Hz, 7H), 2.99-2.81 (m, 4H), 2.75-2.63 (m, 5H), 2.55 (dt, J=28.9, 7.4 Hz, 3H), 2.46-2.20 (m, 2H), 2.00 (d, J=11.9 Hz, 5H), 1.58 (dt, J=12.5, 4.7 Hz, 6H), 1.26 (d, J=5.9 Hz, 81H), 0.94-0.73 (m, 12H). [M+H]⁺=1212.00789.

### Example 1.20: Synthesis of Compound 20

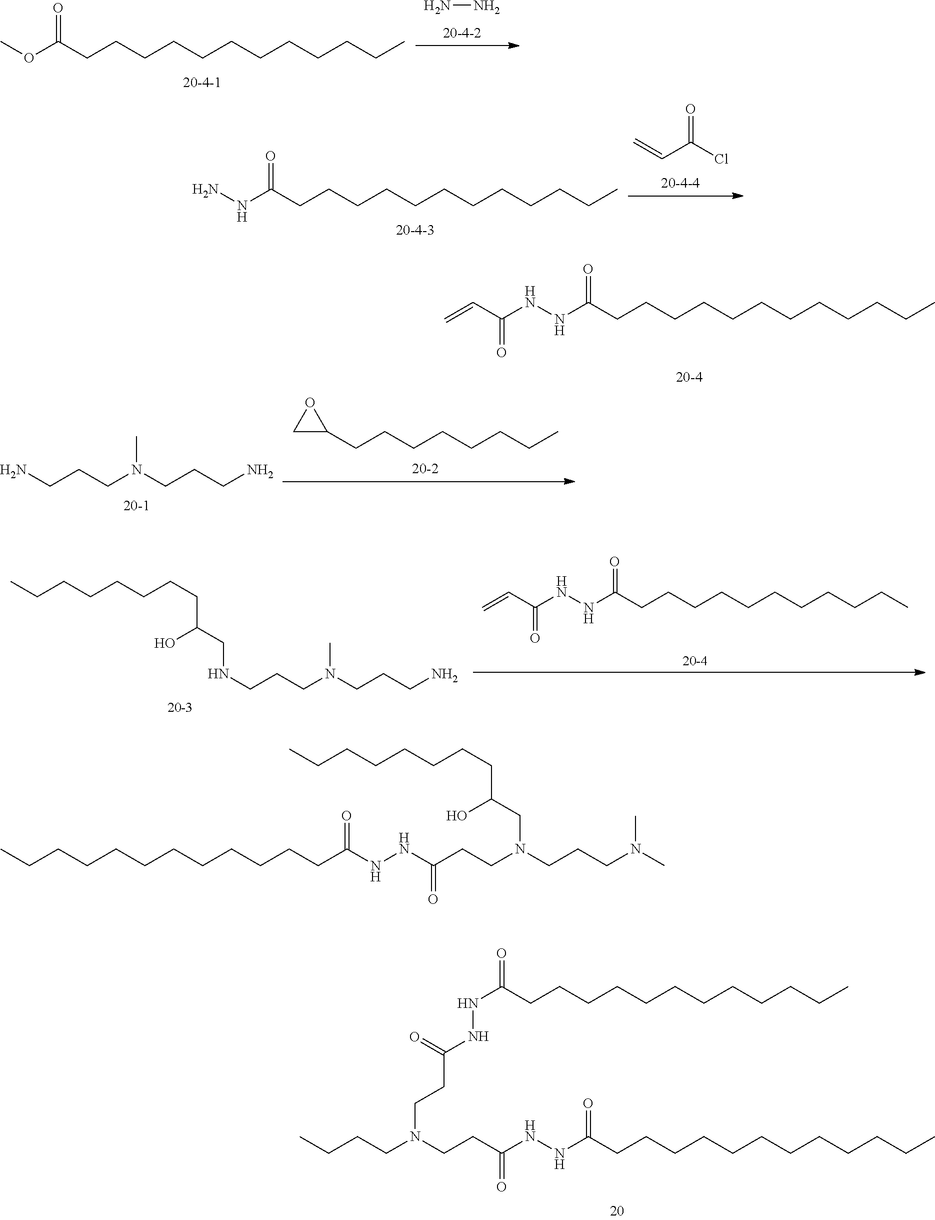

Step 1: to methanol (3 mL) were added compound 20-4-1 (1 g, 4.38 mmol) and compound 20-4-2 (2.8 g, 43.8 mmol) under N₂ atmosphere. The mixture was stirred at room temperature for 40 hours. TLC (eluent: DCM/MeOH=10/1) indicated incompletion consumption of starting materials. Compound 20-4-2 (2.8 g, 43.8 mmol) was added to the mixture. The mixture was heated at 60° C. for 16 hours. TLC (eluent: PE) indicated consumption of starting materials. The mixture was concentrated and diluted with water (10 mL). The mixture was stirred for 30 minutes and filtrated. The residue was washed and dried to afford compound 20-4-3 (540 mg).

¹H NMR of compound 20-4-3: (400 MHz, Chloroform-d) δ 6.82 (s, 1H), 3.90 (s, 2H), 2.14 (t, J=7.6 Hz, 2H), 1.62 (t, J=7.3 Hz, 2H), 1.26 (d, J=10.9 Hz, 18H), 0.87 (t, J=6.7 Hz, 3H). HRMS: [M+H]⁺ 229.22922.

Step 2: to DCM (20 mL) were added compound 20-4-3 (0.54 g, 2.4 mmol) and DIEA (0.62 g, 4.8 mmol) under N₂ atmosphere. The mixture was cooled to 5° C. compound 20-4-4 (0.26 g, 2.9 mmol) was added dropwise at 5-10° C. The mixture was allowed to warm to room temperature and stirred for 2 hours at the same temperature. TLC (eluent: DCM/MeOH=10/1) indicated consumption of starting materials. The mixture was purified by column chromatography on silica to afford crude product. The crude product was added into water (10 mL) and stirred for 30 minutes and dried to afford compound 20-4 (510 mg). HRMS: [M+H]+ 283.24978.

Step 3: compound 20-3 was prepared by the method of Example 1.1 Step 2.

Step 4: to a solution of compound 20-3 (30 mg, 0.096 mmol) compound 20-4 (101.2 mg, 0.36 mmol, prepared by the method of Example 1.16 Step 1 to 2) in ACN (0.2 mL) were added BHT (15 mg) and acetic acid (10 mg) at room temperature under N₂ atmosphere. The mixture was heated at 80° C. for 16 hours. TLC (eluent: DCM/MeOH=10/1+0.5% NH₄OH) indicated substantial consumption of starting materials. The mixture was purified by column chromatography on silica and concentrated under reduced pressure to afford compound 20 (50 mg, 43% yield).

¹H NMR of compound 20: (400 MHz, Chloroform-d) δ 3.70 (s, 1H), 3.07-2.87 (m, 1H), 2.68 (t, J=5.6 Hz, 2H), 2.48-2.31 (m, 6H), 2.31-2.19 (m, 5H), 2.15 (d, J=18.3 Hz, 2H), 1.62 (d, J=8.0 Hz, 7H), 1.26 (d, J=5.9 Hz, 48H), 0.94-0.76 (m, 12H). HRMS: [M+H]+ 1149.01684.

Other compounds of the present disclosure were synthesized by similar methods with modified conditions and different starting materials.

### Example 1.21: Synthesis of Compounds 21-39

Compounds 21-39 were synthesized by the method illustrated by the following scheme.

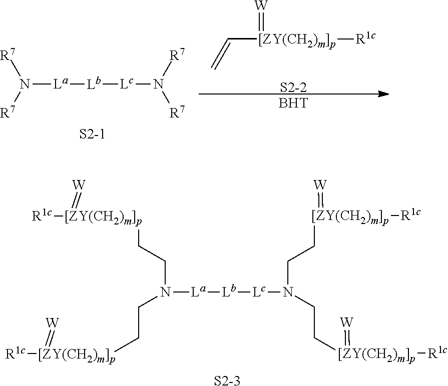

wherein
each W is independently selected from 0, S and NH;
each Y is independently selected from 0, S and NH;
each n is independently 1, 2 or 3;
each m is 0 or 1;
each p is 1 or 2.

### General Procedure

A mixture of compound S2-1, S2-2, 2,6-di-tert-butyl-4-methylphenol (BHT), acetonitrile (ACN) and/or acetic acid was added into a sealed container. The mixture was heated at 60-80° C. for 18-36 hours. Then the mixture was cooled to room temperature and purified by column chromatography to afford desired compound S2-3.

The following compounds were synthesized with modified conditions and different starting materials.

| Compd. | Structure |
|---|---|
| No. | ¹H NMR |
| 21 | 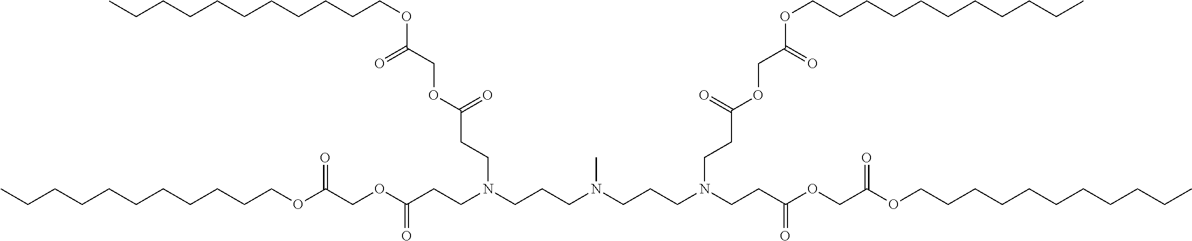 [SMILES: CCCCCCCCCCCOC(=O)COC(=O)CCN(CCCN(C)CCCN(CCC(=O)OCC(=O)OCCCCCCCCCCC)CCC(=O)OCC(=O)OCCCCCCCCCCC)CCC(=O)OCC(=O)OCCCCCCCCCCC] |
| | (400 MHz, Chloroform-d) δ 4.60 (s, 8H), 4.14 (t, J = 6.8 Hz, 8H), |
| | 2.80 (t, J = 7.3 Hz, 8H), 2.55 (t, J = 7.0 Hz, 8H), 2.52-2.15 (m, 8H), |
| | 1.63 (p, J = 6.9 Hz, 11H), 1.34-1.23 (m, 68H), 0.88 (t, J = 6.7 Hz, |
| | 12H). |
| 22 | 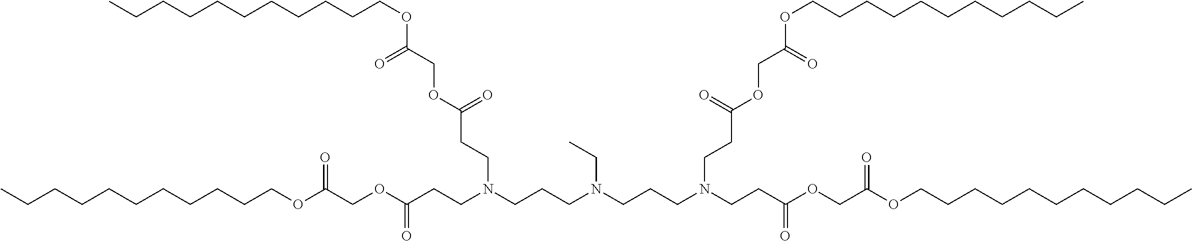 [SMILES: CCCCCCCCCCCOC(=O)COC(=O)CCN(CCCN(CC)CCCN(CCC(=O)OCC(=O)OCCCCCCCCCCC)CCC(=O)OCC(=O)OCCCCCCCCCCC)CCC(=O)OCC(=O)OCCCCCCCCCCC] |
| | (400 MHz, Chloroform-d) δ 4.60 (s, 8H), 4.14 (t, J = 6.8 Hz, 8H), |
| | 2.79 (d, J = 7.1 Hz, 8H), 2.68-2.27 (m, 16H), 1.63 (t, J = 7.0 Hz, |
| | 12H), 1.28 (d, J = 16.5 Hz, 74H), 0.88 (t, J = 6.7 Hz, 14H) |
| 23 | 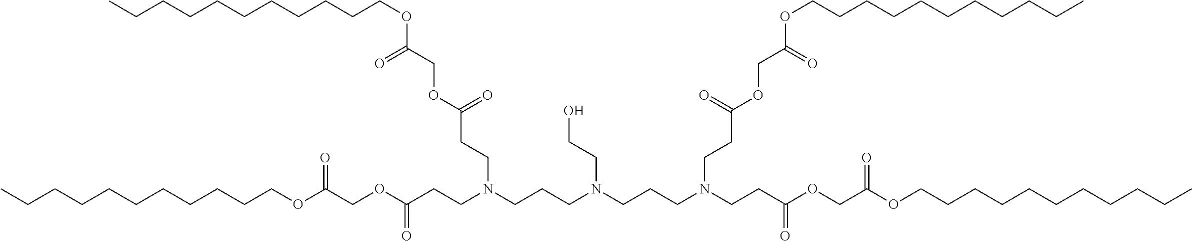 [SMILES: CCCCCCCCCCCOC(=O)COC(=O)CCN(CCCN(CCO)CCCN(CCC(=O)OCC(=O)OCCCCCCCCCCC)CCC(=O)OCC(=O)OCCCCCCCCCCC)CCC(=O)OCC(=O)OCCCCCCCCCCC] |
| | (400 MHz, Chloroform-d) δ 4.53 (s, 8H), 4.07 (t, J = 6.8 Hz, 9H), |
| | 2.73 (t, J = 6.9 Hz, 9H), 2.49 (t, J = 6.9 Hz, 10H), 2.40 (dt, J = 19.4, |
| | 6.8 Hz, 7H), 1.57 (q, J = 7.0 Hz, 12H), 1.20 (d, J = 8.0 Hz, 79H), 0.81 |
| | (t, J = 6.7 Hz, 15H) |
| 24 | 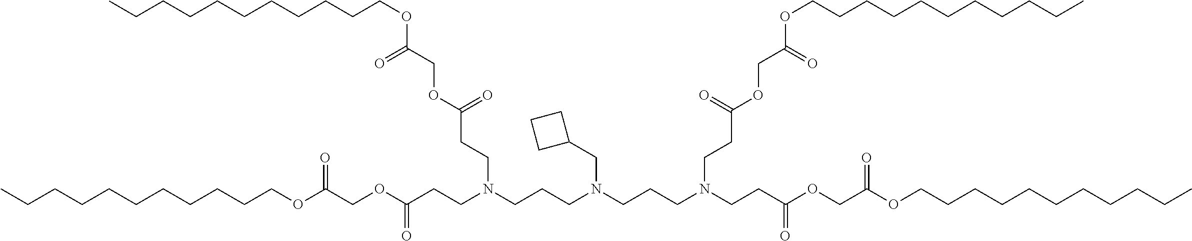 [SMILES: CCCCCCCCCCCOC(=O)COC(=O)CCN(CCCN(CCCN(CCC(=O)OCC(=O)OCCCCCCCCCCC)CCC(=O)OCC(=O)OCCCCCCCCCCC)CC1CCC1)CCC(=O)OCC(=O)OCCCCCCCCCCC] |
| | (400 MHz, Chloroform-d) δ 4.60 (s, 8H), 4.14 (t, J = 6.8 Hz, 8H), |
| | 2.98 (s, 2H), 2.79 (p, J = 7.0 Hz, 8H), 2.55 (t, J = 6.8 Hz, 9H), 2.38 |
| | (dt, J = 32.7, 7.2 Hz, 7H), 1.99 (s, 3H), 1.63 (p, J = 6.9 Hz, 12H), |
| | 1.28 (d, J = 16.2 Hz, 70H), 0.88 (t, J = 6.7 Hz, 13H) |
| 25 | 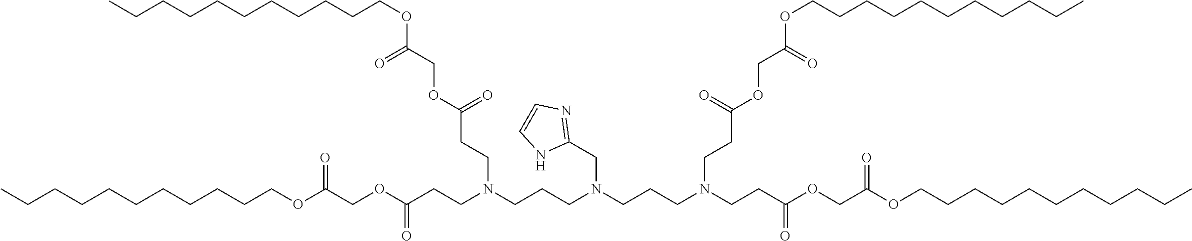 [SMILES: CCCCCCCCCCCOC(=O)COC(=O)CCN(CCCN(CCCN(CCC(=O)OCC(=O)OCCCCCCCCCCC)CCC(=O)OCC(=O)OCCCCCCCCCCC)Cc1ncc[nH]1)CCC(=O)OCC(=O)OCCCCCCCCCCC] |
| | (400 MHz, Chloroform-d) δ 6.96 (s, 1H), 6.91 (s, 1H), 4.60 (d, J = |
| | 6.9 Hz, 8H), 4.38 (t, J = 6.8 Hz, 1H), 4.20 (t, J = 6.8 Hz, 1H), 4.13 (t, |
| | J = 6.8 Hz, 9H), 3.66 (d, J = 10.7 Hz, 2H), 2.95 (t, J = 6.7 Hz, 2H), |
| | 2.78 (t, J = 7.3 Hz, 6H), 2.52 (t, J = 7.2 Hz, 6H), 2.39 (q, J = 10.6, 7.8 |
| | Hz, 6H), 1.63 (p, J = 6.9 Hz, 12H), 1.35-1.24 (m, 72H), 0.88 (t, J = |
| | 6.7 Hz, 14H) |
| 26 | 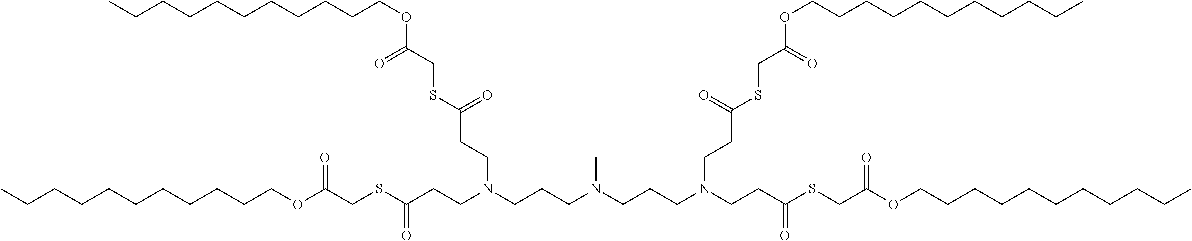 [SMILES: CCCCCCCCCCCOC(=O)CSC(=O)CCN(CCCN(C)CCCN(CCC(=O)SCC(=O)OCCCCCCCCCCC)CCC(=O)SCC(=O)OCCCCCCCCCCC)CCC(=O)SCC(=O)OCCCCCCCCCCC] |
| | (400 MHz, Chloroform-d) δ 4.10 (t, J = 6.9 Hz, 8H), 3.70 (s, 6H), |
| | 3.23 (s, 1H), 2.95 (s, 2H), 2.84-2.68 (m, 12H), 2.50 (q, J = 7.5, 6.9 |
| | Hz, 4H), 1.63 (t, J = 7.0 Hz, 9H), 1.36-1.19 (m, 75H), 0.91-0.75 |
| | (m, 15H) |
| 27 | 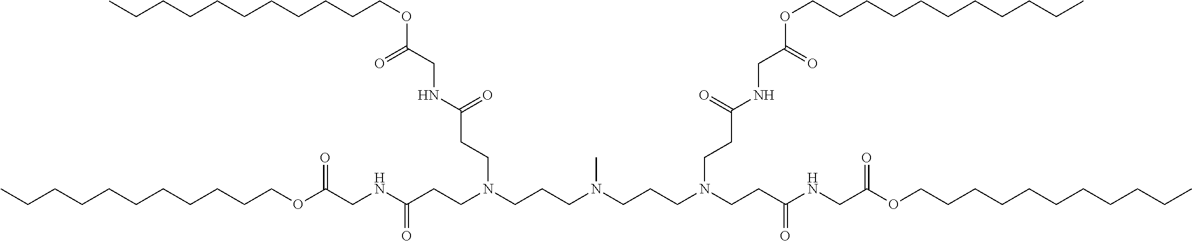 [SMILES: CCCCCCCCCCCOC(=O)CNC(=O)CCN(CCCN(C)CCCN(CCC(=O)NCC(=O)OCCCCCCCCCCC)CCC(=O)NCC(=O)OCCCCCCCCCCC)CCC(=O)NCC(=O)OCCCCCCCCCCC] |
| | (400 MHz, Chloroform-d) δ 7.71 (t, J = 5.6 Hz, 4H), 4.09 (t, J = 6.8 |
| | Hz, 8H), 3.98 (d, J = 5.6 Hz, 8H), 2.75 (t, J = 6.1 Hz, 8H), 2.51 (t, J = |
| | 7.1 Hz, 5H), 2.44 (t, J = 6.0 Hz, 9H), 1.71 (s, 3H), 1.63 (q, J = 7.0 |
| | Hz, 9H), 1.28 (d, J = 15.9 Hz, 67H), 0.88 (t, J = 6.8 Hz, 12H). |
| | HR-MS: [M + H]⁺ = 1279.03769 |
| 28 | 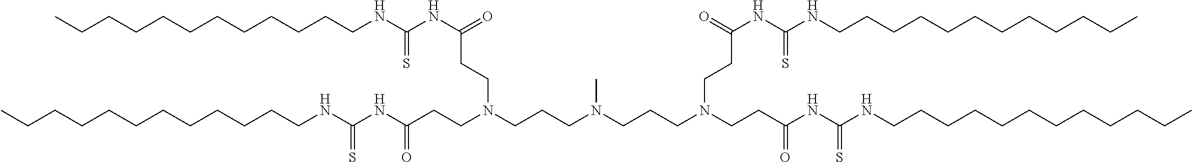 [SMILES: CCCCCCCCCCCCNC(=S)NC(=O)CCN(CCCN(C)CCCN(CCC(=O)NC(=S)NCCCCCCCCCCCC)CCC(=O)NC(=S)NCCCCCCCCCCCC)CCC(=O)NC(=S)NCCCCCCCCCCCC] |
| | (400 MHz, Chloroform-d) δ 10.05 (s, 4H), 8.41 (t, J = 5.7 Hz, 4H), |
| | 3.23 (q, J = 7.4, 6.9 Hz, 8H), 2.68 (t, J = 5.6 Hz, 8H), 2.53 (t, J = 5.6 |
| | Hz, 8H), 2.42 (s, 4H), δ 1.74 (m, 7H), 1.53 (p, J = 7.2 Hz, 8H), 1.27 |
| | (d, J = 11.4 Hz, 70H), 0.88 (t, J = 6.7 Hz, 12H). |
| | HR-MS: [M + H]⁺ = 1275.20592, |
| 29 | 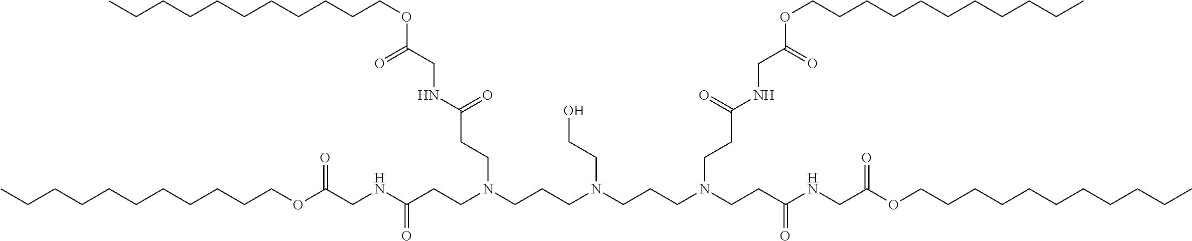 [SMILES: CCCCCCCCCCCOC(=O)CNC(=O)CCN(CCCN(CCO)CCCN(CCC(=O)NCC(=O)OCCCCCCCCCCC)CCC(=O)NCC(=O)OCCCCCCCCCCC)CCC(=O)NCC(=O)OCCCCCCCCCCC] |
| | (400 MHz, Chloroform-d) δ 7.22-7.65 (m, 4H), 4.09 (t, J = 6.7 Hz, |
| | 8H), 3.98 (d, J = 5.5 Hz, 8H), 3.70-3.61 (m, 2H), 2.81-2.68 (m, |
| | 10H), 2.52 (t, J = 7.1 Hz, 4H), 2.44 (t, J = 6.1 Hz, 8H), 1.70-1.66 (m, |
| | 4H), 1.63 (p, J = 6.9 Hz, 8H), 1.38-1.19 (m, 64H), 0.88 (t, J = 6.7 |
| | Hz, 12H) |
| 30 | 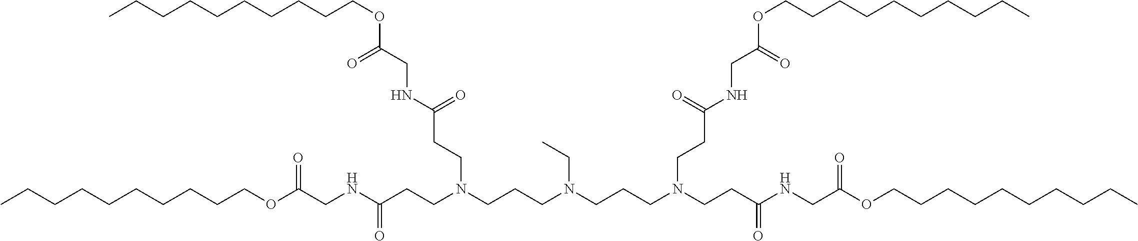 [SMILES: CCCCCCCCCCOC(=O)CNC(=O)CCN(CCCN(CC)CCCN(CCC(=O)NCC(=O)OCCCCCCCCCC)CCC(=O)NCC(=O)OCCCCCCCCCC)CCC(=O)NCC(=O)OCCCCCCCCCC] |
| | (400 MHz, Chloroform-d) δ 7.74 (t, J = 5.6 Hz, 4H), 4.10 (t, J = 6.8 |
| | Hz, 8H), 3.99 (d, J = 5.4 Hz, 8H), 2.76 (t, J = 6.1 Hz, 8H), 2.47 (dt, J = |
| | 25.8, 6.7 Hz, 12H), 1.63 (q, J = 7.1 Hz, 10H), 1.31 (d, J = 7.7 Hz, |
| | 20H), 1.26 (s, 45H), 1.07-0.98 (m, 3H) 0.88 (t, J = 6.7 Hz, 12H). |
| | HR-MS: [M + H]⁺ = 1293.06248 |
| 31 | 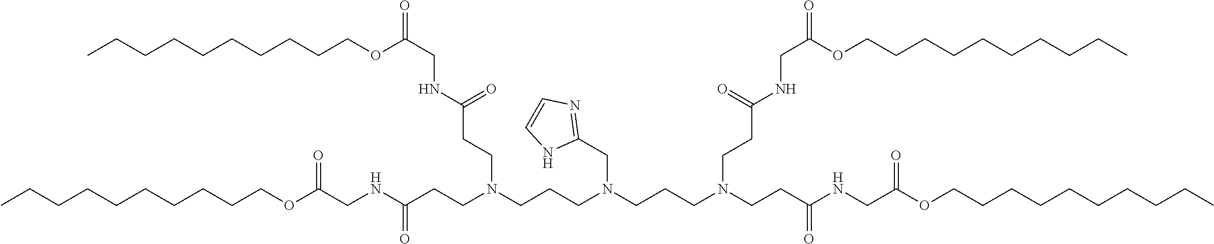 [SMILES: CCCCCCCCCCOC(=O)CNC(=O)CCN(CCCN(CCCN(CCC(=O)NCC(=O)OCCCCCCCCCC)CCC(=O)NCC(=O)OCCCCCCCCCC)Cc1ncc[nH]1)CCC(=O)NCC(=O)OCCCCCCCCCC] |
| | (400 MHz, Chloroform-d) δ 7.91 (t, J = 5.7 Hz, 3H), 7.01 (s, 2H), |
| | 4.07 (t, J = 6.8 Hz, 8H), 3.97 (d, J = 5.5 Hz, 8H), 3.86 (s, 1H), 2.75 |
| | (t, J = 6.1 Hz, 8H), 2.62 (s, 4H), 2.45 (dt, J = 16.3, 6.5 Hz, 12H), 1.77- |
| | 1.66 (m, 3H), 1.60 (q, J = 7.1 Hz, 9H), 1.28 (d, J = 12.6 Hz, 61H), |
| | 0.88 (t, J = 6.7 Hz, 12H). |
| | HR-MS: [M + H]⁺ = 1290.00711 |
| 32 | 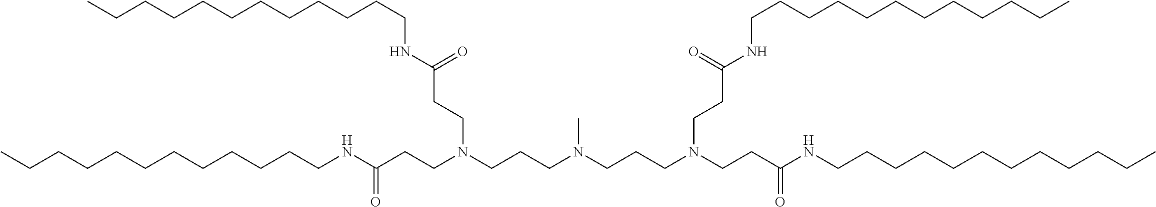 [SMILES: CCCCCCCCCCCCNC(=O)CCN(CCCN(C)CCCN(CCC(=O)NCCCCCCCCCCCC)CCC(=O)NCCCCCCCCCCCC)CCC(=O)NCCCCCCCCCCCC] |
| | (400 MHz, Chloroform-d) δ 7.07 (t, J = 5.6 Hz, 4H), 3.28-3.11 (m, |
| | 8H), 2.70 (t, J = 6.2 Hz, 7H), 2.42 (dt, J = 17.1, 7.0 Hz, 8H), 2.33 (t, |
| | J = 6.1 Hz, 9H), 2.24 (s, 3H), 1.61 (q, J = 7.1 Hz, 4H), 1.49 (p, J = 7.4 |
| | Hz, 8H), 1.27 (d, J = 9.1 Hz, 68H), 0.88 (t, J = 6.7 Hz, 12H). |
| | HR-MS: [M + H]⁺ = 1103.08503 |
| 33 | 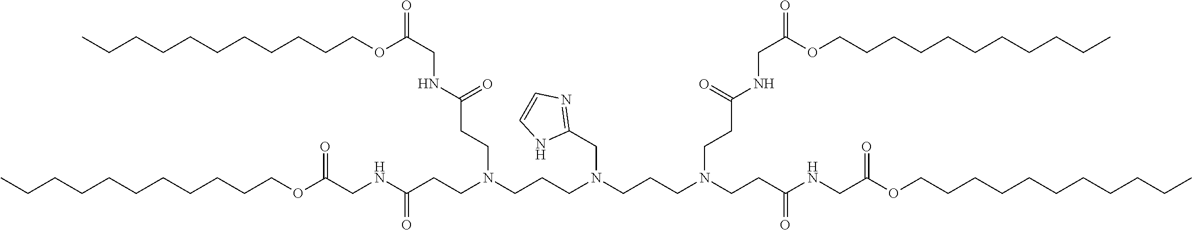 [SMILES: CCCCCCCCCCCOC(=O)CNC(=O)CCN(CCCN(CCCN(CCC(=O)NCC(=O)OCCCCCCCCCCC)CCC(=O)NCC(=O)OCCCCCCCCCCC)Cc1ncc[nH]1)CCC(=O)NCC(=O)OCCCCCCCCCCC] |
| | (400 MHz, Chloroform-d) δ 7.90 (t, J = 5.7 Hz, 2H), 7.02 (s, 2H), |
| | 4.07 (t, J = 6.8 Hz, 8H), 3.97 (d, J = 5.7 Hz, 9H), 2.75 (t, J = 6.0 Hz, |
| | 7H), 2.70 (s, 3H), 2.45 (dt, J = 12.4, 6.5 Hz, 14H), 1.73 (t, J = 6.7 |
| | Hz, 3H), 1.60 (q, J = 6.8 Hz, 9H), 1.28 (d, J = 11.9 Hz, 63H), 0.88 (t, |
| | J = 6.8 Hz, 12H). |
| | HR-MS: [M + H]⁺ = 1345.06869 |
| 34 | 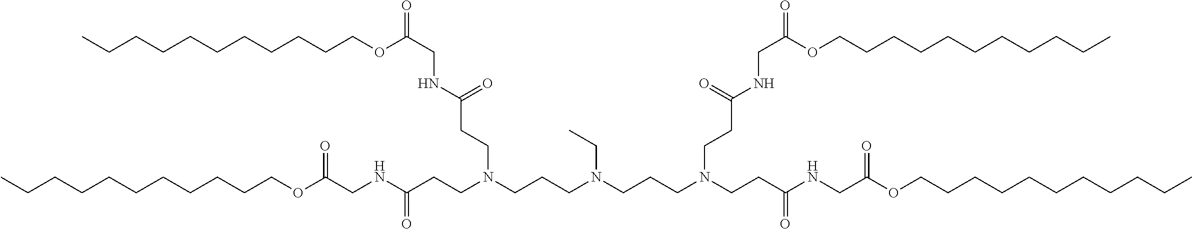 [SMILES: CCCCCCCCCCCOC(=O)CNC(=O)CCN(CCCN(CC)CCCN(CCC(=O)NCC(=O)OCCCCCCCCCCC)CCC(=O)NCC(=O)OCCCCCCCCCCC)CCC(=O)NCC(=O)OCCCCCCCCCCC] |
| | (400 MHz, Chloroform-d) δ 7.74 (t, J = 5.6 Hz, 4H), 4.10 (t, J = 6.8 |
| | Hz, 8H), 3.99 (d, J = 5.4 Hz, 8H), 2.76 (t, J = 6.1 Hz, 8H), 2.47 (d,t J = |
| | 25.8, 6.7 Hz, 14H), 1.63 (q, J = 7.1 Hz, 10H), 1.31 (d, J = 7.7 Hz, |
| | 20H), 1.26 (s, 45H), 1.07-0.98 (m, 3H) 0.88 (t, J = 6.7 Hz, 12H). |
| | HR-MS: [M + H]⁺ = 1293.06248 |
| 35 | 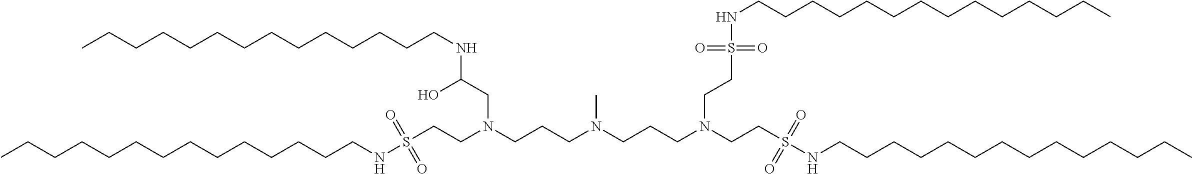 [SMILES: CCCCCCCCCCCCCCNC(O)CN(CCCN(C)CCCN(CCS(=O)(=O)NCCCCCCCCCCCCCC)CCS(=O)(=O)NCCCCCCCCCCCCCC)CCS(=O)(=O)NCCCCCCCCCCCCCC] |
| | (400 MHz, Chloroform-d) δ 5.47 (s, 3H), 3.19 (t, J = 6.0 Hz, 8H), |
| | 3.08 (q, J = 6.6 Hz, 8H), 2.92 (t, J = 6.0 Hz, 8H), 2.80 (s, 3H), 2.56 |
| | (t, J = 6.8 Hz, 5H), 2.48 (s, 3H), 2.38-2.09 (m ,6H), 2.03 (s, 3H), |
| | 1.84 (s, 4H), 1.57 (p, J = 7.5 Hz, 8H), 1.25 (s, 90H), 0.97-0.81 (m, |
| | 12H) |
| 36 | 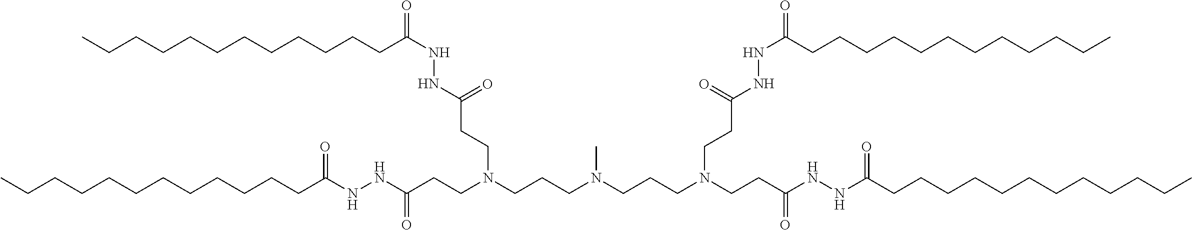 [SMILES: CCCCCCCCCCCCC(=O)NNC(=O)CCN(CCCN(C)CCCN(CCC(=O)NNC(=O)CCCCCCCCCCCC)CCC(=O)NNC(=O)CCCCCCCCCCCC)CCC(=O)NNC(=O)CCCCCCCCCCCC] |
| | (400 MHz, Chloroform-d) δ 2.89-2.56 (m, 5H), 2.45 (d, J = 6.1 Hz, |
| | 8H), 2.26 (q, J = 6.8, 5.9 Hz, 8H), 2.16-2.05 (m, 2H), 1.63 (t, J = |
| | 7.5 Hz, 7H), 1.56-1.43 (m, 2H), 1.37-1.11 (m, 59H), 0.95-0.73 |
| | (m, 12H). |
| | HRMS: [M + H]⁺ 1275.09531 |
| 37 | 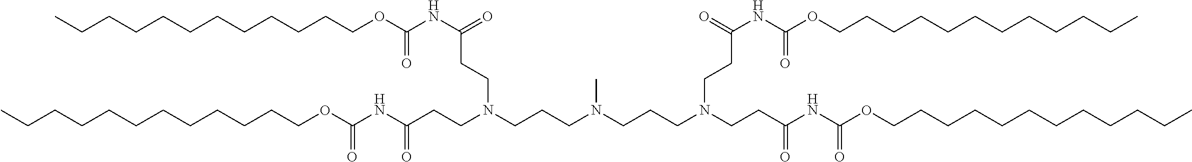 [SMILES: CCCCCCCCCCCCOC(=O)NC(=O)CCN(CCCN(C)CCCN(CCC(=O)NC(=O)OCCCCCCCCCCCC)CCC(=O)NC(=O)OCCCCCCCCCCCC)CCC(=O)NC(=O)OCCCCCCCCCCCC] |
| | (400 MHz, Chloroform-d) δ 9.00 (s, 3H), 4.06 (t, J = 6.9 Hz, 8H), |
| | 2.71 (dt, J = 29.1, 6.5 Hz, 15H), 2.46 (t, J = 7.2 Hz, 4H), 2.28 (d, J = |
| | 10.9 Hz, 4H), 2.11 (s, 4H), 1.59 (q, J = 6.9 Hz, 12H), 1.20 (d, J = 6.5 |
| | Hz, 72H), 0.81 (t, J = 6.7 Hz, 12H). |
| | HR-MS: [M + H]⁺ = 1279.04739 |
| 38 | 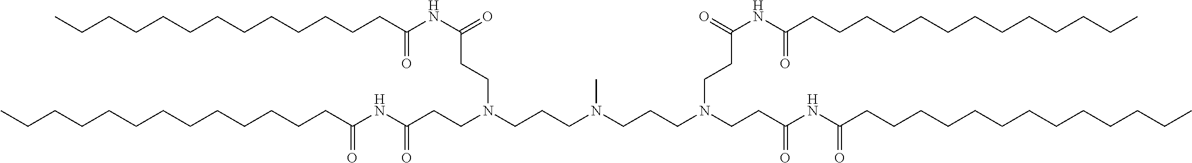 [SMILES: CCCCCCCCCCCCCC(=O)NC(=O)CCN(CCCN(C)CCCN(CCC(=O)NC(=O)CCCCCCCCCCCCC)CCC(=O)NC(=O)CCCCCCCCCCCCC)CCC(=O)NC(=O)CCCCCCCCCCCCC] |
| | (400 MHz, Chloroform-d) δ 10.00 (s, 3H), 2.77 (t, J = 6.0 Hz, 8H), |
| | 2.67 (h, J = 6.0 Hz, 15H), 2.52 (t, J = 7.1 Hz, 4H), 2.32 (s, 4H), 2.25- |
| | 2.10 (m, 4H), 1.60 (q, J = 9.9, 7.2 Hz, 12H), 1.25 (s, 76H), 0.93- |
| | 0.81 (m, 12H). |
| | HR-MS: [M/2 + H]⁺ = 636.06442, [M + H]⁺ = 1271.12962, |
| | [M+ Na]⁺ = 1294.11383 |
| 39 | 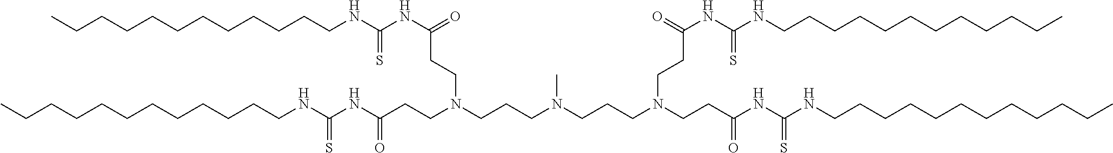 [SMILES: CCCCCCCCCCCCNC(=S)NC(=O)CCN(CCCN(C)CCCN(CCC(=O)NC(=S)NCCCCCCCCCCCC)CCC(=O)NC(=S)NCCCCCCCCCCCC)CCC(=O)NC(=S)NCCCCCCCCCCCC] |
| | (400 MHz, Chloroform-d) δ 10.70-10.29 (m, 8H), 3.57 (td, J = 7.3, |
| | 5.3 Hz, 8H), 3.00-2.83 (m, 4H), 2.73 (t, J = 5.8 Hz, 8H), 2.60 (t, J = |
| | 5.7 Hz, 8H), 2.53 (d, J = 8.6 Hz, 7H), 1.96 (q, J = 7.7 Hz, 4H), 1.63 |
| | (t, J = 7.3 Hz, 8H), 1.48-1.10 (m, 80H), 0.87 (t, J = 6.7 Hz, 13H). |
| | HRMS: [M + H]⁺ = 1339.01158. |

### Example 2: Preparation of mRNA

Preparation of mRNA

An mRNA can be prepared in vitro by any means well-known in the art. For example, the mRNA used in the exemplary examples can be prepared by using TranscriptAid T7 High Yield Transcription Kit (Thermo K0441). In vitro transcription was performed using the TranscriptAid T7 High Yield Transcription Kit, using linear DNA as a template for generating a mRNA of interest, with addition of a certain proportion of pseudouridine and capping reagents. In vitro transcription conditions: the reaction system was configured according to the kit instructions, reaction at 37° C. for 0.5-2 hours, the transcript was digested for 30 minutes by DNase, and the transcript was purified with Monarch RNA Cleanup Kit (NEB T2040L).

The exemplary mRNA constructs were shown in Table 7 below. The signal peptide in each construct can be substituted with alternative sequences that achieve the same or similar function. The exemplary signal peptide has an amino acid sequence of SEQ ID NO: 185, which is encoded by the nucleic acid sequence of SEQ ID NO: 184.

TABLE 7
Description and Sequence of test mRNAs

| Nucleic | SEQ ID | Description of the constructs |
|---|---|---|
| acid ID | NOs | (the corresponding polypeptides) |
| WT1 (wild | SEQ ID | Signal peptide-wild type RSV F |
| type | NO: 1 | |
| sequence) | | |
| WT2 or WT(v2) | SEQ ID | Signal peptide-wild type RSV F |
| (codon | NO: 2 | |
| optimized | | |
| sequence) | | |
| P1 | SEQ ID | Signal peptide-RSV F protein variant |
| | NO: 3 | (K209P, Q210P) |
| P2 | SEQ ID | Signal peptide-RSV F protein variant |
| | NO: 4 | (Q210P, S211P) |
| P3 | SEQ ID | Signal peptide-RSV F protein variant |
| | NO: 5 | (S211P, C212P) |
| P4 | SEQ ID | Signal peptide-RSV F protein variant |
| | NO: 6 | (L481P) |
| P5 | SEQ ID | Signal peptide-RSV F protein variant |
| | NO: 7 | (L481P, V482P) |
| P6 | SEQ ID | Signal peptide-RSV F protein variant |
| | NO: 8 | (K209P, Q210P, L481P, V482P) |
| P7 | SEQ ID | Signal peptide-RSV F protein variant |
| | NO: 9 | (Q210P, S211P, L481P, V482P) |
| P8 | SEQ ID | Signal peptide-RSV F protein variant |
| | NO: 10 | (S211P, C212P, L481P, V482P) |
| P9 | SEQ ID | Signal peptide-RSV F protein variant |
| | NO: 11 | (K209P, Q210P, L481P) |
| P10 | SEQ ID | Signal peptide-RSV F protein variant |
| | NO: 12 | (Q210P, S211P, L481P) |
| P11 | SEQ ID | Signal peptide-RSV F protein variant |
| | NO: 13 | (S211P, C212P, L481P) |
| 210-T-S-V | SEQ ID | Signal peptide-RSV F protein variant |
| | NO: 14 | (S55C, P102A, T103C, I148C, L188C, |
| | | V207L Q210P, I379V, M447V) |
| 210-T-S | SEQ ID | Signal peptide-RSV F protein variant |
| | NO: 15 | (S55C, P102A, T103C, I148C, L188C, |
| | | Q210P, I379V, M447V) |
| 210-T-V | SEQ ID | Signal peptide-RSV F protein variant |
| | NO: 16 | (P102A, T103C, I148C, V207L, Q210P, |
| | | I379V, M447V) |
| 210-S-V | SEQ ID | Signal peptide-RSV F protein variant |
| | NO: 17 | (S55C, P102A, L188C, V207L, Q210P, |
| | | I379V, M447V) |
| 210-T | SEQ ID | Signal peptide-RSV F protein variant |
| | NO: 18 | (P102A, T103C, I148C, Q210P, I379V, |
| | | M447V) |
| 210-S | SEQ ID | Signal peptide-RSV F protein variant |
| | NO: 19 | (S55C, P102A, L188C, Q210P, I379V, |
| | | M447V) |
| 210-V-3 | SEQ ID | Signal peptide-RSV F protein variant |
| | NO: 20 | (P102A, V207L, Q210P, I379V, M447V) |
| 210-V | SEQ ID | Signal peptide-RSV F protein variant |
| | NO: 21 | (V207L, Q210P) |
| 210-213-T-S-V | SEQ ID | Signal peptide-RSV F protein variant |
| | NO: 22 | (S55C, P102A, T103C, I148C, L188C, |
| | | V207L, Q210C, S213C, I379V, M447V) |
| 210-213-T-S | SEQ ID | Signal peptide-RSV F protein variant |
| | NO: 23 | (S55C, P102A, T103C, I148C, L188C, |
| | | Q210C, S213C, I379V, M447V) |
| 210-213-T-V | SEQ ID | Signal peptide-RSV F protein variant |
| | NO: 24 | (P102A, T103C, I148C, V207L, Q210C, |
| | | S213C, I379V, M447V) |
| 210-213-S-V | SEQ ID | Signal peptide-RSV F protein variant |
| | NO: 25 | (S55C, P102A, L188C, V207L, Q210C, |
| | | S213C, I379V, M447V) |
| 210-213-T | SEQ ID | Signal peptide-RSV F protein variant |
| | NO: 26 | (P102A, T103C, I148C, Q210C, S213C, |
| | | I379V, M447V) |
| 210-213-S | SEQ ID | Signal peptide-RSV F protein variant |
| | NO: 27 | (S55C, P102A, L188C, Q210C, S213C, |
| | | I379V, M447V) |
| 210-213-V-3 | SEQ ID | Signal peptide-RSV F protein variant |
| | NO: 28 | (P102A, V207L, Q210C, S213C, I379V, |
| | | M447V) |
| 210-213-V | SEQ ID | Signal peptide-RSV F protein variant |
| | NO: 29 | (V207L, Q210C, S213C) |
| S55C + L188C | SEQ ID | Signal peptide-RSV F protein variant |
| | NO: 30 | (S55C, P102A, L188C, I379V, M447V) |
| T103C + I148C | SEQ ID | Signal peptide-RSV F protein variant |
| | NO: 31 | (P102A, T103C, I148C, I379V, M447V) |
| P102A + I379V + | SEQ ID | Signal peptide-RSV F protein variant |
| M447V | NO: 32 | (102A, 379V, M447V) |
| V207L | SEQ ID | Signal peptide-RSV F protein variant |
| | NO: 33 | (V207L) |

### Example 3: In Vitro Expression Assay

### Western Blot and ELISA Assay

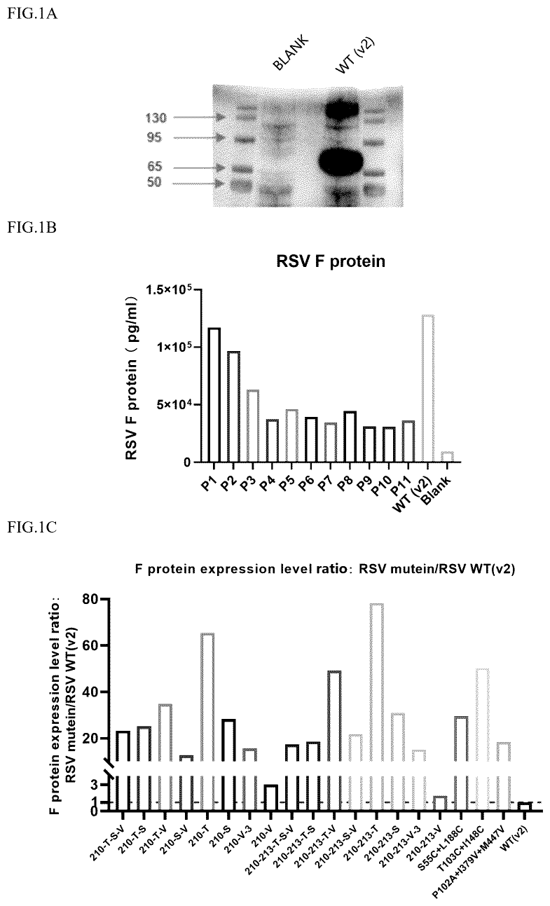

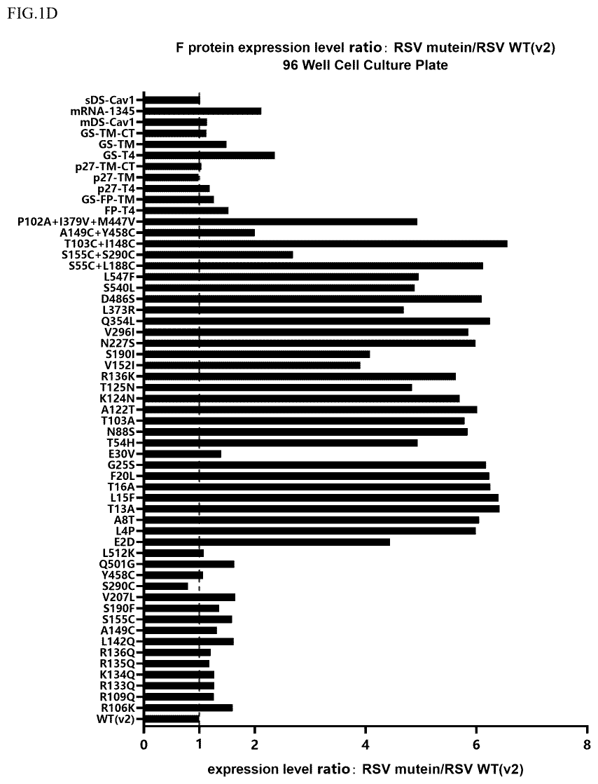

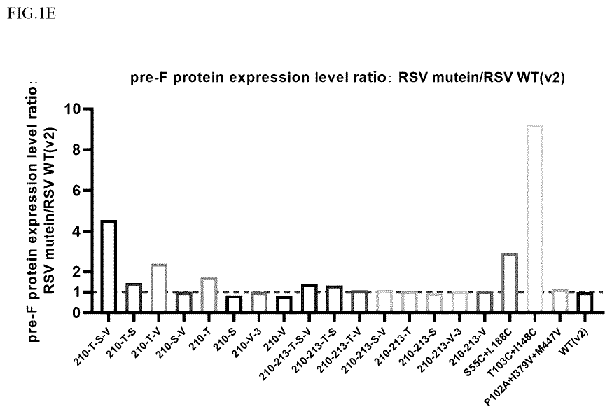

HEK293 cells were transfected with test mRNAs. HEK293 cells at log phase were employed, the cell suspension concentration was adjusted to 5×10⁵ cells/ml, and inoculated into 6-well plates at 1 ml per well. The cells were placed in an incubator at 5% CO₂ and 37° C. cultured overnight for adherence. 4 μg test mRNA and 8 μL Lipofectamine 2000 (ThermoFisher Scientific) were diluted in 250 μL serum-free Opti-MEM respectively, and then incubated at room temperature for 5 min. The above mRNA solution was mixed with the above Lipofectamine 2000, and stood at room temperature for 20 min. 500 μL oligonuclium-liposome complex was added to the cell culture plate, and 500 μL of serum-free Opti-MEM was added to the 6-well plate, to fill transfection system to 1 mL. 24 h after transfection, HEK293 cells were lysed by M-PER Mammalian Protein Extraction Reagent (Thermo) for protein extraction, and the obtained protein was used for Western Blot and ELISA assay. FIG. 1A-1E indicated the RSV F protein and pre-fusion RSV-F protein (“pre-F protein”) expression in cell lines detected by Western Blot and ELISA.

### FACS Flow Cytometry Assay

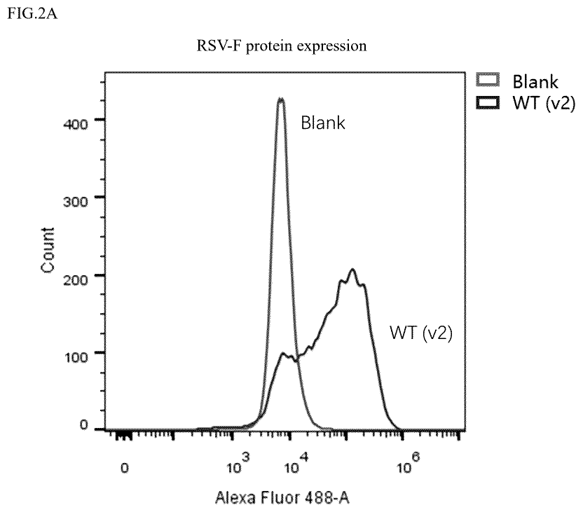

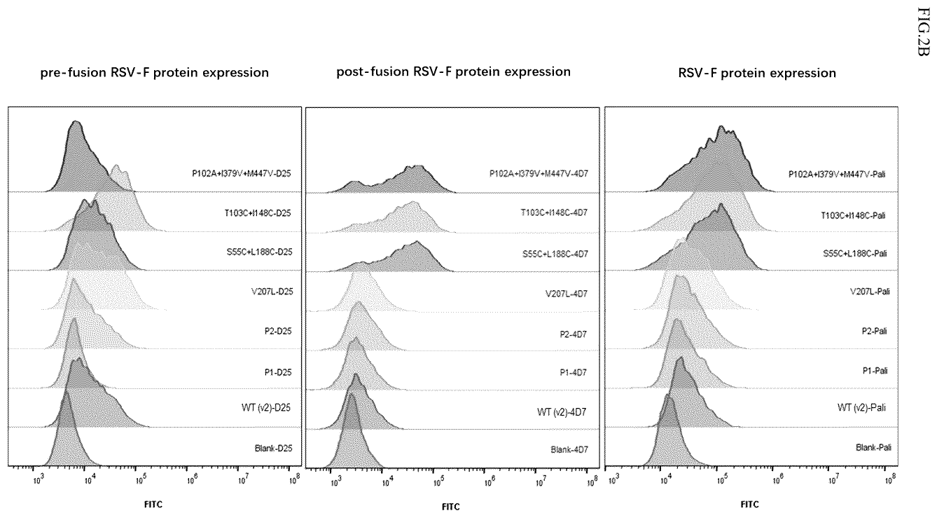

COS7 cells were transfected with test RSV-F mRNAs. COS7 cells at log phase were employed, the cell suspension concentration was adjusted to 5×10⁵ cells/ml, and inoculated into 6-well plates at 1 ml per well. The cells were placed in an incubator at 5% CO₂ and 37° C. cultured overnight for adherence. 4 μg test mRNA and 8 μL Lipofectamine 2000 (ThermoFisher Scientific) were diluted in 250 μL serum-free Opti-MEM respectively, and then incubated at room temperature for 5 min. The above mRNA solution was mixed with the above Lipofectamine 2000, and stood at room temperature for 20 min. 500 μL oligonuclium-liposome complex was added to the cell culture plate, and 500 μL of serum-free Opti-MEM was added to the 6-well plate, to fill transfection system to 1 mL. RSV F specific antibodies (sinobiological; #11049-R302) and Alexa Fluor®488 labeled Goat anti-mouse IgG antibody (abcam, #ab150113) or FITC labeled hIgG antibody D25 (“D25”, which recognizes the pre-fusion RSV-F specific antigenic site Ø), antibody 4D7 (“4D7”, which recognizes the post-fusion RSV-F specific antigenic site I) and Palivizumab (“Pali”, which recognizes the common antigenic site between pre-fusion RSV-F and post-fusion RSV-F) were used for staining. The analysis was performed by flow cytometry (FACS). Flow cytometric data were quantitatively evaluated using FlowJo software. Results shown in FIG. 2A-2B indicated the RSV F protein, pre-fusion RSV-F protein and post-fusion RSV-F protein expression in cell lines detected by FACS.

### Example 4: Preparation of Lipid Nanoparticle Composition

Step 1: mRNA was dissolved in citrate buffer (pH 4) and adjusted the concentration of mRNA to 0.2 mg/mL, so that obtaining aqueous layer thereby.

Step 2: Test Compound, 1,2-distearoyl-sn-glycero-3-phosphocholine (DSPC), cholesterol and DMG-PEG2000 were dissolved in desired molar fractions (shown in below Table 8) in dry ethanol and the total concentration of lipids was adjusted to 10 mg/mL, thereby obtaining organic layer.

TABLE 8
Molar Fractions of Components in Lipid Particles

| Lipid | | | | |
|---|---|---|---|---|
| Particle | Test Compound | | | |
| No. | (Compound 8) | DSPC | Cholesterol | DMG-PEG2000 |
| 5 | 40% | 6% | 52.4% | 1.6% |
| 6 | 40% | 8% | 50.1% | 1.9% |
| 7 | 43% | 6% | 49.1% | 1.9% |
| 8 | 43% | 8% | 47.4% | 1.6% |
| 9 | 46% | 6% | 45.9% | 2.1% |
| 10 | 46% | 8% | 43.6% | 2.4% |
| 11 | 46% | 10% | 42.4% | 1.6% |
| 12 | 46% | 12% | 40.1% | 1.9% |
| 13 | 49% | 6% | 42.6% | 2.4% |
| 14 | 49% | 8% | 40.9% | 2.1% |
| 15 | 49% | 12% | 37.4% | 1.6% |
| 16 | 44% | 7% | 47.5% | 1.5% |
| 17 | 45% | 6% | 47.5% | 1.5% |
| 18 | 45% | 8% | 45.3% | 1.7% |
| 19 | 47% | 7% | 44.4% | 1.6% |
| 20 | 47% | 8% | 43.5% | 1.5% |

Step 3: The aqueous layer and organic layer were admixed in 3:1 ratio (v/v) by microfluidic device (NanoAssemblr® Ignite™) at total flow rate of 12 mL/min. The mixture was 10-fold diluted with PBS buffer (pH 7.4). Ethanol was separated by tangential flow filtration (Repligen, TEE). The solution was concentrated to 0.1 mg/mL (mRNA concentration) and filtrated by 0.22 μm millipore filter to afford mRNA containing lipid particles.

Mean particle size and polymer dispersity index (PDI) of exemplary lipid particles were measured with DLS by Malvern Zetasizer.

Total RNA concentration and free RNA concentration were measured based on the measurement of fluorescence with RiboGreen® (ThermoFisher Scientific). The efficiency of encapsulation (EE) was calculated by

$$efficiency of encapsulation = \frac{total RNA concentration - free RNA concentration}{total RNA concentration}$$

The total RNA concentration was measured by properly diluting test lipid particle with 1×TE buffer containing 0.2% Triton-X 100. The free RNA concentration was measured by properly diluting test lipid particle with 1×TE buffer.

TABLE 9
Physical Characteristics of Test Lipid Particles

| Formulation | | Polydispersity | Encapsulation |
|---|---|---|---|
| No. | Particle size (nm) | index (PDI) | efficiency (EE, %) |
| 5 | 83.22 | 0.085 | 95 |
| 6 | 77.03 | 0.0945 | 92 |
| 7 | 79.67 | 0.0862 | 93.7 |
| 8 | 86.66 | 0.0749 | 93.1 |
| 9 | 82.52 | 0.067 | 93.1 |
| 10 | 76.1 | 0.0927 | 94.6 |
| 11 | 84.11 | 0.0659 | 94.7 |
| 12 | 77.97 | 0.0475 | 93.5 |
| 13 | 81.79 | 0.0719 | 96.1 |
| 14 | 81.25 | 0.0874 | 96.1 |
| 15 | 87.02 | 0.0664 | 95.2 |
| 16 | 103.8 | 0.0279 | 97.6 |
| 17 | 106.7 | 0.0384 | 97.5 |
| 18 | 96.22 | 0.0502 | 97 |
| 19 | 113.3 | 0.0223 | 97 |
| 20 | 115.8 | 0.0482 | 97.1 |

### Example 5: In Vivo Delivery Assay

Lipid nanoparticle compositions comprising FLuc mRNA or test RSV mRNA (WT(v2)) were prepared according to the method of Example 4. The components and their molar fraction in test lipid nanoparticles were shown in below Table 10. The solution was concentrated to 0.5 mg/mL (mRNA concentration) and filtrated by 0.22 μm millipore filter to afford mRNA containing lipid nanoparticle compositions.

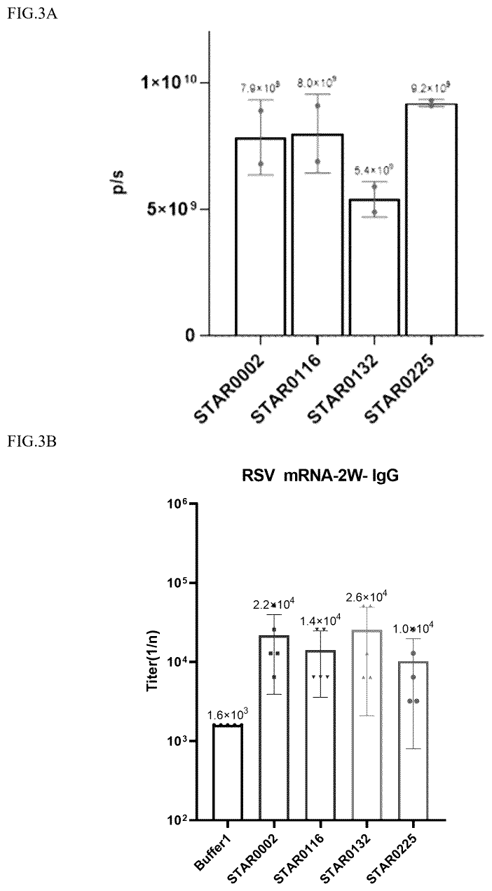

The resulted mRNA containing lipid nanoparticles were injected to C57 mice or Balb/C mice, respectively. Expression of fluorescent protein was measured by fluorescent imaging (FIG. 3A). Titers of RSV binding antibodies in serum were measured 2 weeks after injection (FIG. 3B). It can be seen that the test lipid nanoparticles effectively deliver the mRNAs to the mice and the mRNAs stably expressed in vivo.

TABLE 10
Molar Fractions of Components in Lipid Nanoparticles

| Lipid | | | | DMG-PEG |
|---|---|---|---|---|
| Nanoparticles | Lipid | DSPC | Cholesterol | 2000 |
| STAR0002 | 50% | 10% | 38.5% | 1.5% |
| | (SM-102, | | | |
| | CAS No: | | | |
| | 2089251-47-6) | | | |
| STAR0116 | 46% | 8% | 44.5% | 1.5% |
| | (Compound 1) | | | |
| STAR0132 | 46% | 8% | 43.9% | 2.1% |
| | (Compound 1) | | | |
| STAR0225 | 46% | 10% | 42.4% | 1.6% |
| | (Compound 8) | | | |

### Example 6: In Vivo Immunogenicity Assay

Lipid nanoparticle compositions comprising RSV mRNA (WT(v2)) and STAR0116 were prepared according to the method of Example 4. The solution was concentrated to 0.5 mg/mL (mRNA concentration) and filtrated by 0.22 μm millipore filter to afford mRNA containing lipid nanoparticle compositions. The lipid nanoparticle compositions were tested with female Balb/C mice (6 to 8 weeks old). 10 mice were employed for each group, and injected 50 μL per mouse according to below Table 11. Normal saline injection of equal volume was used as negative control. Immunization was performed on Day 0 and Day 28. On Day 14 (2W), Day 21 (3W), Day 35 (5W), and Day 56 (8W), blood was collected and used for serological tests. On Day 49, 3 mice in each group were killed and their spleens were harvested for IFN-γ ELISPOT assay.

TABLE 11
Immunization Dosage and Schedule

| | | Vaccine | | Immunization | |
|---|---|---|---|---|---|
| Group | Mice | dose | | schedule | Blood collection |
| 1 | Balb/C, | 50 | μg | Day 0 and Day 28 | Day 14, Day 21, Day 35, |
| | female | | | | and Day 56 |
| 2 | Balb/C, | 10 | μg | Day 0 and Day 28 | Day 14, Day 21, Day 35, |
| | female | | | | and Day 56 |
| 3 | Balb/C, | 0.5 | μg | Day 0 and Day 28 | Day 14, Day 21, Day 35, |
| | female | | | | and Day 56 |
| 4 | Balb/C, | 0 | | Day 0 and Day 28 | Day 14, Day 21, Day 35, |
| | female | | | | and Day 56 |

### RSV-F Protein Antibodies Assay

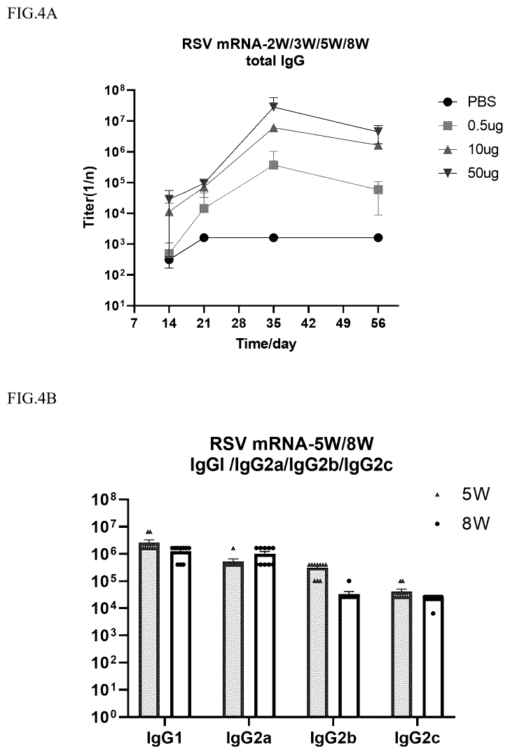

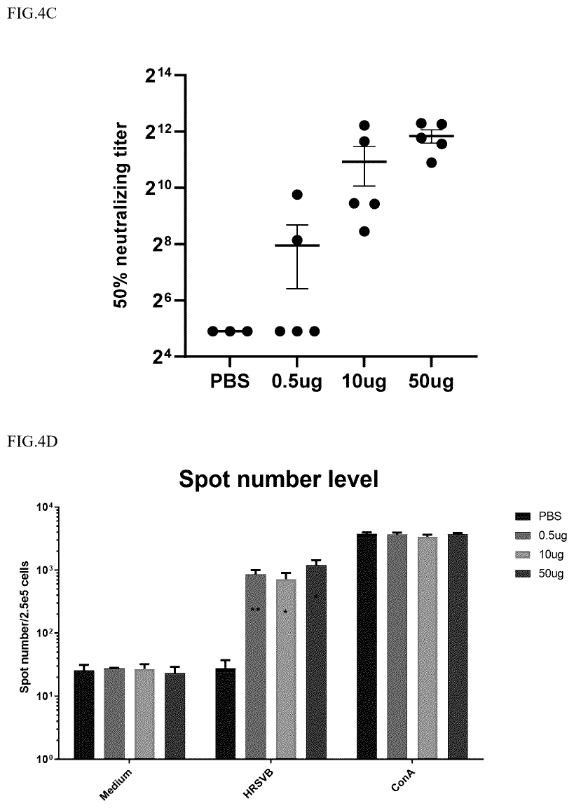

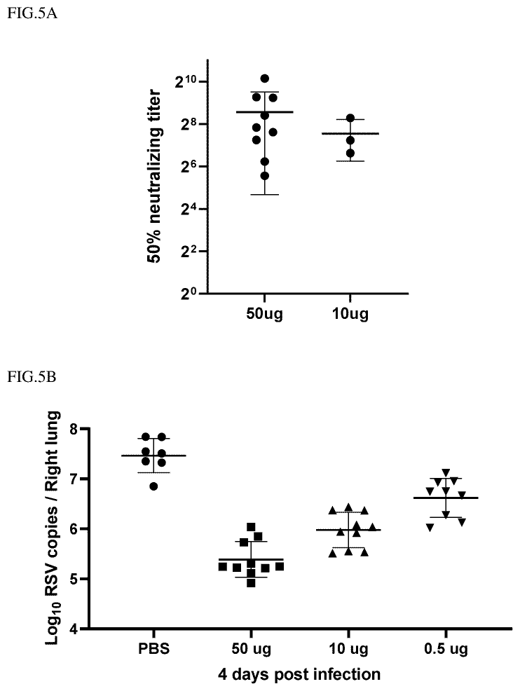

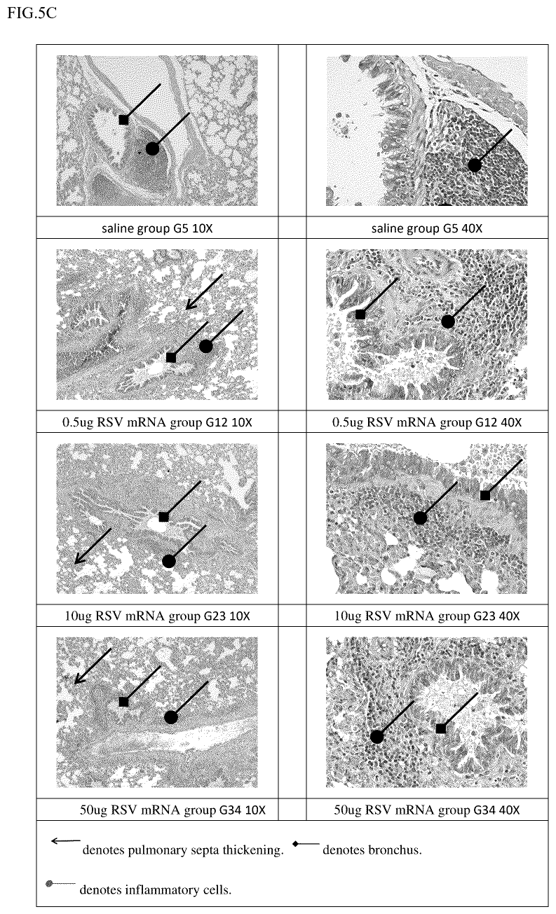

ELISA plates were coated with recombinant human RSV fusion glycoprotein (rec.hu F protein, final concentration: 0.2 μg/mL) (SinoBiological Inc.). The coated plates were incubated with a given serum dilution, and anti-mouse antibodies labeled with HRP were used to measure the binding of antibodies specifically to RSV-F protein using TMB substrate. FIGS. 4A and 4B indicated that the lipid nanoparticle compositions (RSV mRNA vaccine) induce high RSV-F antibody titers (total IgG, IgG1, IgG2a, IgG2b, and IgG2c).

### RSV Neutralizing Antibody Assay

Mouse serum collected at Day 42 was inactivated in 56° C. water bath for 0.5 hour before the test. Gradient dilution of the samples was used. The diluted samples (8 concentrations, double and double wells) were incubated with RSV A long virus at 37° C. and 5% CO₂ for 2 hours. HEp-2 cells were then inoculated into the test wells and cultured for 5 days at 37° C. and 5% CO₂. Cell control (cells with no sample treatment nor virus infection) and virus control (cells with virus infection but no sample treatment) wells were established. Viral protein in each well was measured by ELISA, and the original data were used to calculate the neutralizing activity of the sample at different concentrations. Serum neutralization titers were then calculated using a 4-parameter curve fit in the Graphpad Prism. As shown in FIG. 4C, the test lipid nanoparticle compositions induce higher level antibody titers.

### Mouse Spleen IFN-γ ELISPOT Assay

Mouse spleens were ground on a 70 μm cell screen. Dulbecco's phosphate-buffered saline (DPBS) with Fetal bovine serum (FBS) was added to wash cells on the screen. After centrifuge, erythrocyte lysis buffer was added. After the lysis was complete, DPBS with 5% FBS was added to stop the lysis. After centrifugation, the cells were resuspended with RPMI-1640 medium (containing 10% FBS and 1% Penicillin-Streptomycin (PS)). Cells were counted by cell counter.

Mouse IFN-γ ELISpot plates (pre-coated with IFN-γ antibody) was washed with DPBS and sealed with medium containing serum and penicillin-streptomycin solution. The medium in the sealed plates were discarded, and then spleen cells and stimulation conditions were added to wells (Control group: medium; Positive Control group: concanavalin A (ConA); Test group: PepMix™ HRSVB (Fusion Protein F0)). The plate was placed in a cell incubator at 37° C. for 40 h. After culturing, it was washed with DPBS, and then added to test antibodies and incubated for 2 h. Discard medium, washed with DPBS, added alkaline phosphatase marker and incubate for 1 h. Discard medium, washed with DPBS, and added BCIP-NBT staining substrate. After staining, washed with water to quench, and dried. The spots were counted by ELISPOT.

As shown in FIG. 4D, under the stimulation of PepMix™ HRSVB (Fusion Protein F0) polypeptide library, the average spot number in 0.5 μg, 10 μg and 50 μg groups increase significantly compared with the average spot number in PBS group (the average spot number is 860, 715, 1197 and 28, respectively). The results indicated that significant F protein-specific T cell response in spleen cells of mice immunized with the test lipid nanoparticle compositions were detected under the condition of stimulation of RSVB (Fusion protein F0) polypeptide.

### Example 7: In Vivo Immunogenicity Assay and Challenge Trial

Lipid nanoparticle compositions comprising RSV mRNA (WT(v2)) and STAR0116 were prepared according to the method of Example 4. The solution was concentrated to 0.5 mg/mL (mRNA concentration) and filtrated by 0.22 μm millipore filter to afford mRNA containing lipid nanoparticle compositions. The lipid nanoparticle compositions were tested with female Balb/C mice (6 to 8 weeks old). 10 Mice were employed for each group, and injected 50 μL per mouse with the dose gradient set as 0.5 μg, 10 μg and 50 μg. The negative control was equal volume normal saline injection. The animals were immunized on Day 0 and Day 21, and the body weight of the mice after administration was monitored. On Day 35, blood was collected and used for serological tests. On Day 42, mice were challenged intranasally with 1.7×10⁶ PFU RSV A2 (Stain No.: Lot20220414, deposited in the State Key Laboratory of Virology), weighed daily after infection. Mice were euthanized on Day 4 after infection. The viral load of the right lung was measured. The left lung was fixed with 4% paraformaldehyde.

### Clinical Observation

During the immune stage, the mice in each group were normal in climbing for food, drinking water, exercise and stress, and there was no reaction such as vertical hair, arching back and lethargy. The body weight of mice in each group showed an upward trend. During the challenge stage, all mice in each group showed weight loss after infection, and there was no other abnormal clinical manifestations.

### Neutralizing Antibody Assay

The titer of neutralizing antibody was measured as mentioned above in Example 6, and the titer was shown in FIG. 5A. As demonstrated in previous Examples, the results indicated that the test lipid nanoparticle compositions have high immunogenicity and induce higher level antibody titers.

### Virus Challenge Trial

Add 1 ml PBS to the tissue grinding tube. The tissue grinding tube was placed on the tissue cell crusher at 5000 rpm for 40 s twice, and the lung tissue was observed to be broken into uniform suspension. Centrifuged the tissue grinding tube at 1000 g for 1 min, centrifuged the foam and tissue residue, and transfered to the biosafety cabinet. 200 μL supernatant was used for RNA extraction with Vazyme nucleic acid extraction kit (RM201-02 96rxn). After RNA extraction, cDNA was obtained by reverse transcription using ZOMANBIO reverse transcription kit, and then the virus was quantitatively measured by Monad qPCR kit. The follow-up assay system and procedure was carried out according to the specification of the selected qPCR kit. The viral load in the sample was calculated using the standard curve external standard method. As shown in FIG. 5B, the viral load in the right lung of vaccine groups (including low, medium, and high dose groups) on Day 4 after infection was lower than that of control group, and the viral load in the high-dose and medium-dose groups was significantly different from that in control group, and the tissue viral load tended to decline with the increase of vaccine dose. The results indicated that the lipid nanoparticle compositions were protective against RSV A2 infection.

### Pathology of Lung Tissue After Challenge

The left lung tissue of mice was fixed with 4% paraformaldehyde for histopathological assay. Pathological analysis of lung tissues was performed, mainly to evaluate inflammatory cell infiltration, alveolar inflammation, telangiectasis, sectionalized bronchial count and sectionalized inflammatory cell infiltration bronchial count. The scoring criteria were shown in Table 12, and the results were shown in Table 13. Part of the microscopic examination results were shown in FIG. 5C. The above results indicated that the immunized groups and the negative control group showed similar lung histopathologic conditions. The lipid nanoparticle compositions of present invention does not cause significant vaccine-associated enhanced respiratory diseases (VAERD) phenomenon, that is the aggravated lower respiratory tract symptoms in vaccine-immune population after pathogen infection.

TABLE 12
Pathological Scoring Criteria

| Catalog | Scoring | Descriptions |
|---|---|---|
| inflammatory | 0 | No inflammatory cell infiltration was observed |
| cell | 1 | A few inflammatory cell infiltration was |
| | | observed |
| infiltration | 2 | Diffuse inflammatory cell infiltration was |
| | | observed |
| | 3 | Large amount of inflammatory cell infiltration |
| | | was observed |
| | 4 | Large amount of inflammatory cell |
| | | aggregate infiltration was observed |
| allergic | 0 | No alveolitis was observed |
| alveolitis | 1 | Alveolar wall interstitial edema and capillary |
| | | congestion causing slight thickening of alveolar |
| | | wall |
| | 2 | Moderately thickened alveolar walls and |
| | | smaller alveolar cavities |
| | 3 | Alveolar wall is severely thickened and the |
| | | alveolar cavity is narrow or enlarged |
| | 4 | lung consolidation |
| telangiectasis | 0 | No telangiectasis hyperemia was observed |
| hyperemia | 1 | A few telangiectasis hyperemia was observed |
| | 2 | Focal telangiectasis hyperemia was observed |
| | 3 | More than half of telangiectasis hyperemia was |
| | | observed |
| | 4 | Large amount of telangiectasis hyperemia was |
| | | observed |

TABLE 13
Pathological Score of Lung Tissue After Challenge

| | | | | | | inflammatory cell |
|---|---|---|---|---|---|---|
| | | | allergic | telangiectasis | bronchial | infiltration |
| group | mice | inflammation | alveolitis | hyperemia | count | bronchial count |
| negative | G1 | 4 | 2 | 2 | 8 | 4 |
| control | G2 | 3 | 2 | 3 | 11 | 5 |
| | G3 | 2 | 1 | 1 | 12 | 5 |
| | G4 | 2 | 1 | 4 | 8 | 3 |
| | G5 | 4 | 2 | 4 | 10 | 4 |
| | G6 | 2 | 1 | 1 | 13 | 6 |
| | G7 | 1 | 2 | 4 | 13 | 5 |
| | G8 | 4 | 2 | 4 | 11 | 7 |
| | G9 | 4 | 2 | 2 | 9 | 9 |
| | G10 | 3 | 3 | 2 | 8 | 6 |
| 0.5 μg | G11 | 3 | 3 | 4 | 11 | 5 |
| group | G12 | 4 | 4 | 4 | 7 | 7 |
| | G13 | 2 | 2 | 3 | 12 | 5 |
| | G14 | 3 | 3 | 4 | 10 | 6 |
| | G15 | 4 | 3 | 4 | 11 | 9 |
| | G16 | 4 | 3 | 3 | 14 | 12 |
| | G17 | 4 | 3 | 2 | 11 | 8 |
| | G18 | 4 | 3 | 2 | 15 | 9 |
| | G19 | 2 | 1 | 2 | 8 | 5 |
| | G20 | 4 | 3 | 3 | 11 | 6 |
| 10 μg | G21 | 4 | 4 | 2 | 8 | 7 |
| group | G22 | 3 | 2 | 2 | 10 | 5 |
| | G23 | 4 | 4 | 2 | 16 | 13 |
| | G24 | 4 | 3 | 2 | 10 | 6 |
| | G25 | 4 | 2 | 3 | 14 | 8 |
| | G26 | 3 | 1 | 2 | 12 | 9 |
| | G27 | 3 | 4 | 3 | 13 | 5 |
| | G28 | 3 | 4 | 4 | 13 | 8 |
| | G29 | 4 | 3 | 3 | 14 | 12 |
| | G30 | 3 | 3 | 4 | 6 | 4 |
| 50 μg | G31 | 4 | 4 | 3 | 9 | 7 |
| group | G32 | 3 | 4 | 2 | 4 | 3 |
| | G33 | 4 | 3 | 3 | 4 | 4 |
| | G34 | 4 | 4 | 1 | 12 | 9 |
| | G36 | 4 | 4 | 4 | 15 | 9 |
| | G37 | 4 | 3 | 2 | 9 | 8 |
| | G38 | 4 | 4 | 3 | 4 | 2 |
| | G39 | 4 | 4 | 2 | 15 | 11 |
| | G40 | 4 | 4 | 3 | 7 | 6 |

### Example 8: Immune Program Optimization

Lipid nanoparticle compositions comprising RSV mRNA (WT(v2)) and STAR0116 were prepared according to the method of Example 4. The solution was concentrated to 0.5 mg/mL (mRNA concentration) and filtrated by 0.22 m millipore filter to afford mRNA containing lipid nanoparticle compositions. The lipid nanoparticle compositions were tested with female Balb/C mice (6 to 8 weeks old). 5 mice were employed for each group, and injected 50 μL per mouse at 10 μg vaccine dose according to below Table 14. Normal saline injection of equal volume was used as negative control. Blood was collected on Day 14 after the second injection and used for serological tests.

TABLE 14
Immunization Dosage and Schedule

| | | Vaccine | Immunization | Blood |
|---|---|---|---|---|
| Group | Mice | dose | schedule | collection |
| 1 (0/2 W) | Balb/C, | 10 μg | Day 0 and Day 14 | Day 28 |
| | female | | | |
| 2 (0/3 W) | Balb/C, | 10 μg | Day 0 and Day 21 | Day 35 |
| | female | | | |
| 3 (0/4 W) | Balb/C, | 10 μg | Day 0 and Day 28 | Day 42 |
| | female | | | |
| 4 (PBS) | Balb/C, | 0 (saline) | Day 0 and Day 28 | Day 42 |
| | female | | | |

### RSV F Antibodies Assay

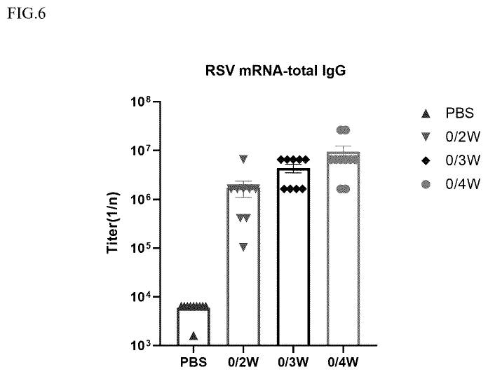

ELISA plates were coated with recombinant human RSV fusion glycoprotein (rec.hu F protein, final concentration: 0.2 μg/mL) (SinoBiological Inc.). The coated plates were incubated with a given serum dilution, and anti-mouse antibodies labeled with HRP were used to measure the specifically binding of antibodies to RSV-F protein using TMB substrate. FIG. 6 indicated that the test lipid nanoparticle compositions induce high RSV-F antibody titers, and Day 0/Day 21 two-dose immunization schedule has better results, which is used in following Examples.

### Example 9: In Vivo Immunogenicity Assay for RSV mRNA Vaccine (1)

Lipid nanoparticle compositions comprising RSV mRNAs and STAR0002 were prepared according to the method of Example 4. The solution was concentrated to 0.5 mg/mL (mRNA concentration) and filtrated by 0.22 μm millipore filter to afford mRNA containing lipid nanoparticle compositions. The lipid nanoparticle compositions were tested with female Balb/C mice (6 to 8 weeks old). The mRNA sequences and the lipid nanoparticles used in RSV mRNA vaccines were shown in below Table 15. 5 mice were employed for each group, and injected 50 μL per mouse at 10 μg vaccine dose. Normal saline injection of equal volume was used as negative control. Immunization was performed on Day 0 and Day 21. On Day 14 (“2W”), Day 21 (“3W”), Day 28 (“4W”), Day 35 (“5W”), Day 42 (“6W”), Day 49 (“7W”), Day 56 (“8W”) and/or Day 63 (“9W”), blood was collected and used for serological tests.

TABLE 15
RSV Vaccines

| RSV Vaccine | mRNA Sequence | Lipid Nanoparticle |
|---|---|---|
| P1 | SEQ ID NO: 3 | STAR0002 |
| P2 | SEQ ID NO: 4 | STAR0002 |
| WT(v2) | SEQ ID NO: 2 | STAR0002 |
| V207L | SEQ ID NO: 33 | STAR0002 |
| S55C + L188C | SEQ ID NO: 30 | STAR0002 |
| T103C + I148C | SEQ ID NO: 31 | STAR0002 |
| P102A + I379V + M447V | SEQ ID NO: 32 | STAR0002 |
| 210-T-S-V | SEQ ID NO: 14 | STAR0002 |
| 210-T-V | SEQ ID NO: 16 | STAR0002 |
| 210-T | SEQ ID NO: 18 | STAR0002 |
| 210-213-T-V | SEQ ID NO: 24 | STAR0002 |
| 210-213-T | SEQ ID NO: 26 | STAR0002 |

### RSV F Antibodies Assay

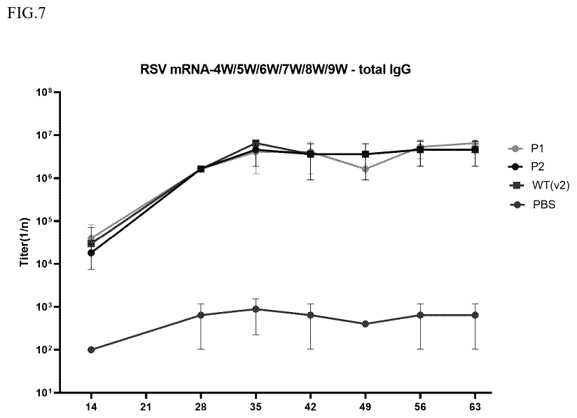

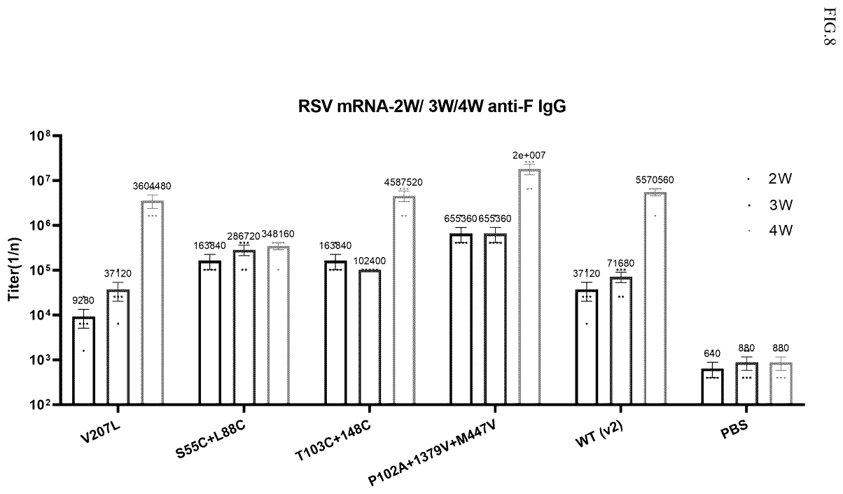

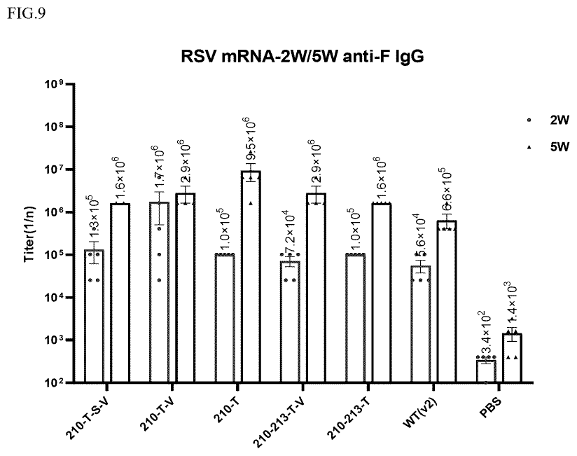

ELISA plates were coated with recombinant human RSV fusion glycoprotein (rec.hu F protein, final concentration: 0.2 μg/mL) (SinoBiological Inc.). The coated plates were incubated with a given serum dilution, and anti-mouse antibodies labeled with HRP were used to measure the specifically binding of antibodies to RSV-F protein using TMB substrate. FIG. 7, FIG. 8 and FIG. 9 indicated that the RSV mRNA vaccines induce higher RSV-F antibody titers.

### Example 10: In Vivo Immunogenicity Assay for RSV mRNA Vaccine (2)

Lipid nanoparticle compositions comprising RSV mRNAs and STAR0225 were prepared according to the method of Example 4. The solution was concentrated to 0.5 mg/mL (mRNA concentration) and filtrated by 0.22 μm millipore filter to afford mRNA containing lipid nanoparticle compositions. The lipid nanoparticle compositions were tested with female Balb/C mice (6 to 8 weeks old). The mRNA sequences and the lipid nanoparticles used in RSV mRNA vaccines were shown in below Table 16. 5 mice were employed for each group, and injected 50 μL per mouse at 10 μg vaccine dose. Normal saline injection of equal volume was used as negative control. Immunization was performed on Day 0 and Day 21. On Day 14 (“2W”) and Day 35 (“5W”), blood was collected and used for serological tests.

TABLE 16
RSV Vaccines

| RSV Vaccine | mRNA Sequence | Lipid Nanoparticle |
|---|---|---|
| 210-T-S-V | SEQ ID NO: 14 | STAR0225 |
| 210-T-V | SEQ ID NO: 16 | STAR0225 |
| 210-T | SEQ ID NO: 18 | STAR0225 |
| 210-213-T-V | SEQ ID NO: 24 | STAR0225 |
| 210-213-T | SEQ ID NO: 26 | STAR0225 |
| WT(v2) | SEQ ID NO: 2 | STAR0225 |

### RSV F Antibodies Assay

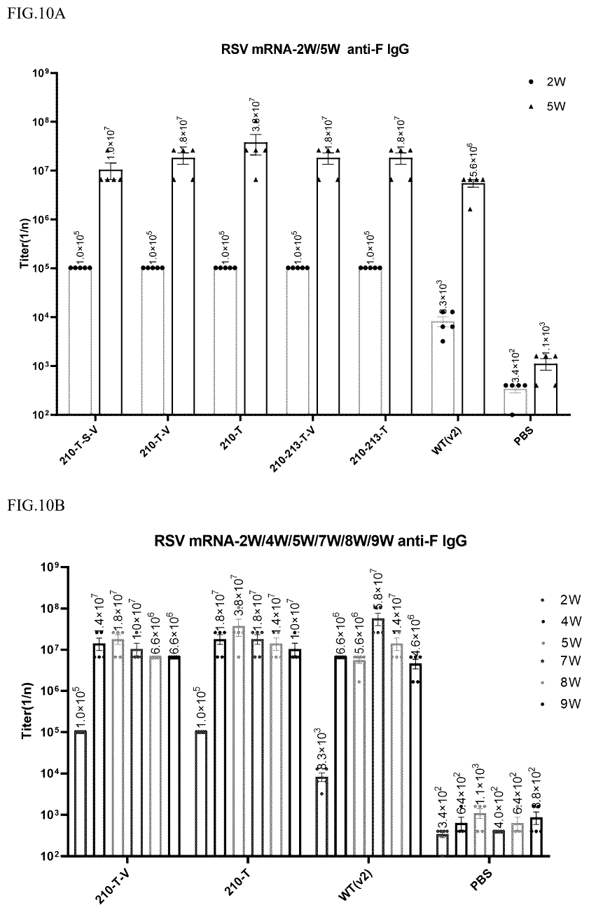

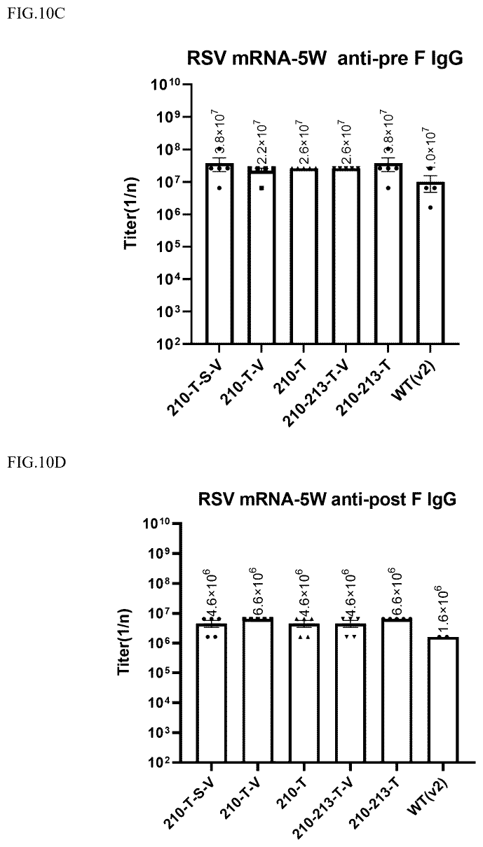

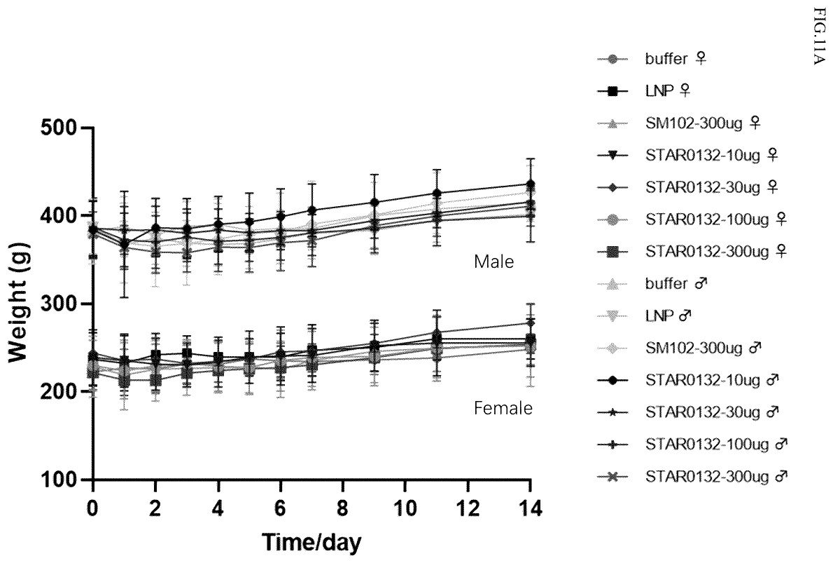

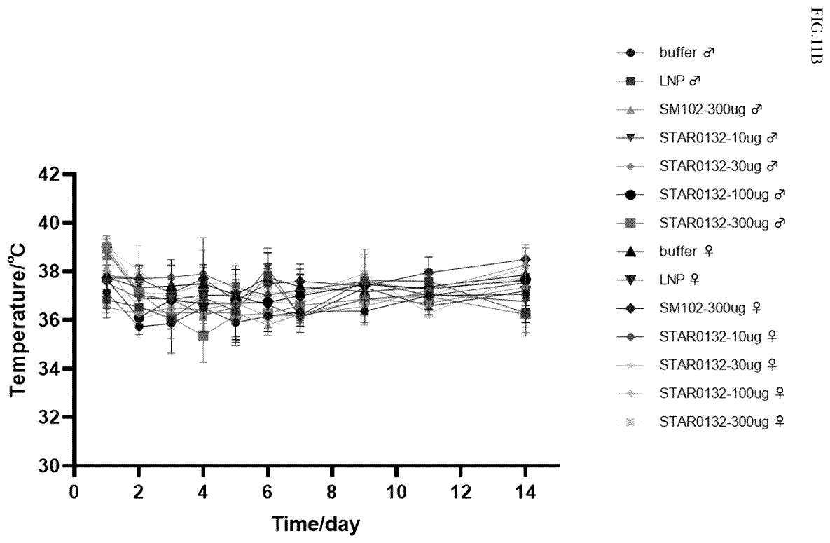

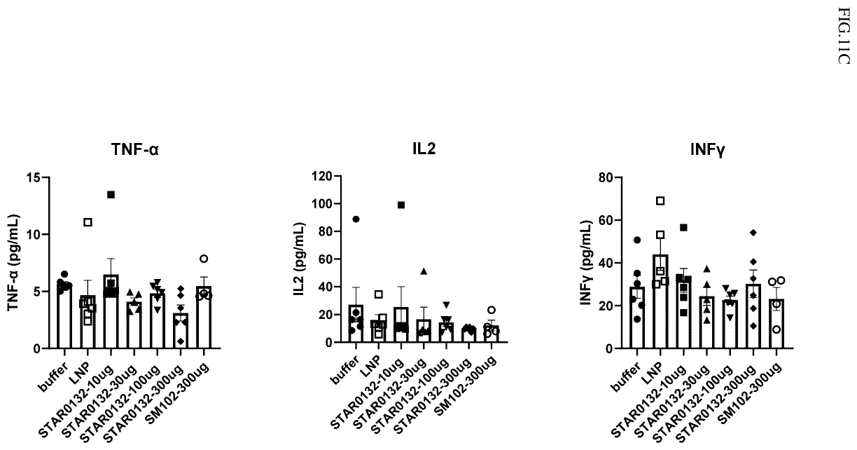

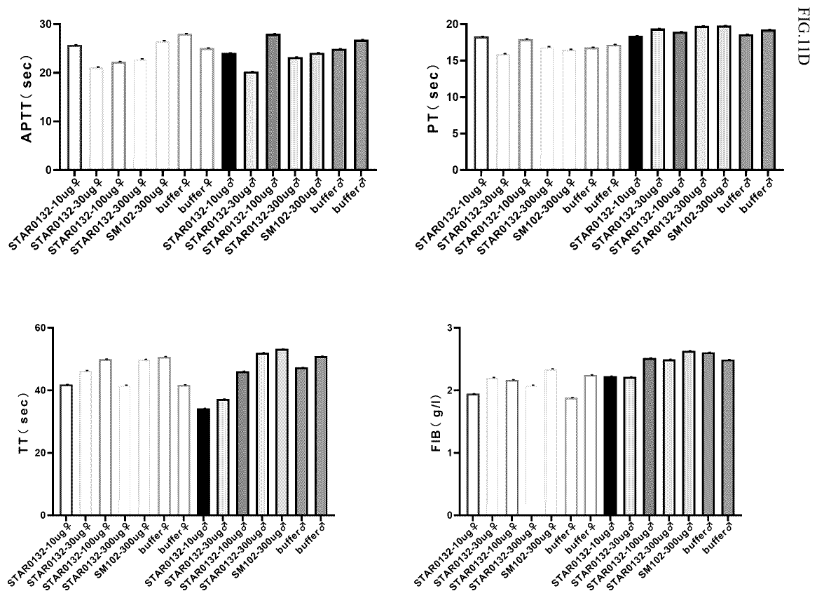

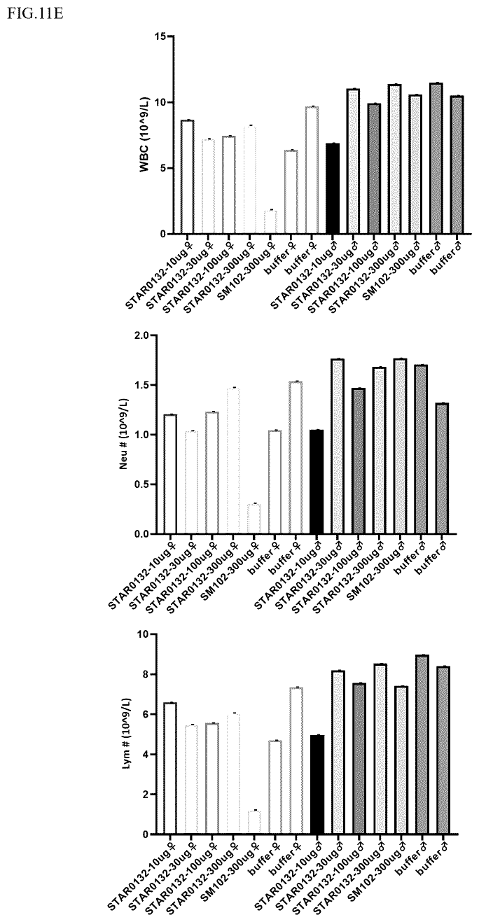

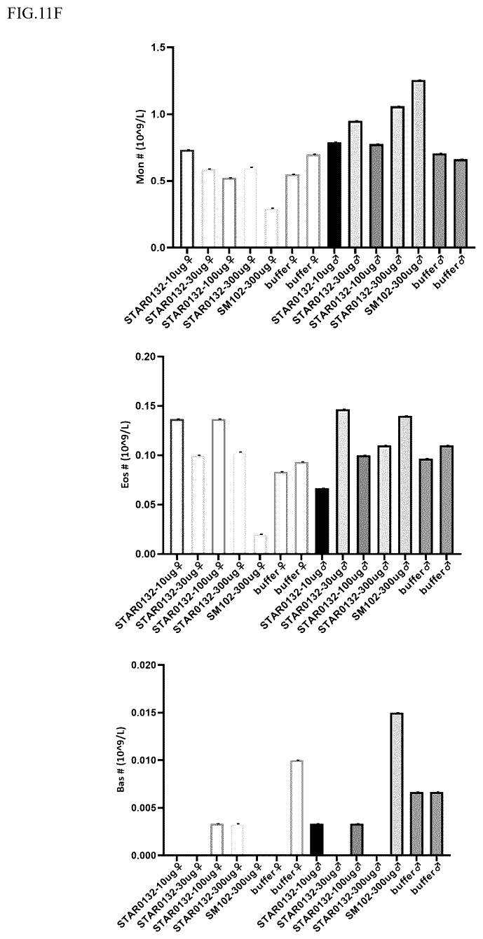

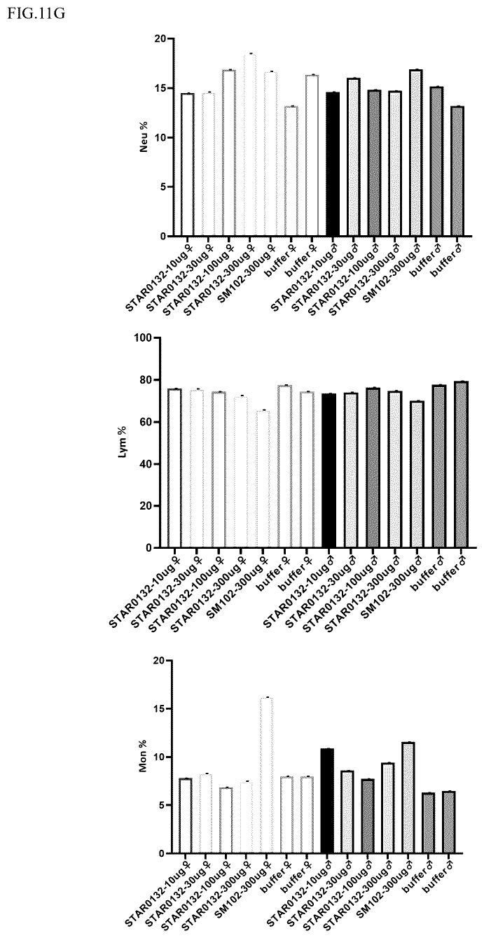

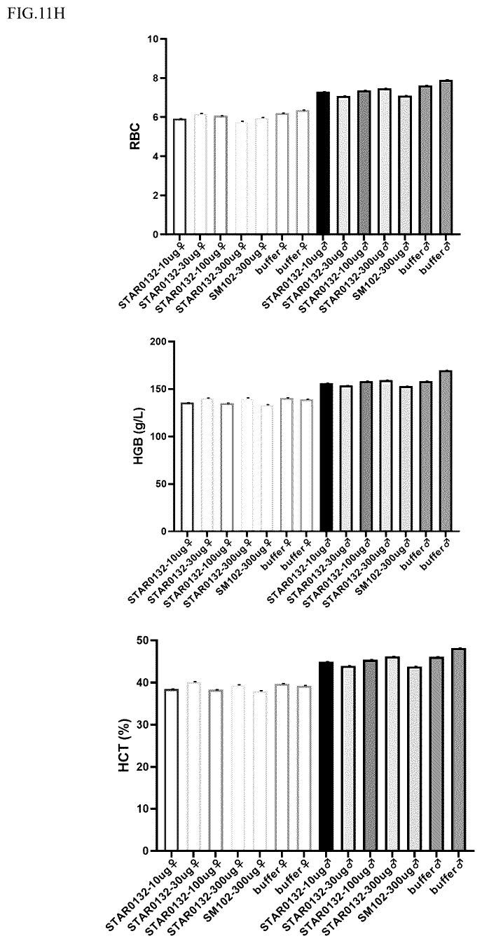

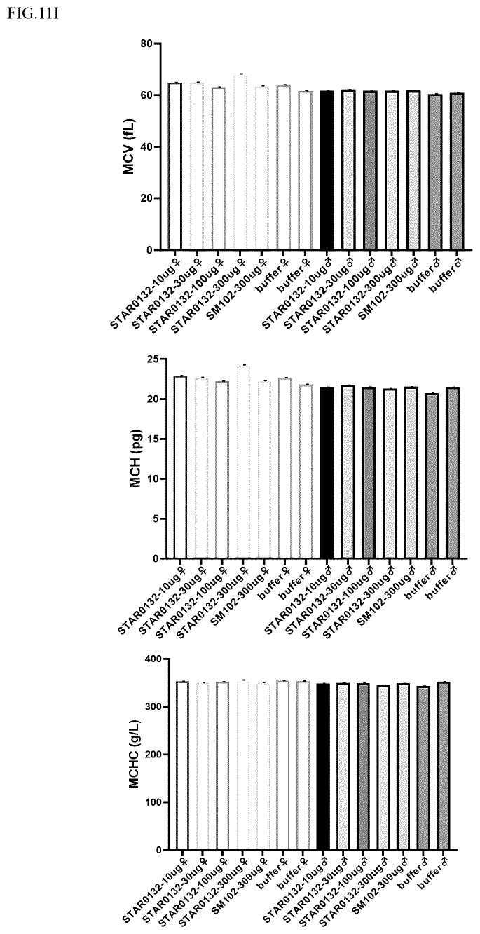

ELISA plates were coated with recombinant human RSV fusion glycoprotein (rec.hu F protein, final concentration: 0.2 μg/mL) (SinoBiological Inc.). The coated plates were incubated with a given serum dilution, and anti-mouse antibodies labeled with HRP were used to measure the specifically binding of antibodies to RSV-F protein using TMB substrate. FIG. 10A-10B indicated that the RSV mRNA vaccines induce higher RSV-F antibody titers.

### Pre-Fusion RSV-F Antibodies and Post-Fusion RSV-F Antibodies Assay

The pre-fusion RSV-F protein and post-fusion RSV-F protein in serum were detected by pre-fusion RSV-F Antibody (IgG) Quantitative and Qualitative Detection Kit (ELISA) (Vazyme, Cat. No.: DD3910-01) and post-fusion RSV-F Antibody (IgG) Quantitative and Qualitative Detection Kit (ELISA) (Vazyme, Cat. No.: DD3911-01). The detection was performed according to the specification of the kits, and the results were shown in FIG. 10C and FIG. 10D. It can be seen that the RSV mRNA vaccines can induce higher RSV pre-F protein antibody titers and RSV post-F protein antibody titers.

### Example 11: Safety Study (1)

Safety for lipid nanoparticle compositions comprising RSV mRNA (WT(v2)) and STAR0116 were tested with cynomolgus macaques. Lipid nanoparticle compositions were prepared according to the method of Example 4. The solution was concentrated to 0.5 mg/mL (mRNA concentration) and filtrated by 0.22 μm millipore filter to afford mRNA containing lipid nanoparticle compositions. Immunization dosage and schedule was shown in below Table 17. The lipid nanoparticle compositions were administered to cynomolgus macaques by intramuscular injection at 125 μg dose for each monkey in the upper arm, and the injection volume was 500 μl/monkey. Normal saline injection of equal volume was used as negative control. Immunization was performed on Day 0 and Day 21. The injection site was observed once a day after administration until Day 49 (for palpation pain, erythema, swelling, etc.). 1 h before (−1 h), upon (0 h) each administration, and 0.5 h, 2 h, 6 h, 24 h after each administration, body temperature was measured. Body weight was measured once a week. On Day 0 (DO), Day 1 (D1), and Day 22 (D22), blood was collected for CBA assay to assess vaccine safety. On Day 28, blood was collected for biochemical assay to assess vaccine safety.

TABLE 17
Immunization Dosage and Schedule

| Group | Sex and ID | Dosage | Schedule |
|---|---|---|---|
| 1 | 1 female (1F001); | normal saline, | Day 0 and Day 21 |
| | 2 male (1M001 and | 500 μL | |
| | 1M002) | | |
| 2 | 3 female (2F001, | 125 μg, 500 μL | Day 0 and Day 21 |
| | 2F002 and 2F003); | | |
| | 2 male (2M001 and | | |
| | 2M002) | | |

### Clinical Observation of Cynomolgus Macaques

During the experiment, there were no adverse clinical manifestations in the test group and the body weight of animals in each group was not significantly changed. During the experiment, compared with negative control group, the body temperature of cynomolgus macaques in RSV mRNA group increased slightly at 2 h, 6 h and 24 h after the first administration, but there was no statistical difference. After the second administration, the RSV mRNA group showed a slight increase in body temperature at 6 h and 24 h, and there was a statistical difference in body temperature increase at 6 h, where the body temperature of the negative control group and RSV mRNA group were 38.80±0.26° C. and 40.24±0.63° C., respectively.

### Cytokine Level in Plasma Sample Measured by CBA

The plasma sample was thawed, 4-fold diluted set aside, operated according to the LEGENDplex™ NHP Th Cytokine Panel (10-plex) (BioLegend-740387) specification, The cytokines (IL-2, IL-5, IL-6, IL-10, IL-13, TNF-α, IFN-γ, IL-4, IL-17A, IL-21) in plasma sample were measured.

### Date Process:

Standard curve: standard concentration as X-axis, log(median value of standard) as Y-axis, and 4-parameter curve fit was (Y=(A−D)/[1+(x/C)\hat{ }B]+D) used.

$$Concentration of the sample was calculated using the standard curve , x ( sample concentration ) = {[ ( A - D ) / ( y - D ) - 1 ]}^{∧} ( 1 / B ) \times C$$

$$Cytokine concentration ( pg / mL ) = 1 L - 2 / 1 L - 5 / 1 L - 6 / 1 L - 10 / 1 L - 13 / 1 FN - γ / 1 L - 4 / 1 L - 17 A / 1 L - 21 / TNF - α concentration \times 4 ( s ample dilution ratio )$$

### Cytokine-Release Curve was Fitted by GraphPad Prism.

The results were as shown in Table 18. Compared with the level of IL-6 on Day 0 before administration, on Day 1 and Day 22 post administration, the level of IL-6 in the plasma of cynomolgus macaques in RSV mRNA group 2F001, 2F002, 2F003, 2M001 and 2M002 were significantly increased. There is no obvious change in the overall level of IL-2, IL-5, IL-10, IL-13, IFN-γ, IL-4, IL-17A, IL-21 and TNF-α.

TABLE 18
Cytokine Level in Plasma Sample

| Cytokines | | | | | | | | | | | |
|---|---|---|---|---|---|---|---|---|---|---|---|
| Conc.(pg/mL) | | IL-2 | IL-5 | IL-6 | IL-10 | IL-13 | IFN-gamma | IL-4 | IL-17A | IL-21 | TNF-α |
| D 0 | 1F001 | — | 2.9 | — | 16.2 | 2.1 | — | 0.2 | — | — | 0 |
| | 1M001 | — | 45.1 | — | 9.9 | 2.1 | 14.5 | 0.2 | — | — | — |
| | 1M002 | — | 58.1 | — | 22.1 | 0.1 | 91.1 | 1.2 | 0.6 | — | 0.4 |
| | 2F001 | — | 16.3 | — | 16.2 | — | — | 0.7 | 0.6 | — | — |
| | 2F002 | — | — | — | 16.2 | 0.1 | 91.1 | 0 | — | — | — |
| | 2F003 | — | 31.2 | 3.8 | 3.6 | 0.1 | 164.5 | 1.2 | 0.6 | — | 1.2 |
| | 2M001 | — | 45.1 | 0.1 | — | 0.1 | 14.5 | 0.7 | — | — | 0.1 |
| | 2M002 | — | 71 | 3.8 | 9.9 | — | — | 0.7 | 0.6 | — | 0 |
| D 1 | 1F001 | — | 2.9 | — | 3.6 | 0.5 | 52.6 | 1.2 | — | — | 0.1 |
| | 1M001 | — | — | — | 3.6 | 0.1 | — | 0.2 | — | — | — |
| | 1M002 | — | 16.3 | — | 9.9 | 0.1 | — | — | 1.4 | — | 0 |
| | 2F001 | — | 16.3 | 635.5 | 9.9 | 0.1 | — | 0.7 | — | — | — |
| | 2F002 | — | 16.3 | 365.2 | — | 0.1 | — | 0.2 | 0.6 | 10.5 | — |
| | 2F003 | — | 45.1 | 344.4 | 28.3 | 1.3 | — | 0.7 | 2.1 | — | 0.1 |
| | 2M001 | 20 | 31.2 | 1485.7 | 46.4 | — | — | 0.7 | 0.6 | 59 | 0.7 |
| | 2M002 | — | 31.2 | 357.4 | 40 | — | — | 2.5 | 3.7 | — | 0.4 |
| D 22 | 1F001 | 4.8 | 71 | — | 40 | 0.5 | — | — | 2.1 | — | — |
| | 1M001 | — | 58.1 | 0.1 | 16.2 | 0.5 | — | 0.2 | — | — | — |
| | 1M002 | — | 31.2 | — | 9.9 | 0.5 | — | 0.7 | — | — | 0 |
| | 2F001 | — | 2.9 | 935.5 | — | 2.1 | — | 1.2 | — | — | — |
| | 2F002 | — | 58.1 | 169 | 9.9 | 0.5 | — | — | — | — | — |
| | 2F003 | — | — | 176.4 | 28.3 | — | — | 0 | 1.4 | — | — |
| | 2M001 | — | 99.4 | 122.6 | 22.1 | 0.1 | 14.5 | 1.2 | 1.4 | — | 0.1 |
| | 2M002 | — | 45.1 | 310.1 | 40 | 1.3 | — | 0.7 | 1.4 | — | — |

“—” denotes the cytokine concentration is lower than the test line.

### Blood Biochemical Assay

The serum collected on Day 28 was defrosted and 27 blood biochemical indexes in the serum were measured by Hitachi 7100 automatic biochemical analyzer. The results were shown in Table 19. Compared with the negative control group, the serum level of NEFA (non-esterified fatty acid) in the RSV mRNA group was significantly increased, while there was no significant difference in other indexes.

TABLE 19
Blood Biochemical Indexes

| | group | | | | | | | |
|---|---|---|---|---|---|---|---|---|
| | negative control | | | RSV mRNA group | | | | |
| index | 1F001 | 1M001 | 1M002 | 2F001 | 2F002 | 2F003 | 2M001 | 2M002 |
| [ALT] | 29 | 34 | 31 | 26 | 24 | 42 | 27 | 24 |
| [AST] | 31 | 27 | 32 | 29 | 31 | 43 | 29 | 31 |
| [TP] | 66.5 | 69.4 | 65.9 | 67.5 | 74.9 | 69.6 | 72.6 | 75.2 |
| [ALB] | 33.8 | 39.4 | 36.4 | 35 | 38.9 | 34.4 | 39.6 | 35.8 |
| [TBIL] | 1.1 | 1.5 | 1.3 | 1.4 | 1.9 | 1 | 1.2 | 1.3 |
| [ALP] | 238 | 634 | 437 | 245 | 383 | 360 | 791 | 532 |
| [GLU] | 3.92 | 4.09 | 4.06 | 4.09 | 4.84 | 4.03 | 5.41 | 3.67 |
| [BUN] | 5.81 | 6.64 | 5.75 | 6.57 | 8.67 | 4.68 | 6.19 | 8.26 |
| [CREA] | 51.7 | 50.5 | 54.9 | 59.5 | 60.5 | 68.6 | 44.1 | 60.4 |
| [UA] | 30.5 | 35 | 12.8 | 32.2 | 49.4 | 41.3 | 42.4 | 20.4 |
| [Ca] | 2.5 | 2.55 | 2.48 | 2.5 | 2.75 | 2.49 | 2.65 | 2.53 |
| [P] | 0.89 | 1.61 | 1.12 | 1.35 | 1.19 | 1.26 | 1.63 | 0.97 |
| [CH01] | 3.54 | 2.86 | 3.32 | 2.14 | 1.86 | 2.8 | 2.52 | 3.28 |
| [TG] | 1.52 | 1.98 | 1.53 | 1.55 | 1.73 | 1.57 | 2.07 | 1.89 |
| [HDL] | 1.61 | 1.65 | 1.47 | 1.15 | 1.01 | 1.12 | 1.31 | 1.31 |
| [LDL] | 1.74 | 0.98 | 1.69 | 0.84 | 0.62 | 1.44 | 0.96 | 1.84 |
| [CK] | 99 | 137 | 82 | 69 | 126 | 137 | 108 | 93 |
| [LDH] | 302 | 235 | 404 | 349 | 357 | 355 | 562 | 491 |
| [K] | 4.27 | 4.19 | 4.51 | 4.57 | 4.47 | 5.03 | 4.1 | 4.16 |
| [Na] | 146.2 | 145.9 | 143.8 | 146.3 | 146.8 | 150 | 146.7 | 144.9 |
| [CL] | 104.9 | 108.1 | 105.5 | 106.8 | 106.9 | 110 | 106.9 | 105.2 |
| [Fe] | 20 | 25 | 31 | 25 | 34 | 35 | 19 | 28 |
| [CRP] | 0.28 | 1.3 | 0.48 | 0.34 | 4.17 | 0.45 | 0.97 | 0.53 |
| [Mg] | 0.79 | 0.86 | 0.82 | 0.88 | 0.88 | 0.87 | 0.79 | 0.89 |
| [NEFA1] | 203.8 | 246.7 | 245.4 | 694.8 | 1090.4 | 847.8 | 1132.7 | 954.5 |
| [UIBC] | 56.3 | 39.2 | 34.5 | 43.9 | 31.1 | 30.6 | 48.1 | 37.1 |
| [AMY] | 541 | 608 | 360 | 616 | 520 | 575 | 307 | 383 |

### Example 12: Safety Study (2)

Safety for lipid nanoparticle composition comprising RSV mRNA (WT (v2)) and STAR0132 were tested with cynomolgus macaques. Lipid nanoparticle compositions were prepared according to the method of Example 4. The solution was concentrated to 0.5 mg/mL (mRNA concentration) and filtrated by 0.22 μm millipore filter to afford mRNA containing lipid nanoparticle compositions. Immunization dosage was shown in below Table 20. The lipid nanoparticle compositions were injected to rats at 300 μL/rat. Solvent of equal volume was used as negative control. SM102 contained in STAR0002 was used as positive control.

Immunization was performed on Day 0. The body temperature and weight after immunization were measured. On Day 3 and Day 14, blood was collected for cytokines, blood routine and coagulation function assay. On Day 14, rats in Groups 4, 5, 6 and 7 were killed and their hearts, livers, lungs and kidneys were harvested for tissue pathological assay.

TABLE 20
Immunization Dosage of Rat

| Group | Rats | Vaccine Dosage |
|---|---|---|
| 1 | SD rats, 3 male and 3 female | 10 μg, STAR0132 |
| 2 | SD rats, 3 male and 3 female | 30 μg, STAR0132 |
| 3 | SD rats, 3 male and 3 female | 100 μg, STAR0132 |
| 4 | SD rats, 3 male and 3 female | 300 μg, STAR0132 |
| 5 | SD rats, 2 male and 2 female | 300 μg, STAR0002 |
| 6 | SD rats, 3 male and 3 female | LNP without mRNA, |
| | | STAR0002 |
| 7 | SD rats, 3 male and 3 female | negative control |

### Clinical Observation

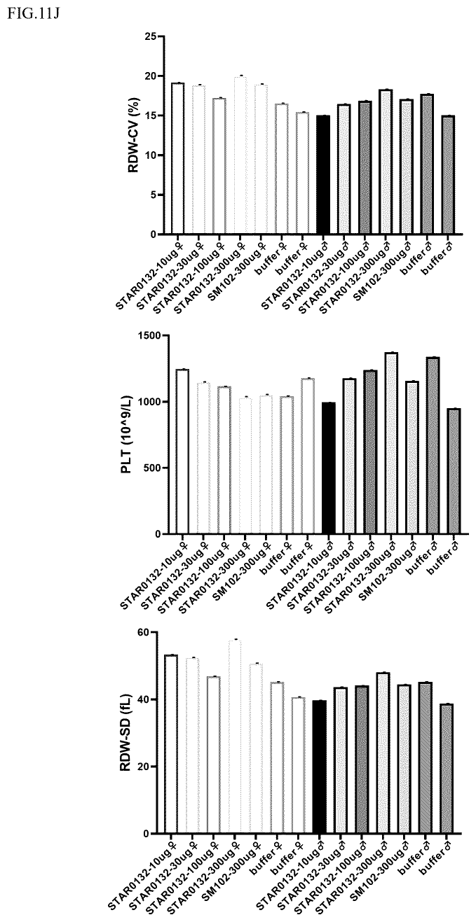

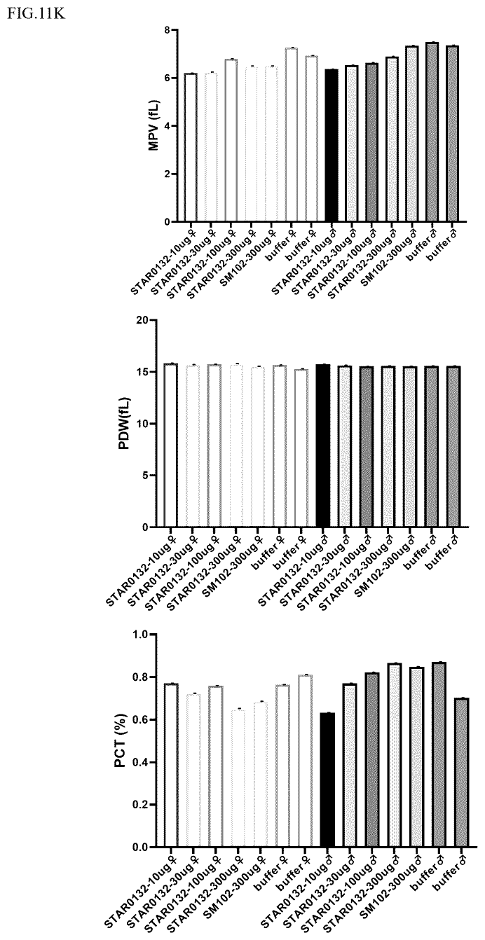

As shown in FIG. 11A and FIG. 11B, during the experiment, the animals in RSV mRNA groups showed no adverse clinical symptoms, and compared with the negative control group, the body weight and body temperature of the rats in the RSV mRNA group had no significant changes after administration.

### Cytokine Assay

Blood samples from rats on Day 3 after administration were measured using LiankeBio rat IL-2 ELISA kit (EK302), rat TNF-α ELISA kit (EK382) and rat IFN-γ ELISA kit (EK380). As shown in FIG. 11C, compared with the negative control group, RSV mRNA groups showed no significant changes in the level of IL-2, TNF-α and IFN-γ.

### Coagulation Function Assay

Blood samples of rats on Day 14 after administration were measured for coagulation function. The results were shown in FIG. 11D. Compared with the negative control group, there was no significant change in the level of coagulation function in the RSV mRNA groups.

### Blood Routine Test

Blood samples of rats on Day 14 after administration were measured for blood routine test. The results were shown in FIG. 11E-11K. Compared with the negative control group, there was no significant change in the level of blood routine test in the RSV mRNA groups.

### Organ Histopathology Assay

The heart, liver, lung and kidney tissues of Groups 4, 5, 6 and 7 were fixed with 4% paraformaldehyde for histopathological assay. The pathological analysis of heart, liver, lung and kidney tissues was performed to evaluate the related indexes of acute toxicity in rats. A small number of inflammatory cells were observed in the heart tissues of the above four groups. A small number of inflammatory cells and minor vacuolar degeneration of liver cells were observed in the liver tissues of the above four groups. Moderate to severe inflammatory cell infiltration and pulmonary septum thickening were observed in most of the lung tissues in the above four groups. Slight to mild degeneration and necrosis of renal tubular epithelial cells were observed in the above four groups, with a small amount of inflammatory cell infiltration. No significant difference in organ abnormalities was found among the negative control group 7 and the other three RSV mRNA groups.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents maybe considered to fall within the scope of the invention as defined by the claims that follow.

What is claimed is:

1. A lipid nanoparticle composition comprising:
a target polynucleotide that comprises a nucleic acid encoding a Respiratory Syncytial Virus antigenic polypeptide or a variant thereof, and
a lipid nanoparticle comprising a compound of Formula (I) 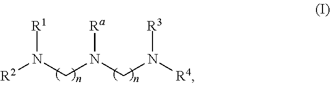
or a pharmaceutically acceptable salt thereof, wherein
R^{a} is selected from the group consisting of hydrogen, R⁵, alkyl, alkenyl, alkynyl, heteroalkyl, heteroalkenyl, heteroalkynyl, cycloalkyl, heterocyclyl, aryl and heteroaryl, wherein the alkyl, alkenyl, alkynyl, heteroalkyl, heteroalkenyl, heteroalkynyl, cycloalkyl, heterocyclyl, aryl and heteroaryl are optionally substituted by one or more groups independently selected from the group consisting of halogen, hydroxyl, cyano, cycloalkyl, heterocyclyl, aryl, heteroaryl and R⁶;
R¹ is or OH 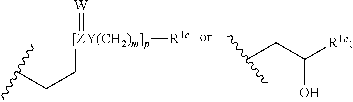
R² is or 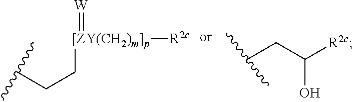
R³ is 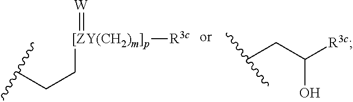
R⁴ is 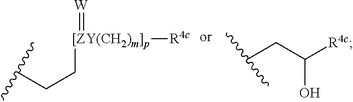
R⁵, if exists, is 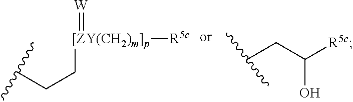
R⁶, if exists, is 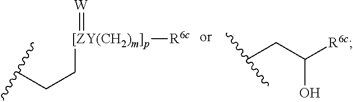
each W is independently selected from O, S or NR^{b}, and each R^{b} is independently selected from hydrogen, alkyl, alkyloxycarbonyl, acyl or sulfonyl;
each Y is independently selected from O, S, NR^{c}, N(R^{c})Z(W), N(R^{c})N(R^{c}) or N(R^{c})N(R^{c})Z(W), and each R^{c} is independently selected from hydrogen, alkyl, alkyloxycarbonyl, acyl or sulfonyl;
each Z is independently selected from C, S or S(O);
each n is independently 0, 1, 2, 3, 4 or 5;
each m is independently 0, 1, 2 or 3;
each p is independently 1, 2, 3 or 4; and
each of R^{1c}, R^{2c}, R^{3c}, R^{4c}, R^{5c} and R^{6c} is independently selected from the group consisting of alkyl, alkenyl, alkynyl, heteroalkyl, heteroalkenyl and heteroalkynyl, wherein the alkyl, alkenyl, alkynyl, heteroalkyl, heteroalkenyl and heteroalkynyl are optionally substituted by one or more groups independently selected from the group consisting of halogen, hydroxyl, oxo, cyano, cycloalkyl, heterocyclyl, aryl and heteroaryl, and the alkyl, alkenyl, alkynyl, heteroalkyl, heteroalkenyl and heteroalkynyl are optionally interrupted by one or more groups independently selected from the group consisting of cycloalkyl, heterocyclyl, aryl and heteroaryl.

2. The lipid nanoparticle composition of claim 1, wherein the compound is of Formula (A) or Formula (B):

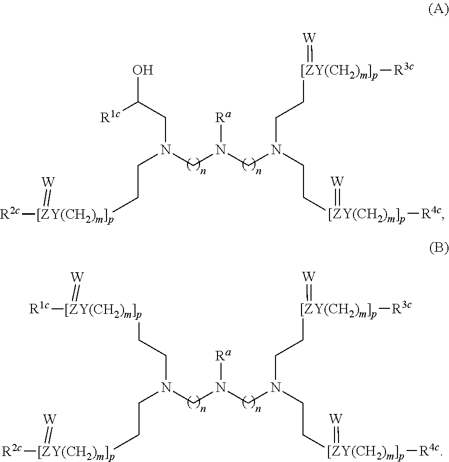

3. The lipid nanoparticle composition of claim 1, wherein at least two of R², R³ and R⁴ are the same.

4. The lipid nanoparticle composition of claim 1, wherein at least three of R², R³ and R⁴ are the same.

5. The lipid nanoparticle composition of claim 1, wherein one or more of Y is O, NR^{c} or N(R^{c})N(R^{c})Z(W).

6. The lipid nanoparticle composition of claim 1, wherein one or more of W is O.

7. The lipid nanoparticle composition of claim 5, wherein one or more of R^{c} is hydrogen.

8. The lipid nanoparticle composition of claim 1, wherein one or more of Z is C or S(O).

9. The lipid nanoparticle composition of claim 1, wherein one or more of R^{2c}, R^{3c} and R^{4c}, is alkyl or alkenyl.

10. The lipid nanoparticle composition of claim 1, wherein one or more of R^{2c}, R^{3c} and R^{4c}, is C₈₋₂₄ alkyl or alkenyl.

11. The lipid nanoparticle composition of claim 1, wherein one or more of R^{2c}, R^{3c} and R^{4c}, is C₁₀₋₂₄ alkyl or alkenyl.

12. The lipid nanoparticle composition of claim 1, wherein one or more of R^{2c}, R^{3c} and R^{4c} is alkenyl comprising one, two or three C═C double bond.

13. The lipid nanoparticle composition of claim 1, wherein one or more of R^{2c}, R^{3c} and R^{4c} is alkenyl comprising one or more Z-olefin.

14. The lipid nanoparticle composition of claim 1, wherein R^{1c} is alkyl.

15. The lipid nanoparticle composition of claim 1, wherein R^{1c} is C₁₋₁₂ alkyl.

16. The lipid nanoparticle composition of claim 1, wherein R^{1c} is C₄₋₁₀ alkyl.

17. The lipid nanoparticle composition of claim 1, wherein R^{a} is C₁₋₆ alkyl optionally substituted by one or more groups independently selected from the group consisting of hydroxyl, cycloalkyl and heteroaryl.

18. The lipid nanoparticle composition of claim 1, wherein R^{a} is methyl, ethyl, propyl, butyl or pentyl.

19. The lipid nanoparticle composition of claim 1, wherein one or more of 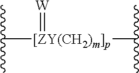
is independently selected from the group consisting of

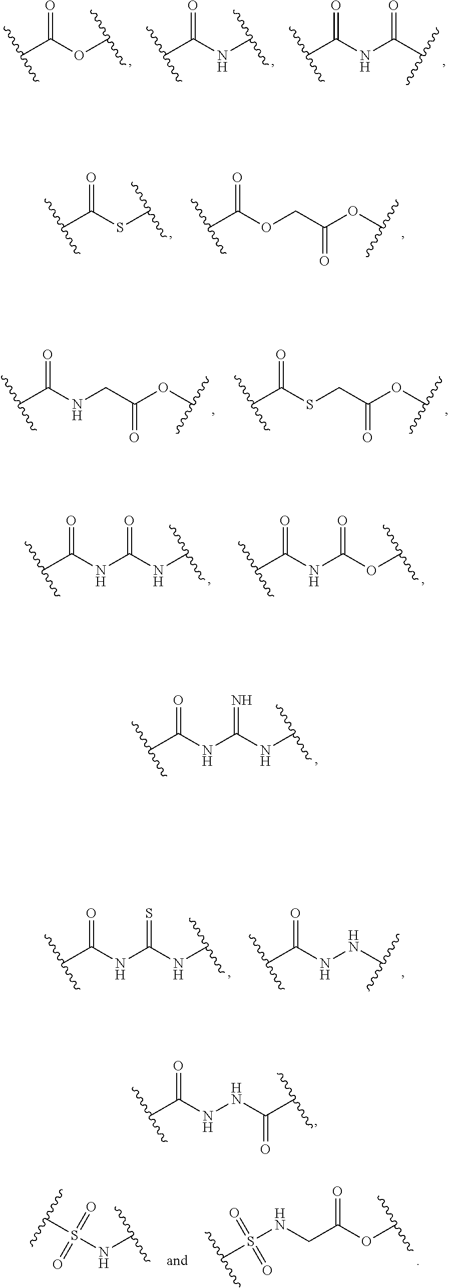

20. The lipid nanoparticle composition of claim 1, wherein one or more of R^{1c}, R^{2c}, R^{3c} and R^{4c} is independently selected from the group consisting of

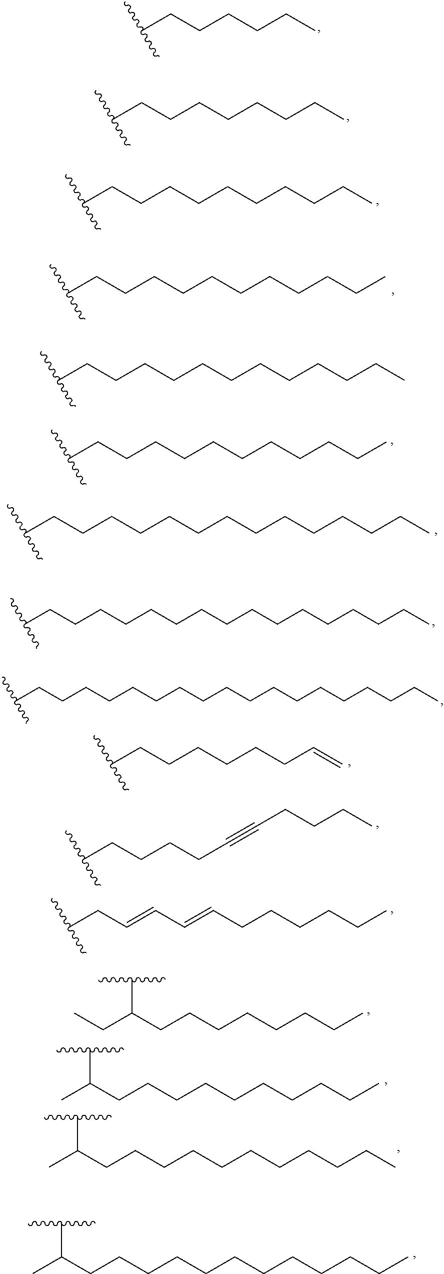

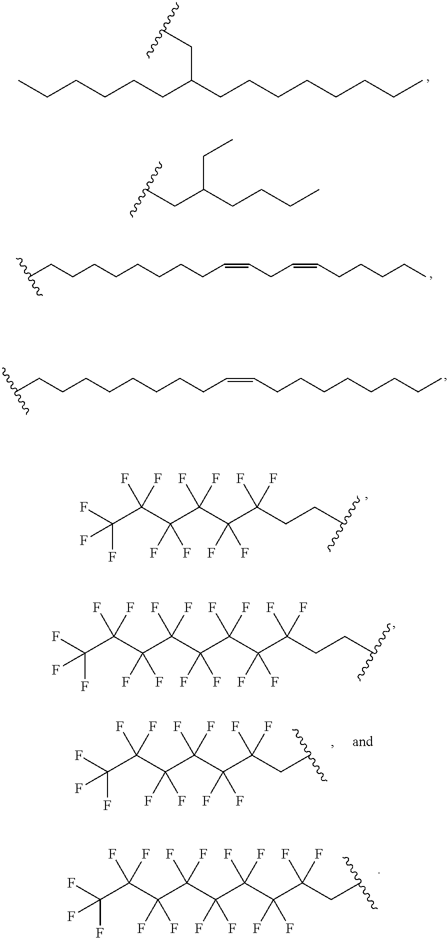

21. The lipid nanoparticle composition of claim 1, wherein each of R^{1c}, R^{2c}, R^{3c}, R^{4c}, R^{5c} and R^{6c}, if exists, does not comprise two heteroatoms directly bonded to each other.

22. The lipid nanoparticle composition of claim 1, wherein each of R^{1c}, R^{2c}, R^{3c}, R^{4c}, R^{5c} and R^{6c}, if exists, comprises —N(R^{c})—N(R^{c})— or —S(O)₂—N(R^{c})—.

23. The lipid nanoparticle composition of claim 1, wherein the compound has a structure listed in the table below:

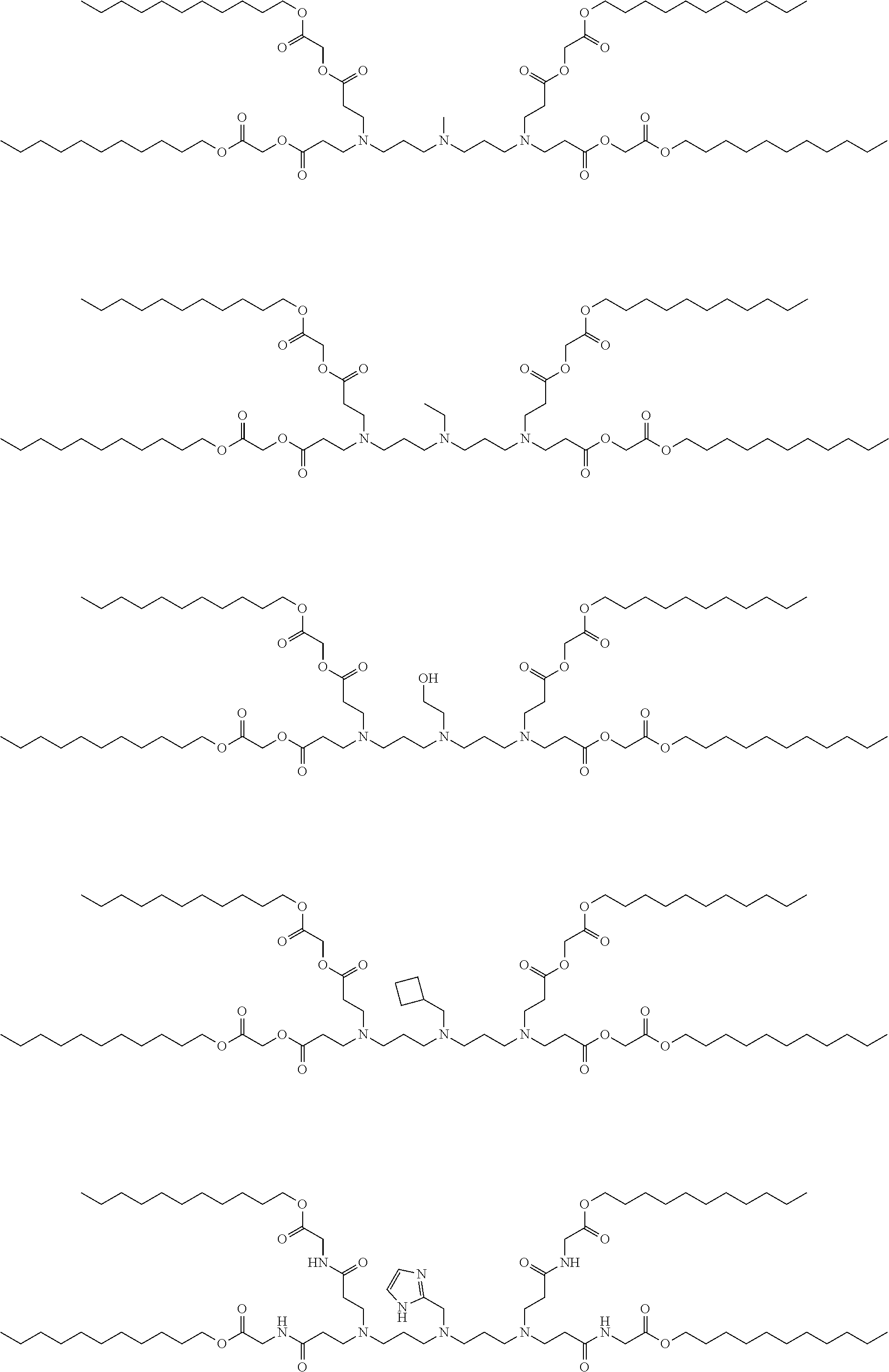

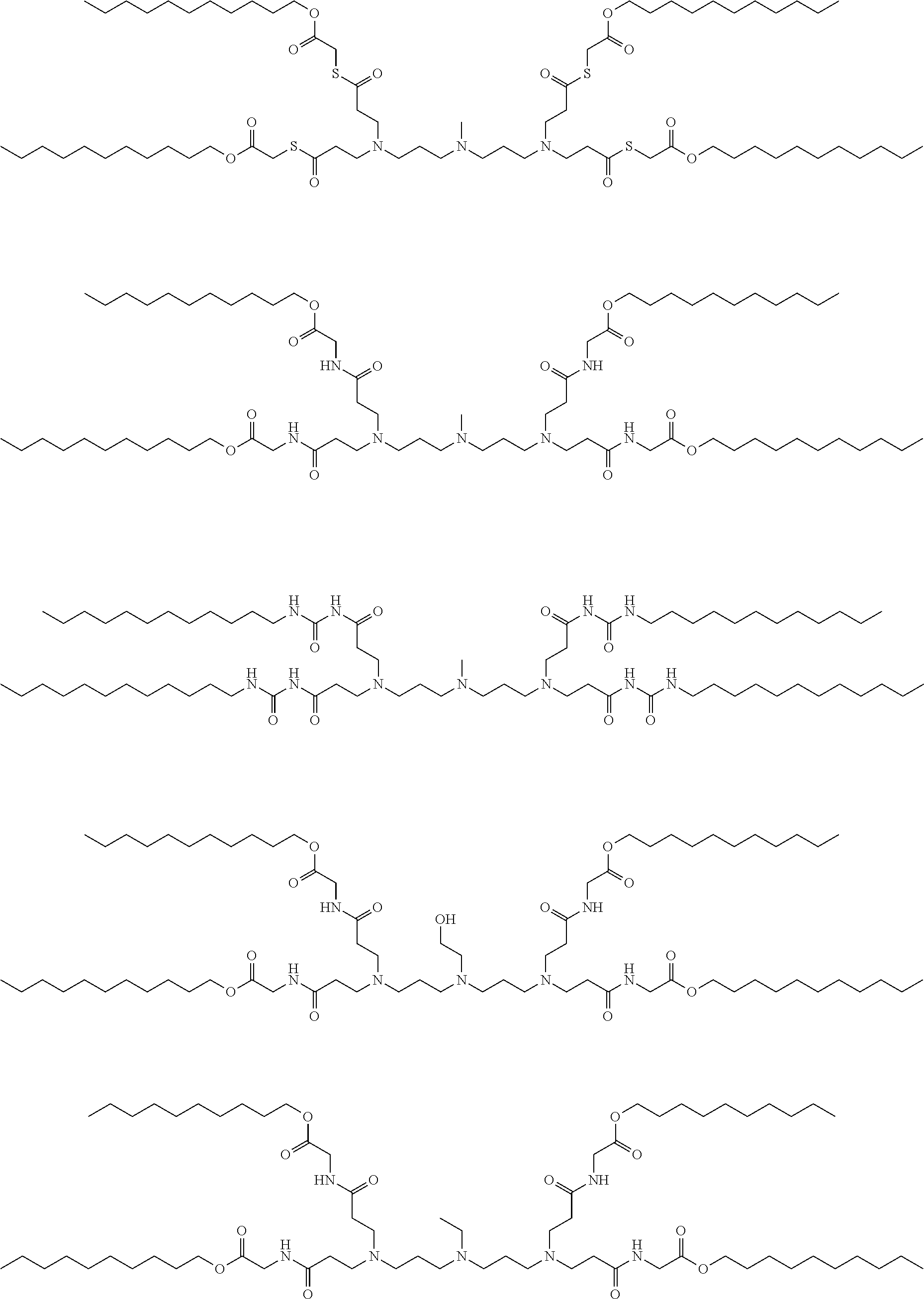

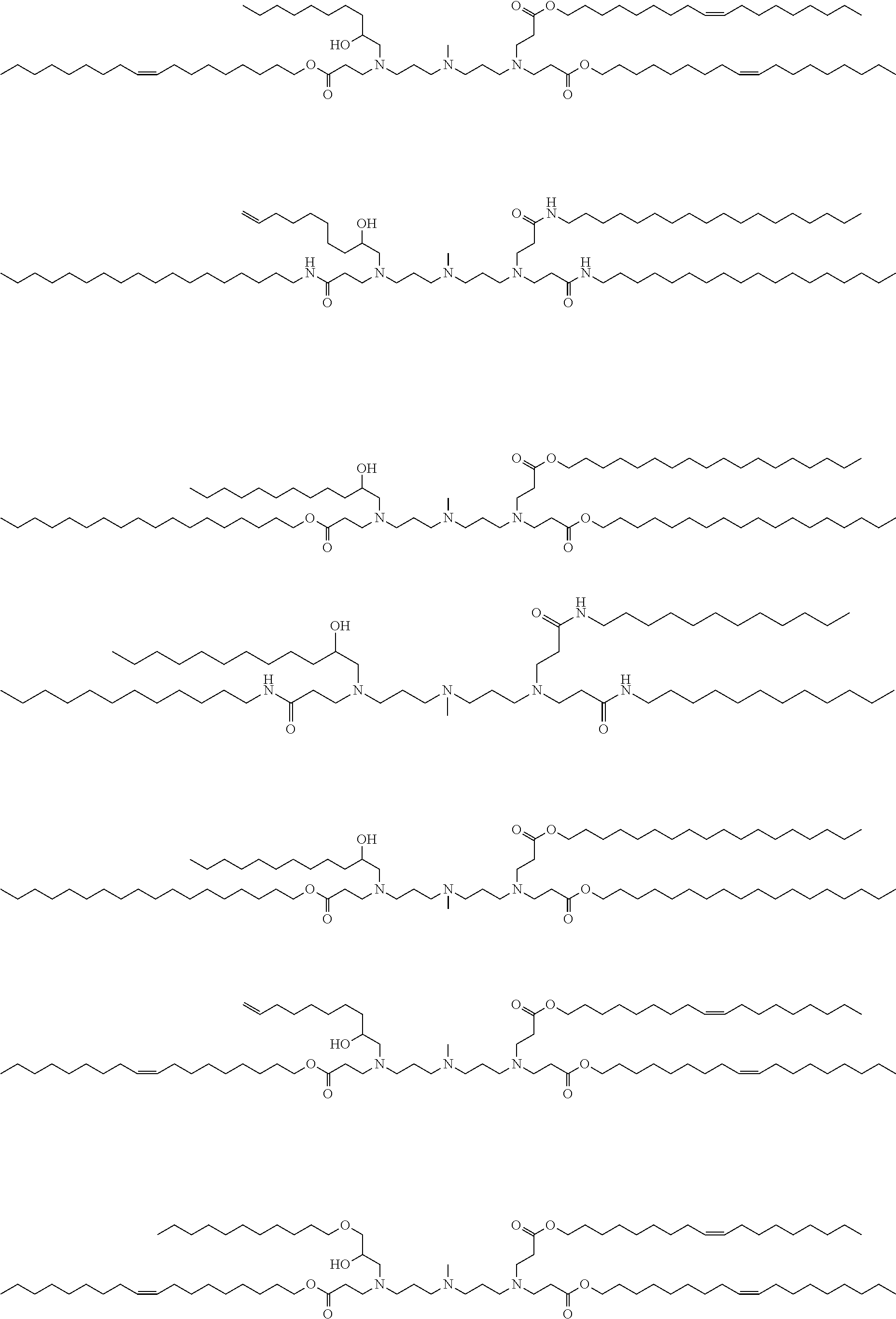

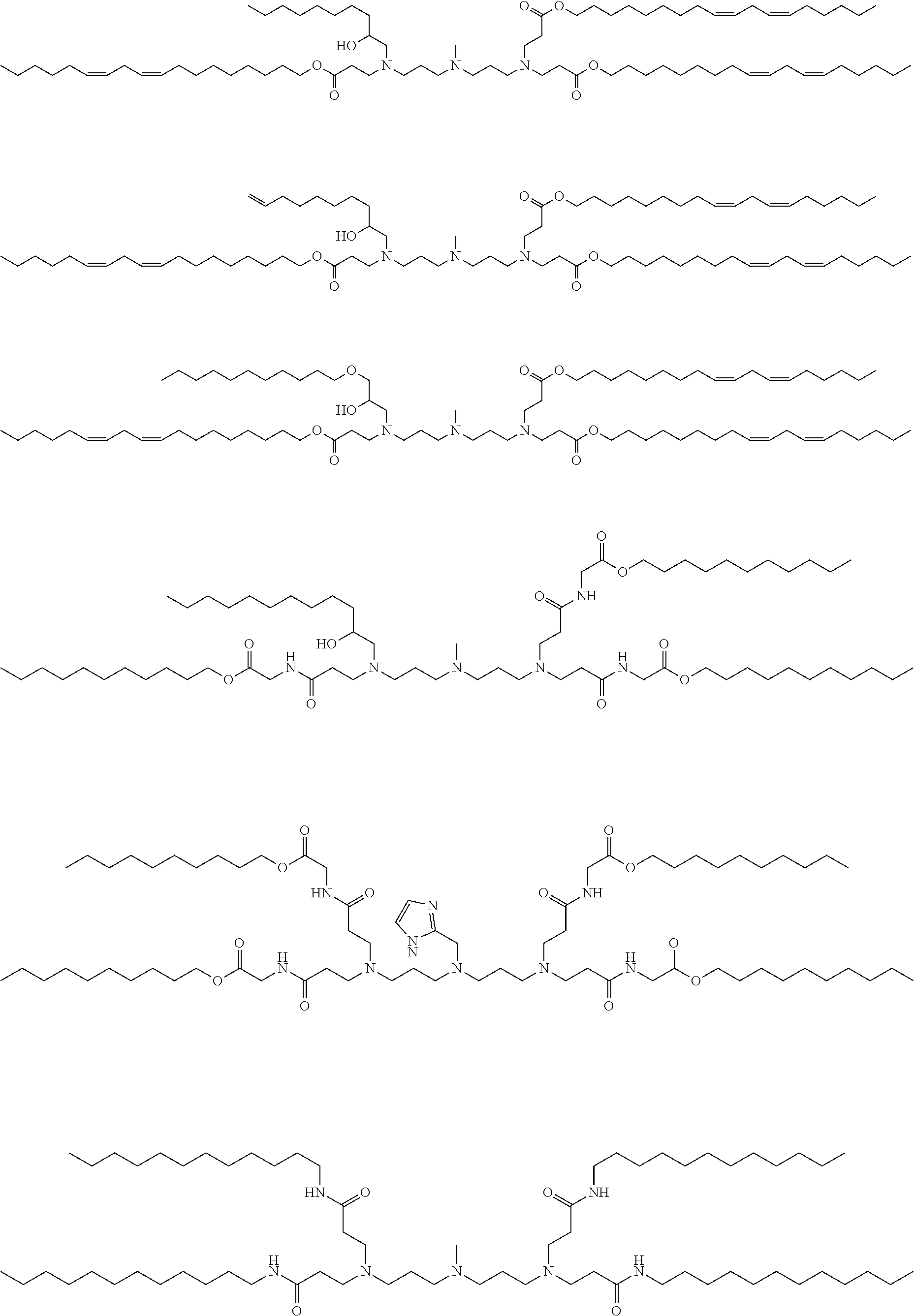

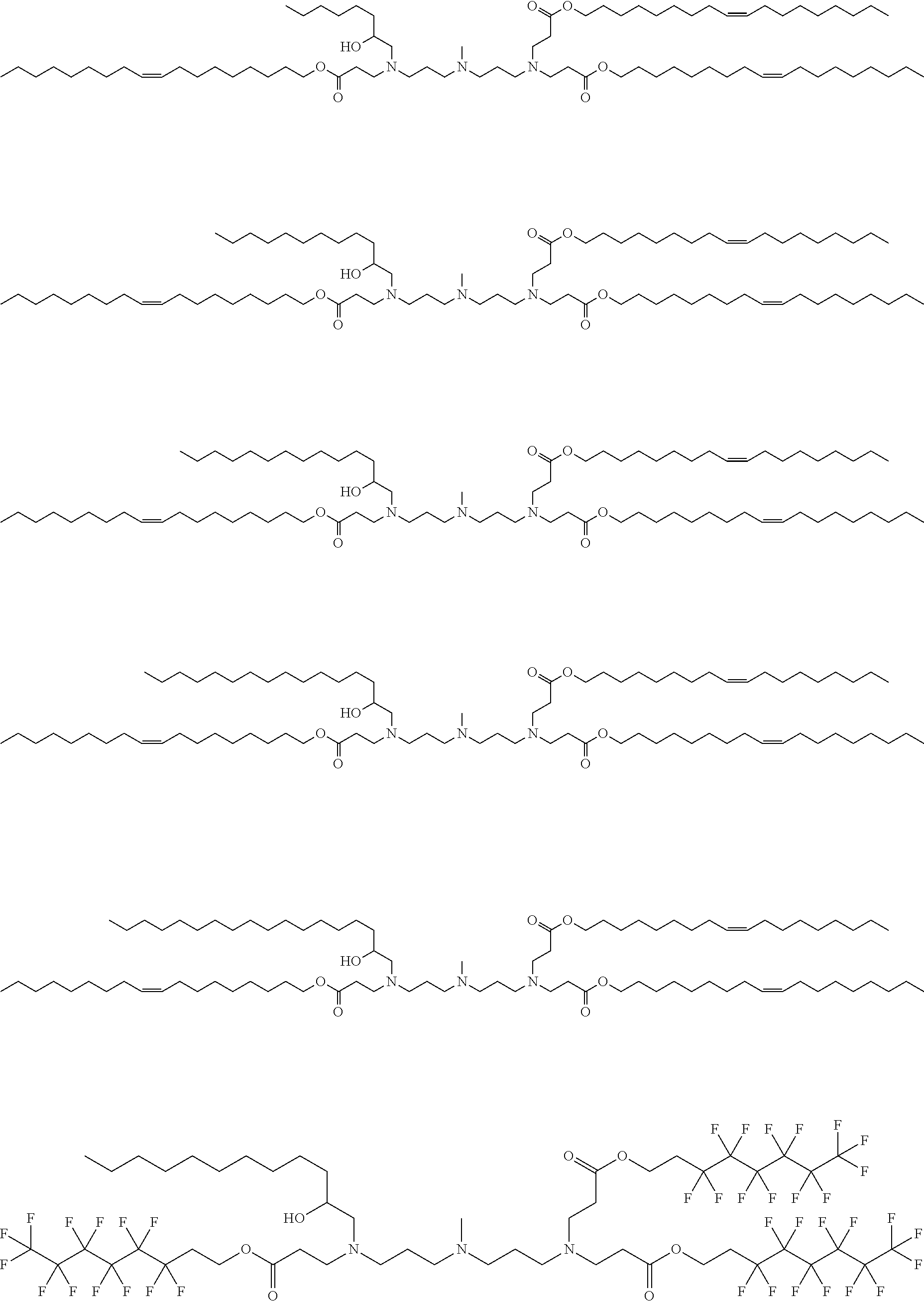

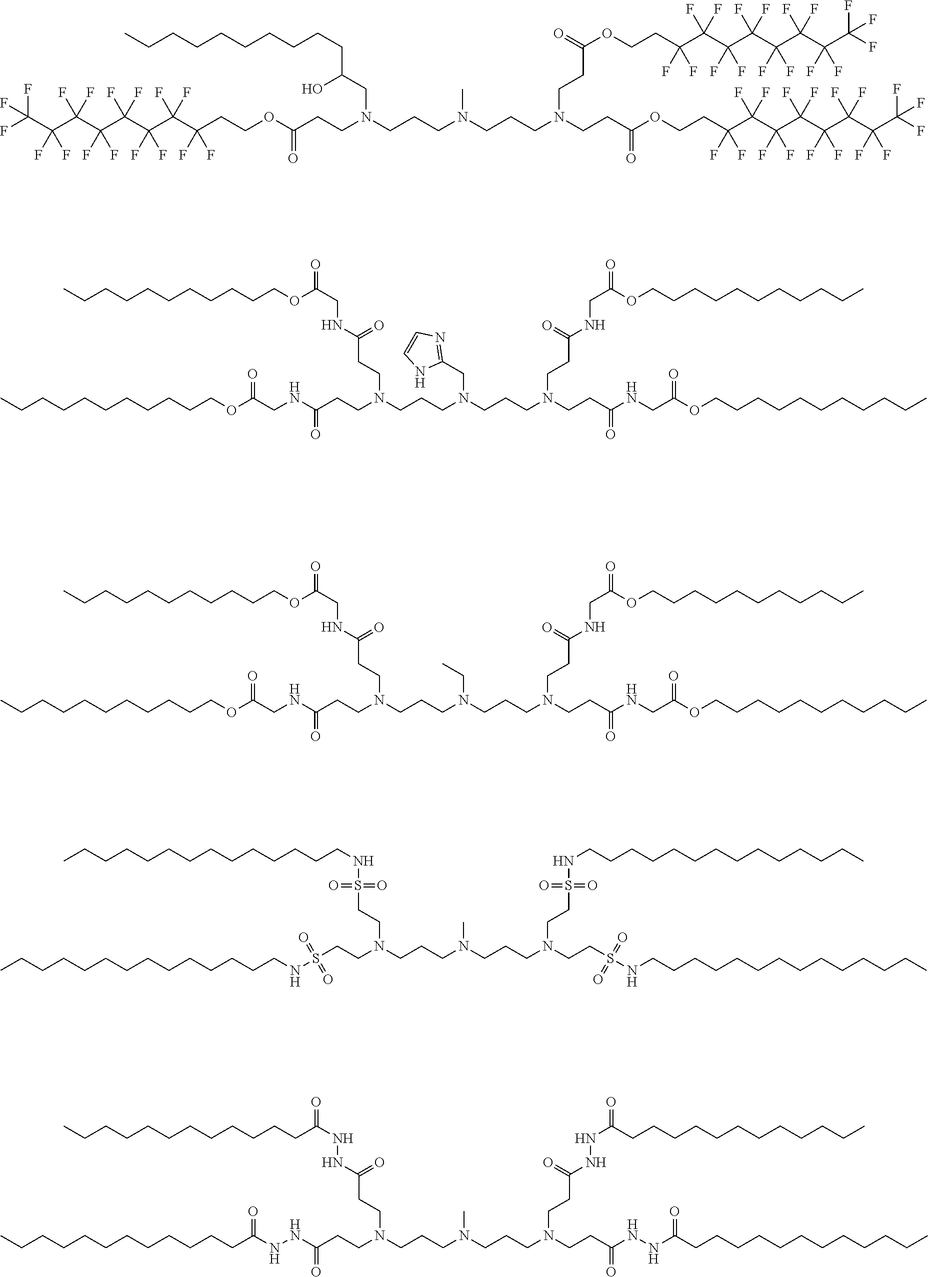

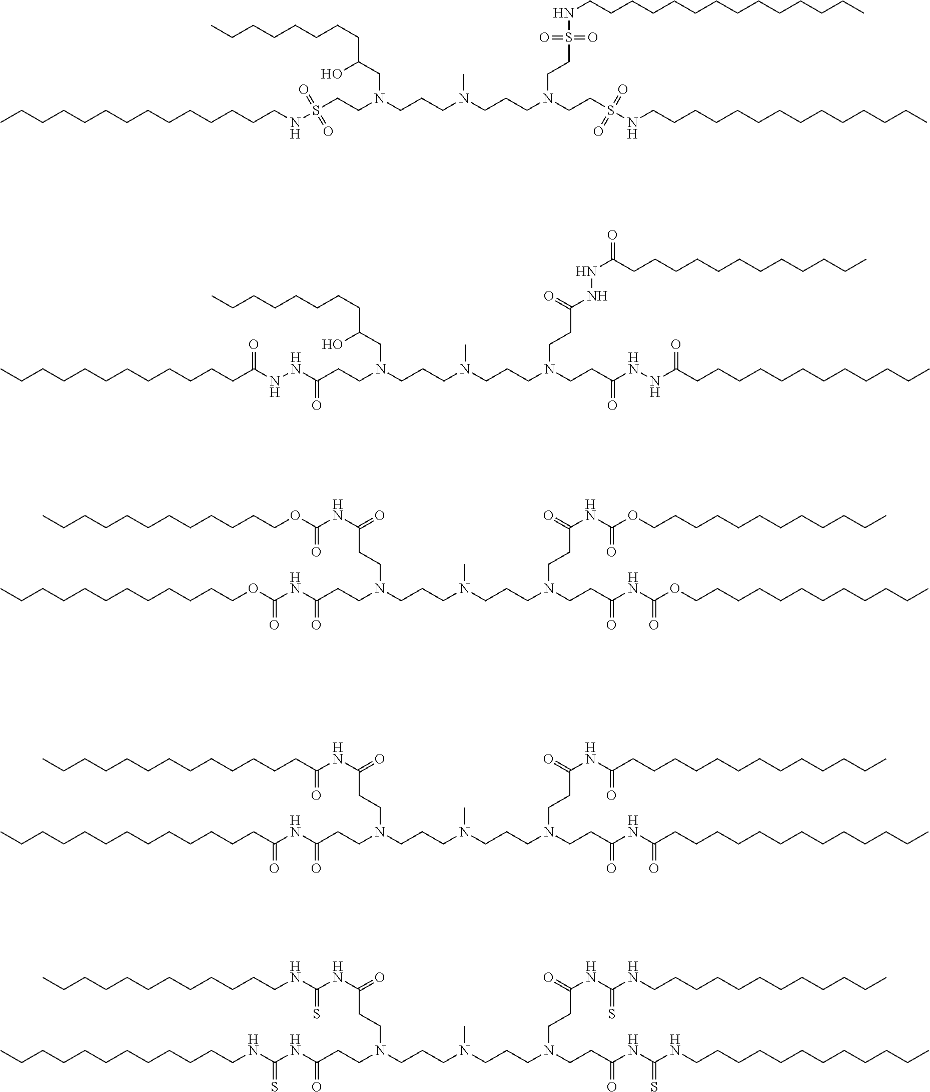

24. The lipid nanoparticle composition of claim 1, wherein the lipid nanoparticle further comprises a neutral lipid, a sterol or a sterol derivative, and a surfactant.

25. The lipid nanoparticle composition of claim 24, wherein
(i) the neutral lipid is selected from the group consisting of 1,2-dioleoyl-sn-glycero-3-phosphoethanolamine (DOPE), 1,2-distearoyl-sn-glycero-3-phosphocholine (DSPC), 1,2-dimyristoyl-sn-glycero-3-phosphocholine (DMPC), 1-palmitoyl-2-oleoyl-glycero-3-phosphocholine (POPC), 1,2-dioleoyl-sn-glycero-3-phosphocholine (DOPC), 1,2-dipalmitoyl-sn-glycero-3-phosphocholine (DPPC), 1,2-Distearoyl-sn-glycero-3-phosphorylethanolamine (DSPE), 1,2-dilauroyl-sn-glycero-3-phosphocholine (DLPC), 1,2-diundecanoyl-sn-glycero-phosphocholine (DUPC), 1,2-dioleoyl-sn-glycero-3-phospho-rac-(1-glycerol) sodium salt (DOPG), 1,2-di-O-octadecenyl-5-glycero-3-phosphocholine (18:0 Diether PC), 1-hexadecyl-sn-glycero-3-phosphocholine (C16 Lyso PC), 1-oleoyl-2-cholesterylhemisuccinoyl-5-glycero-3-phosphocholine (OChemsPC), and any mixtures thereof;
(ii) the sterol is cholesterol, 0-sitosterol, stigmasterol, ergosterol, brassicasterol, fecosterol or campesterol; and/or
(iii) the surfactant is selected from the group consisting of 1,2-dimyristoyl-rac-glycero-3-methoxypolyethylene glycol (DMG-PEG), 1,2-distearoyl-rac-glycero-3-methoxypolyethylene glycol (DSG-PEG), N-(methylpolyoxyethylene-carbonyl)-1,2-distearoyl-sn-glycero-3-phosphoethanolamine (PEG-DSPE), 1,2-dioleoyl-sn-glycero-3-phosphoethanolamine-N-[(polyethylene glycol)](DOPE-PEG), and any mixtures thereof.

26. The lipid nanoparticle composition of claim 25, wherein the neutral lipid is 1,2-distearoyl-sn-glycero-3-phosphocholine (DSPC), the sterol is cholesterol, and the surfactant is DMG-PEG.

27. The lipid nanoparticle composition of claim 24, wherein the lipid nanoparticle comprises a molar ratio of:
(i) 10-65% of the compound of Formula (I);
(ii) 5-30% of the neutral lipid;
(iii) 15-50% of the sterol or the sterol derivative; and
(iv) 0.5-5% of the surfactant,
based on the total amount of the lipid nanoparticle.

28. The lipid nanoparticle composition of claim 24, wherein the lipid nanoparticle comprises a molar ratio of:
(i) 40-65% of the compound of Formula (I);
(ii) 5-15% of the neutral lipid;
(iii) 25-50% of the sterol or the sterol derivative; and
(iv) 1-5% of the surfactant,
based on the total amount of the lipid nanoparticle.

29. The lipid nanoparticle composition of claim 24, wherein the lipid nanoparticle comprises a molar ratio of:
(i) 40-65% of the compound of Formula (I);
(ii) 5-15% of DSPC;
(iii) 25-50% of cholesterol; and
(iv) 1-5% of DMG-PEG,
based on the total amount of the lipid nanoparticle.

30. The lipid nanoparticle composition of claim 24, wherein the lipid nanoparticle comprises a molar ratio of:
(i) 40-50% of the compound of Formula (I);
(ii) 5-15% of DSPC;
(iii) 25-50% of cholesterol; and
(iv) 1.5-2.55% of DMG-PEG,
based on the total amount of the lipid nanoparticle.

31. The lipid nanoparticle composition of claim 1, wherein the nucleic acid encodes the Respiratory Syncytial Virus antigenic polypeptide comprising a) an amino acid sequence of amino acid residues 26-574 of SEQ ID NO: 38, or b) a variant of (a) having at least one amino acid substitution selected from the group consisting of S55C, P102A, T103C, R106K, R109Q, R133Q, K134Q, R135Q, R136Q, L142Q, I148C, A149C, S155C, L188C, S190F, V207L, K209P, Q210P, Q210C, S211P, C212P, S290C, I379V, M447V, Y458C, L481P, V482P, Q501G and L512K according to SEQ ID NO: 38, wherein the variant has the Respiratory Syncytial Virus antigenic activity.

32. The lipid nanoparticle composition of claim 1, wherein the nucleic acid encodes the Respiratory Syncytial Virus antigenic polypeptide comprising an amino acid sequence selected from the group consisting of SEQ ID NOs: 38-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183 and 287-370, or an amino acid sequence having at least 95% sequence identity to an amino acid sequence selected from the group consisting of SEQ ID NOs: 38-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183 and 287-370.

33. The lipid nanoparticle composition of claim 1, wherein the nucleic acid comprises a nucleic acid sequence selected from the group consisting of SEQ ID NOs: 1-33, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 182, 174, 176, 178, 180, 182 and 202-286.

34. The lipid nanoparticle composition of claim 1, wherein the target polynucleotide comprises a nucleic acid of SEQ ID NO: 2, 4, 14, 16, 18, 24, 26, 30, 31, 32, 33, 203, 205, 215, 217, 219, 225, 227, 231, 232, 233 or 234, or a nucleic acid sequence at least 85% identical to 2, 4, 14, 16, 18, 24, 26, 30, 31, 32, 33, 203, 205, 215, 217, 219, 225, 227, 231, 232, 233 or 234 which encodes a polypeptide having Respiratory Syncytial Virus antigenic activity.

35. The lipid nanoparticle composition of claim 1, wherein the target polynucleotide comprises:
(i) a 5′ untranslated region (UTR) comprising a nucleic acid selected from the group consisting of SEQ ID NOs: 34, 37 and 70-71; and/or
(ii) a 3′ untranslated region (UTR) comprising a nucleic acid of selected from the group consisting of SEQ ID NOs: 35 and 72-74; and/or
(iii) a poly-A region of 50-120 nucleotides in length.

36. A Respiratory Syncytial Virus polypeptide variant, comprising an amino acid sequence of amino acid residues 26-574 of SEQ ID NO: 38 with at least one amino acid substitution to the amino acid sequence of amino acid residues 26-574 of SEQ ID NO: 38, wherein the at least one amino acid substitution is selected from the group consisting of S55C, P102A, T103C, R106K, R109Q, R133Q, K134Q, R135Q, R136Q, L142Q, I148C, A149C, S155C, L188C, S190F, V207L, K209P, Q210P, Q210C, S211P, C212P, S290C, I379V, M447V, Y458C, L481P, V482P, Q501G and L512K according to SEQ ID NO: 38, wherein the variant has the Respiratory Syncytial Virus antigenic activity.

37. The Respiratory Syncytial Virus polypeptide variant of claim 36, comprising at least two amino acid substitutions selected from the group consisting of S55C, P102A, T103C, R106K, R109Q, R133Q, K134Q, R135Q, R136Q, L142Q, I148C, A149C, S155C, L188C, S190F, V207L, K209P, Q210P, Q210C, S211P, C212P, S290C, I379V, M447V, Y458C, L481P, V482P, Q501G and L512K according to SEQ ID NO: 38, wherein the variant has the Respiratory Syncytial Virus antigenic activity.

38. The Respiratory Syncytial Virus polypeptide variant of claim 36, comprising amino acid substitutions selected from the group consisting of:
i) K209P and Q210P;
ii) Q210P and S211P;
iii) S211P and C212P;
iv) L481P;
v) L481P and V482P;
vi) K209P, Q210P, L481P and V482P;
vii) Q210P, S211P, L481P and V482P;
viii) S211P, C212P, L481P and V482P;
ix) K209P, Q210P and L481P;
x) Q210P, S211P and L481P;
xi) S211P, C212P and L481P;
xii) S55C, P102A, T103C, I148C, L188C, V207L, Q210P, I379V and M447V;
xiii) S55C, P102A, T103C, I148C, L188C, Q210P, I379V and M447V;
xiv) P102A, T103C, I148C, V207L, Q210P, I379V and M447V;
xv) S55C, P102A, L188C, V207L, Q210P, I379V and M447V;
xvi) P102A, T103C, I148C, Q210P, I379V and M447V;
xvii) S55C, P102A, L188C, Q210P, I379V and M447V;
xviii) P102A, V207L, Q210P, I379V and M447V;
xix) V207L and Q210P;
xx) S55C, P102A, T103C, I148C, L188C, V207L, Q210C, S213C, I379V and M447V;
xxi) S55C, P102A, T103C, I148C, L188C, Q210C, S213C, I379V and M447V;
xxii) P102A, T103C, I148C, V207L, Q210C, S213C, I379V and M447V;
xxiii) S55C, P102A, L188C, V207L, Q210C, S213C, I379V and M447V;
xxiv) P102A, T103C, I148C, Q210C, S213C, I379V and M447V;
xxv) S55C, P102A, L188C, Q210C, S213C, I379V and M447V;
xxvi) P102A, V207L, Q210C, S213C, I379V and M447V;
xxvii) V207L, Q210C and S213C;
xxviii) S55C, P102A, L188C, I379V and M447V;
xxix) P102A, T103C, I148C, I379V and M447V;
xxx) P102A, I379V and M447V;
xxxi) P102A and M447V;
xxxii) E30V, P102A, I379V and M447V;
xxxiii) T54H, P102A, I379V and M447V;
xxxiv) N88S, P102A, I379V and M447V;
xxxv) P102A, T103A, I379V and M447V;
xxxvi) P102A, A122T, I379V and M447V;
xxxvii) P102A, K124N, I379V and M447V;
xxxviii) P102A, T125N, I379V and M447V;
xxxix) P102A, R136K, I379V and M447V;
xxxx) P102A, V152I, I379V and M447V;
xxxxi) P102A, S190I, 1379V and M447V;
xxxxii) P102A, N227S, I379V and M447V;
xxxxiii) P102A, V296, I379V I and M447V;
xxxxiv) P102A, Q354L, I379V and M447V;
xxxxv) P102A, L373R, I379V and M447V;
xxxxvi) P102A, I379V, M447V and D486S;
xxxxvii) P102A, I379V, M447V and S540L;
xxxxviii) P102A, I379V, M447V and L547F;
xxxxix) S155C, P102A, S290C, I379V, and M447V;
xxxxx) P102A, A149C, I379V, M447V and Y458C;
xxxxxi) a 27-amino acid residues fragment from E110 to R136 was substituted by residues -GS-, and a 50-amino acid residues fragment from I525 to N574 was substituted by residues GYIPEAPRDGQAYVRKDGEWVLLSTFL;
xxxxxii) a 27-amino acid residues fragment from E110 to R136 was substituted by residues -GS-, and a deletion of a 24-amino acid residues fragment from K551 to N574;
xxxxxiii) a deletion of a 18-amino acid residues fragment from F137 to V154, and a 50-amino acid residues fragment from I525 to N574 was substituted by residues GYIPEAPRDGQAYVRKDGEWVLLSTFL;
xxxxxiv) a deletion of a 18-amino acid residues fragment from F137 to V154, and a deletion of a 50-amino acid residues fragment from I525 to N574;
xxxxxv) a deletion of a 18-amino acid residues fragment from F137 to V154;
xxxxxvi) a 45-amino acid residues fragment from E110 to V154 was substituted by residues -GS-, and a 50-amino acid residues fragment from I525 to N574 was substituted by residues GYIPEAPRDGQAYVRKDGEWVLLSTFL;
xxxxxvii) a 45-amino acid residues fragment from E110 to V154 was substituted by residues -GS-, and a deletion of a 24-amino acid residues fragment from K551 to N574; and
xxxxxviii) a 45-amino acid residues fragment from E110 to V154 was substituted by residues -GS-.

39. The Respiratory Syncytial Virus polypeptide variant of claim 36, comprising an amino acid sequence selected from the group consisting of SEQ ID NOs: 39-69, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183 and 287-370.

40. An isolated nucleic acid sequence encoding a Respiratory Syncytial Virus polypeptide comprising an amino acid sequence of amino acids of 26-574 of SEQ ID NO: 38, or the Respiratory Syncytial Virus polypeptide variant of claim 36.

41. The isolated nucleic acid sequence of claim 40, wherein the isolated nucleic acid comprises a nucleic acid sequence selected from the group consisting of SEQ ID NOs: 1-33, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 182, 174, 176, 178, 180, 182 and 202-286.

42. A Respiratory Syncytial Virus vaccine, comprising the lipid nanoparticle composition of claim 1.

43. A method of inducing an antigen specific immune response in a subject, comprising administering to the subject the Respiratory Syncytial Virus vaccine of claim 42 in an amount effective to produce an antigen specific immune response.

44. The method of claim 43, wherein the method of inducing an antigen specific immune response involves a single administration of the Respiratory Syncytial Virus vaccine.

45. The method of claim 43, further comprising administering a booster dose of the Respiratory Syncytial Virus vaccine.

46. A Respiratory Syncytial Virus vaccine, comprising the Respiratory Syncytial Virus polypeptide variant of claim 36.

47. A Respiratory Syncytial Virus vaccine, comprising the the isolated nucleic acid sequence of claim 40.
